(19) United States
(12) Patent Application Publication
(10) Pub. No.: US 2015/0038358 A1
(43) Pub. Date: Feb. 5, 2015

(54) METHODS FOR DETECTING TRISOMY 21

(71) Applicant: UNIVERSITY OF KANSAS, Lawrence, KS (US)

(72) Inventors: Carl Philip Weiner, Kansas City, KS (US); Yafeng Dong, Kansas City, KS (US)

(21) Appl. No.: 14/361,895

(22) PCT Filed: Nov. 30, 2012
(86) PCT No.: PCT/US2012/067328

§ 371 Date: May 30, 2014

Related U.S. Application Data
(60) Provisional application No. 61/565,761, filed on Dec. 1, 2011.

(51) Int. Cl. C12Q 1/68

(52) U.S. Cl. CPC C12Q 1/6883; C12Q 2600/16; C12Q 2600/158; C12Q 2600/156
USPC 506/9

(57) ABSTRACT

Methods for determining whether a subject, such as a fetus, has Down syndrome are described. In one embodiment, the methods include detecting one or more biomarkers in a biological sample, and determining whether the expression of the biomarkers is altered when compared to expression of the biomarkers in one or more subjects that do not have trisomy 21. In one embodiment, the biological sample is a blood sample, and the biomarkers are cell free plasma RNAs.

### CROSS-REFERENCE TO RELATED APPLICATIONS

This application claims the benefit of U.S. Provisional Application Ser. No. 61/565,761 filed Dec. 1, 2011, which is incorporated by reference herein.

### SEQUENCE LISTING

This application contains a Sequence Listing which has been submitted in ASCII format via EFS-Web and is hereby incorporated by reference in its entirety. The ASCII copy, created on Nov. 29, 2012, is named 42020201.txt and is 768,465 bytes in size.

### BACKGROUND

Trisomy 21, also referred to as Down Syndrome and mongolism, is the result of a chromosomal abnormality. A human cell has two types of chromosomes. One type is the autosomal chromosomes (chromosomes 1-22), and the other type is the sex chromosome (the X and Y chromosomes). In a normal human cell there are 46 chromosomes, and they are present in the cell as 23 pairs. Thus each normal human cell has two of each autosomal chromosome (two copies of chromosome 1, two copies of chromosome 2, etc.) and one pair of sex chromosomes (an X and a Y chromosome for a male, or two X chromosomes for a female). A karyotype of a normal male is referred to as 46XY, and that of a normal female is 46XX. The chromosomal abnormality in a person having trisomy 21 is an extra chromosome 21. The karyotype of a male having trisomy 21 is 47XY+21, and the karyotype of a female having trisomy 21 is 47XX+21.

Trisomy 21 is typically caused by a meiotic nondisjunction event. With nondisjunction, a gamete (either a sperm or egg cell) is produced with an extra copy of the chromosome 21; thus, the gamete has 24 and not the normal 23 chromosomes. When combined with a normal gamete from the other parent, the resulting embryo has 47 chromosomes, with three copies of chromosome 21. An analogous process accounts for most cases of trisomy 13 and 18. Trisomy 21 is the cause of approximately 95% of Down syndromes, with 88% resulting from nondisjunction in the maternal gamete and 8% from nondisjunction in the paternal gamete. The actual Down syndrome “critical region” encompasses chromosome bands 21q22.1-q22.3.

Since trisomy 21 is usually caused by nondisjunction in the gametes prior to conception, all cells in the resulting conceptus are affected. However, when some of the cells in the body are normal and other cells have trisomy 21, it is called mosaic Down syndrome (46,XX/47,XX,+21). This can occur in one of main two ways: in the first, a nondisjunctional event in a normal embryo during an early cell division results in a fraction of the cells having trisomy 21; in the second, a Down syndrome embryo secondary to nondisjunction loses the extra 21 chromosome with the result of a normal cell line. There is considerable variability in the fraction of trisomy 21 cells among mosaic trisomy 21, both as a whole and among tissues. Mosaicism causes 1-2% of Down syndrome.

Down syndrome is the result of a random event during the formation of sex cells or pregnancy. There is no evidence that it is due to parental behavior (other than age) or environmental factors. In 2006, the Centers for Disease Control and Prevention estimated the overall rate for Down syndrome in the United States was one per 733 live births (5429 new cases per year). Approximately 95% of these are trisomy 21. Down syndrome occurs in all ethnic groups and among all economic classes.

Maternal age affects the probability of conceiving a fetus with Down syndrome. At maternal age 20 to 24, the probability is one in 1562; at age 35 to 39 the probability is one in 214, and above age 45 the probability is one in 19 (Huether et al., 1998, J Med Genet 35 (6): 482-90. doi:10.1136/jmg.35.6.482. PMID 9643290). Although the probability increases with advancing maternal age, 80% of children with Down syndrome are born to women under the age of 35, reflecting the overall fecundity of that age group. Recent information suggests paternal age, especially beyond 42 years increases the risk of Down syndrome too.

A diagnostic test for Down syndrome is typically preceded by some form of screening test (history, ultrasound or blood protein measurement) as the current methods for diagnosis involves an invasive test-amniocentesis, chorionic villus sampling or percutaneous umbilical cord blood sampling (PUBS)—that is then offered to families who are screen positive, i.e.

have an increased likelihood of having a fetus with Down syndrome. In the United States, the American College of Obstetrics and Gynecology guidelines recommend non-invasive screening and invasive testing be offered to all women, regardless of their age, and most likely all physicians follow these guidelines. This recommendation is supported by some insurance plans that only reimburse invasive testing if the woman is >34 years old or if she has received a high-risk score from a non-invasive screening test Amniocentesis, CVS and PUBS are invasive procedures that involve inserting instruments into the uterus, and therefore carry a risk of causing fetal injury or pregnancy loss. The risks of a loss from CVS and amniocentesis are often quoted as 1% and 0.5% respectively. In all likelihood, the fetuses lost as a result are otherwise normal.

There are several common non-invasive screening tests that can identify a fetus at high risk for Down syndrome. These tests are typically performed in the late 1^{st} or early 2^{nd} trimester. Due to the nature of screening, each test has a significant chance of a false positive that is suggesting the fetus has Down syndrome when in fact, the fetus does not. Screen positive tests must be verified by an invasive procedure before a diagnosis of Down syndrome is made. Common screening procedures for Down syndrome are given in Table 1.

TABLE 1
First and Second trimester Down syndrome screens

| | Weeks | | False | |
|---|---|---|---|---|
| | gestation | Detection | positive | |
| Screen | performed | rate | rate | Description |
| Quad screen | 15-20 | 81% | 5% | Measures the maternal |
| | | | | serum α feto protein, |
| | | | | estriol, human chorionic |
| | | | | gonadotropin (hCG) |
| | | | | and inhibin-α. |
| Nuchal | 10-13.5 | 85% | 5% | Uses ultrasound to |
| translucency/ | | | | measure Nuchal |
| free β/ | | | | Translucency (NT) plus |
| PAPP-A | | | | free β hCG and PAPP-A. |
| (1^{st} Trimester | | | | |
| Combined | | | | |
| Test) | | | | |
| Integrated | 10-13.5 | 95% | 5% | The Integrated test uses |
| Test | and 15-20 | | | both the 1^{st} Trimester |
| | | | | Combined test and the |
| | | | | 2^{nd} Trimester Quad test |
| | | | | to yield a more accurate |
| | | | | screening result. |

Even with the best available non-invasive screening tests, the detection rate is 90-95% with a screen positive rate set at 2.5%. Screening tests more commonly used have screen positive rates of 5-7.5%. Inaccuracies may result from undetected multiple fetuses (rare with adequate ultrasound testing), incorrect dating of the pregnancy, normal variation in the ultrasound measurements or maternal protein levels that constitute the screen, or operator error. These ultrasound measurements and protein levels overlap in the healthy and disease groups broadly in all instances.

Confirmation of screen positive status requires a fetal karyotype that is normally accomplished by amniocentesis or chorionic villus sampling (CVS). Amniocentesis involves taking amniotic fluid from the amniotic sac, and CVS a placental biopsy, each to obtain fetal cells. The laboratory work can take several weeks but will detect over 99.8% of all numerical chromosomal problems with a very low false positive rate; however, these methods of confirmation have the risk of miscarriage of a healthy fetus.

The focus of noninvasive diagnosis of Down syndrome has been on the detection and separation of fetal DNA found in the mother's peripheral blood derived from chromosome 21. A few investigators have tried to use RNA based technologies with mixed success.

### SUMMARY OF THE INVENTION

Provided herein are methods for determining whether a subject has trisomy 21. In one embodiment, a method may include screening a fetus for trisomy 21. The method may include measuring a plurality of trisomy 21 biomarkers in a biological sample obtained from a first pregnant female, wherein the plurality of trisomy 21 biomarkers is chosen from any combination of SEQ ID NO:8-3,248, or a complement thereof. In one embodiment, the T21 biomarkers may be chosen from a sequence of at least 15 consecutive nucleotides selected from any combination of SEQ ID NO:8-3,248, or a complement thereof. In one embodiment, the fetus of the first pregnant female is between 8 weeks and 16 weeks post-implantation.

The method may also include identifying the fetus as having trisomy 21 if expression of the plurality of biomarkers is altered to a statistically significant degree in the biological sample compared to a biological sample from a second pregnant female carrying a fetus not having trisomy 21. The method may also include identifying the fetus as not having trisomy 21 if expression of the plurality of biomarkers is not altered to a statistically significant degree in the biological sample compared to a biological sample from a second pregnant female carrying a fetus not having trisomy 21. In one embodiment, expression of a trisomy 21 biomarker is altered to a statistically significant degree if it is outside the 95% confidence interval for that trisomy 21 biomarker. In one embodiment, the method may further include recommending a genetic test chosen from amniocentesis, cordocentesis, and chorionic villus sampling, ultrasound, or a combination thereof.

In one embodiment, the plurality of trisomy 21 biomarkers may include at least 10 trisomy 21 biomarkers, wherein the pregnant mother having at least 6 biomarkers altered to a statistically significant degree is identified as carrying a fetus having trisomy 21. In one embodiment, the plurality of trisomy 21 biomarkers includes at least 10 trisomy 21 biomarkers, and wherein the pregnant mother having no greater than 4 biomarkers altered to a statistically significant degree is identified as carrying a fetus not having trisomy 21. In one embodiment, the first pregnant female and the second pregnant female may be matched with respect to a co-variable selected from, for instance, maternal age, gestational stage, maternal weight, diet, smoking, prior preterm birth, diabetes, use of prophylactic progesterone, ethnicity, or a combination thereof.

In one embodiment, the trisomy 21 biomarkers may be selected from polynucleotides encoded by chromosome 21, or from polynucleotides encoded by any of chromosomes 1-20, 22 or X. In one embodiment, the trisomy 21 biomarkers may be selected from polynucleotides that are up-regulated in the first pregnant female carrying a fetus with trisomy 21 compared to the second pregnant female carrying a fetus not having trisomy 21. In one embodiment, the trisomy 21 biomarkers may be selected from polynucleotides that are down-regulated in the first pregnant female carrying a fetus with trisomy 21 compared to the second pregnant female carrying a fetus not having trisomy 21.

In one embodiment, the method may further include obtaining the biological sample from the first pregnant female. The obtaining may include obtaining a blood sample. The blood sample may be processed to remove cells from the blood sample. The blood sample may be processed to obtain, and optionally isolate, cell-free plasma RNA. In one embodiment, the method may further include converting RNA polynucleotides present in the biological sample into cDNA molecules, and the measuring includes hybridization between a cDNA molecule and a complementary trisomy 21 biomarker. In one embodiment, the complementary trisomy 21 biomarker is in solution during the hybridization, and in one embodiment, the complementary trisomy 21 biomarker is immobilized on a solid support.

In one embodiment, a method may include detecting trisomy 21 in a fetus. The method may include detecting trisomy 21 biomarkers in a biological sample to yield an expression level of each detected trisomy 21 biomarker. In one embodiment, the biological sample includes plasma from a pregnant female. In one embodiment, the T21 biomarkers may be chosen from a sequence of at least 15 consecutive nucleotides selected from any combination of SEQ ID NO:8-3,248, or a complement thereof. In one embodiment, the fetus of the first pregnant female is between 8 weeks and 16 weeks post-implantation. The method may also include comparing the expression level of each detected trisomy 21 biomarker to the expression level of the trisomy 21 biomarker in pregnant females carrying a fetus without trisomy 21. In one embodiment, an expression level of a detected trisomy 21 biomarker that is outside the 95% confidence interval for that trisomy 21 biomarker indicates the expression level of the trisomy 21 biomarker is altered. In one embodiment, at least 10 trisomy 21 biomarkers are detected. In one embodiment, a fetus carried by the pregnant female is identified as carrying a fetus having trisomy 21 when at least 6 biomarkers are outside the 95% confidence interval. In one embodiment, the method may further include recommending a genetic test chosen from amniocentesis, cordocentesis, and chorionic villus sampling, ultrasound, or a combination thereof. In one embodiment, the pregnant female and the pregnant females used to establish the 95% confidence interval for each trisomy 21 biomarker may be matched with respect to a co-variable selected from, for instance, maternal age, gestational stage, maternal weight, diet, smoking, prior preterm birth, diabetes, use of prophylactic progesterone, ethnicity, or a combination thereof.

In one embodiment, the trisomy 21 biomarkers may be selected from polynucleotides encoded by chromosome 21, or from polynucleotides encoded by any of chromosomes 1-20, 22 or X. In one embodiment, the trisomy 21 biomarkers may be selected from polynucleotides that are up-regulated in the pregnant female carrying a fetus with trisomy 21 compared to the pregnant females carrying a fetus not having trisomy 21. In one embodiment, the trisomy 21 biomarkers may be selected from polynucleotides that are down-regulated in the pregnant female carrying a fetus with trisomy 21 compared to the pregnant females carrying a fetus not having trisomy 21.

In one embodiment, the method may further include obtaining the biological sample from the first pregnant female. The obtaining may include obtaining a blood sample. The blood sample may be processed to remove cells from the blood sample. The blood sample may be processed to obtain, and optionally isolate, cell-free plasma RNA. In one embodiment, the method may further include converting RNA polynucleotides present in the biological sample into cDNA molecules, and the measuring includes hybridization between a cDNA molecule and a complementary trisomy 21 biomarker. In one embodiment, the complementary trisomy 21 biomarker is in solution during the hybridization, and in one embodiment, the complementary trisomy 21 biomarker is immobilized on a solid support.

In one embodiment, a method may include detecting trisomy 21 in a fetus. The method may include detecting trisomy 21 biomarkers in a biological sample from a pregnant female to yield a sample expression profile. In one embodiment, the biological sample includes plasma from a pregnant female. In one embodiment, the T21 biomarkers may be chosen from a sequence of at least 15 consecutive nucleotides selected from any combination of SEQ ID NO:8-3,248, or a complement thereof. In one embodiment, the fetus of the first pregnant female is between 8 weeks and 16 weeks post-implantation. The method may also include comparing the sample expression profile with a reference expression profile, wherein a difference between the sample expression profile and the reference expression profile is indicative of the presence or absence of trisomy 21 in the fetus. In one embodiment, the reference expression profile is from at least one second pregnant female carrying a fetus without trisomy 21, and a difference between the sample expression profile and the reference expression profile is indicative of the presence of trisomy 21. In one embodiment, the reference expression profile is from at least one second pregnant female carrying a fetus with trisomy 21, and a difference between the sample expression profile and the reference expression profile is indicative of the absence of trisomy 21. In one embodiment, the method may further include recommending a genetic test chosen from amniocentesis, cordocentesis, and chorionic villus sampling, ultrasound, or a combination thereof.

In one embodiment, the difference between the sample expression profile and the reference expression profile is statistically significant. In one embodiment, the sample expression profile includes at least 10 trisomy 21 biomarkers. In one embodiment, the trisomy 21 biomarkers may be selected from polynucleotides encoded by chromosome 21, or from polynucleotides encoded by any of chromosomes 1-20, 22 or X. In one embodiment, the trisomy 21 biomarkers may be selected from polynucleotides that are up-regulated in the first pregnant female carrying a fetus with trisomy 21 compared to the second pregnant female carrying a fetus not having trisomy 21. In one embodiment, the trisomy 21 biomarkers may be selected from polynucleotides that are down-regulated in the first pregnant female carrying a fetus with trisomy 21 compared to the second pregnant female carrying a fetus not having trisomy 21. In one embodiment, the first pregnant female and the second pregnant female may be matched with respect to a co-variable selected from, for instance, maternal age, gestational stage, maternal weight, diet, smoking, prior preterm birth, diabetes, use of prophylactic progesterone, ethnicity, or a combination thereof.

In one embodiment, the method may further include obtaining the biological sample from the first pregnant female. The obtaining may include obtaining a blood sample. The blood sample may be processed to remove cells from the blood sample. The blood sample may be processed to obtain, and optionally isolate, cell-free plasma RNA. In one embodiment, the method may further include converting RNA polynucleotides present in the biological sample into cDNA molecules, and the measuring includes hybridization between a cDNA molecule and a complementary trisomy 21 biomarker. In one embodiment, the complementary trisomy 21 biomarker is in solution during the hybridization, and in one embodiment, the complementary trisomy 21 biomarker is immobilized on a solid support.

Also provided herein are articles. In one embodiment, an article includes a substrate and a plurality of different polynucleotides. In one embodiment, the polynucleotides are selected from any combination of SEQ ID NO:8-3,248, or a complement thereof. In one embodiment, the T21 biomarkers are selected from a sequence of at least 15 consecutive nucleotides selected from any combination of SEQ ID NO:8-3,248, or a complement thereof The polynucleotides are immobilized onto a surface of the substrate. In one embodiment, the polynucleotides are immobilized on the substrate surface to form a microarray. In one embodiment, at least 10 polynucleotides are immobilized on the substrate surface.

Also provided herein are kits. In one embodiment, a kit includes an article having a substrate, a plurality of different polynucleotides immobilized onto a surface of the substrate, and packaging materials and instructions for use. In one embodiment, the polynucleotides are selected from any combination of SEQ ID NO:8-3,248, or a complement thereof In one embodiment, the T21 biomarkers are selected from a sequence of at least 15 consecutive nucleotides selected from any combination of SEQ ID NO:8-3,248, or a complement thereof In one embodiment, the polynucleotides are immobilized on the substrate surface to form a microarray.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Examples of normalization sequences. Peptidylprolyl isomerase A (SEQ ID NO:1); snRNA:U6:96Aa (SEQ ID NO: 2); snRNA:U6:96Ab (SEQ ID NO: 3); and snRNA:U6:96Ac (SEQ ID NO: 4).

FIG. 2. Examples of T21 Biomarkers Selection: There are 3,143 mRNA biomarkers (FIGS. 2a) and 98 noncoding small RNA sequences (miRNA plus snoRNA) biomarkers (FIG. 2b) selected from around 25,000 genes using Affymetrix microarray technique.

FIG. 3. Examples of 3,143 T21 mRNA biomarker sequences. Each biomarker includes the following: SEQ ID NO, Chromosome location, Gene Symbol, Gene Accession Number, fold-change, and sequence.

FIG. 4. Examples of 98 T21 noncoding small RNA biomarker sequences (miRNA plus snoRNA). Each biomarker includes the following: SEQ ID NO, Chromosome location, Gene Symbol, Gene Accession Number, fold-change, and sequence.

FIG. 5. Examples of 15 T21 mRNA biomarkers confirmed by Real-time PCR in 10 affected pregnancies. The X-axis is the subject number. The figures represent a graphic illustration of marker expression in trisomy 21 (the squares) compared to the normal range for chromosomally normal fetuses. The dotted lines demarcate the 95% confidence interval for normal.

### DETAILED DESCRIPTION OF ILLUSTRATIVE EMBODIMENTS

Provided herein are polynucleotides useful for determining whether a subject, or a subject's fetus, has trisomy 21 (T21), and methods for using the polynucleotides. The methods described herein, and other embodiments disclosed herein such as reagents and kits, are based in part on the surprising discovery of a plurality of molecular markers, the expression levels of which consistently differentiate between healthy subjects and subjects with T21. The molecular markers are derived from coding regions whose altered expression in an affected subject, as measured from an easily obtained biological sample, is indicative of the subject, or the subject's fetus, having T21. As used herein, the term “polynucleotide” refers to a polymeric form of nucleotides of any length, either ribonucleotides or deoxynucleotides, and includes both double- and single-stranded DNA and RNA. A polynucleotide can be obtained directly from a natural source, or can be prepared with the aid of recombinant, enzymatic, or chemical techniques. A polynucleotide can be linear or circular in topology. The terms cDNA, oligonucleotide, probe, and nucleic acid are included within the definition of polynucleotide and these terms are used interchangeably. The term polynucleotide also includes peptide nucleic acids (Nielsen et al., 1991, Science. 254:1497-500), and other nucleic acid analogs and nucleic acid mimetics (see, e.g., McGall et al., U.S. Pat. No. 6,156,501).

In one embodiment, a method provided herein includes detecting one or more T21 biomarkers in a biological sample. As used herein, a “biological sample” refers to a sample of tissue or fluid obtained from a subject, including but not limited to, for example, whole blood, blood plasma, serum, lymph fluid, synovial fluid, cerebrospinal fluid, urine, and saliva. In one embodiment, a biological sample includes serum. In one embodiment the methods provided herein are directed to non-invasive methods of detecting T21, and in such an embodiment a biological sample may be a fluid. In one embodiment, a biological sample includes blood plasma. In one embodiment, a biological sample includes whole blood. As used herein, “subject” refers to a prenatal or postnatal human. A prenatal human includes a fetus. Unless indicated otherwise, as used herein the term “fetus” refers to a human during prenatal development from the time of first cell division until birth. The fetus may be at any age after implantation. For instance, the fetus may be at 2 weeks post-implantation (PI), 4 weeks PI, 6 weeks PI, 8 weeks PI, 10 weeks PI, 12 weeks PI, 14 weeks PI, 16 weeks PI, 18 weeks PI, 20 weeks PI, etc. In one embodiment, the fetus is between 8 weeks and 16 weeks PI, or between 10 weeks and 14 weeks PI. A postnatal human refers to an individual at any stage of development after birth, including a newborn, a child, an adolescent, or an adult, and includes a pregnant human mother. In one embodiment where the subject is a pregnant human mother, the mother does not have T21. In the embodiment where the subject is a pregnant human mother, a method provided herein allows one to determine if the fetus carried by the pregnant mother has T21.

As used herein, a “T21 biomarker” is a polynucleotide that is indicative of T21 in a subject. A T21 biomarker is indicative of T21 when the expression level or quantity of the biomarker is altered more often in a subject having T21 compared to a healthy subject. A T21 biomarker having an altered expression level or quantity is one that is expressed at a greater level (e.g., over-expressed) or expressed at a lower level (e.g., under-expressed) when compared to a healthy subject. Whether the expression level or quantity of a biomarker in a subject having T21 is altered, e.g., greater than or less than the expression level or quantity of the biomarker in a healthy subject, is determined using routine statistical methods by applying accepted confidence levels, and is described in greater detail herein.

It should be understood that the term “biomarker,” can, depending on the context, refer to the physical polynucleotide itself or to a graphical or numerical representation of the polynucleotide such as an amount of fluorescence present at a spot on a microarray, a band on a gel image, a numerical value, and the like. For example, the amount of fluorescence at a particular spot on a microarray may be referred to as a T21 biomarker. This graphical or numerical biomarker reflects the existence of the underlying expressed polynucleotide in the test sample, which gave rise to an expression level.

In one embodiment, the detecting of one or more T21 biomarkers in a biological sample yields an expression level of each detected biomarker. In one embodiment, the detecting of two or more T21 biomarkers in a biological sample yields a sample expression profile. An “expression level” is any physical representation of the amount of a selected T21 biomarker, as determined from one or more biological samples from a subject. A “sample expression profile” is any physical representation of the amounts of a set of two or more selected T21 biomarkers, as determined from one or more biological samples from a subject. The subject may be one known to have T21, known to have T21 of a particular type (for instance, 47XX+21, 47XY+21, or mosiac), known to be free of T21, or the status of T21 in the subject may be unknown. In one embodiment, a sample expression profile for a subject may include information from a single biological sample that has been analyzed for T21 biomarker expression levels. In one embodiment, a sample expression profile for a subject may include information from multiple types of biological samples that have been analyzed separately for T21 biomarker expression levels.

The terms “normal” and “healthy” are used herein interchangeably to refer to a subject or subjects who do not have a chromosomal abnormality associated with T21. A normal or healthy sample refers to a sample or samples obtained from a normal/healthy subject.

One skilled in the art will appreciate that more than one sample from a subject may be examined. The expression level and/or sample expression profile may be represented in visual graphical form, for example on paper or on a computer display, in a three dimensional form such as an array, and/or stored in a computer-readable medium. An expression level and/or sample expression profile may correspond to a particular status of T21 (e.g., presence or absence of T21) or type (e.g., 47XX+21, 47XY+21, or mosiac), and thus provide a template for comparison to a patient sample. A control expression level and/or a control expression profile, also referred to herein as a reference expression level and a reference expression profile, can be obtained by analyzing a biological sample from at least one healthy subject, or multiple samples obtained from a group of healthy subjects, or from one or more subjects identified as having comparable T21 in terms of type. When multiple samples from a group are used, the levels of expression of each detected T21 biomarker may be an average, consensus, or composite derived from the multiple samples. Similarly, comparable profiles can be obtained for age-matched and/or sex-matched subjects, and comparable profiles can be obtained for pregnant mothers at the same or similar stage of pregnancy. In one embodiment, expression levels and/or expression profiles can be obtained from a pregnant mother, and if the fetus is later determined to be healthy, such expression levels and/or expression profiles can be used as control expression levels and/or control expression profiles.

One skilled in the art will appreciate that multiple nontest factors may alter the marker level measured and may be mathematically adjusted by one of several well known and routine approaches. For example, the median level of each T21 biomarker may be determined at each gestational epoch in control women. If there is a statistically significant change with gestation, regression analysis of median on gestation weighted for the number of samples per epoch may be performed to determine the normal median curve that best fits the data. All results, both affected and unaffected pregnancies, may be expressed as multiples of the gestation-specific median (MoM) based on the fitted curve. In controls, potential co-variables may be examined, including maternal weight, smoking, prior preteen birth, diabetes or use of prophylactic progesterone and ethnicity, to see if they are significantly associated with the MoM. As the sample pool grows, it is likely other variables (such maternal medical diseases) may need to be considered. Plasma levels of fetal-placental derived sequences are specifically likely to decline on average with increased adiposity due to a fixed output being diluted into a greater volume of blood. If any co-variables are confirmed, the levels can be adjusted by, for instance, dividing the observed MoM by the expected median according to the co variable level found in unaffected pregnancies. Typically, the non-parametric Wilcoxon Rank Sum Test is used to select the subset of markers where there is a significant difference in the MoM distribution between affected and control pregnancies. As a large number of potential markers are to be tested, an extreme P-value of 0.005 may be used for an initial selection.

The risk of T21 may be modeled by the a priori risk of the disorder expressed as odds (a:b) multiplied by the likelihood ratio (LR) for the marker profile derived from multivariate Gaussian frequency distributions. All current aneuploidy and pre-eclampsia markers follow an approximately log Gaussian distribution over most of their range for both affected and unaffected pregnancies, and it is expected to be true for the T21 biomarkers disclosed herein. These Gaussian distributions are defined by the marker sequence means and standard deviations after log transformation. For a single marker, the LR is calculated by the ratio of the heights of the two overlapping distributions at the specific level. For extreme results that fall beyond the point where the data fits a Gaussian distribution, it is standard practice to use the LR at the end of the acceptable range. The method is the same for more than one marker except that the heights of multivariate log Gaussian distributions are used. These are defined, in addition to means and standard deviations, by the correlation coefficients between markers within affected and unaffected pregnancies.

The method of numerical integration may be used to model the best combination of markers from the initial subset. This involves division of each marker operating range into up to 100 equal units, calculation of the volumes under the affected and unaffected multivariate Gaussian curves risk as well as the risk in the mid-point of the volume. This determines the distribution of risks in affected and unaffected pregnancies. These distributions will be calculated for all marker combinations and the sensitivity compared for a fixed specificity.

A second approach may be considered based on the well-known fact that a strong association does not guarantee effective discrimination between affected and unaffected. Nor does a high AUC guarantee good prediction of actual risk. Hence, model calibration via reclassification can be useful in order to accept only those markers least likely to have been identified at random. Prognostic models may be built for predictive accuracy after confirmed T21 with only non-T21 biomarker variables (age, race, maternal weight, gestation age, maternal comorbidities, etc) and then build prognostic models to include T21 biomarkers. Dimensionality of the models may be reduced by translating the RNA marker contributions into a few components or composite scores. Principal components analysis may be used to derive the principal components of the T21 biomarkers factors. For instance, leading components that explain more than 85 percent of the total variation in genetic predictors may be retained and included in a prognostic model. Models that are more complicated (more predictors) may appear to have better predictive performance even if that is not the case. Therefore, the model performance may be quantified with respect to calibration and discrimination. Calibration may be examined by comparing the observed with the expected frequencies while the discriminatory accuracy may be assessed using the receiver operating characteristic (ROC) curve estimation for survival data. The true positive fraction or sensitivity and the false positive fraction (1-specificity) may be discussed using the derived prognostic models. For instance, the discriminatory accuracy may be compared between the models with and without validated genetic markers using the area under a ROC curve (AUC). Other validation techniques including cross-validation and bootstrap methods can also be carried to shed some insights about a model's adequacy. Alternatively, prognostic models can be constructed for T21 status (affected or unaffected) using logistic regression models. Modeling procedures may be similar to those previously described for routinely used Cox models.

In one embodiment, a T21 biomarker is RNA. In one embodiment, the RNA that is detected is cell-free, and is referred to herein as cell-free RNA. Cell-free RNA includes coding RNA (mRNA) and non-coding RNAs such as siRNA, miRNA, snoRNA, and snRNA. In one embodiment, cell-free RNA is from whole blood, blood plasma, or serum, and is referred to herein as cell-free plasma (CFP) RNA. CFP RNA includes coding RNA (mRNA) and non-coding RNAs such as, but not limited to, siRNA, miRNA, snoRNA, and snRNA. For instance, when the sample is blood, the CFP RNA to be detected is present in the plasma portion of the blood. Thus, in one embodiment, a biological sample is processed to remove cells prior to the detecting. In one embodiment, a biological sample is processed to minimize cell lysis. In one embodiment, the CFP RNA that is detected may be mRNA, non-coding RNA, or the combination thereof. Optionally, the CFP RNA may be isolated.

RNA may be obtained from a biological sample using routine methods. In one embodiment, RNA is obtained using a process based on a phenol/guanidium isothiocyanate/glycerol phase separation. Such a process may result in large quantities of CFP nucleic acid with total RNA yields of 1.5-30 ug or more from only 2 mL of plasma and full range of RNAs including not only mRNA but also small noncoding RNAs such as miRNA and snoRNA. This amount is more than enough for array technology and the performance of numerous PCR reactions using a clinically practical, single patient sample.

The RNA isolation method described herein allows for the isolation of 1.5 micrograms to 7 micrograms of CFP RNA from a 2 mL sample, which is more than enough for both microaarray gene screening and PCR validation. The method may include obtaining 2 mL or more of sample from a subject, such as plasma, and following the steps as described in Example 1.

The analysis of samples of blood obtained from pregnant mothers who later gave birth to healthy infants or gave birth to infants with T21 has led to the discovery of T21 biomarkers. Examples of T21 biomarkers are described at SEQ ID NO:8-3,248. Different combinations of the T21 biomarkers listed at SEQ ID NO:8-3,248, or the complement thereof, allow the skilled person to predict whether the fetus carried by a pregnant mother has T21. Changes in the expression levels of these biomarker polynucleotides in a subject, as measured in a biological sample from the subject, thus may be used to indicate the presence, absence, or type or T21 in a subject, such as a fetus carried by a mother, or an infant, child, adolescent, or adult.

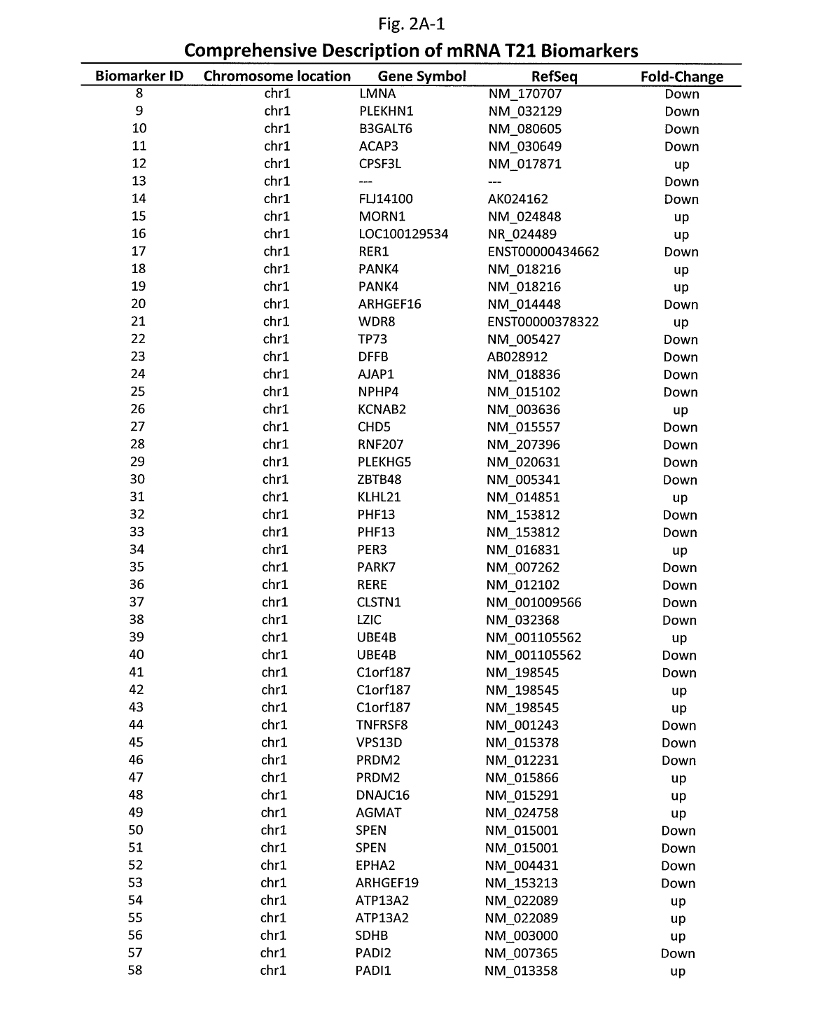

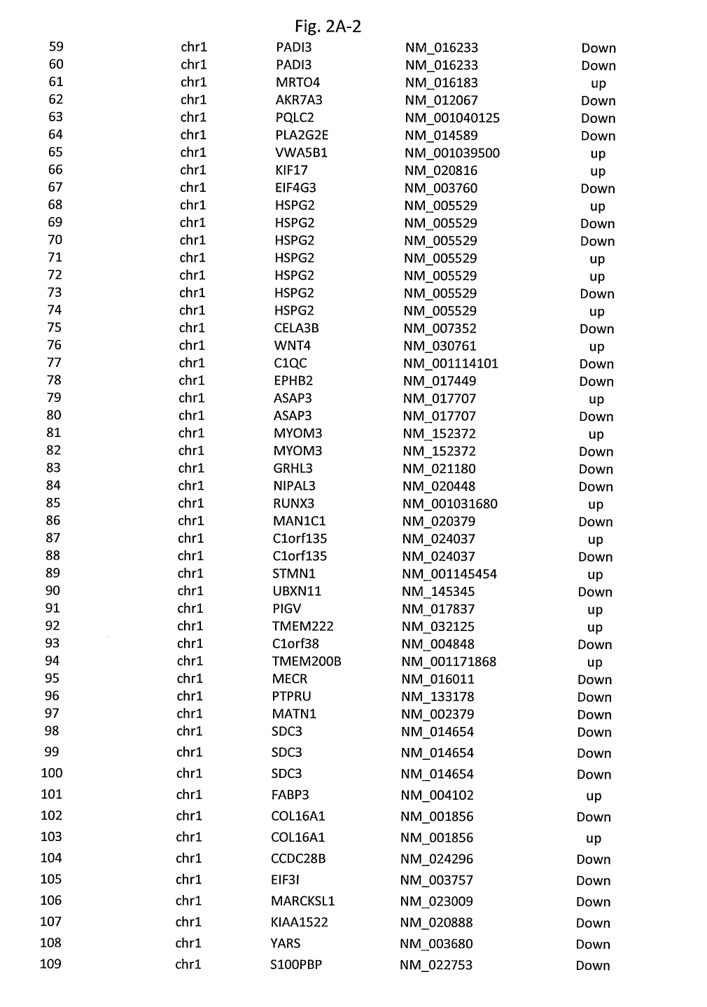

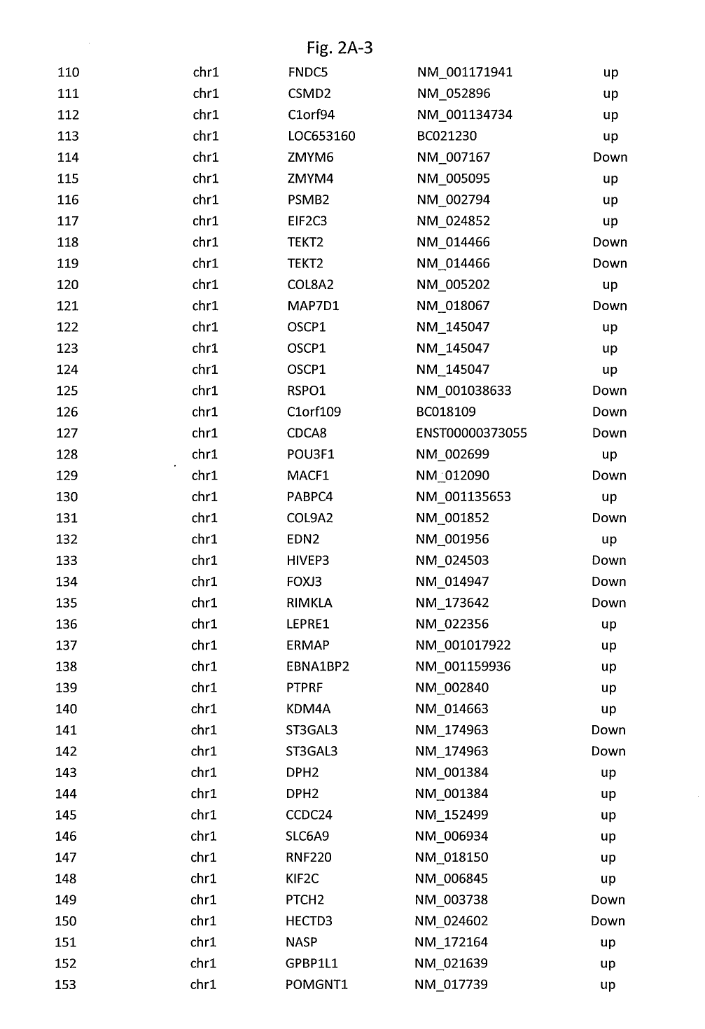

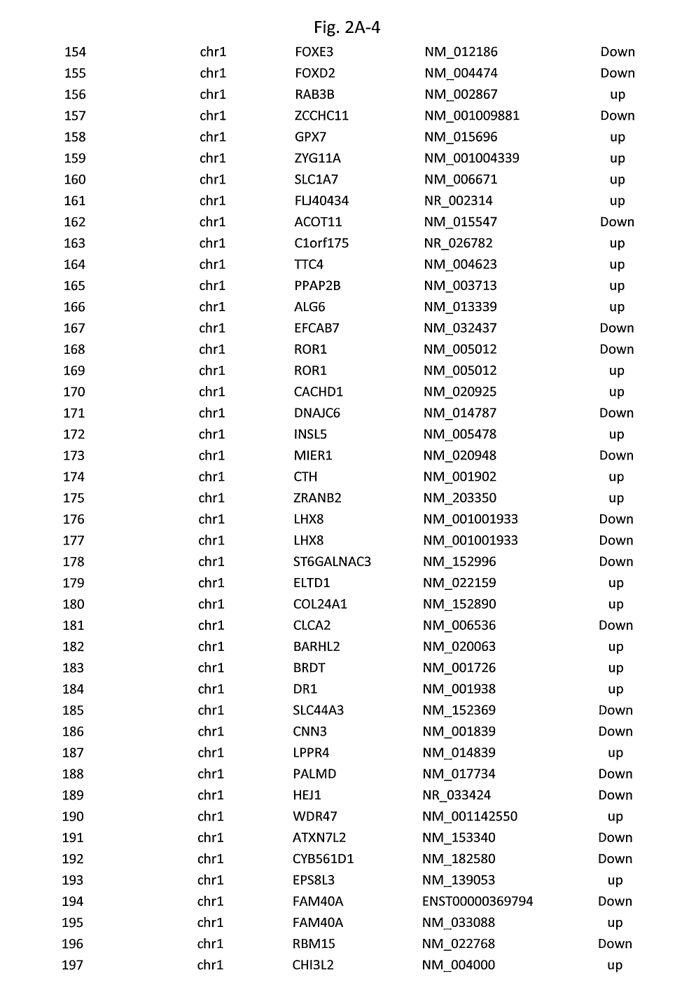

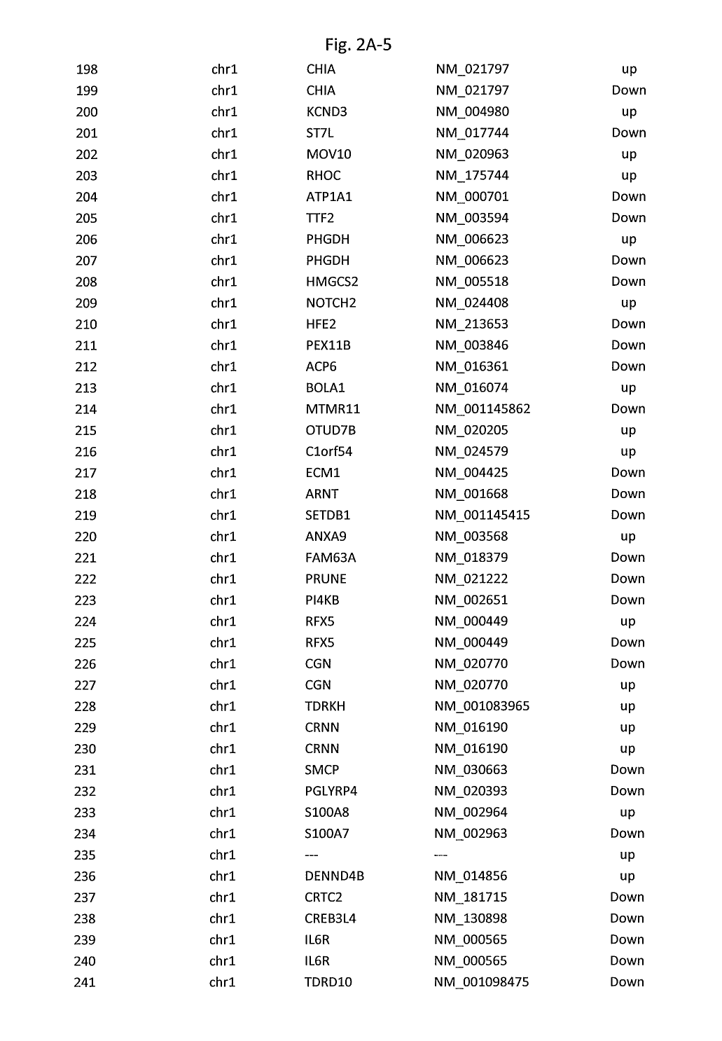

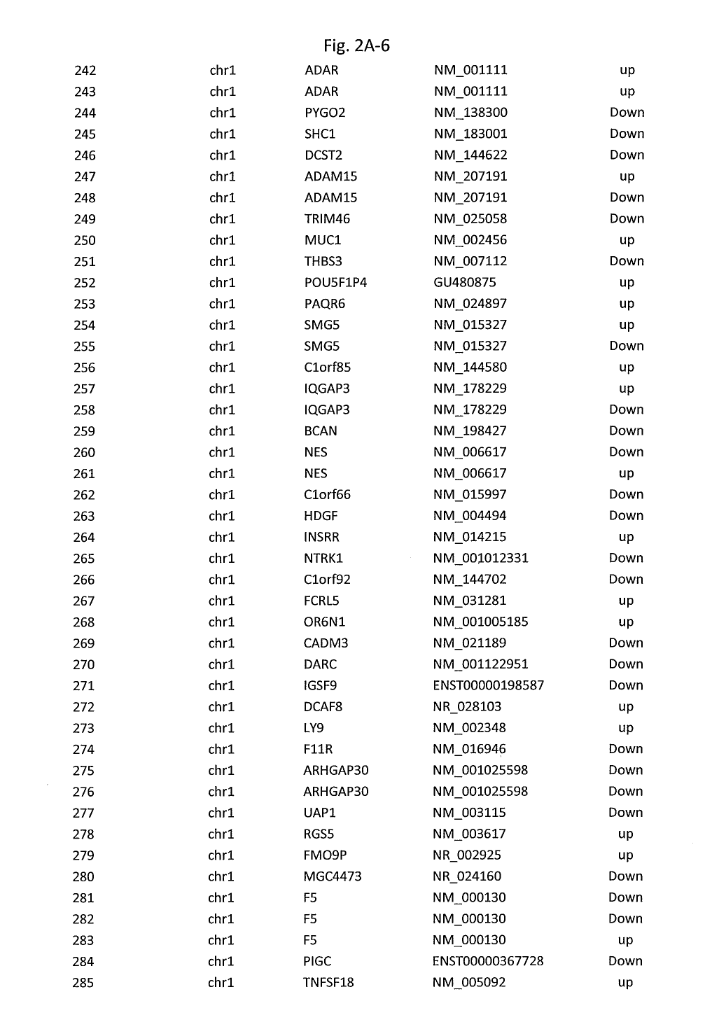

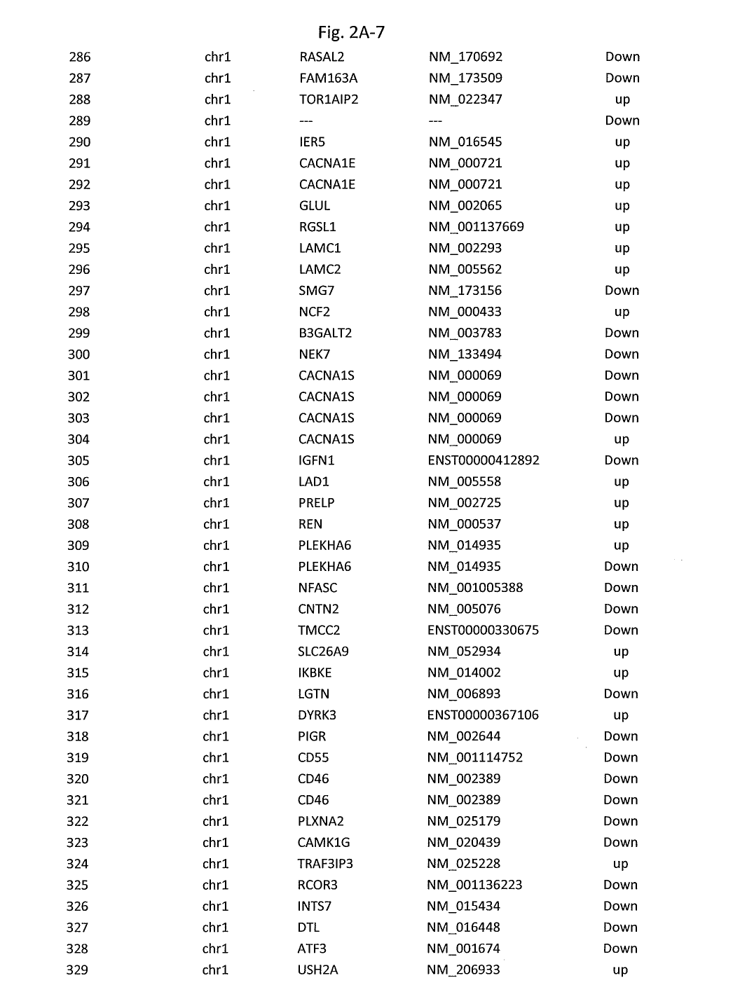

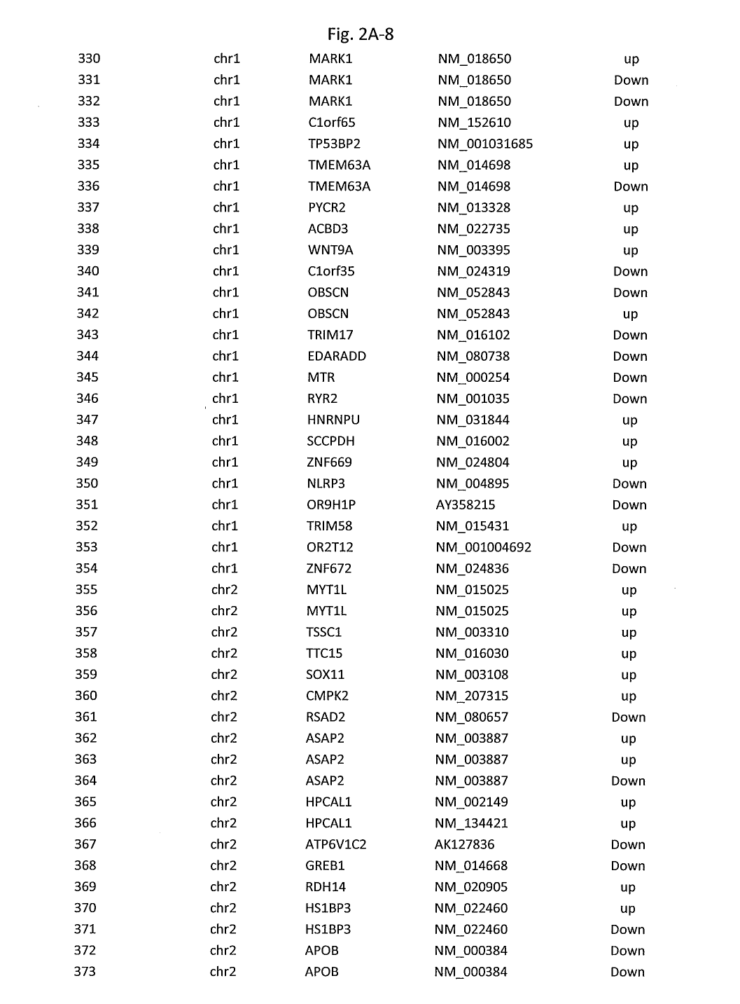

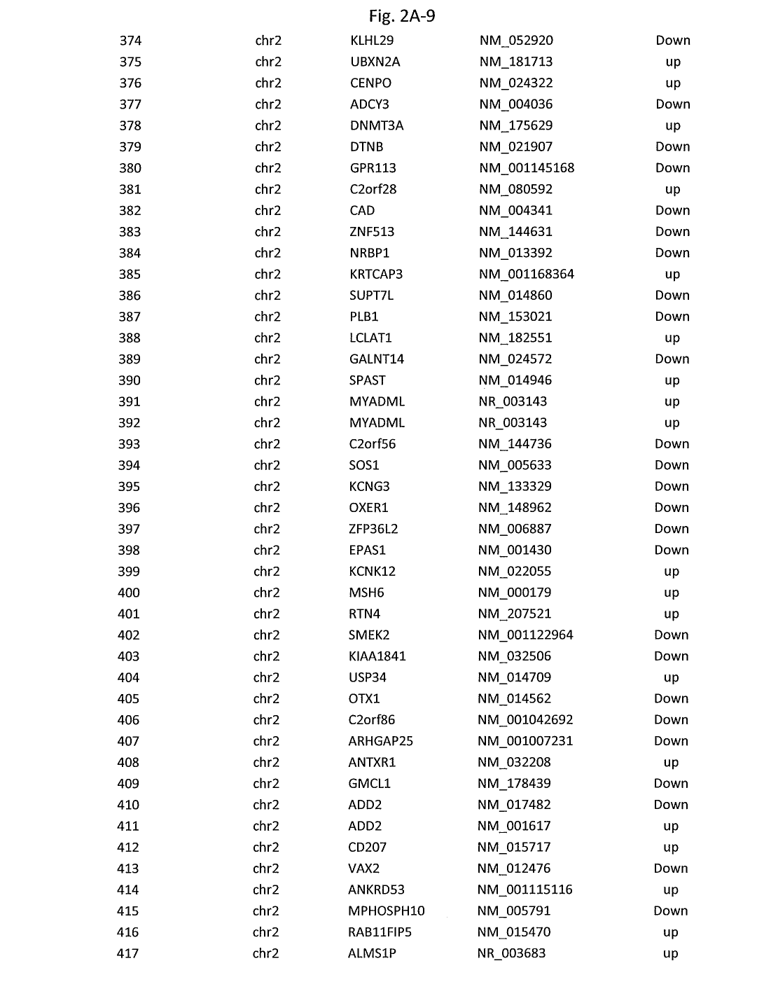

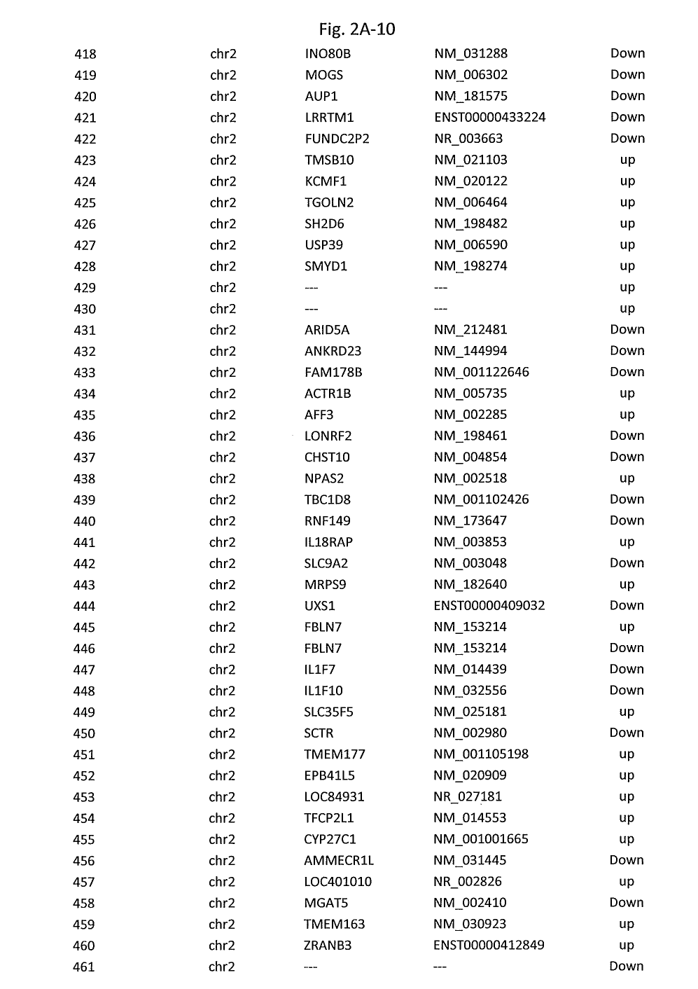

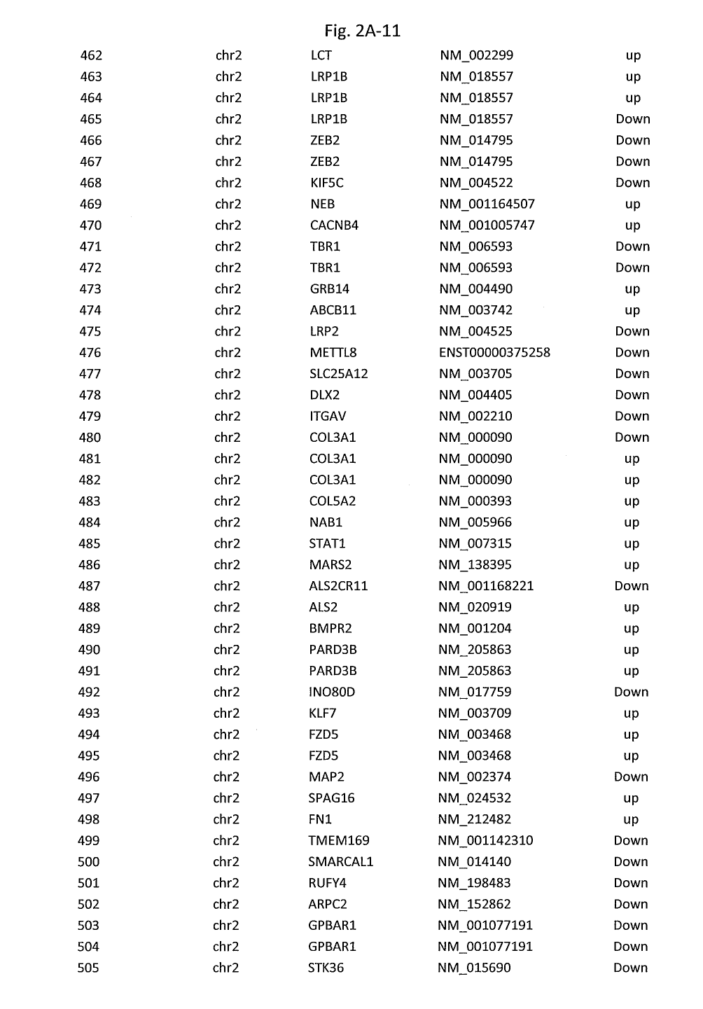

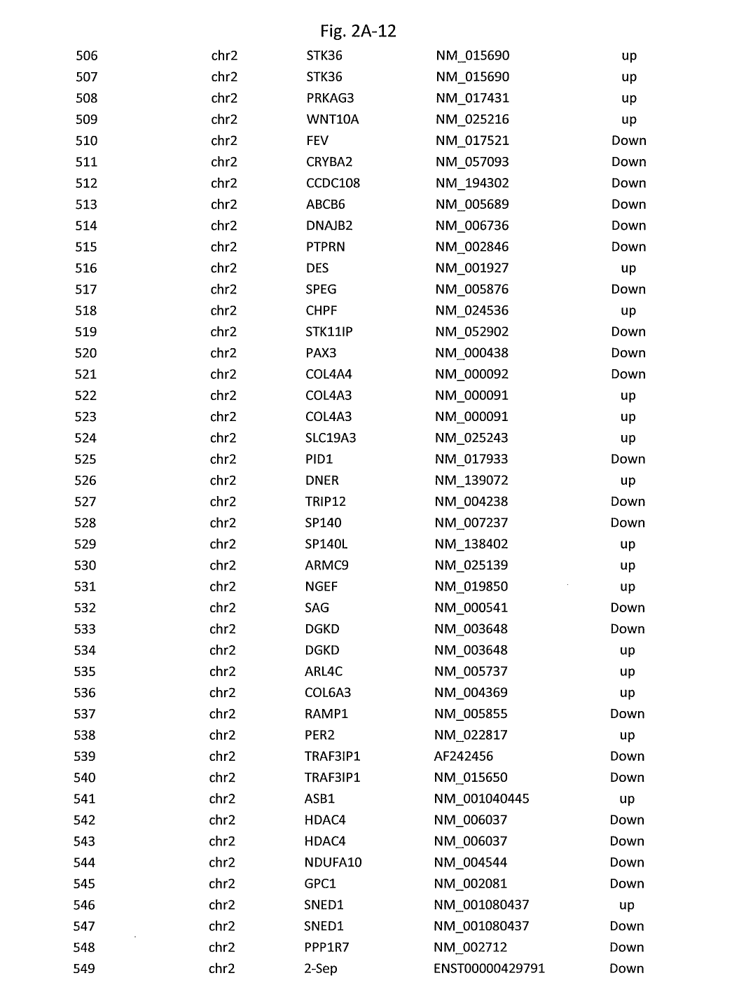

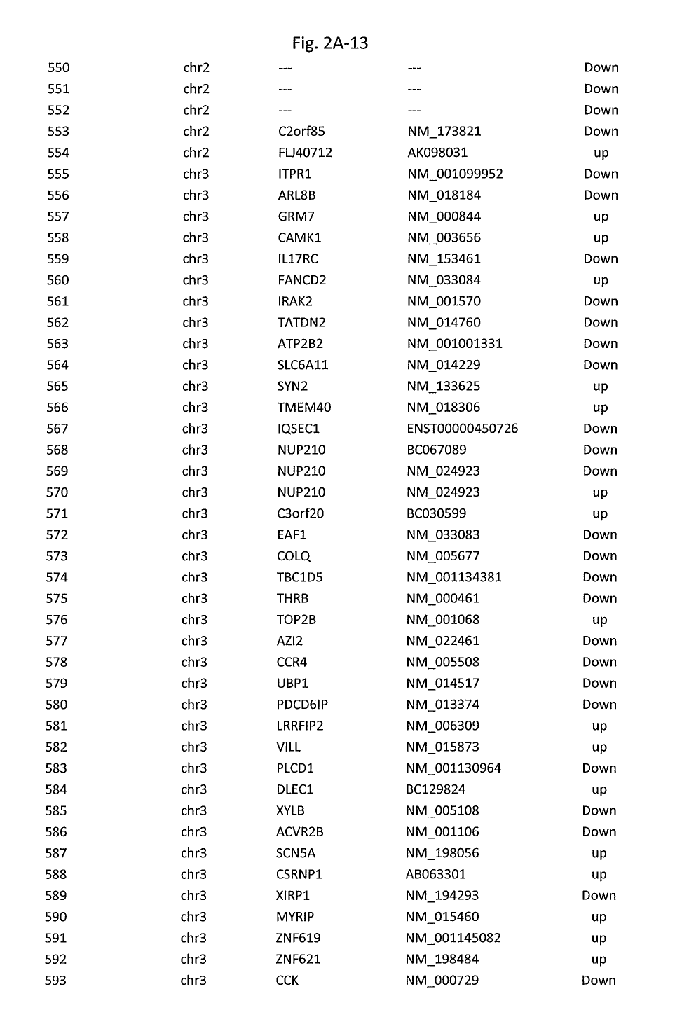

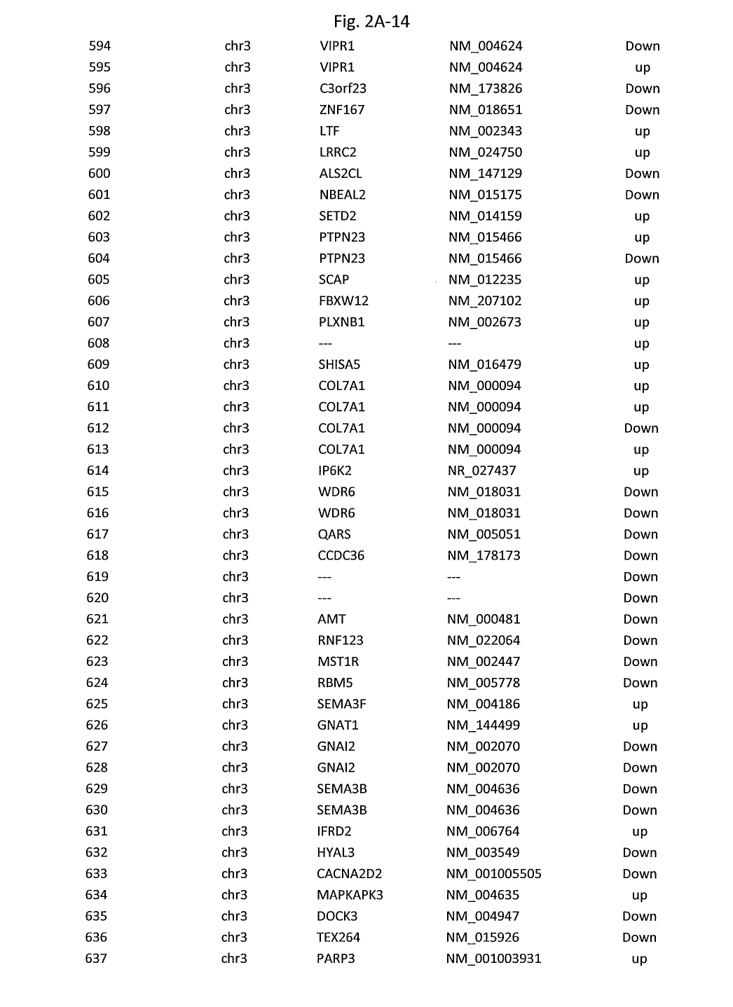

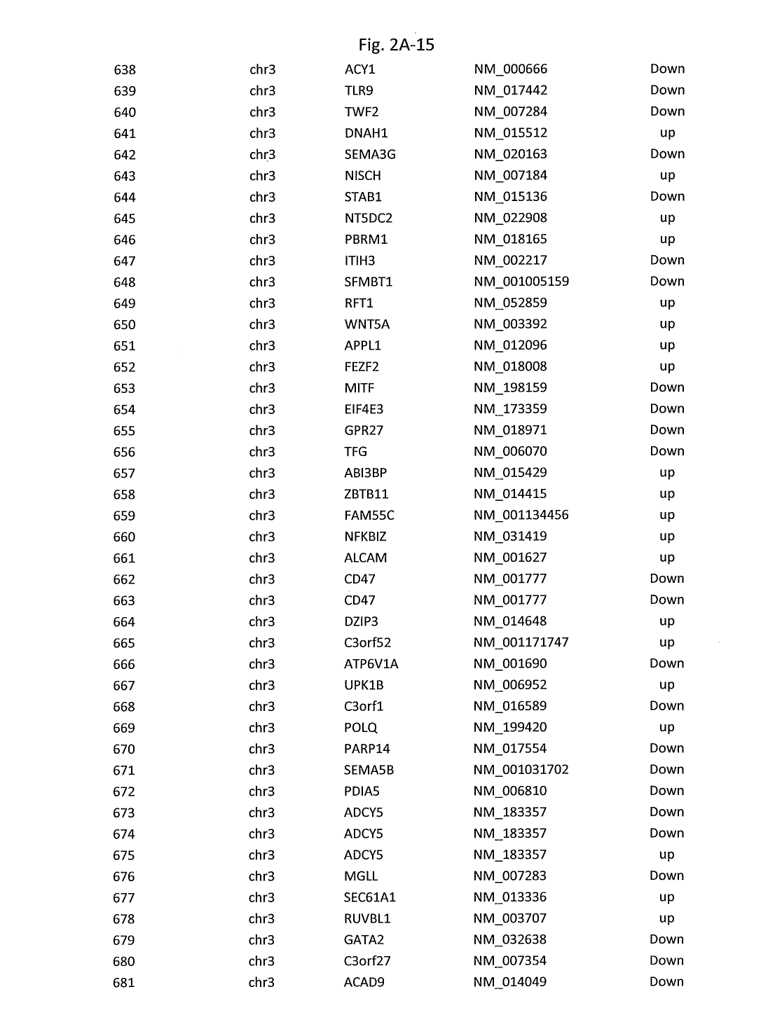

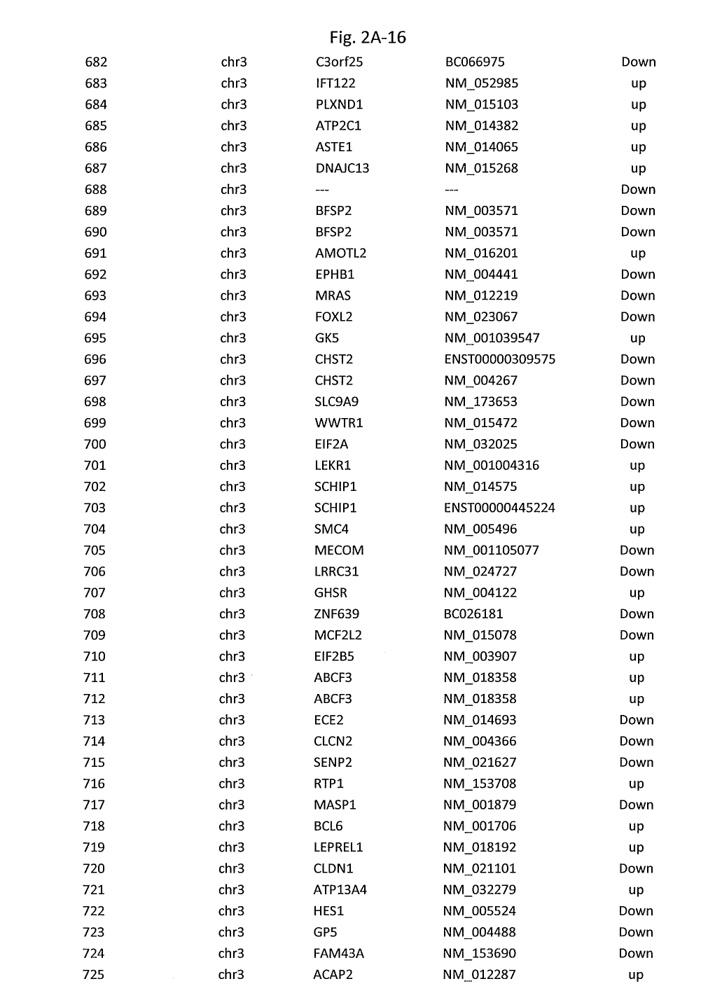

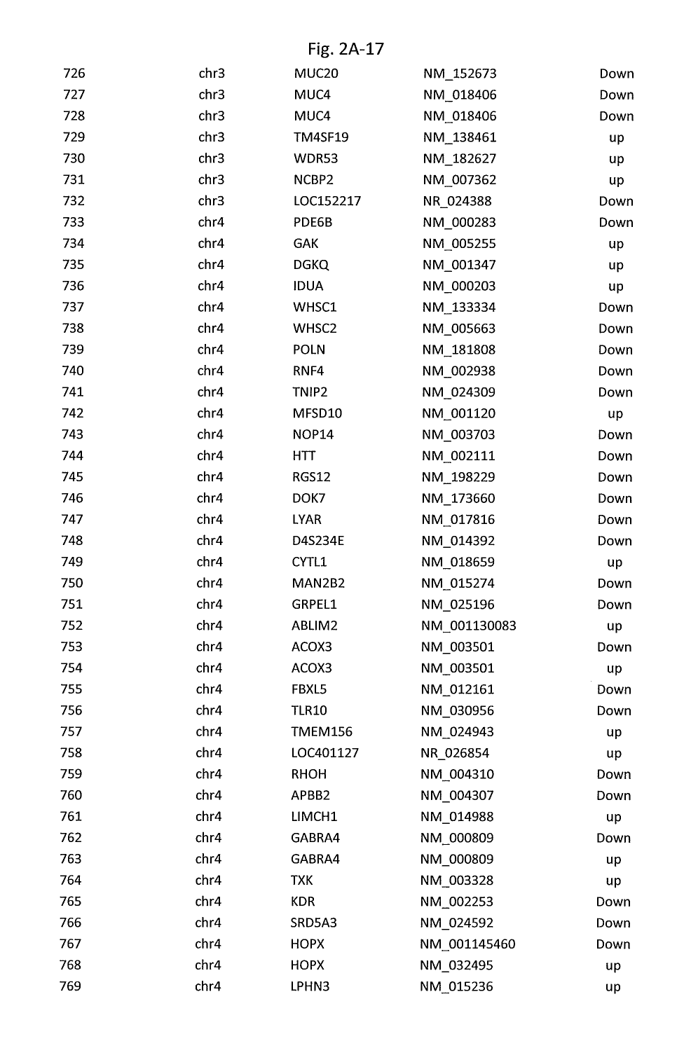

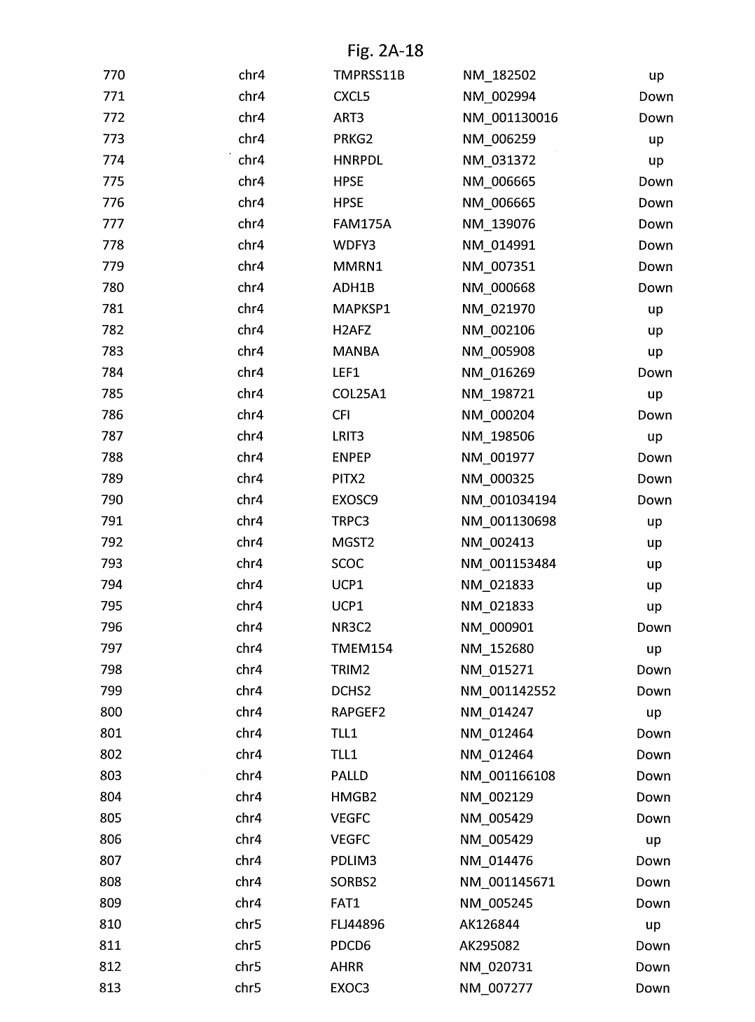

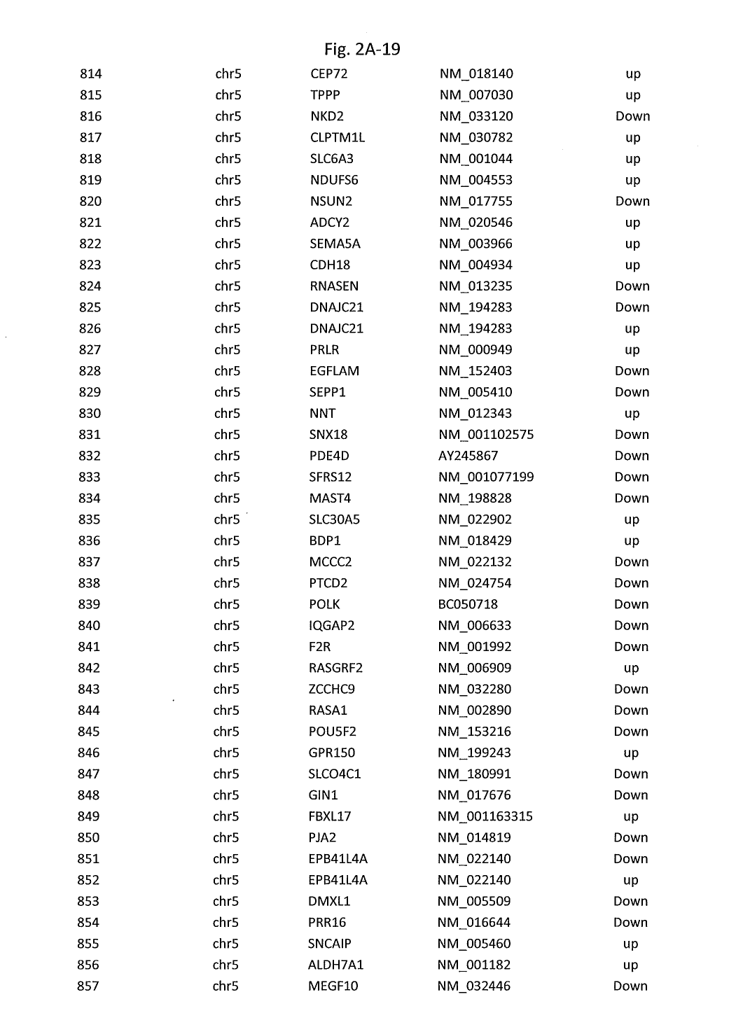

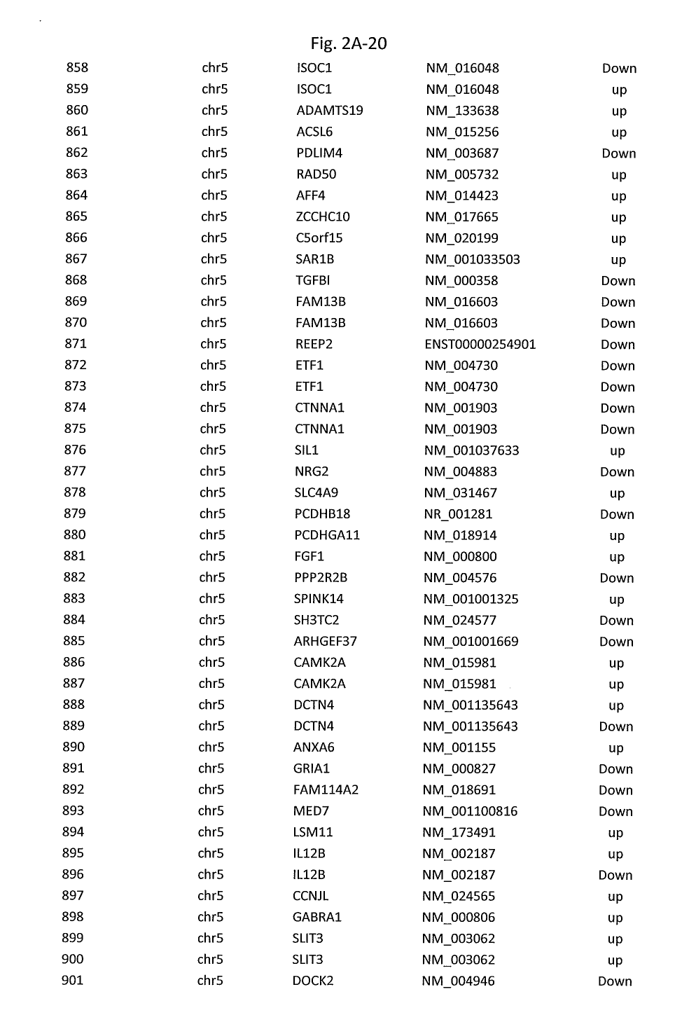

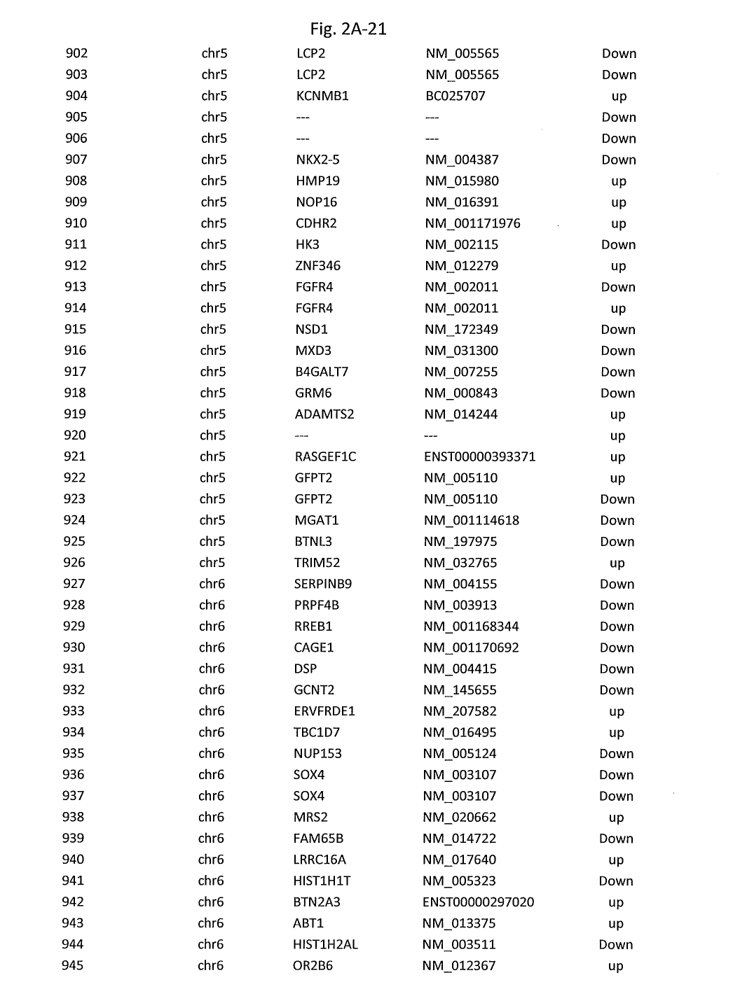

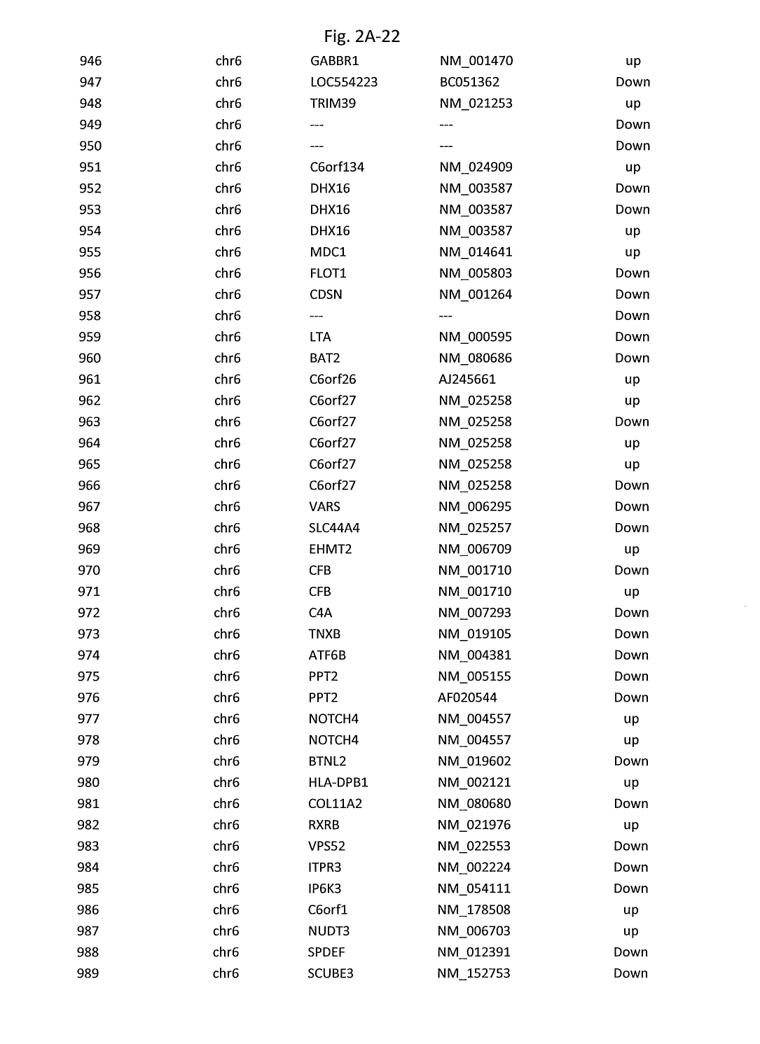

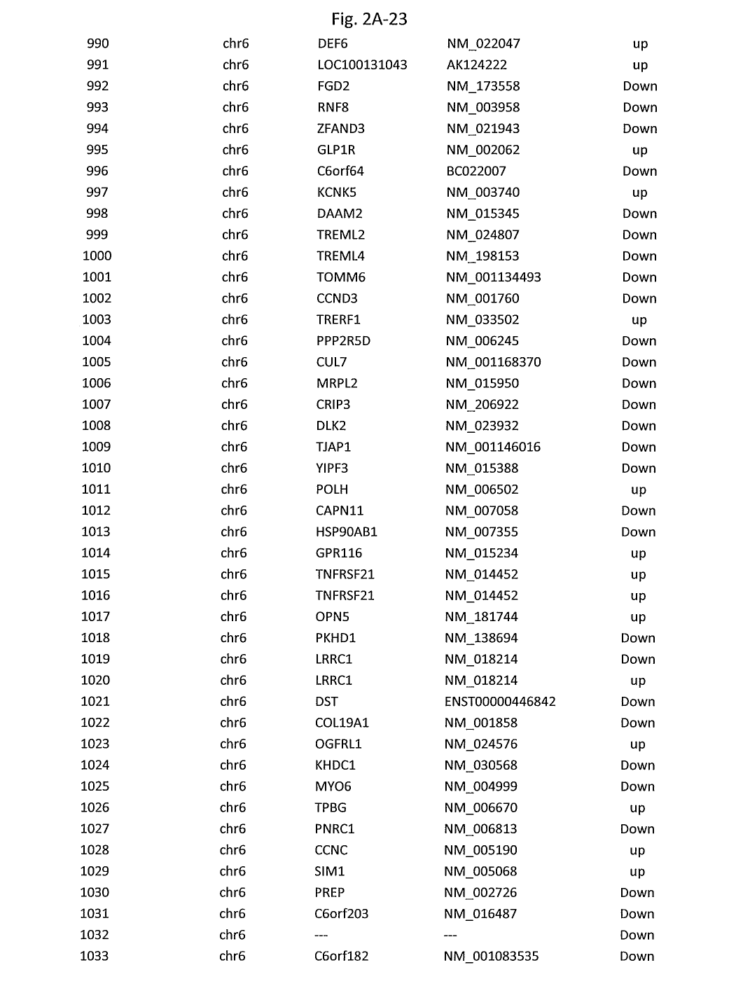

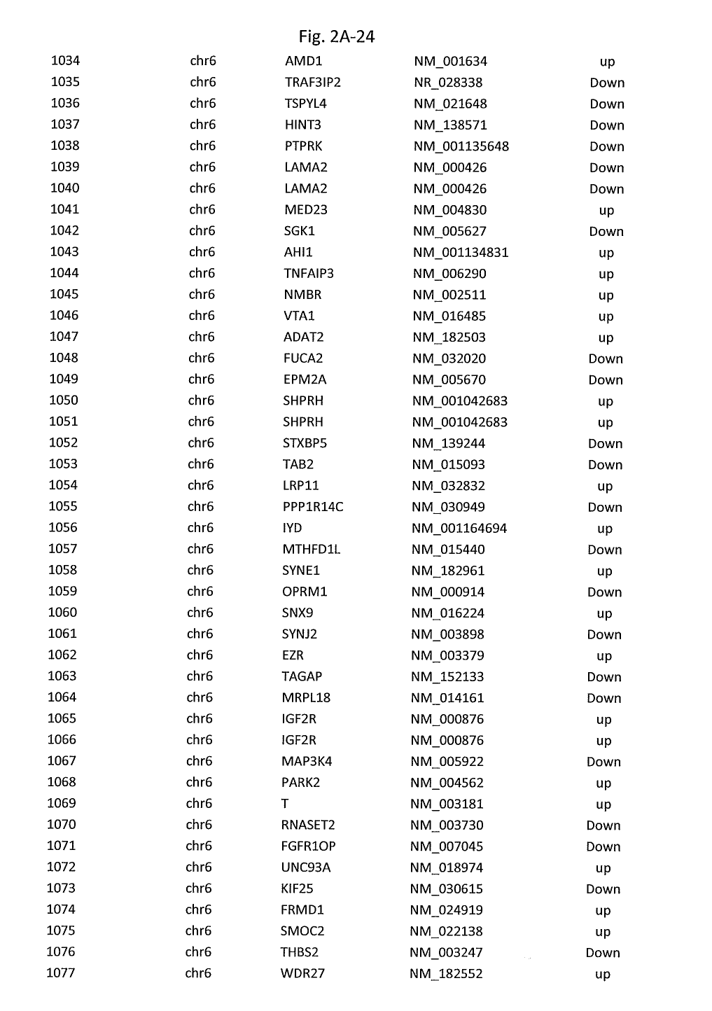

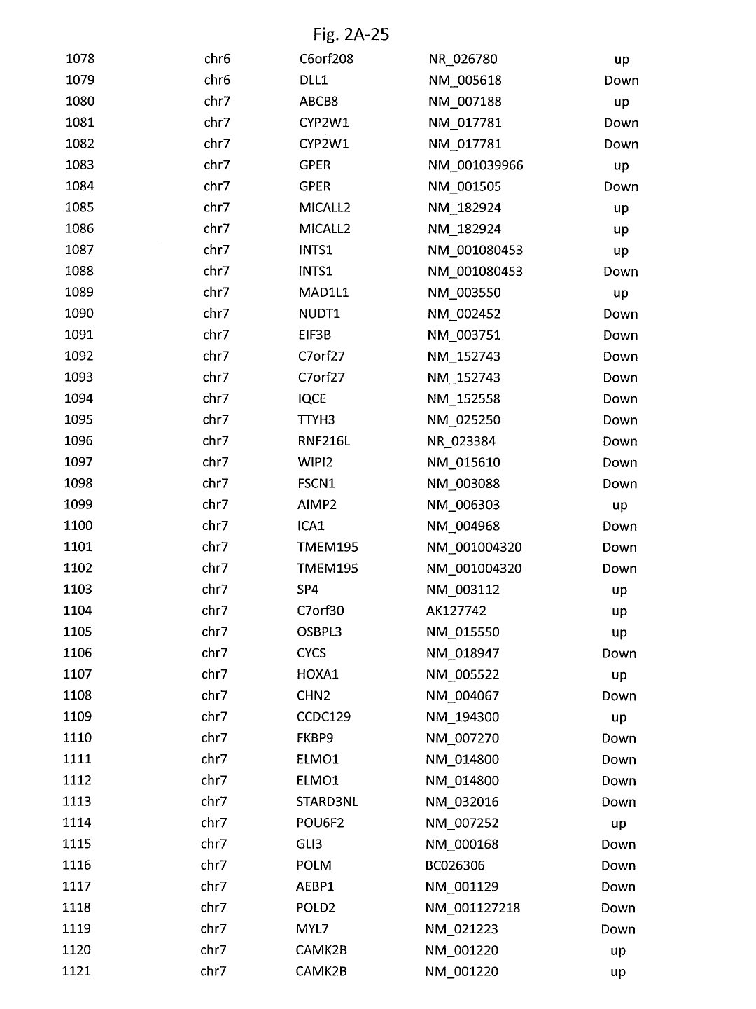

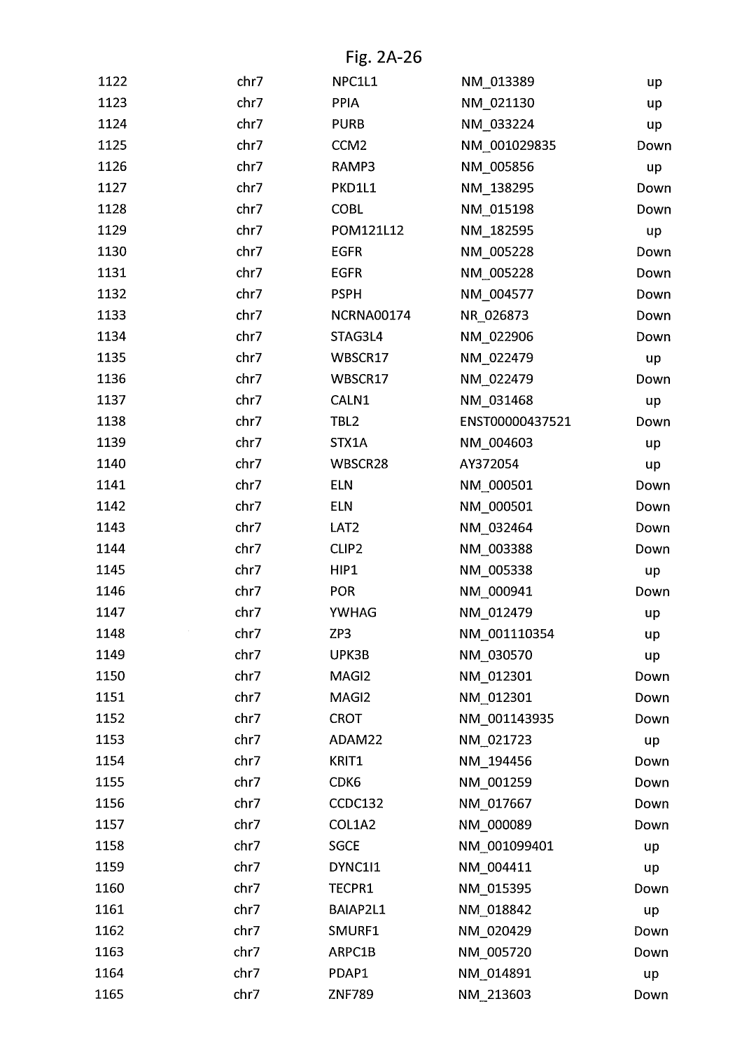

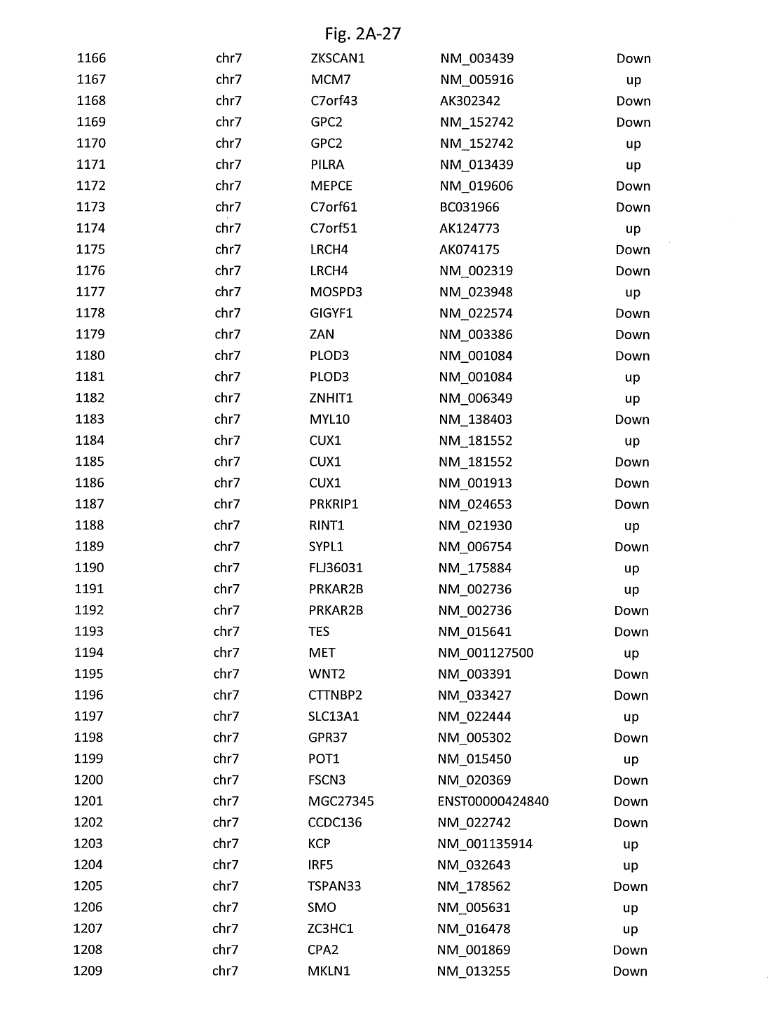

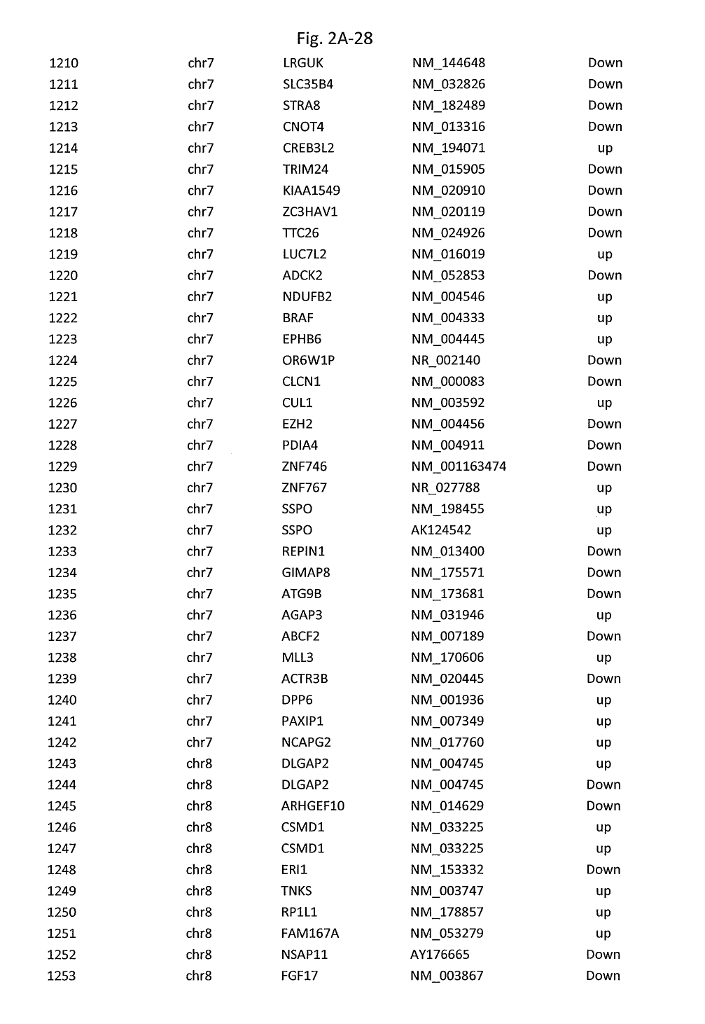

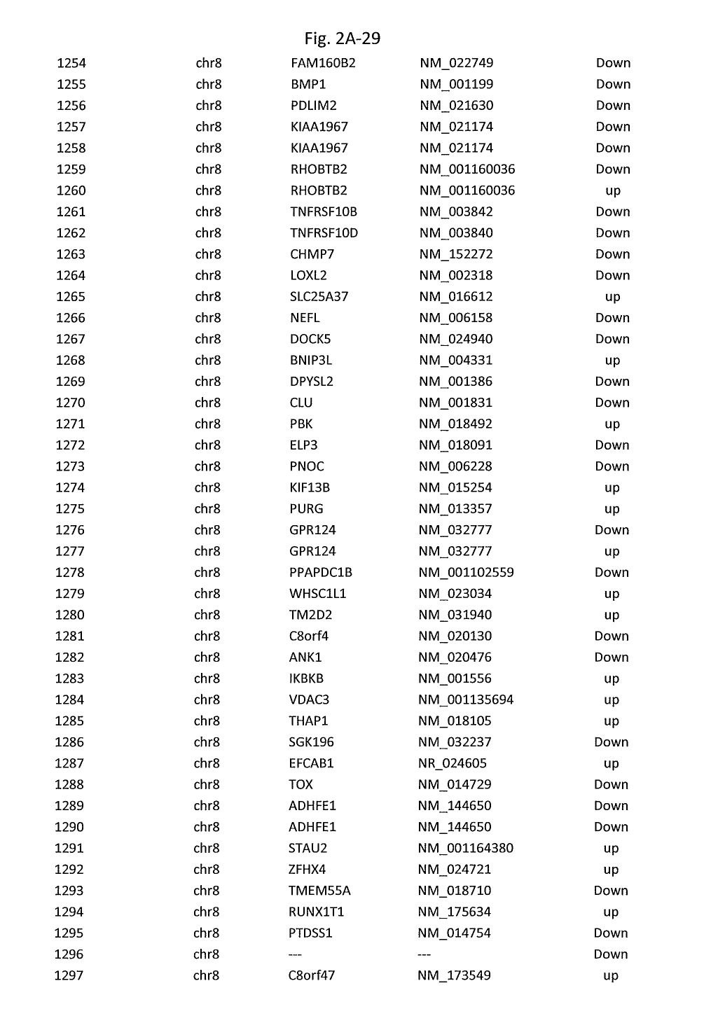

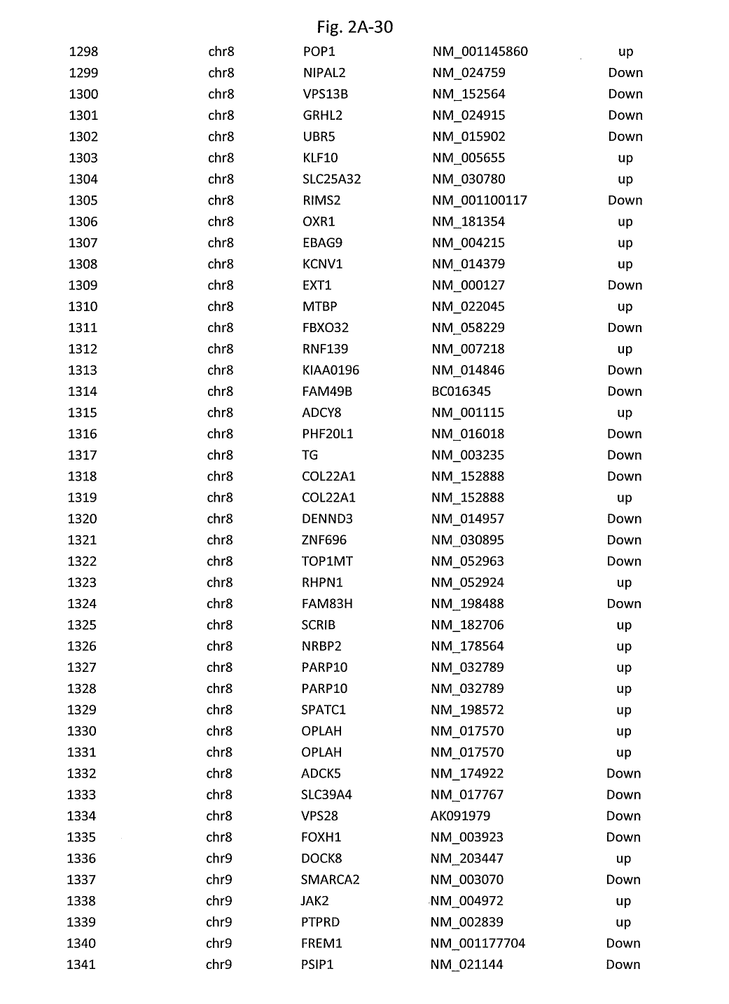

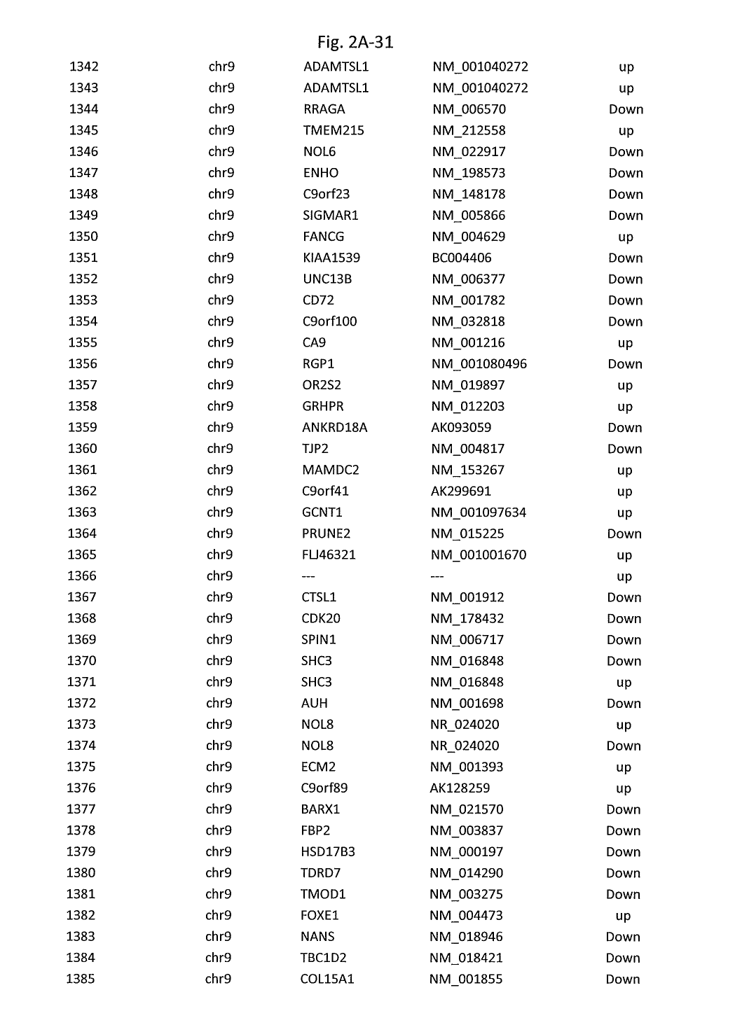

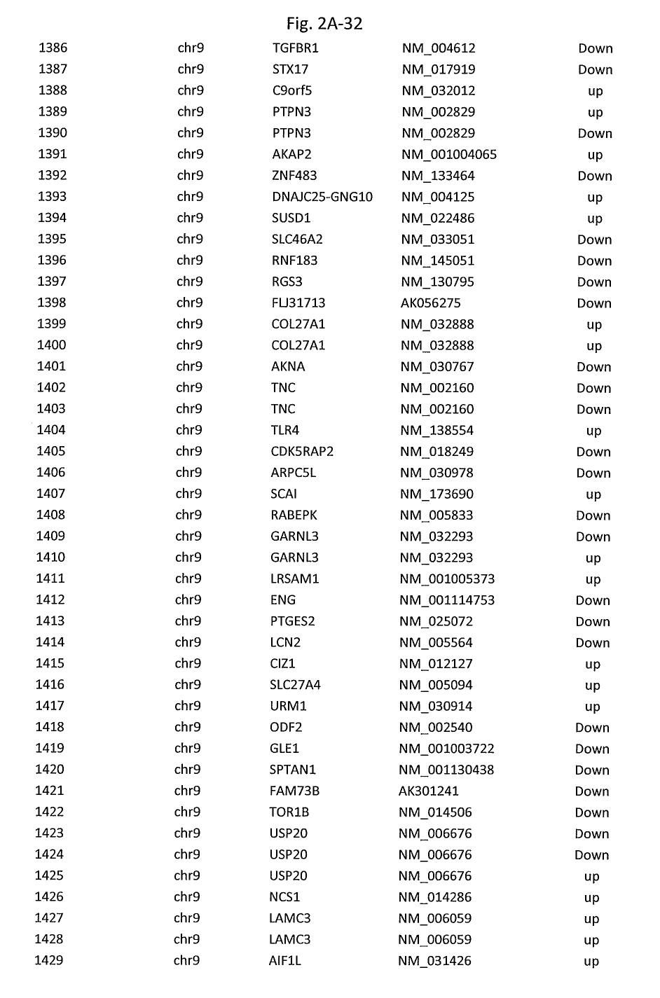

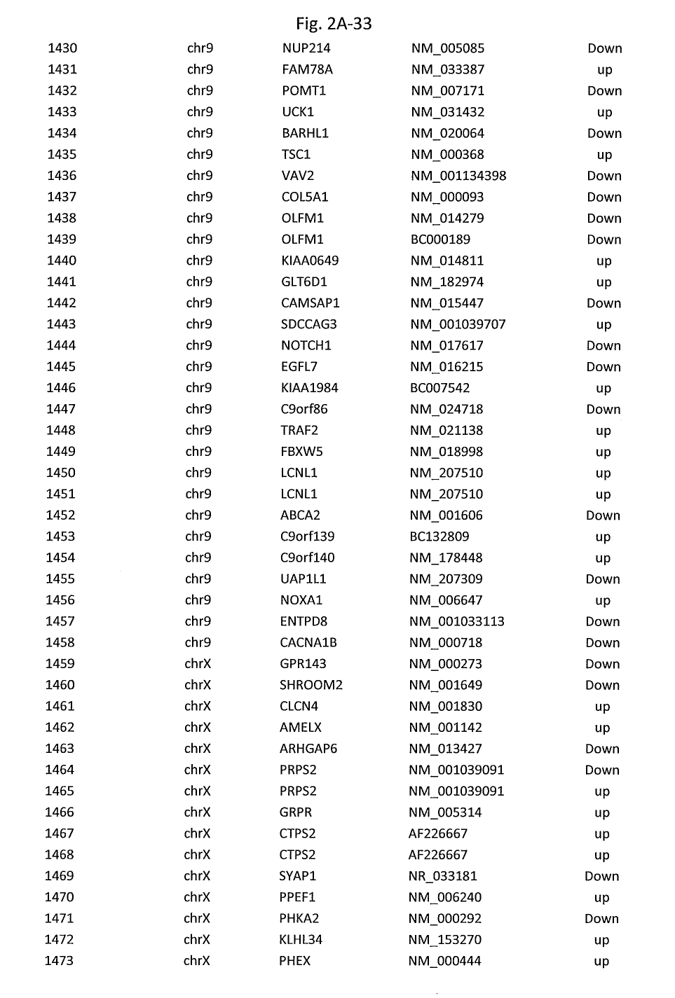

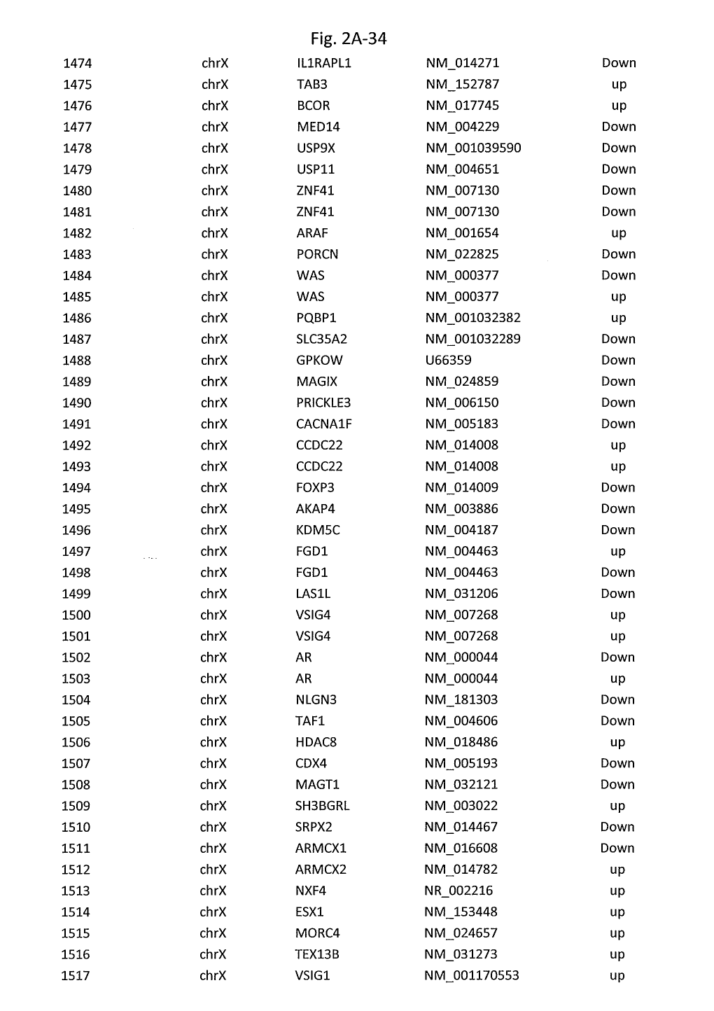

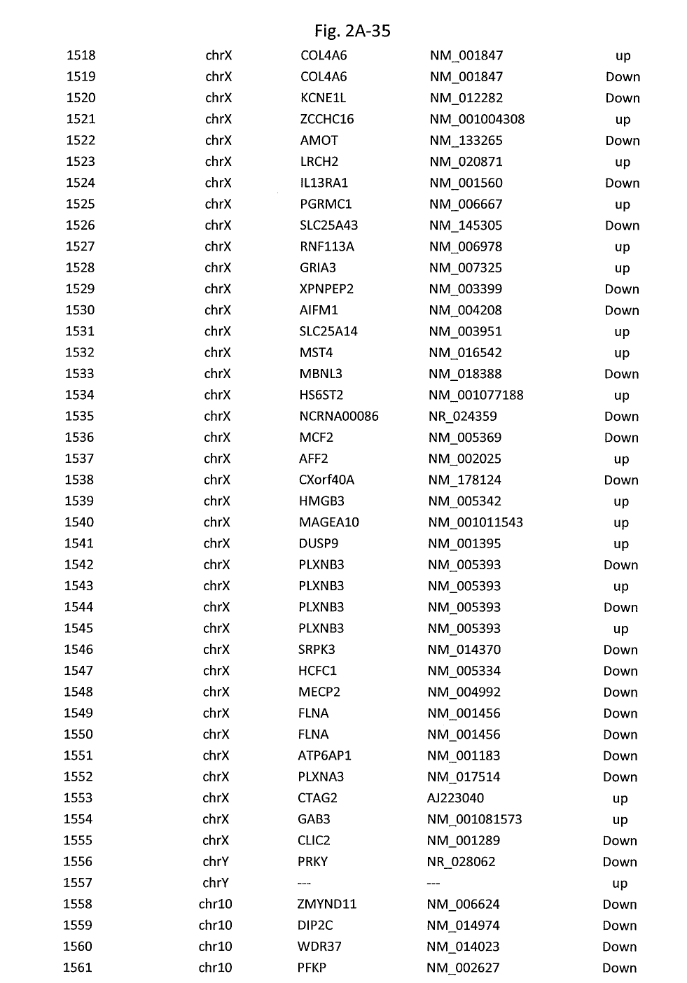

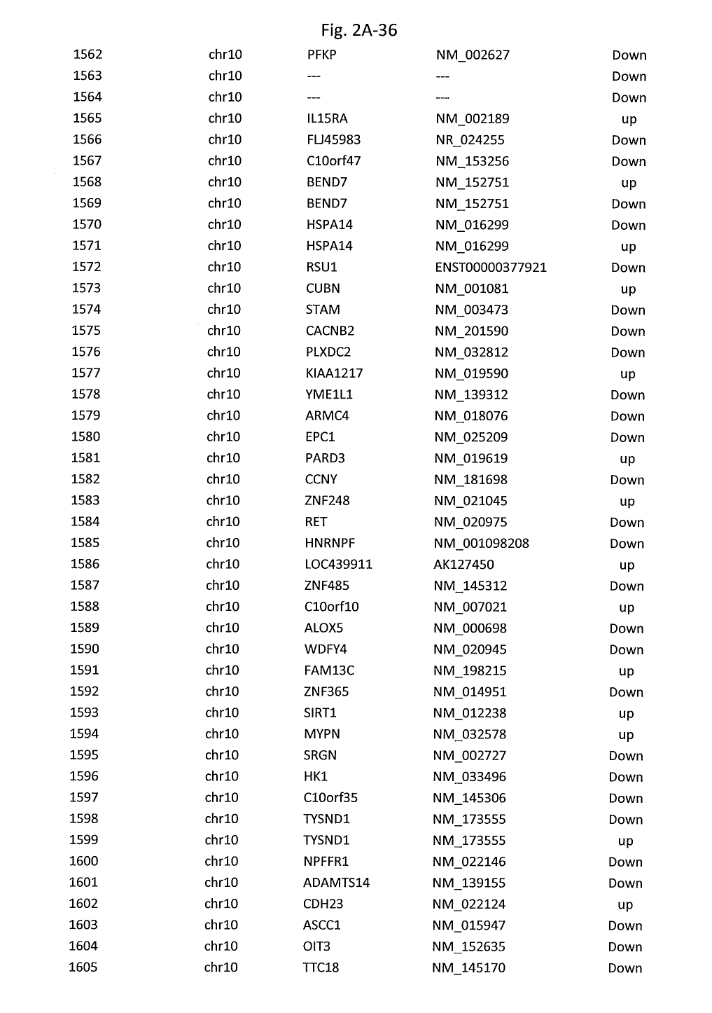

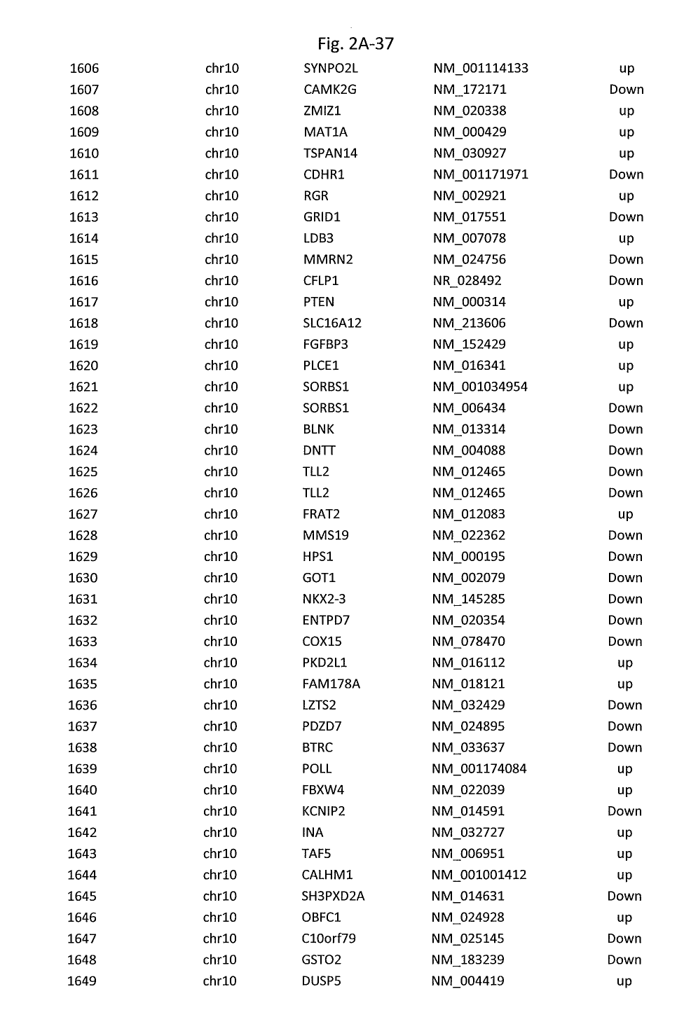

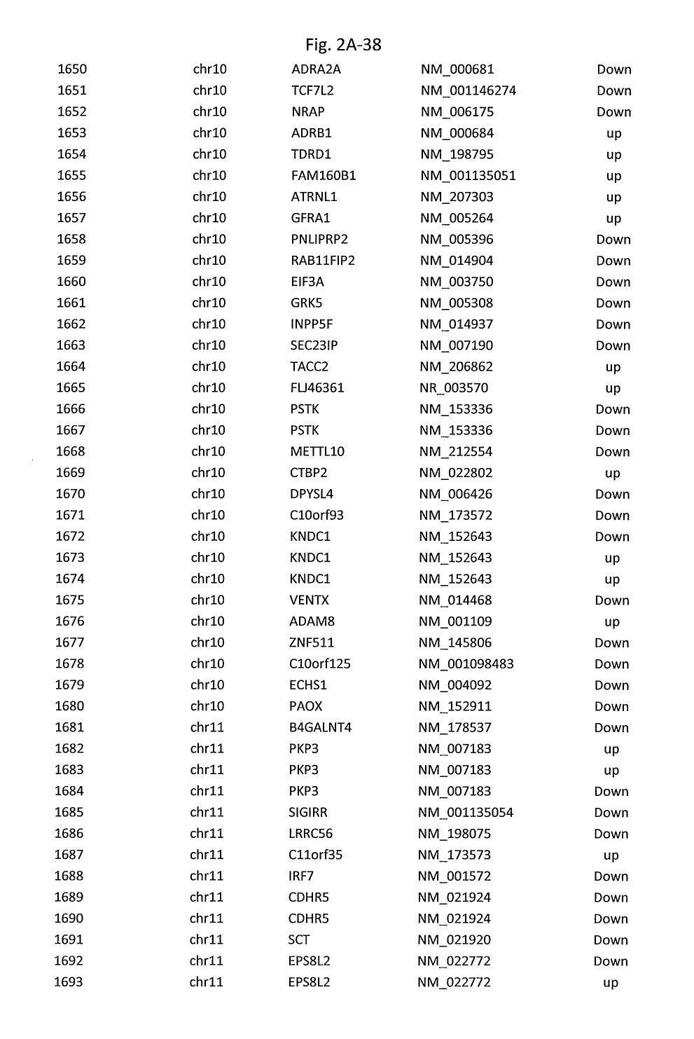

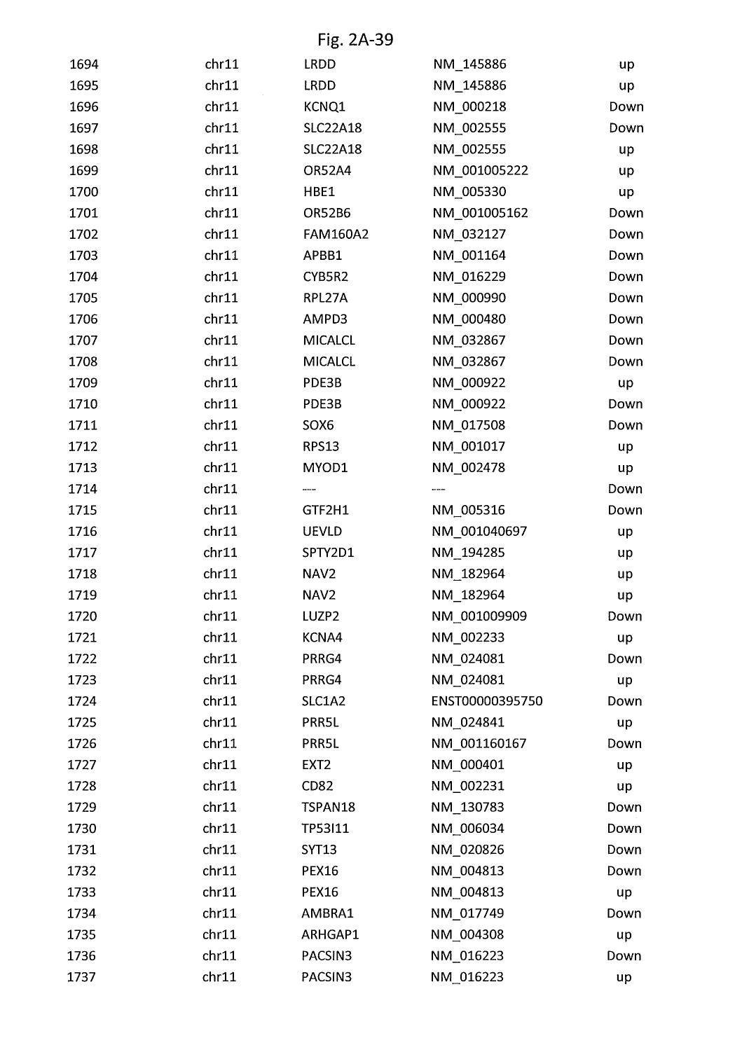

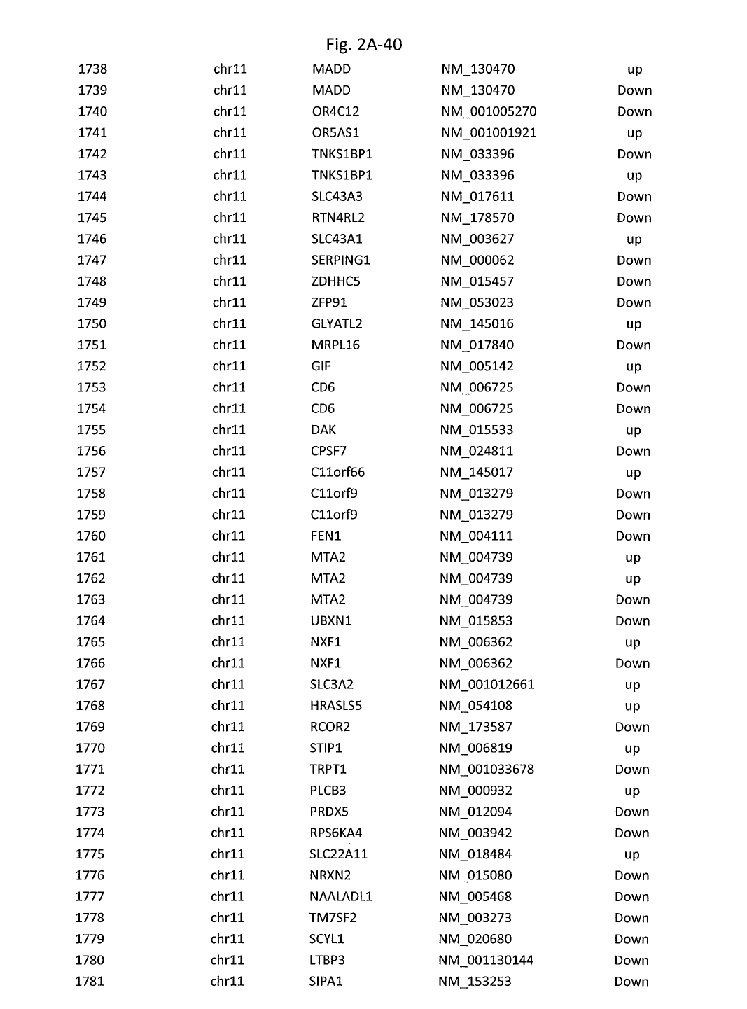

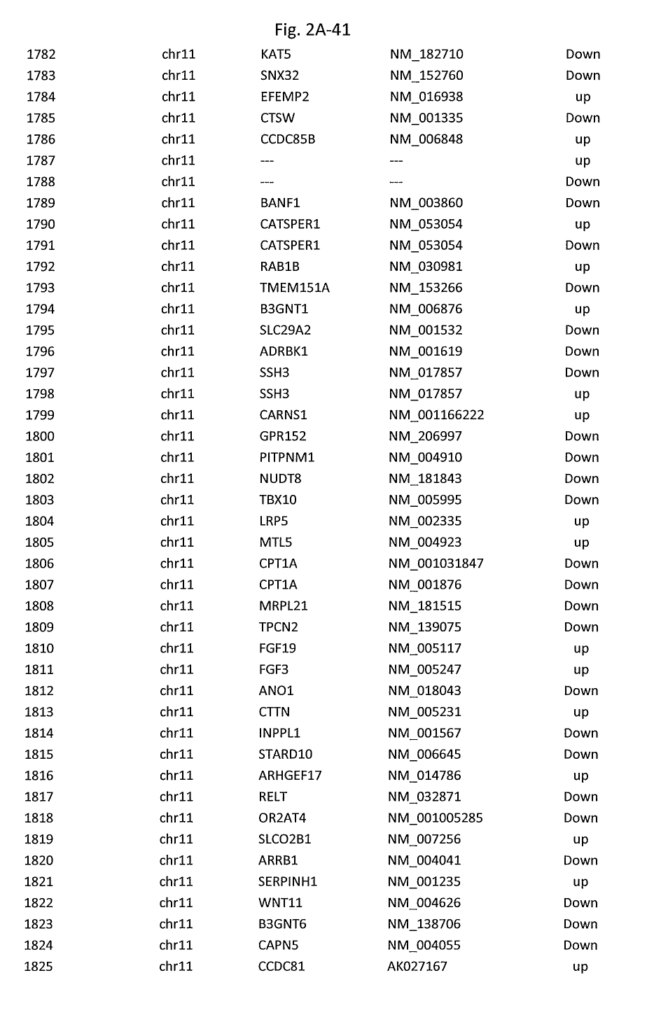

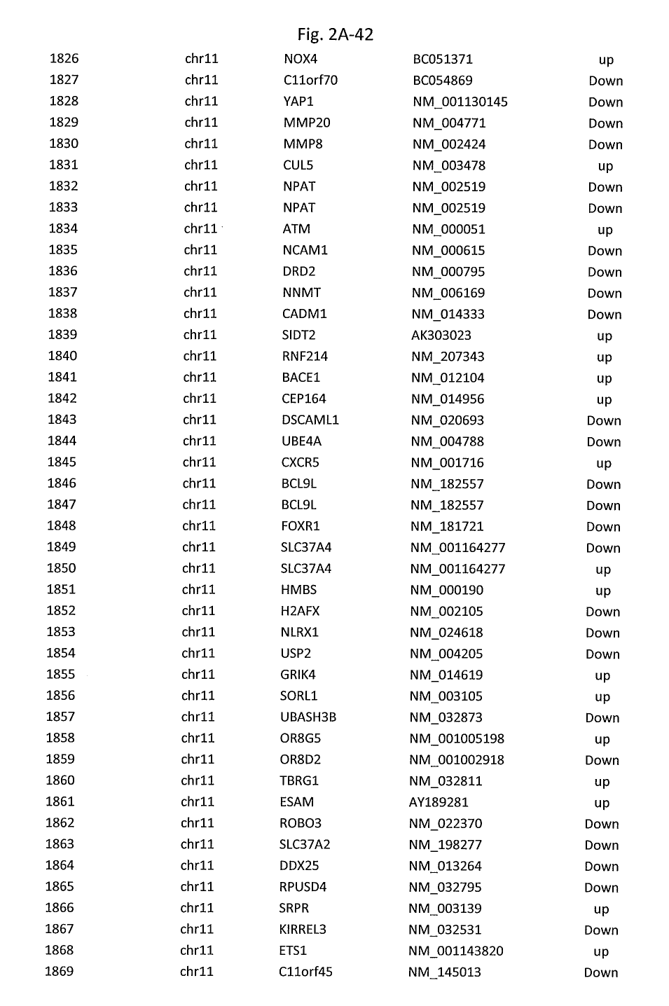

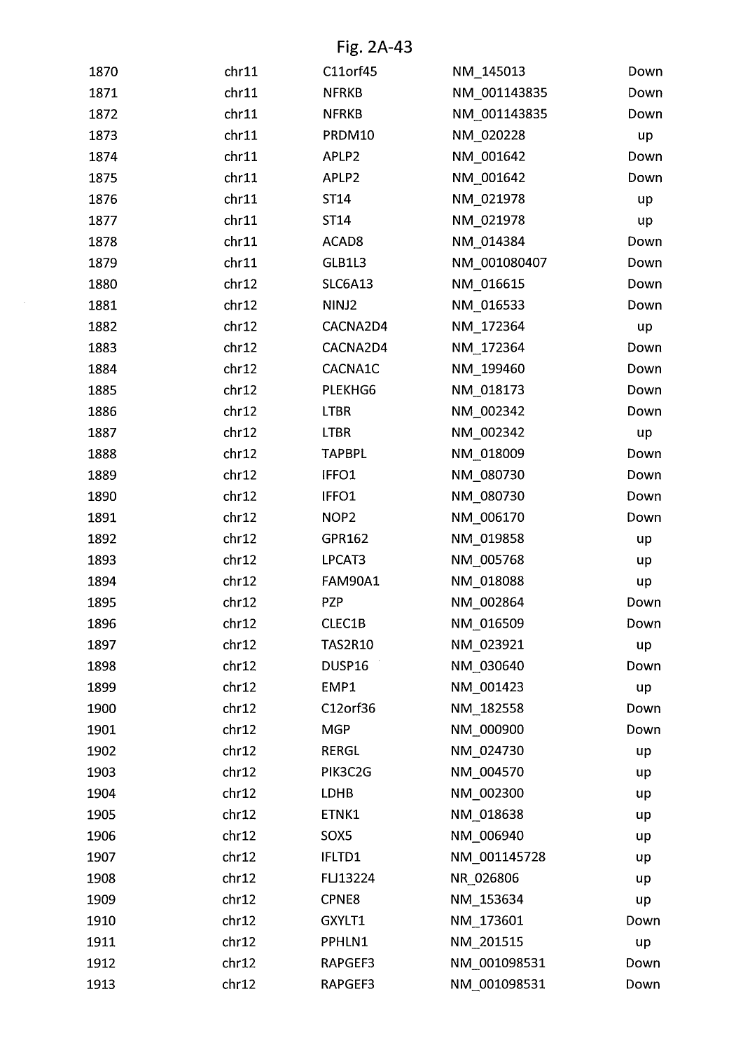

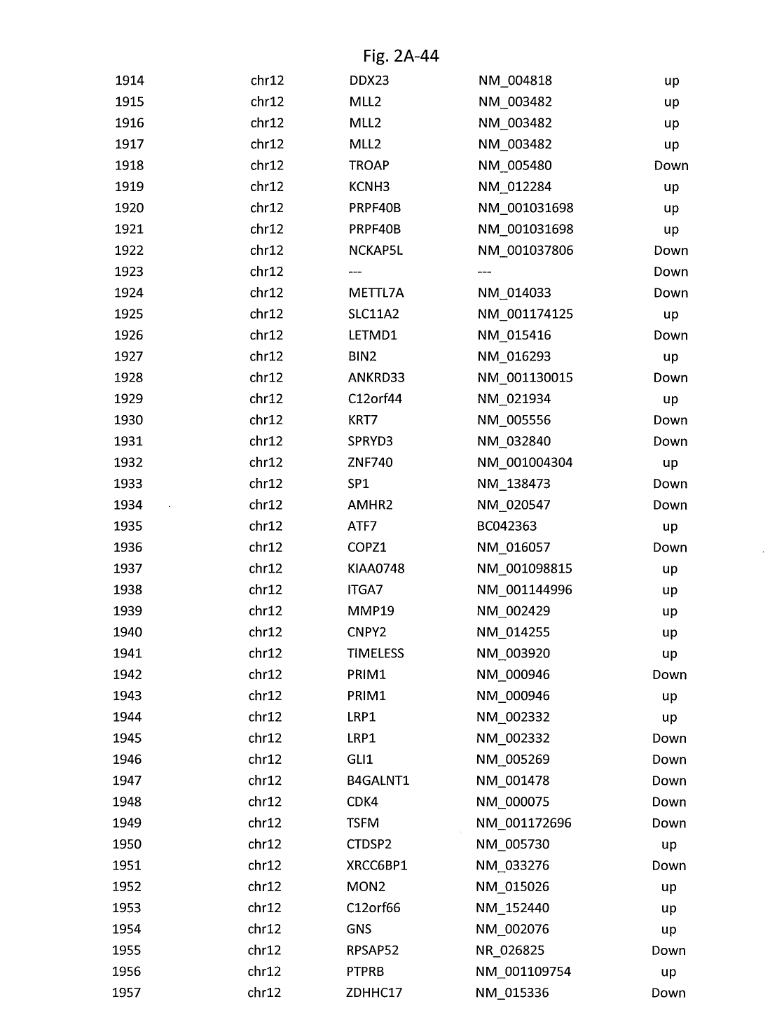

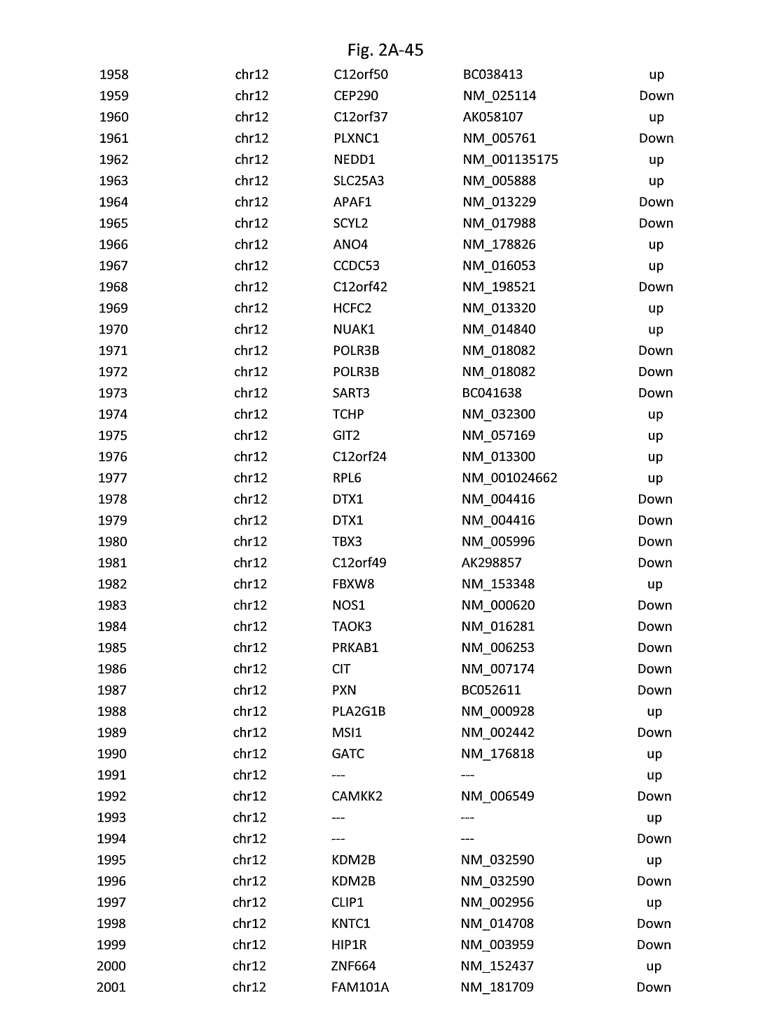

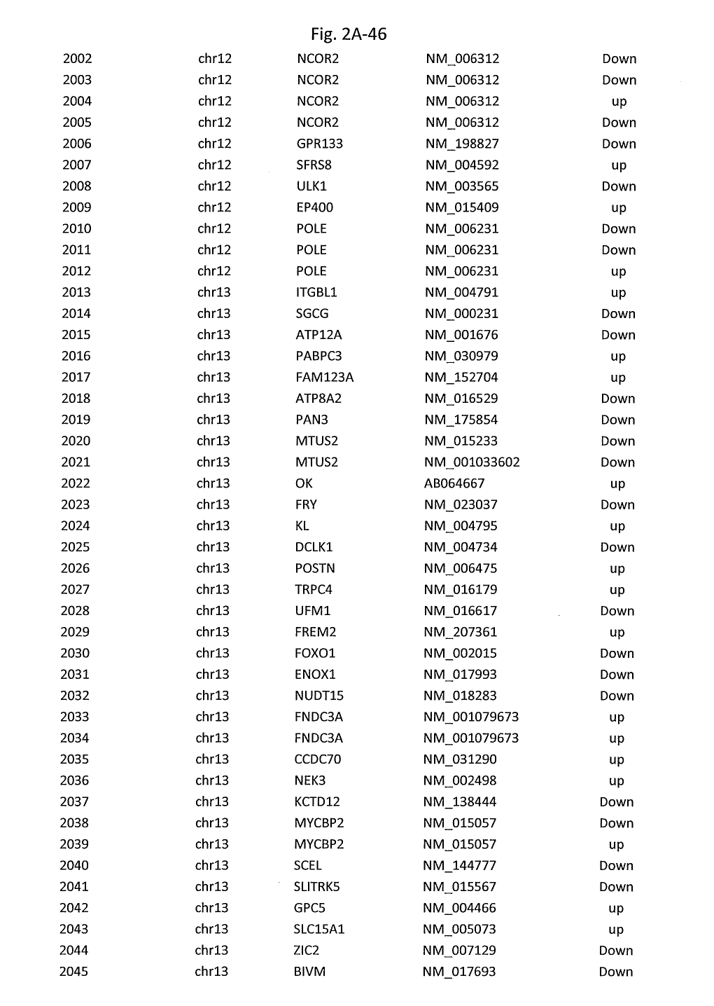

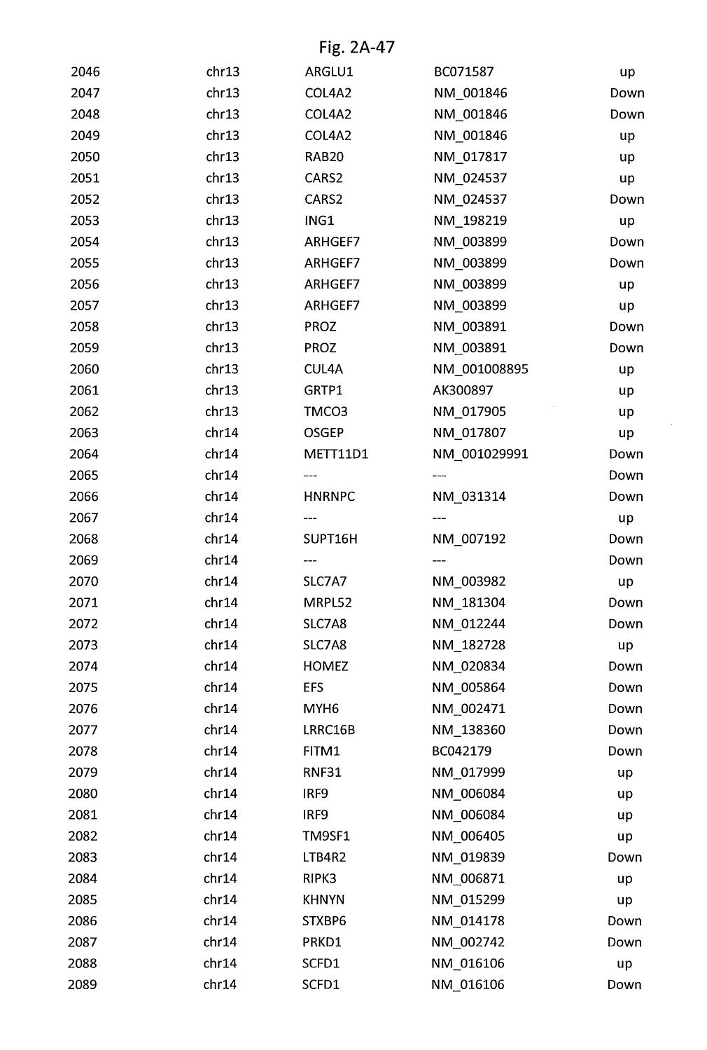

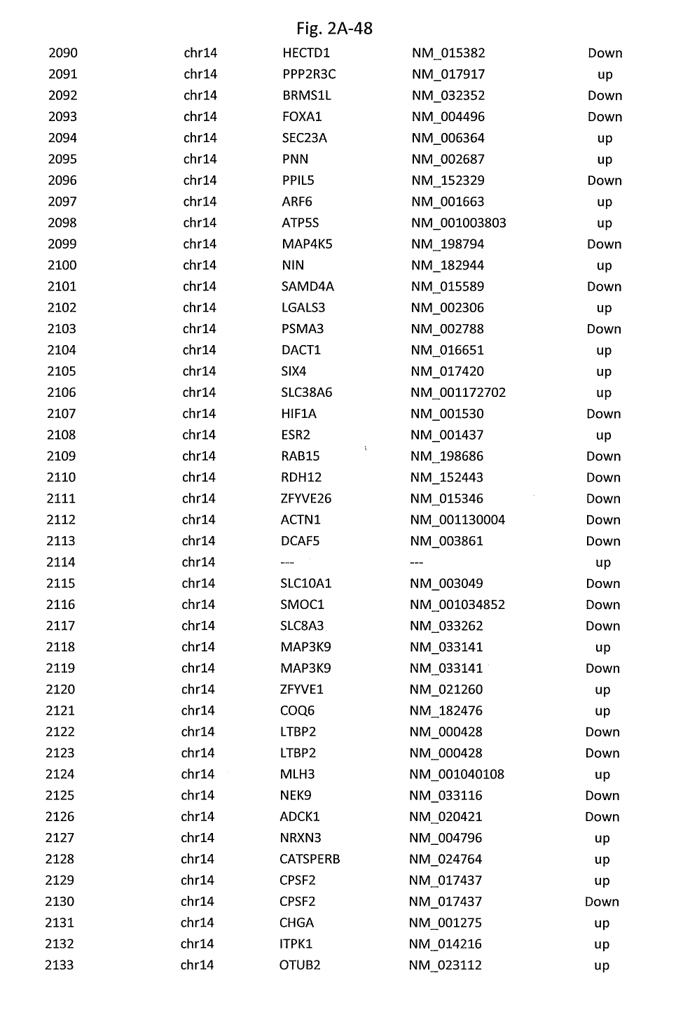

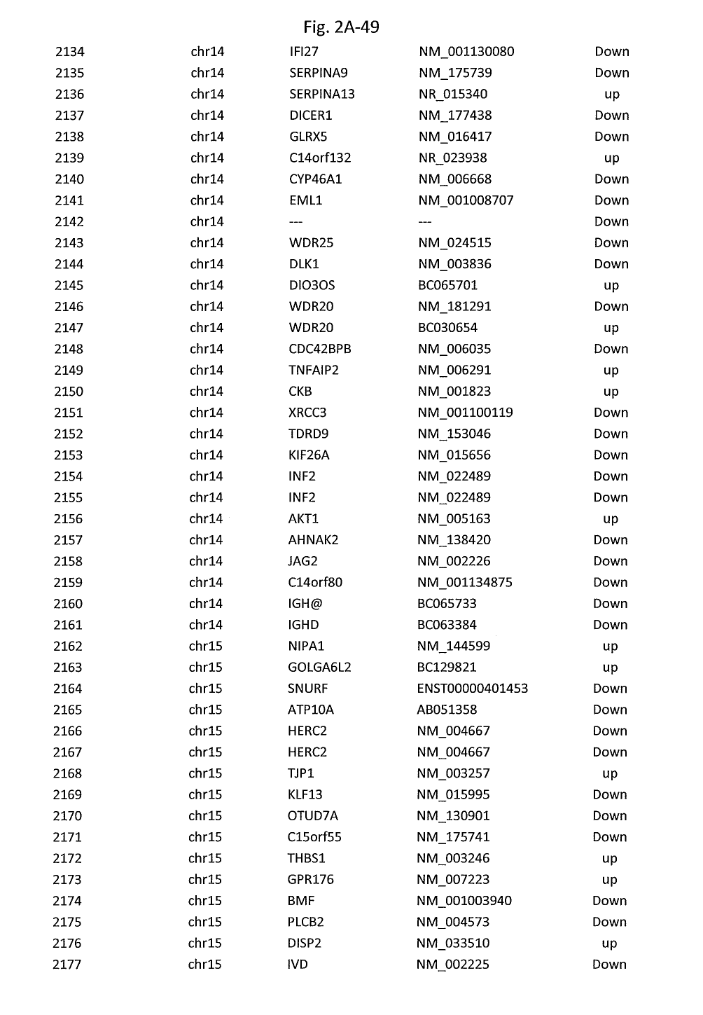

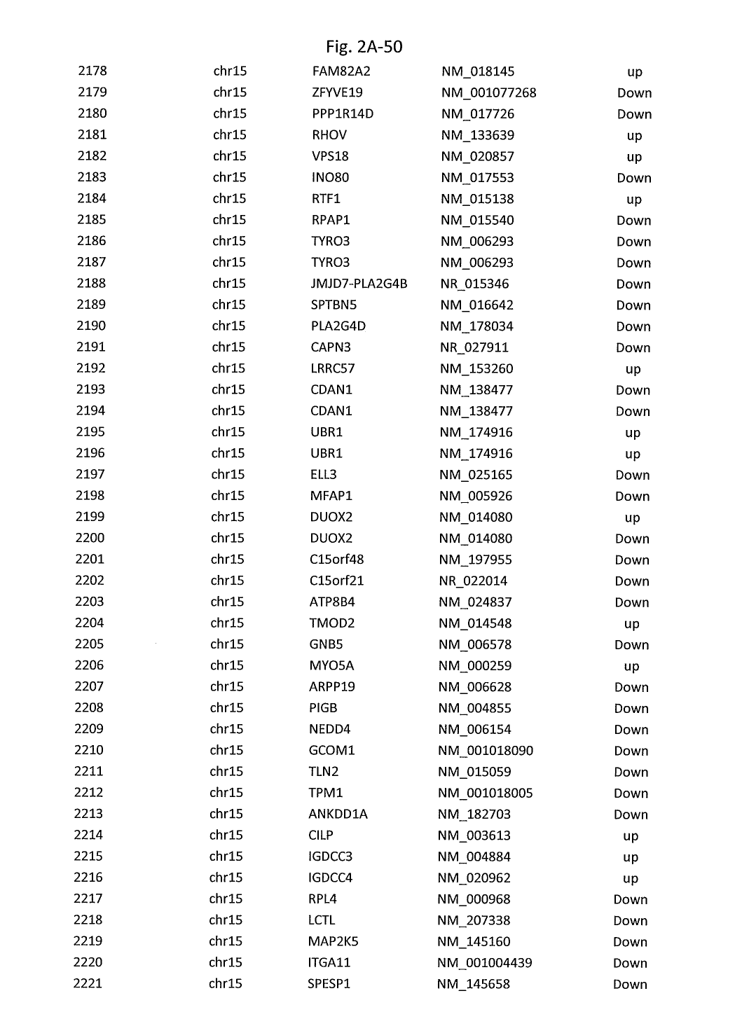

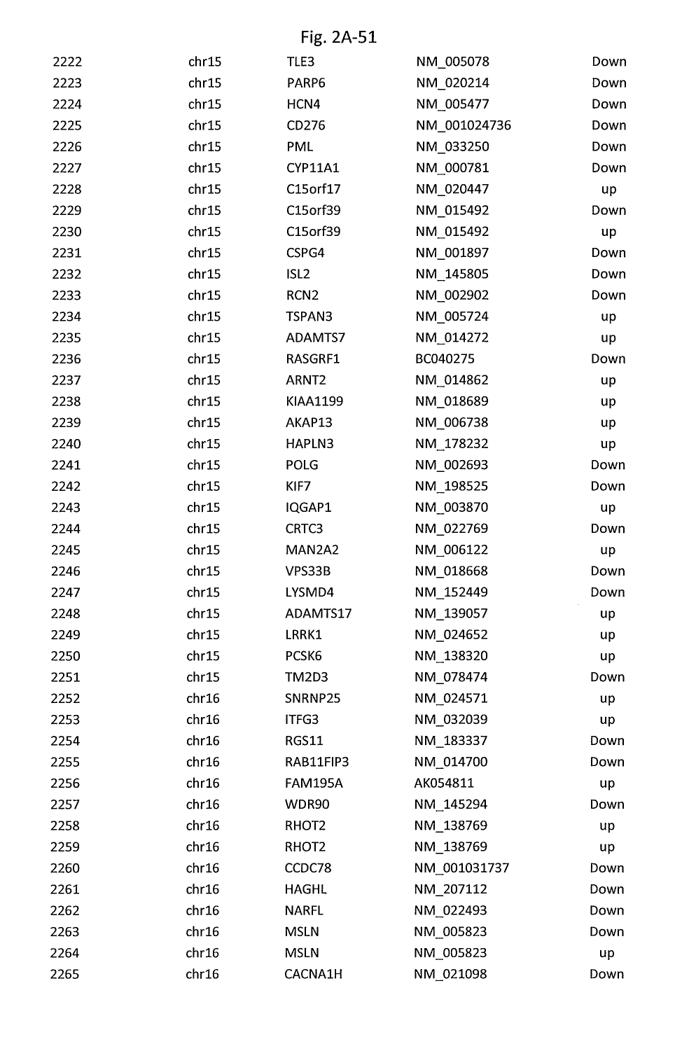

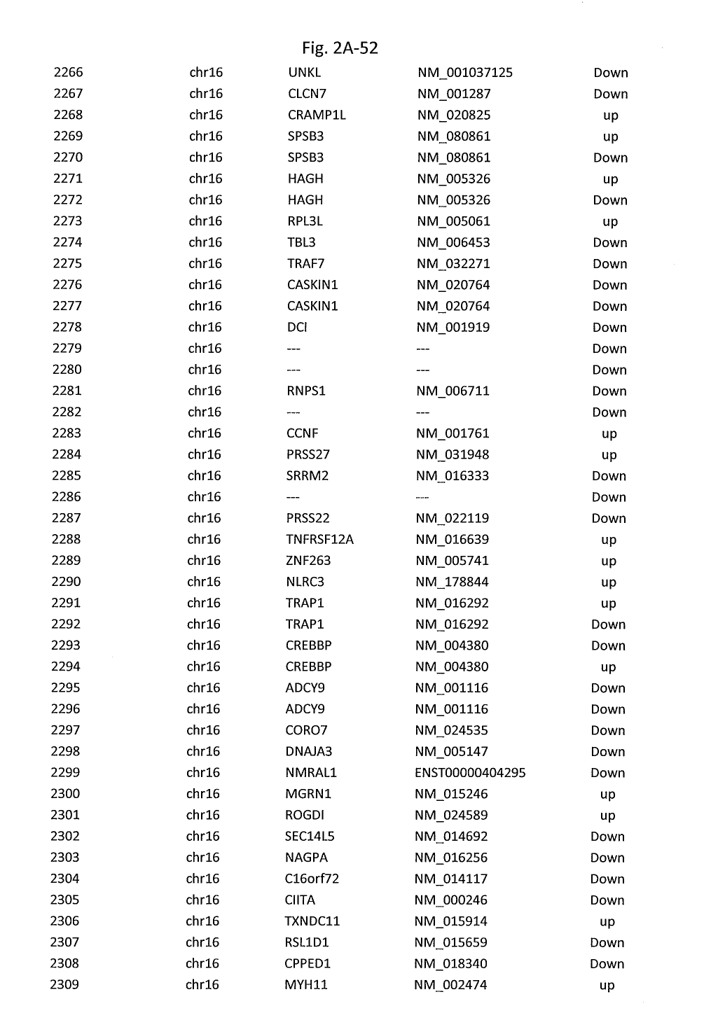

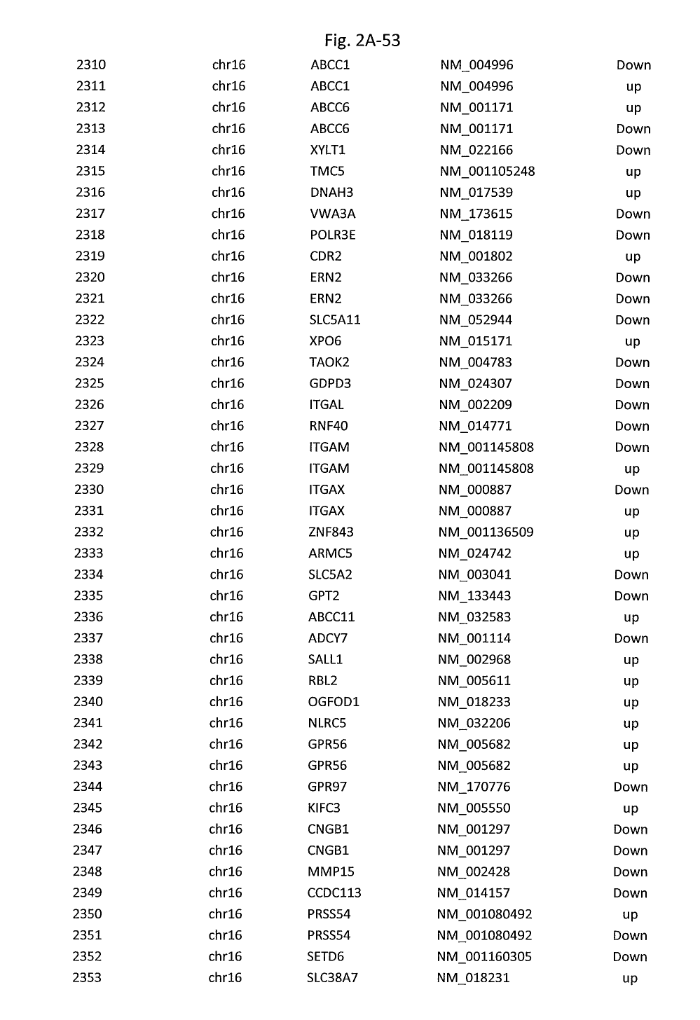

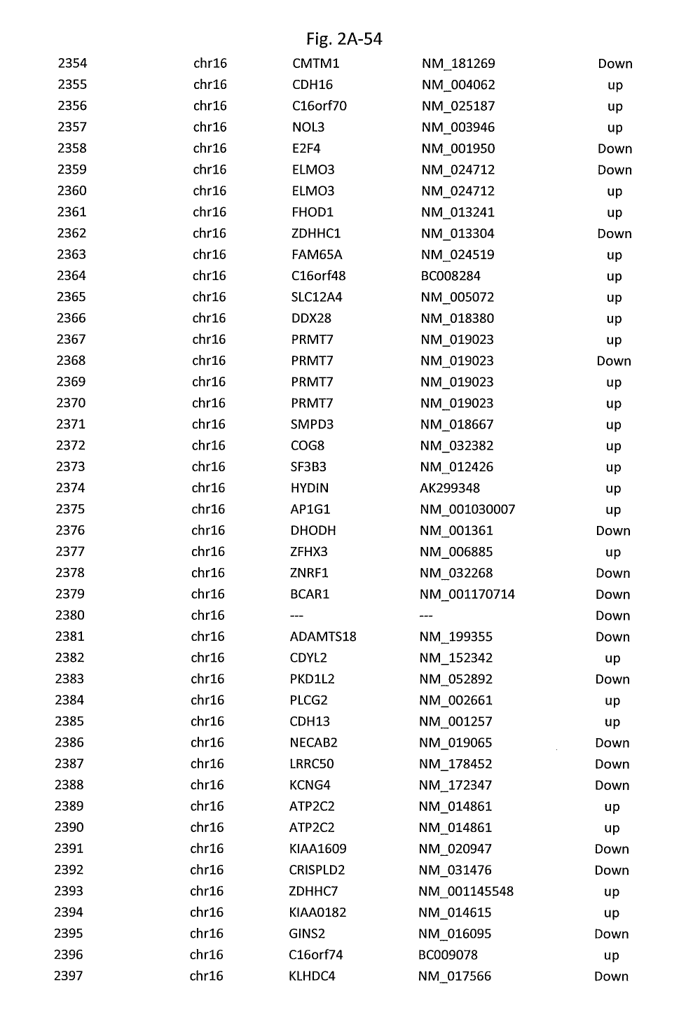

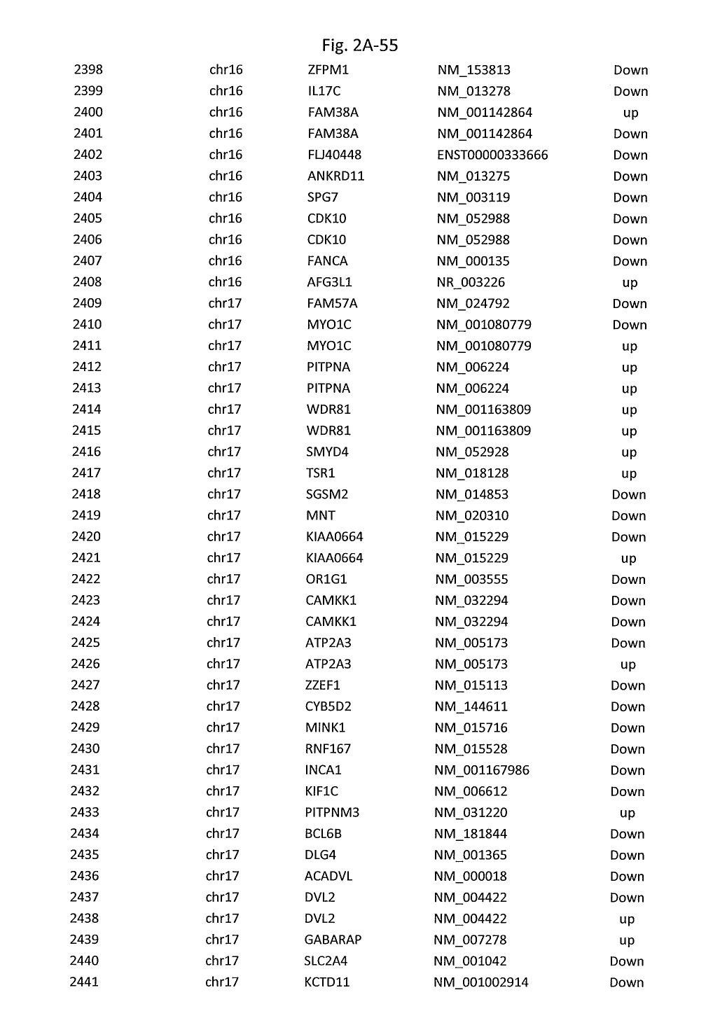

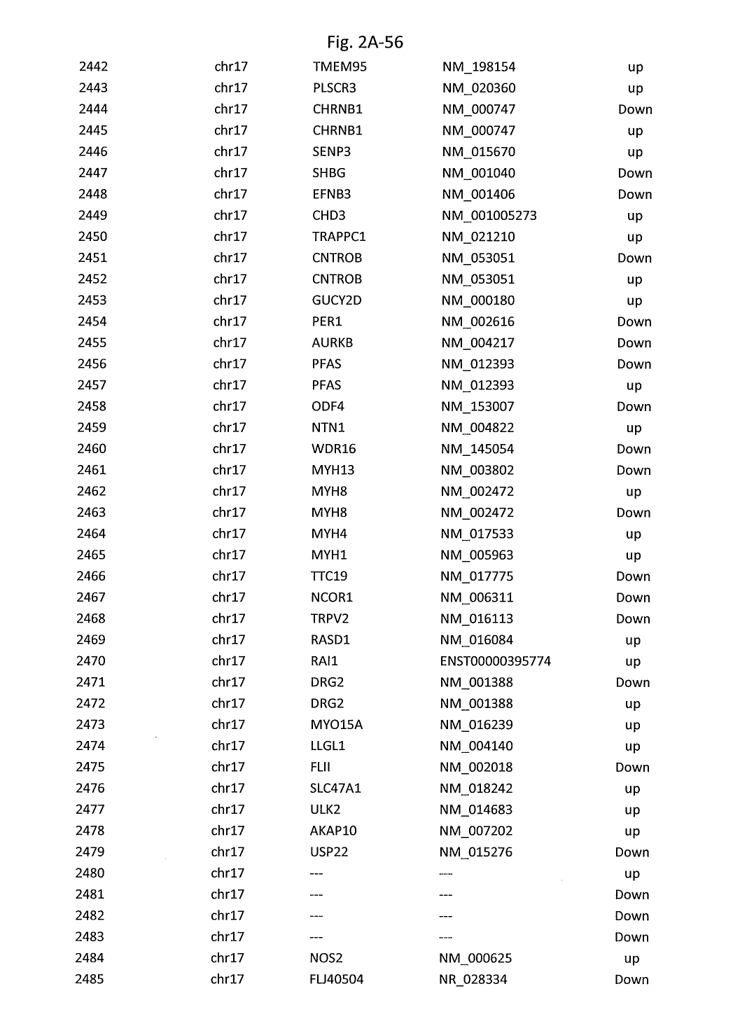

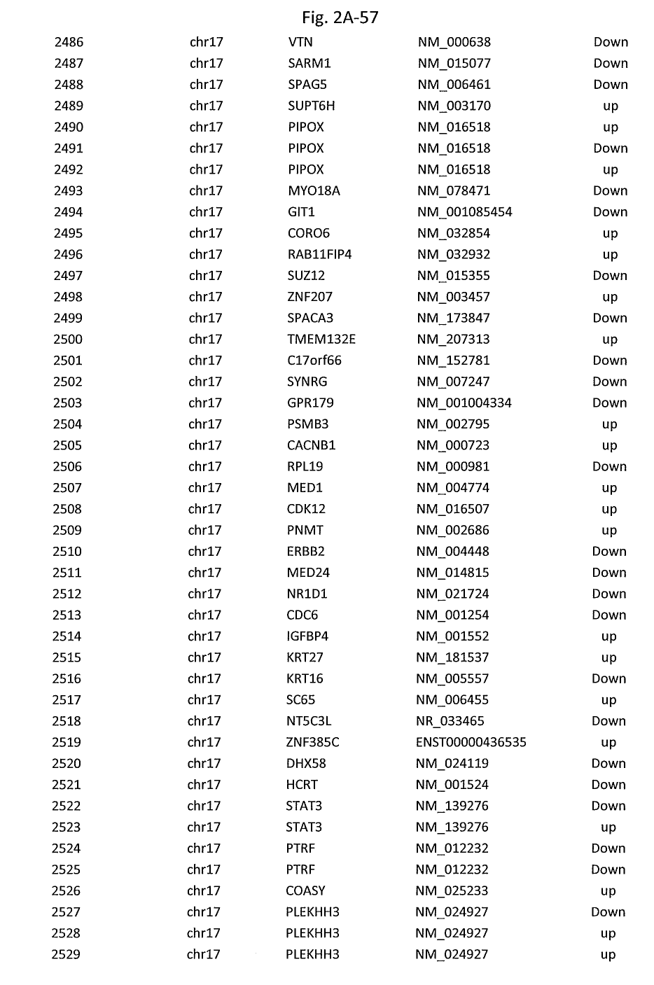

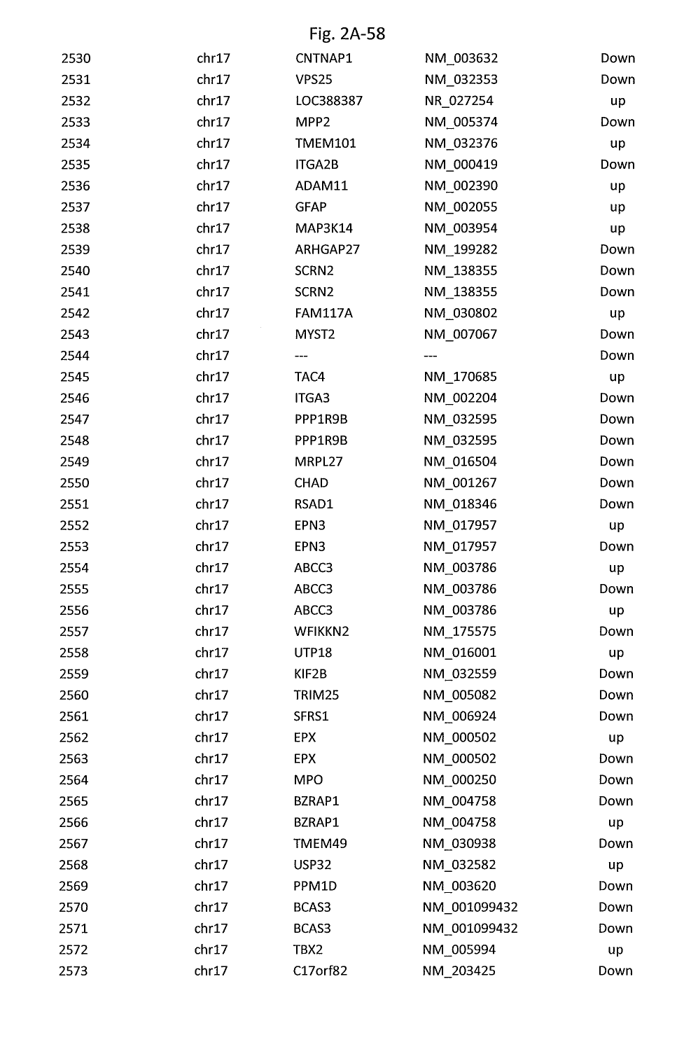

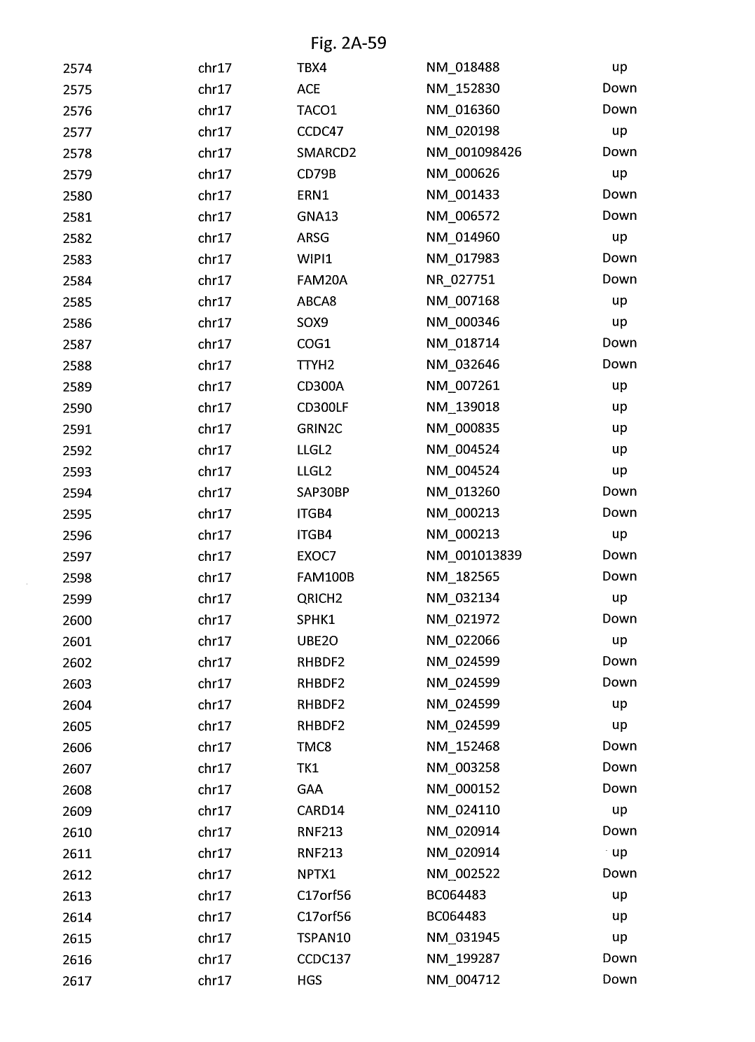

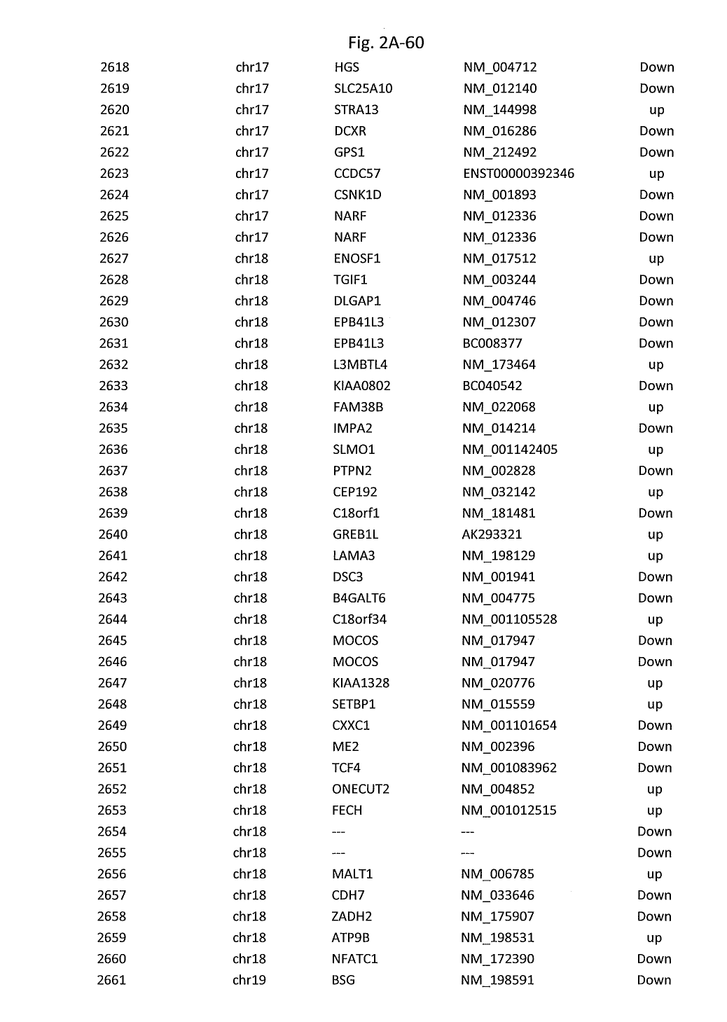

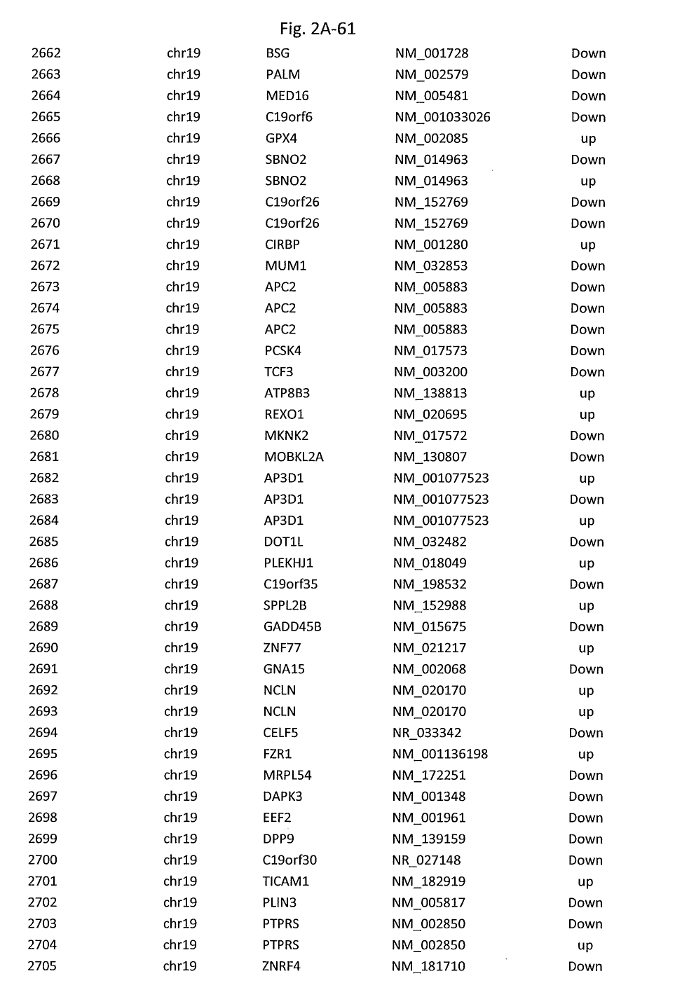

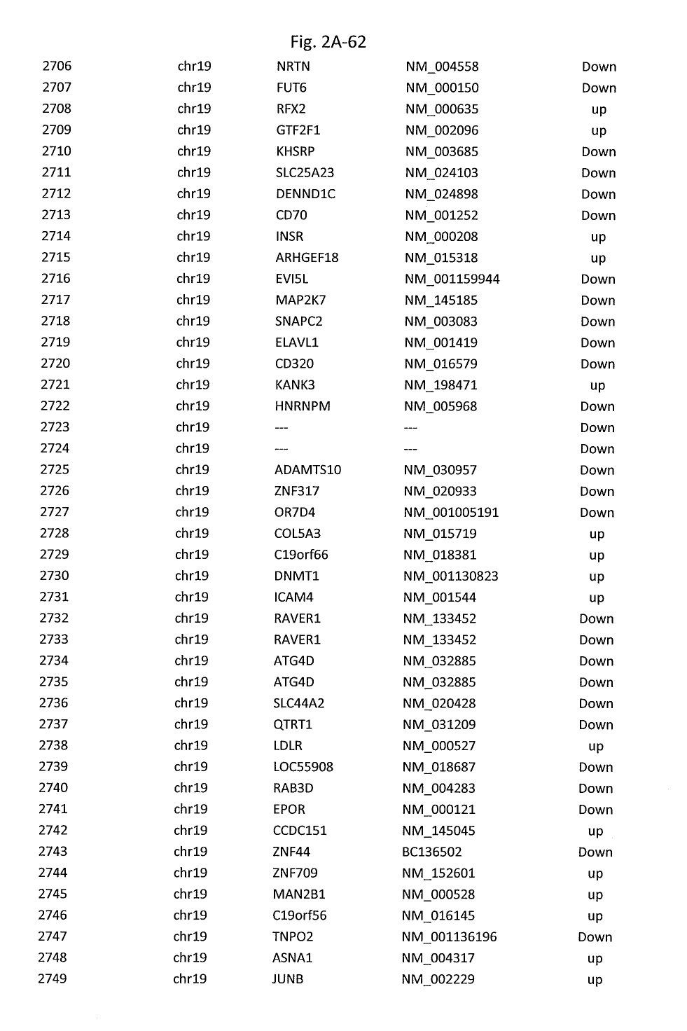

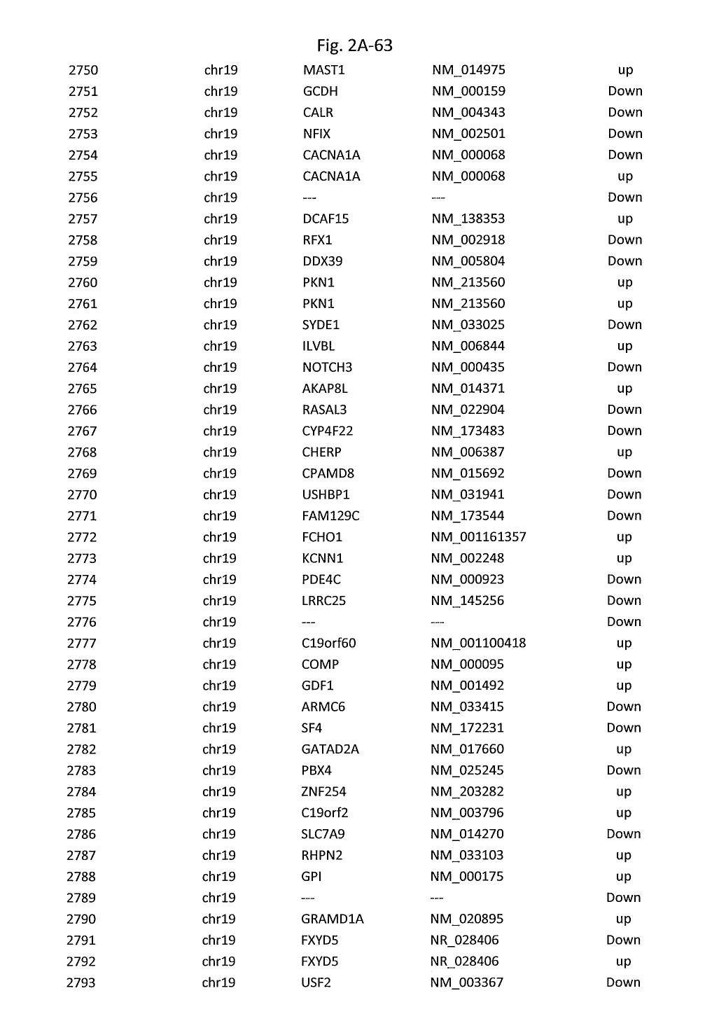

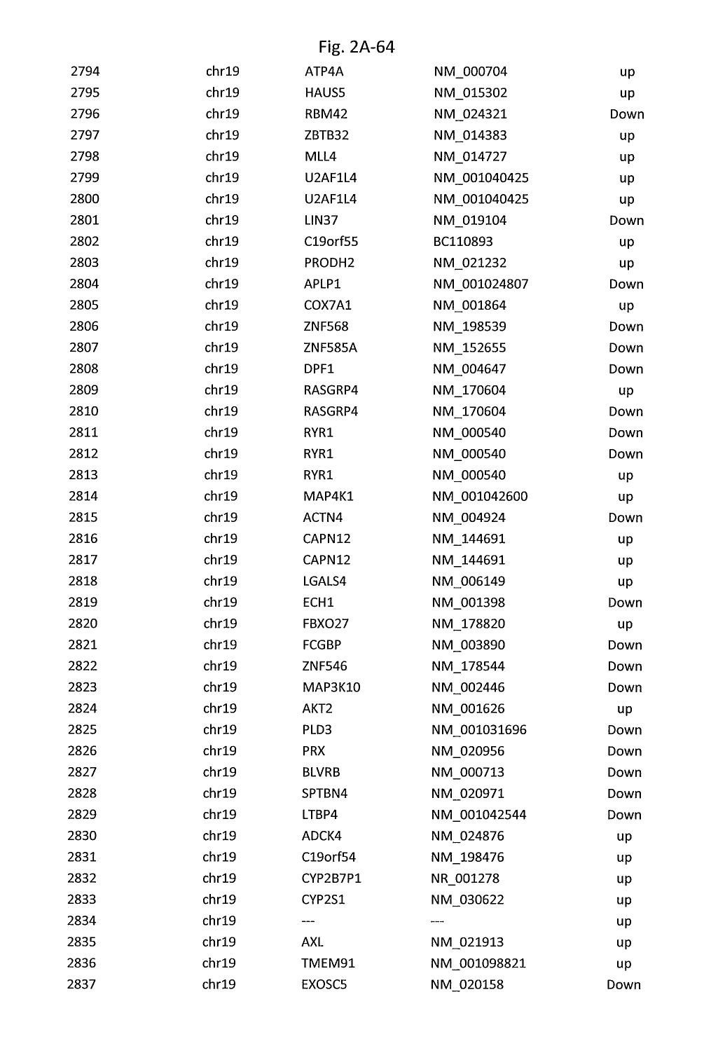

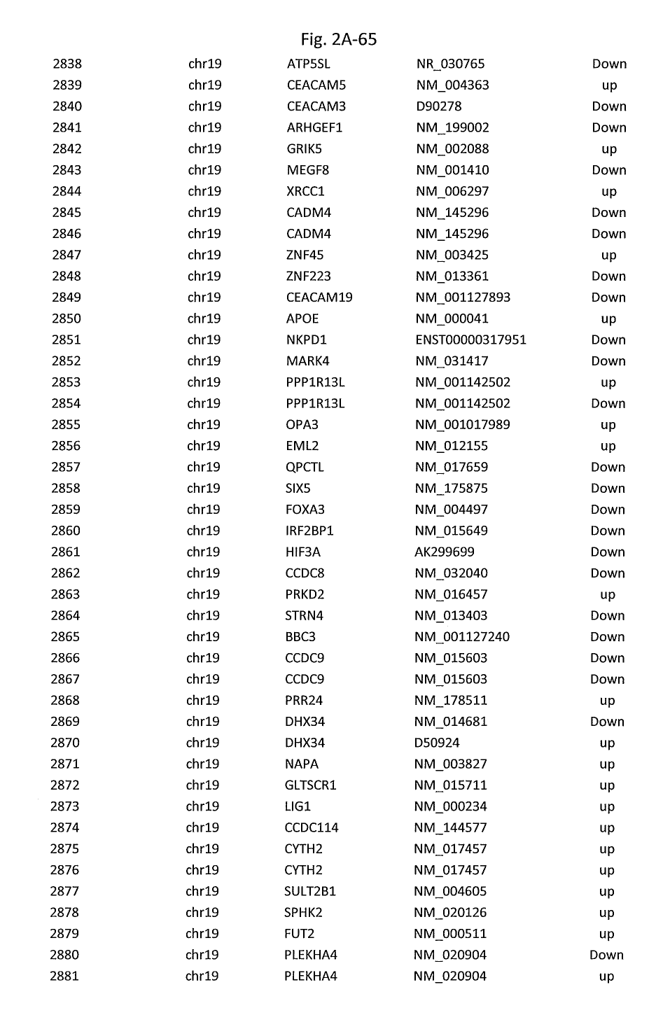

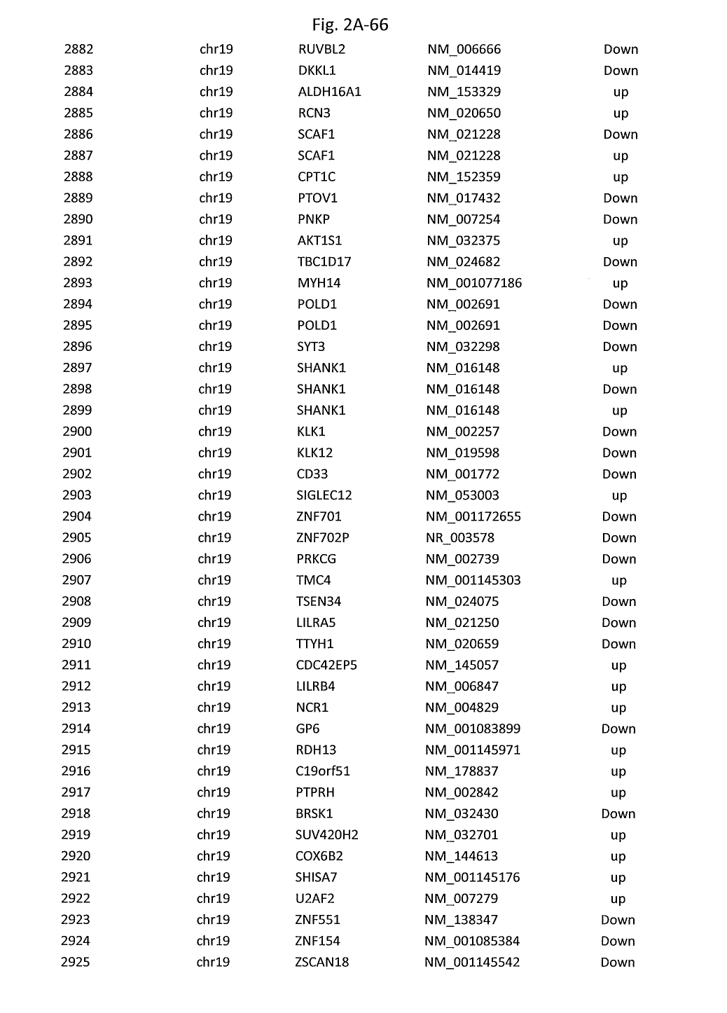

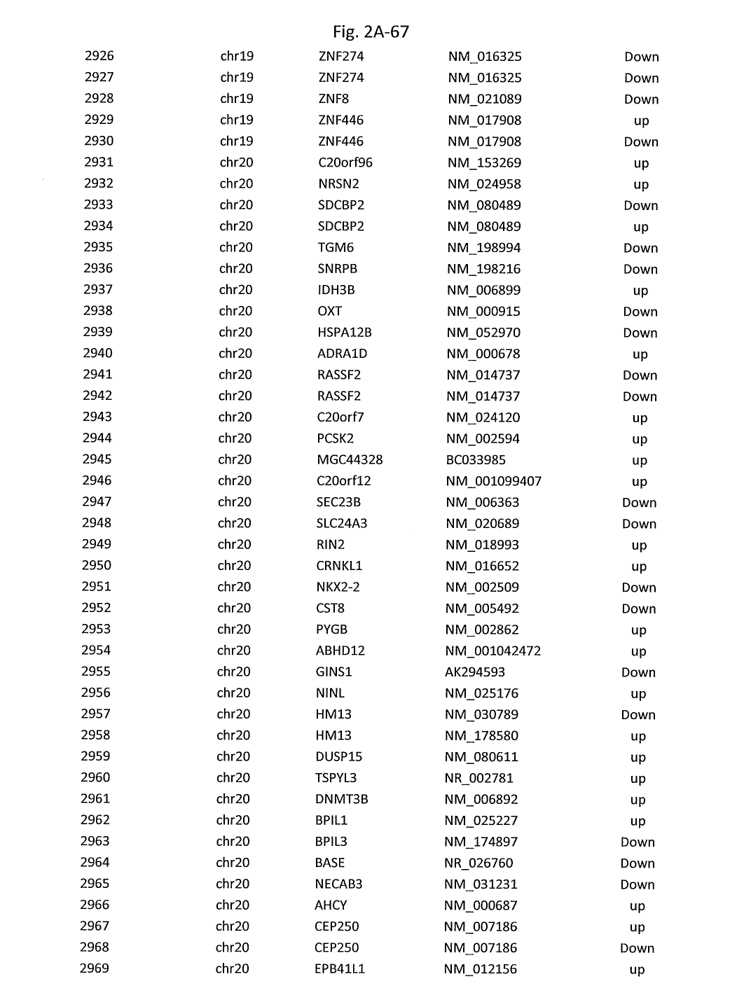

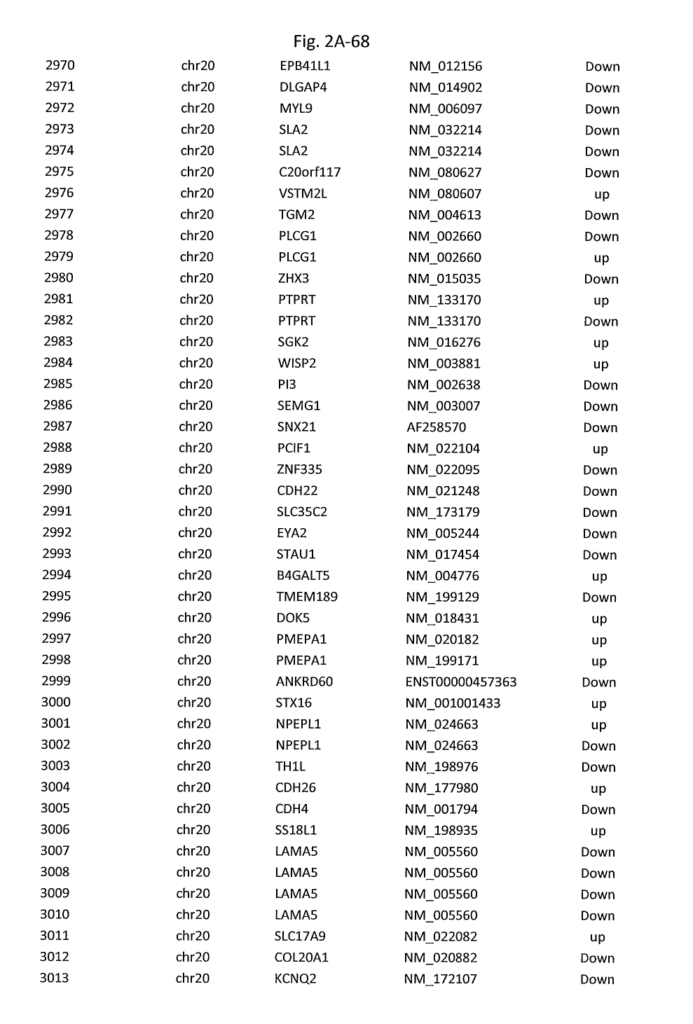

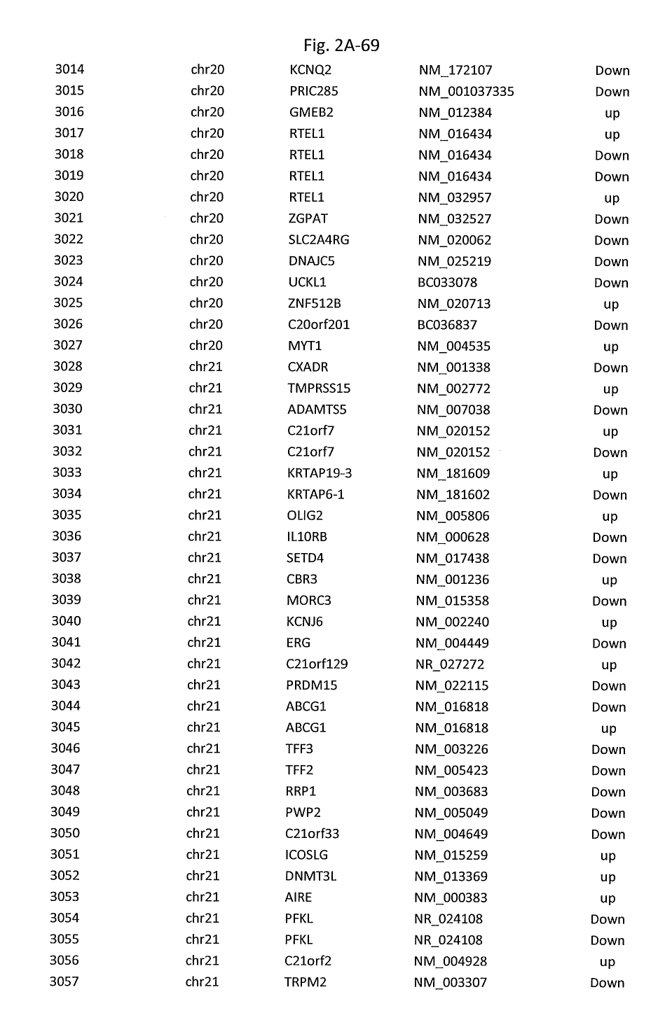

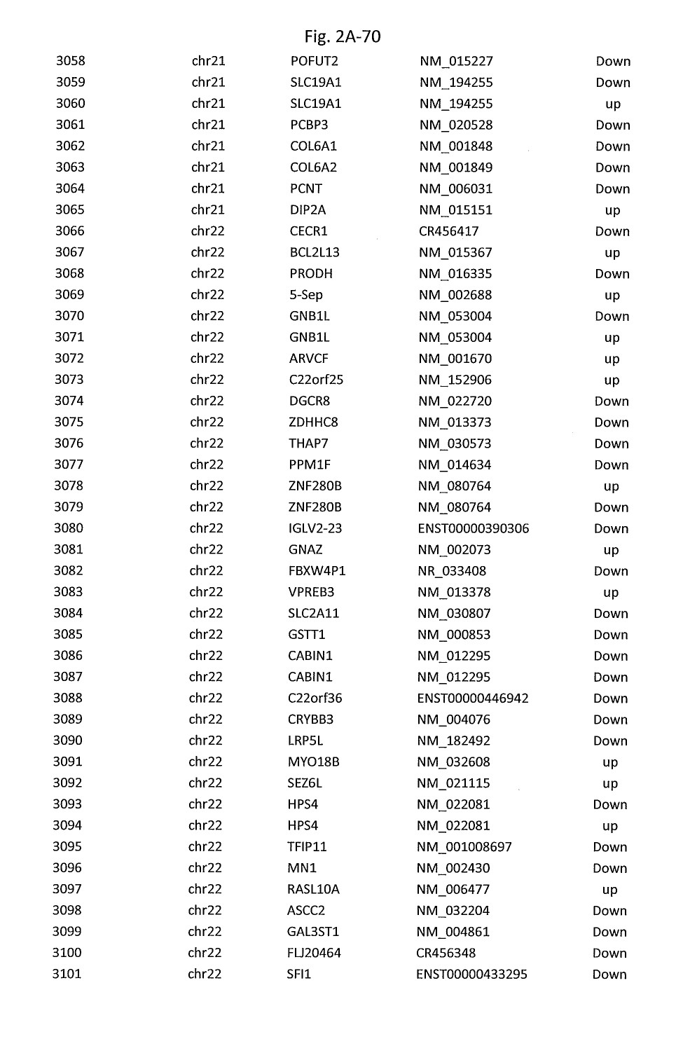

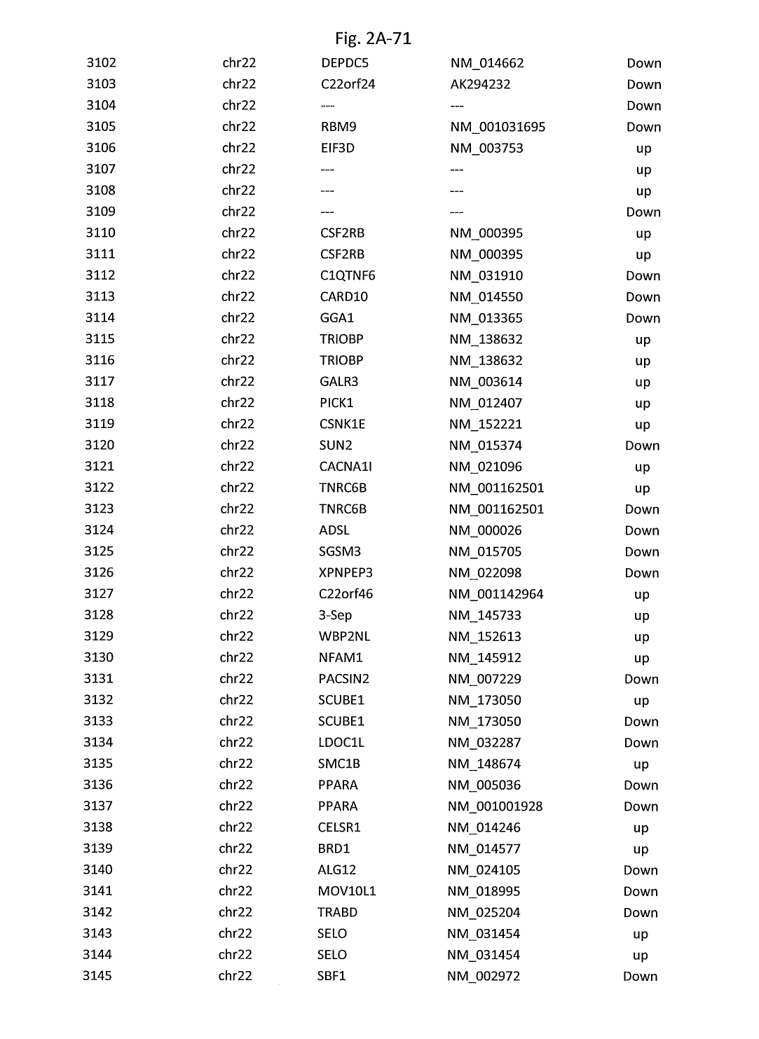

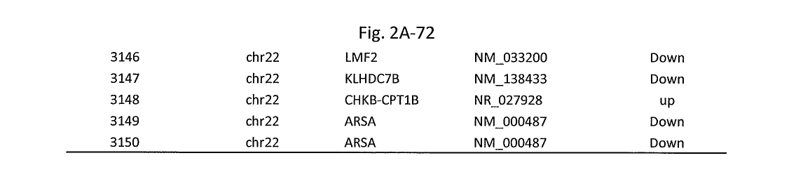

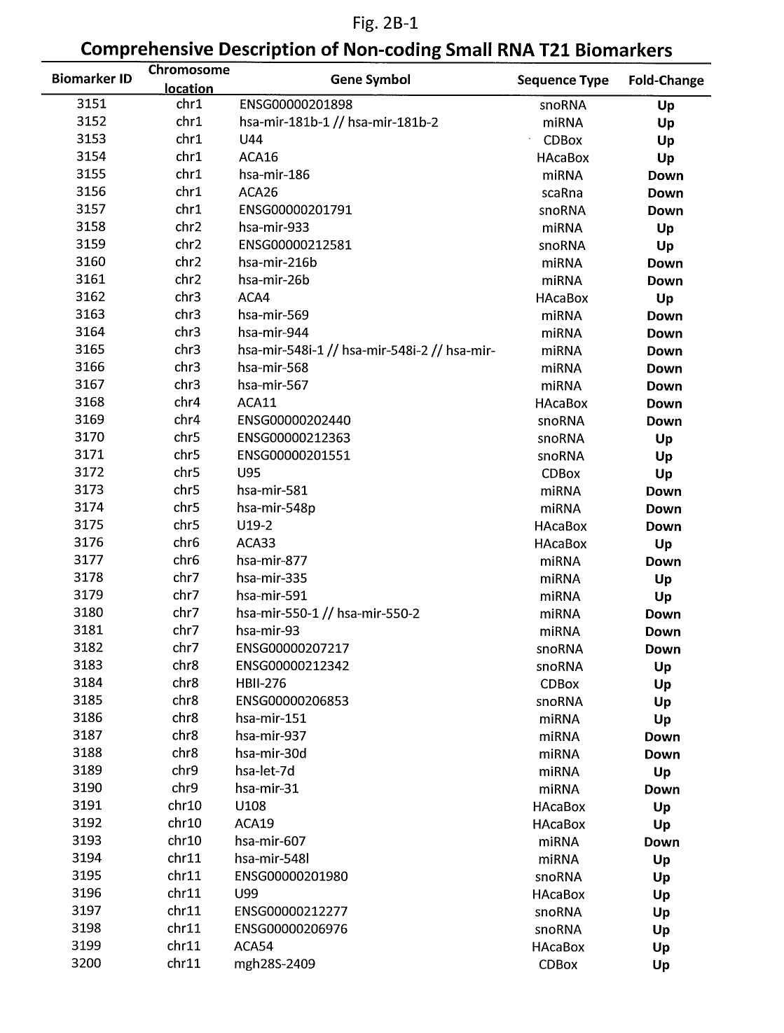

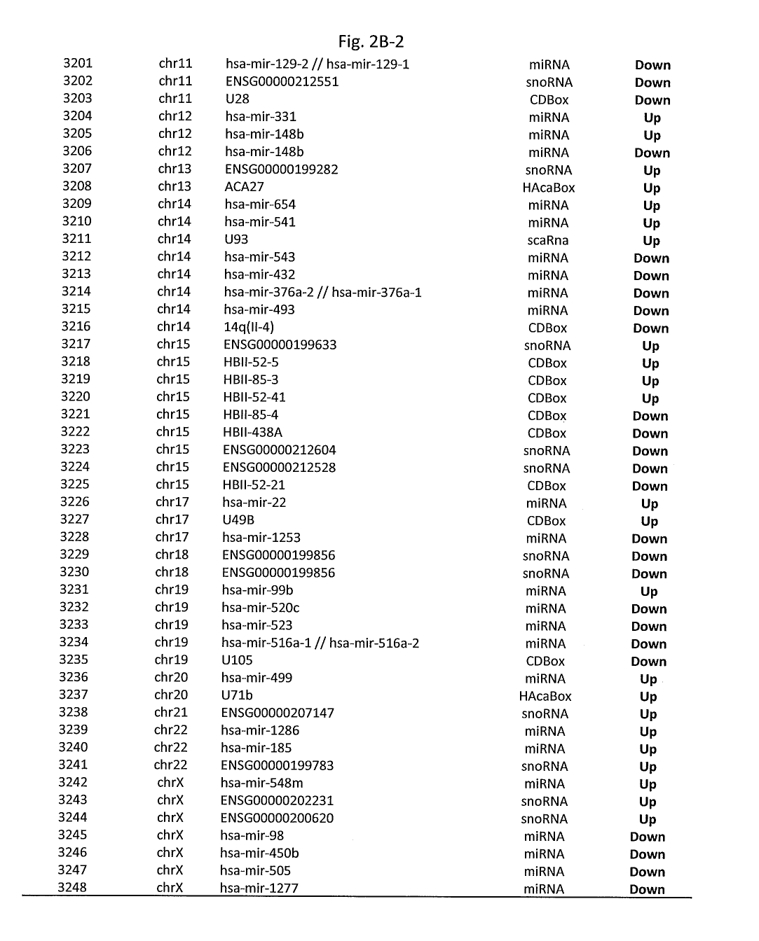

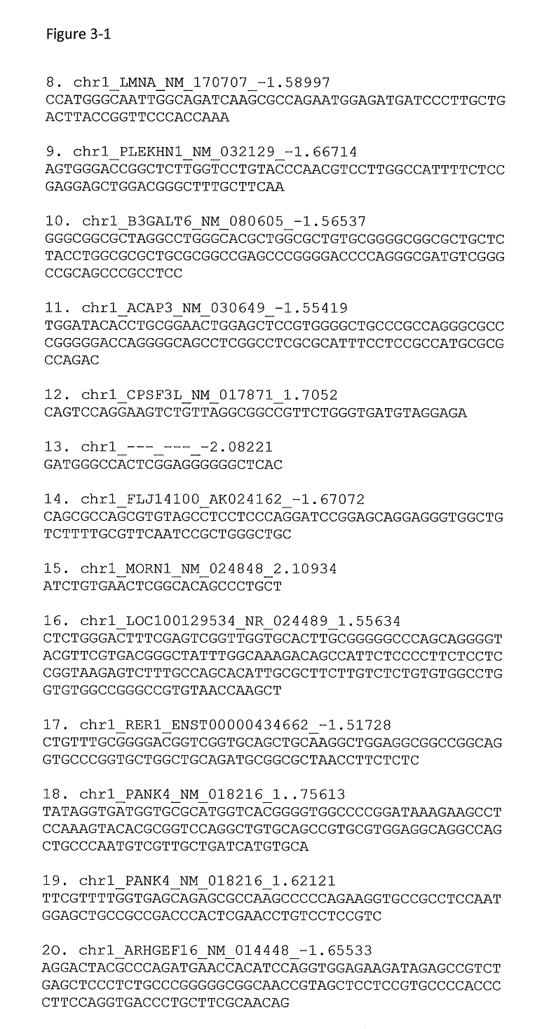

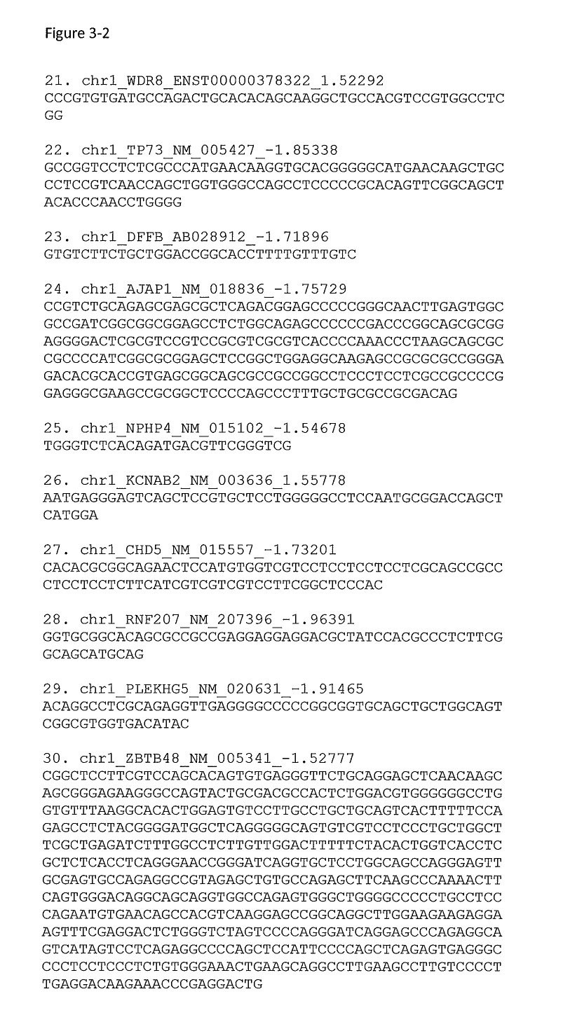

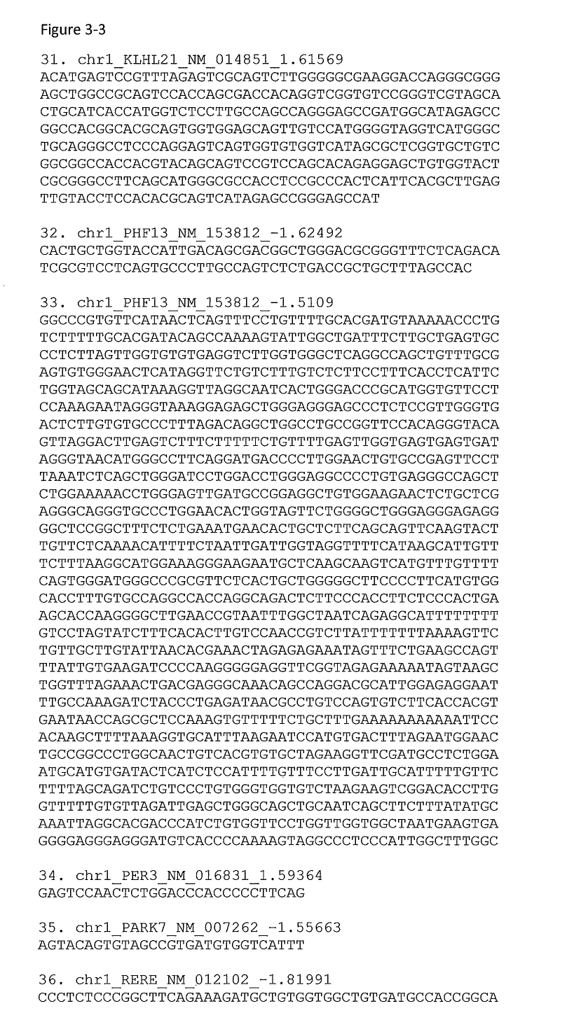

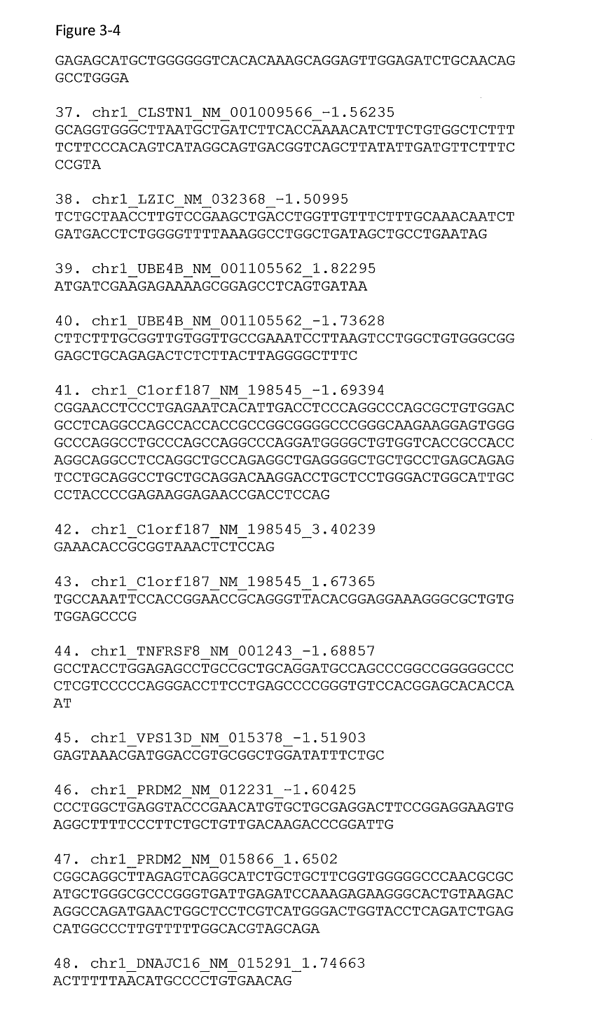

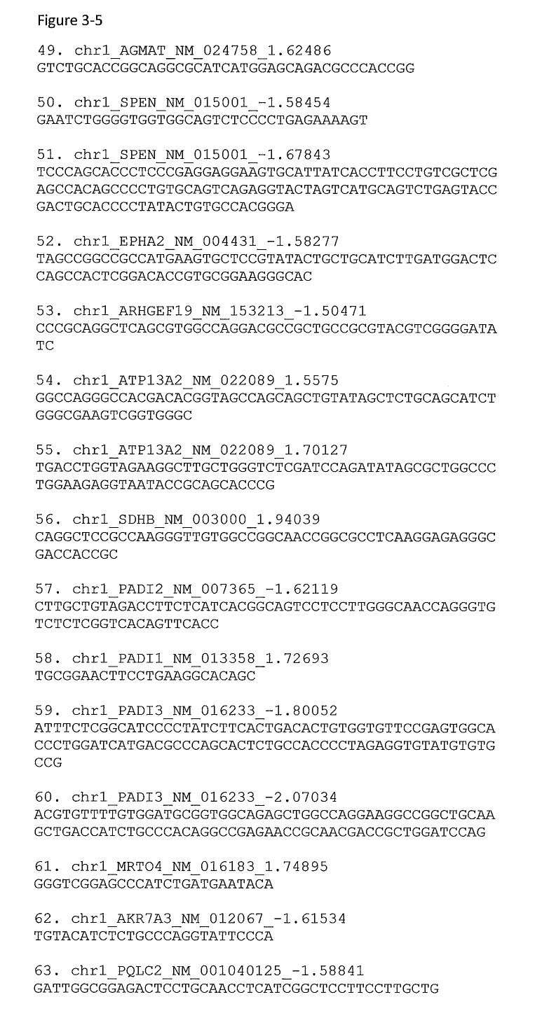

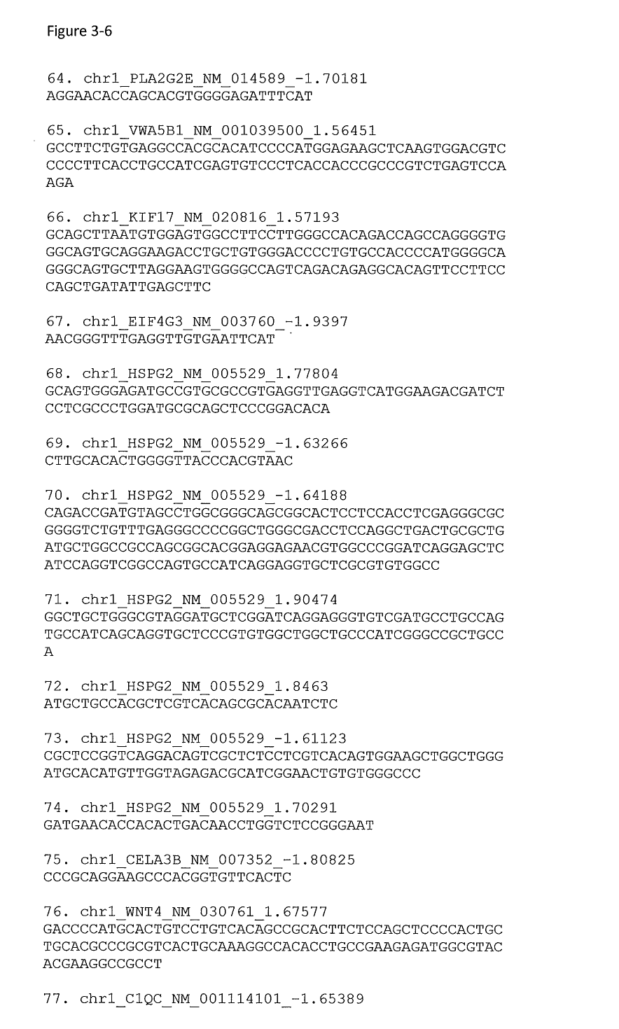

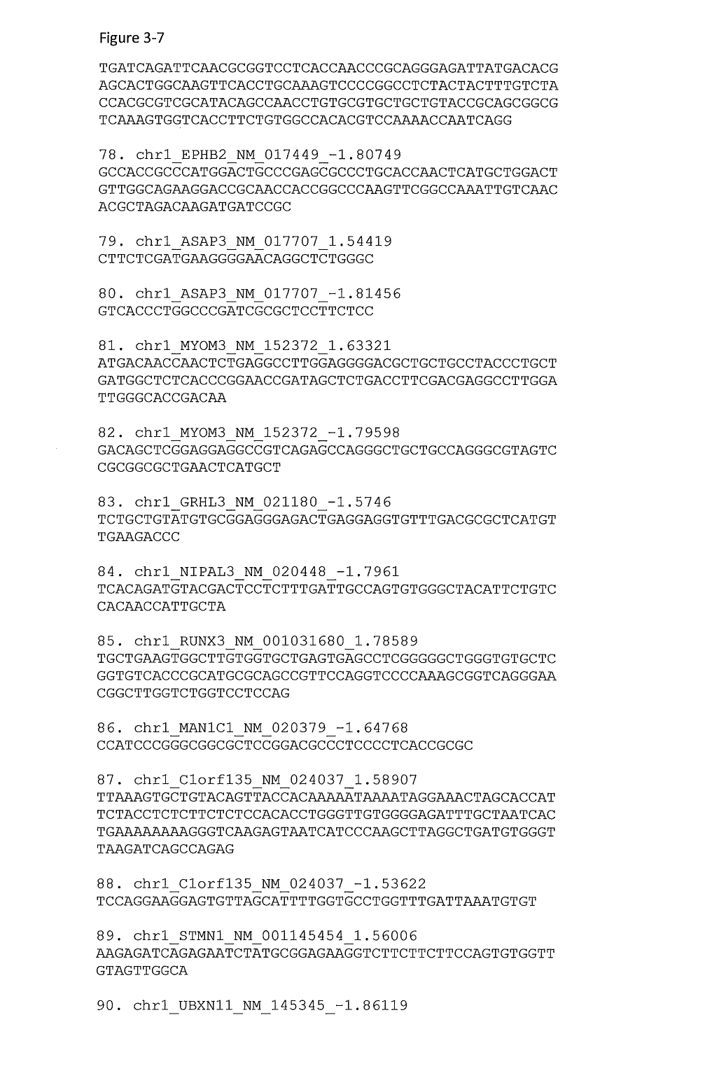

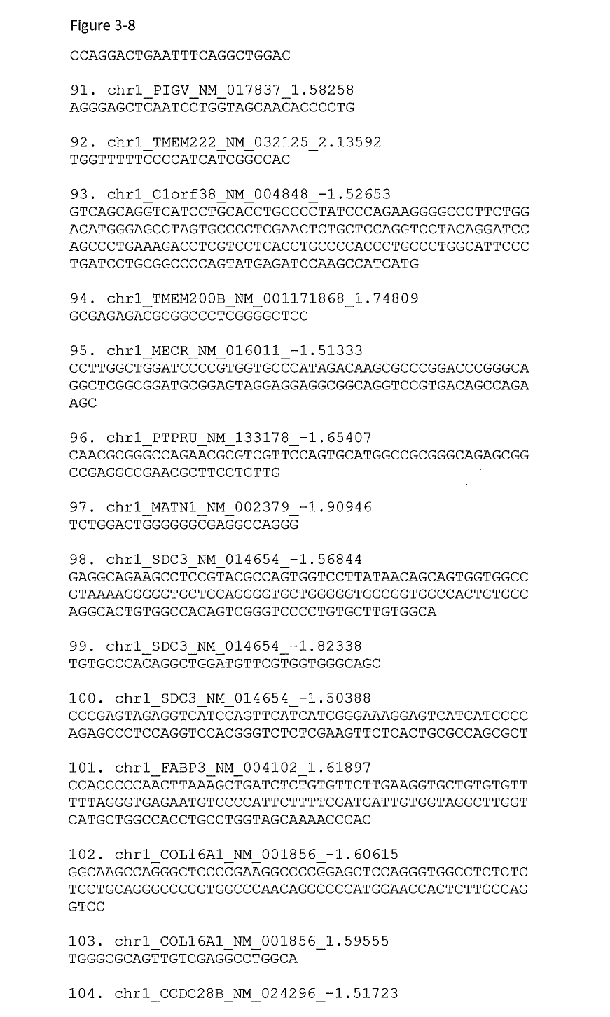

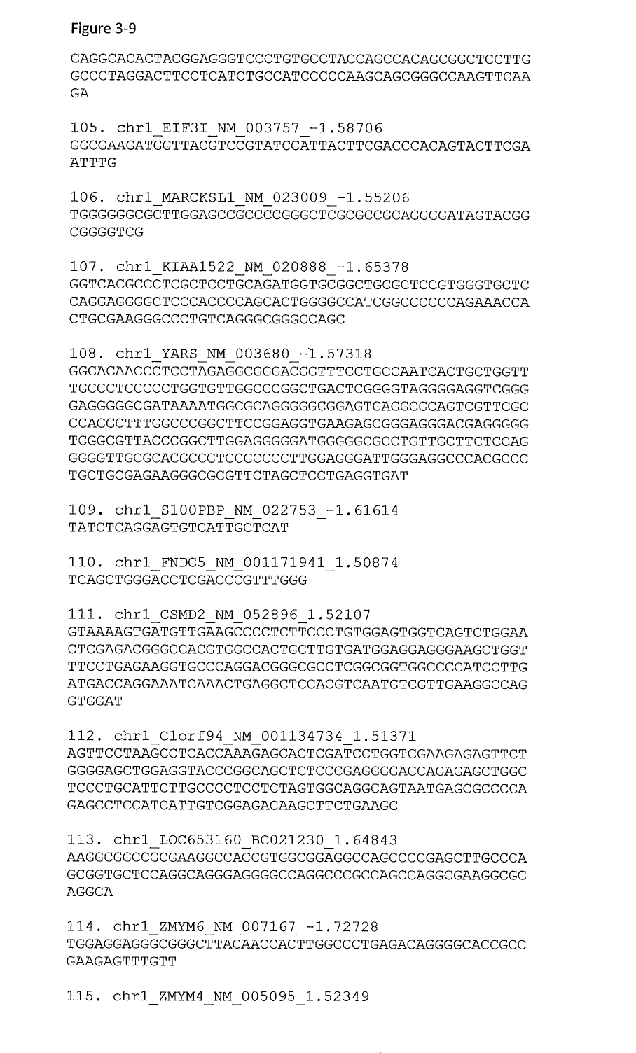

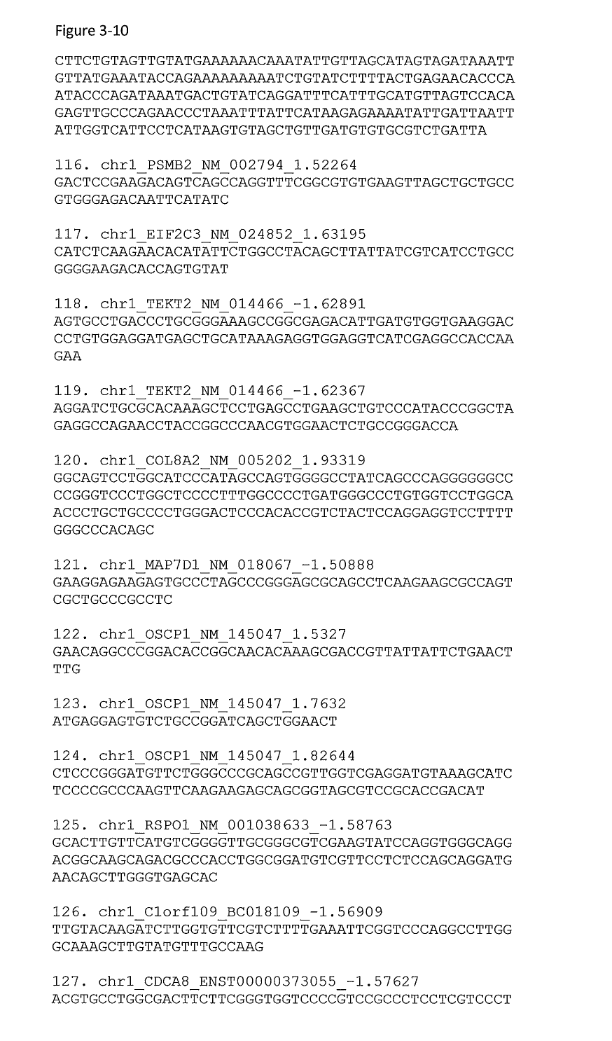

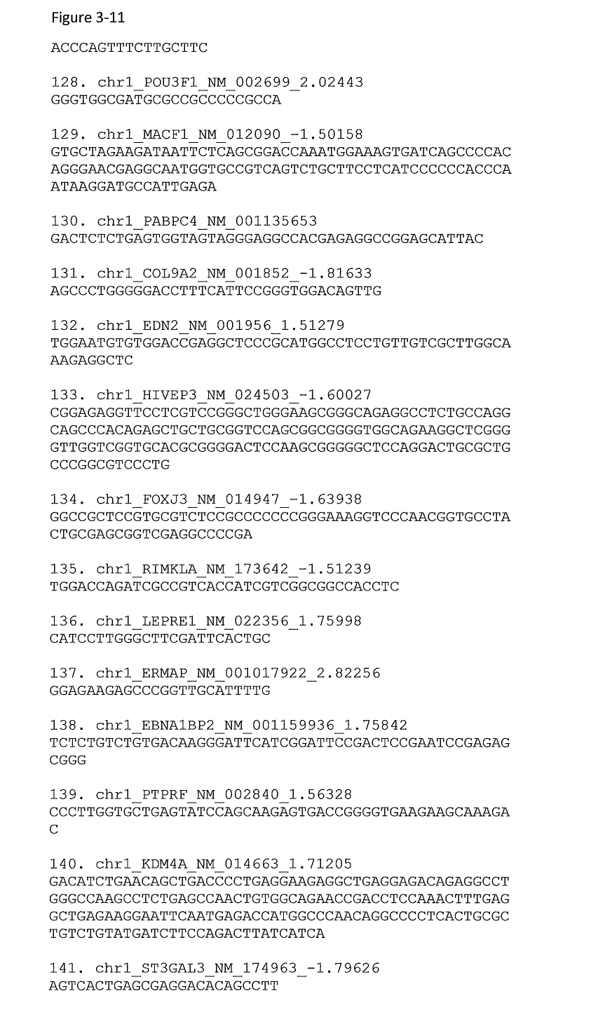

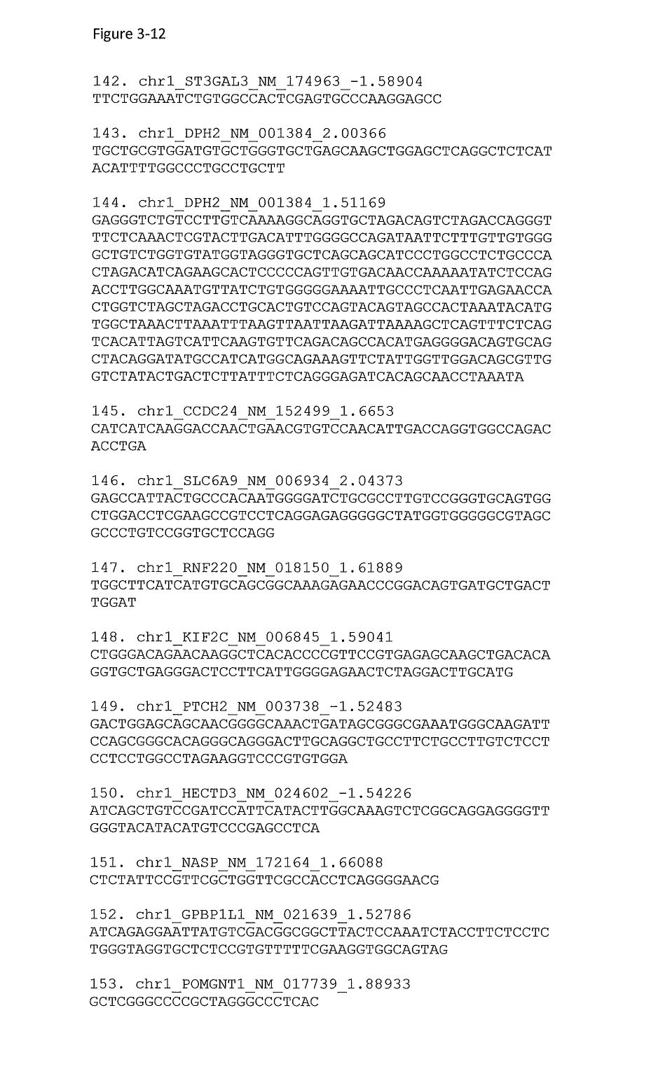

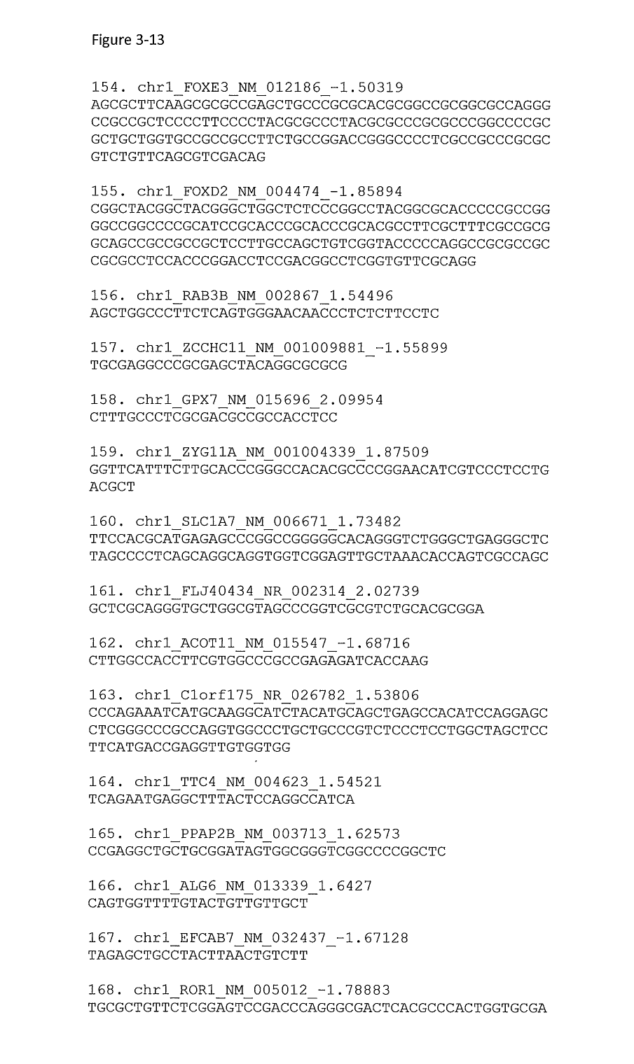

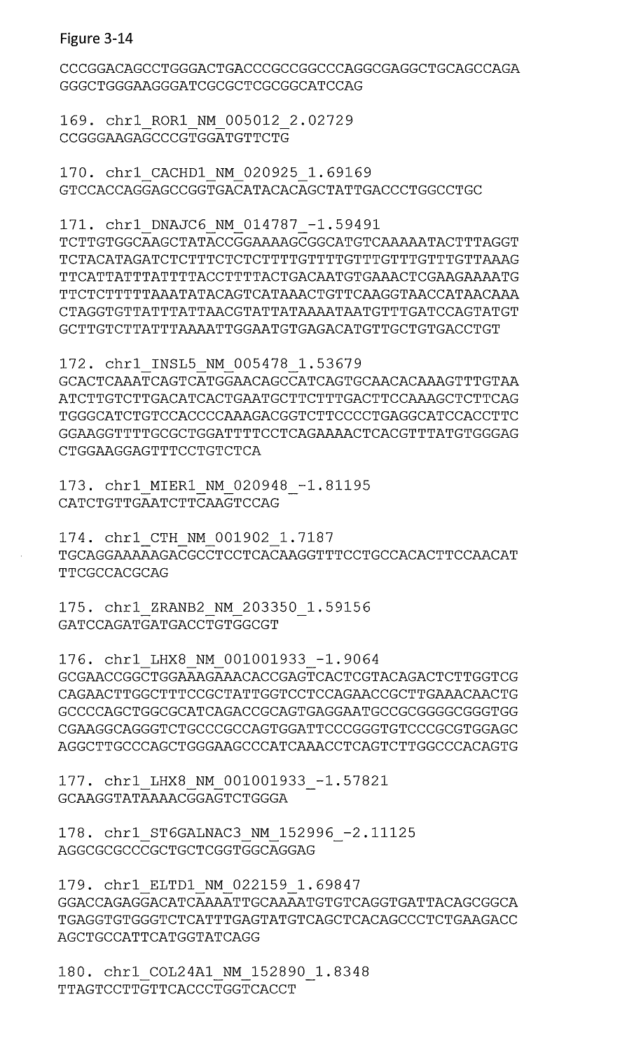

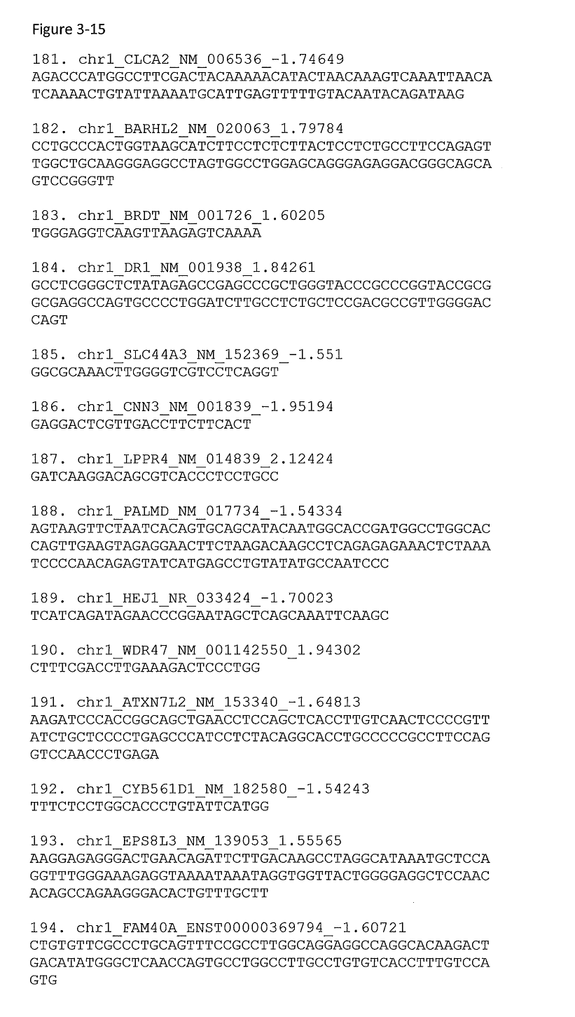

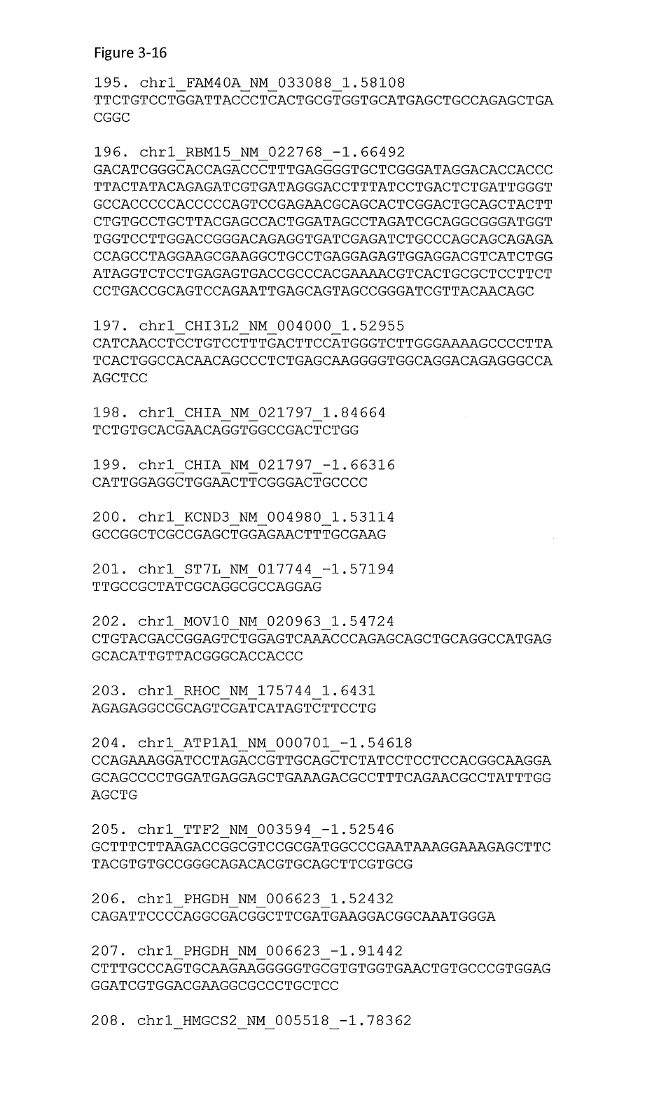

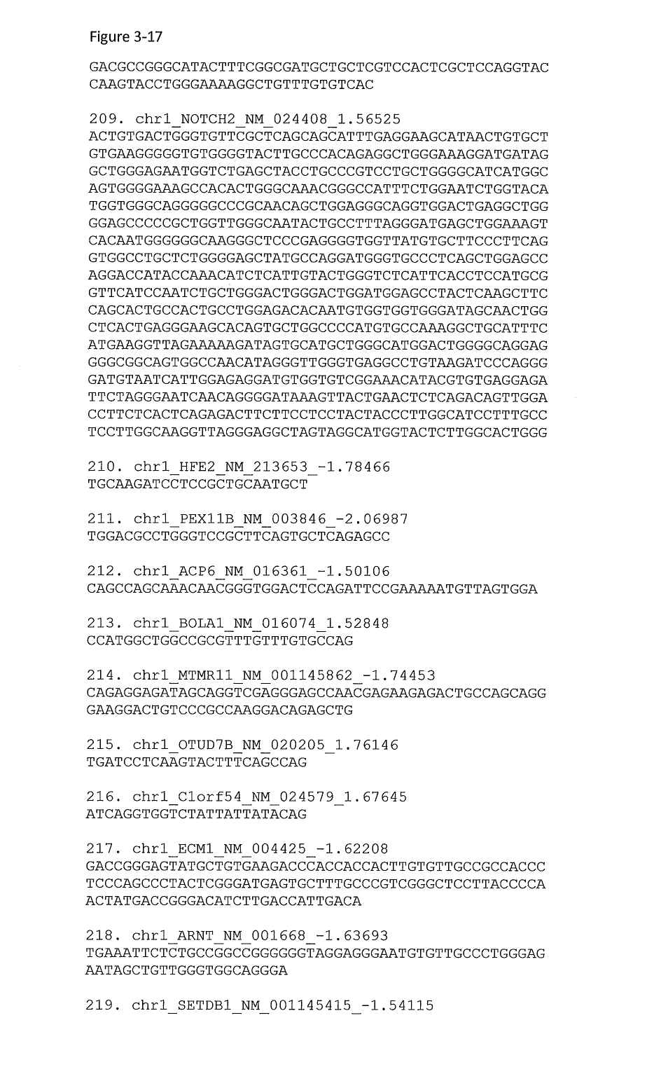

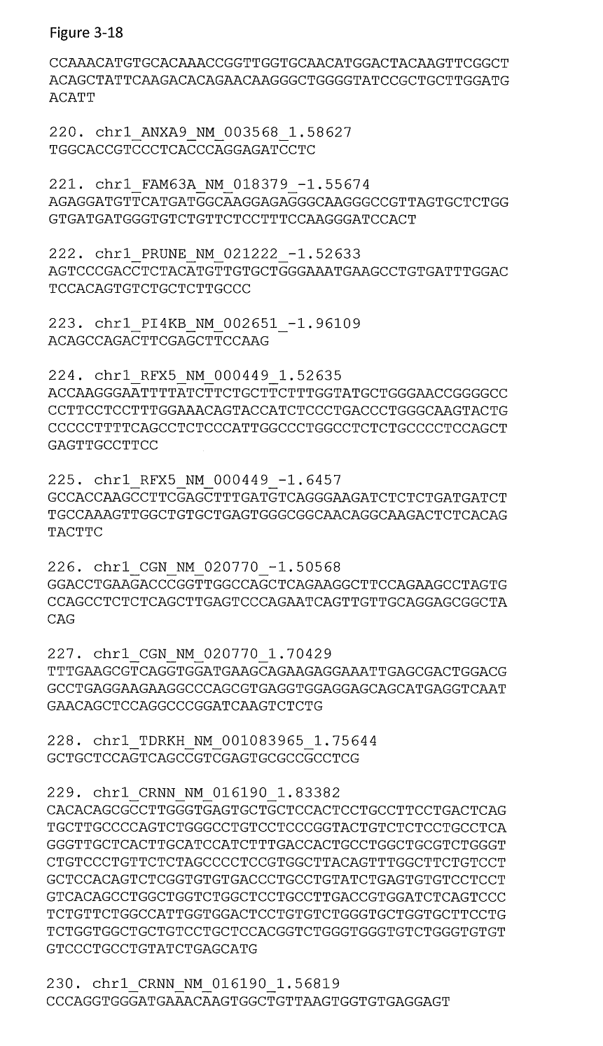

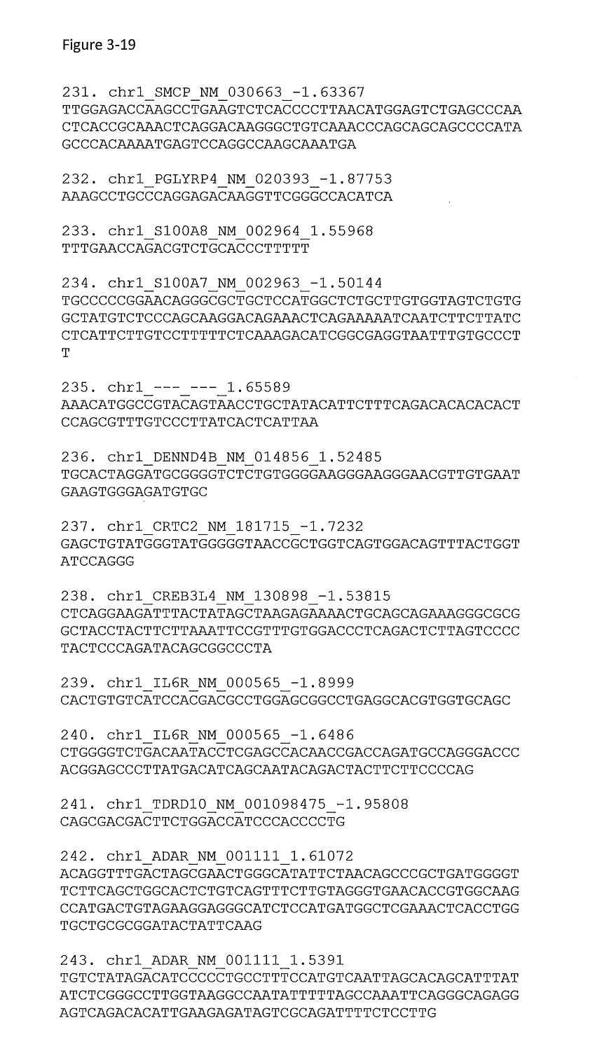

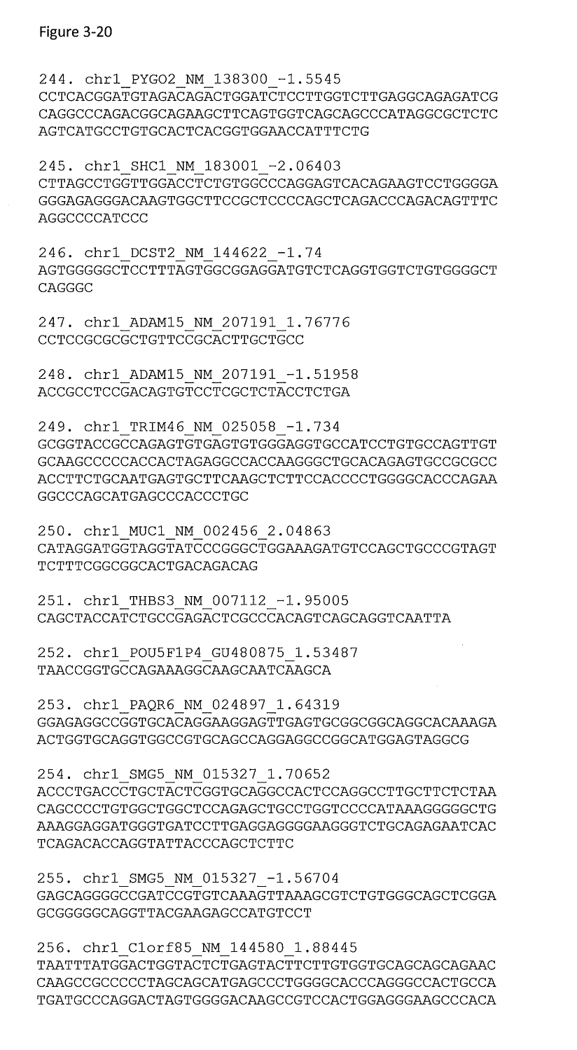

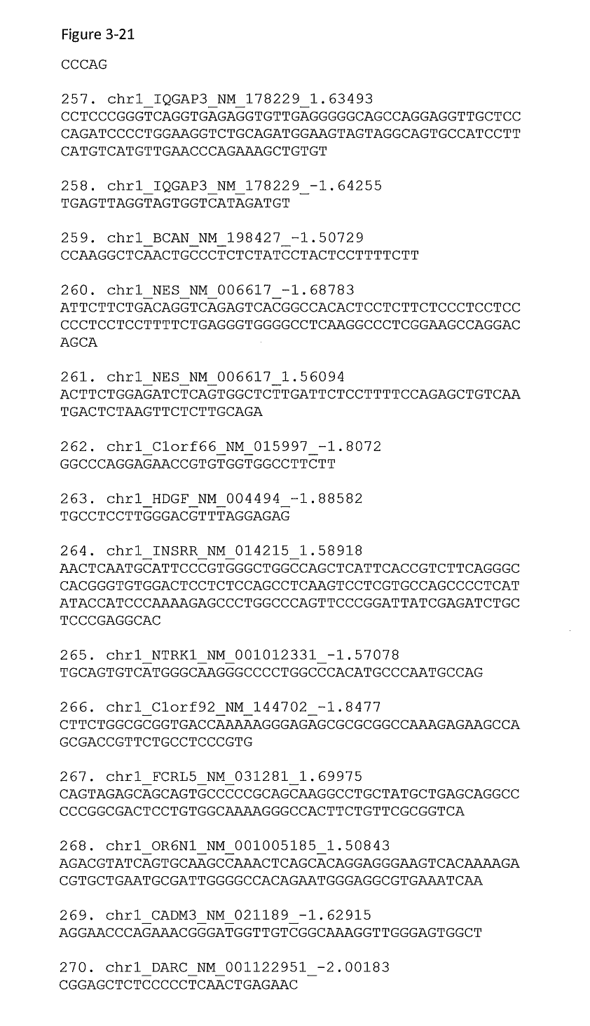

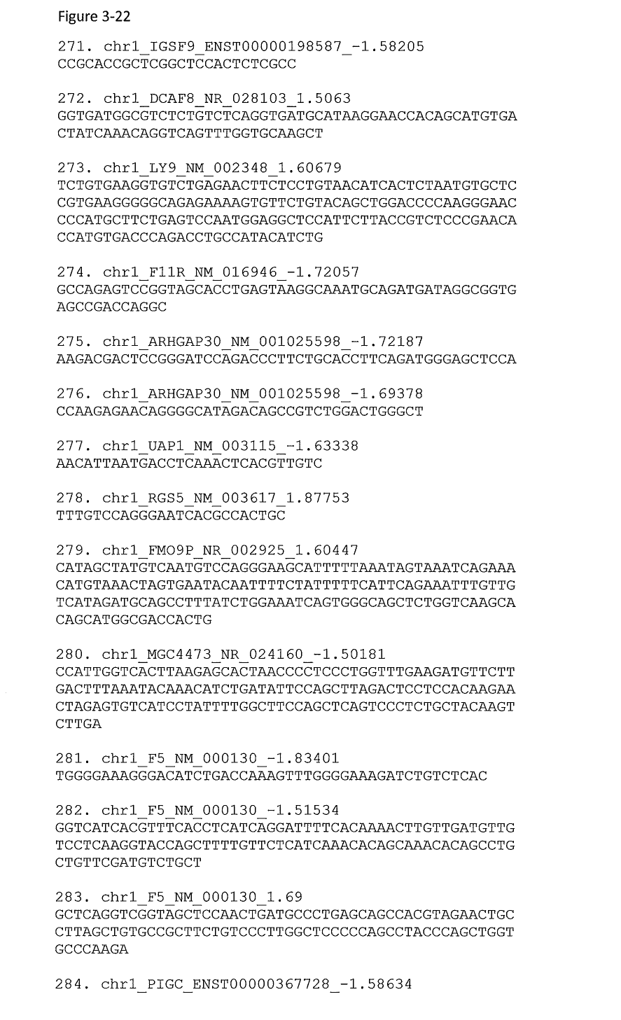

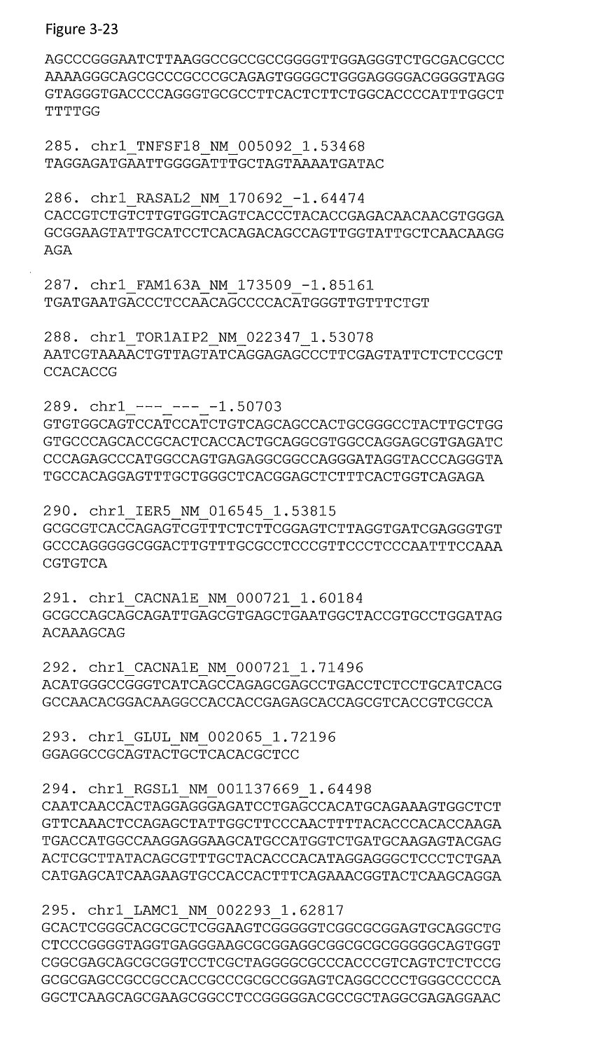

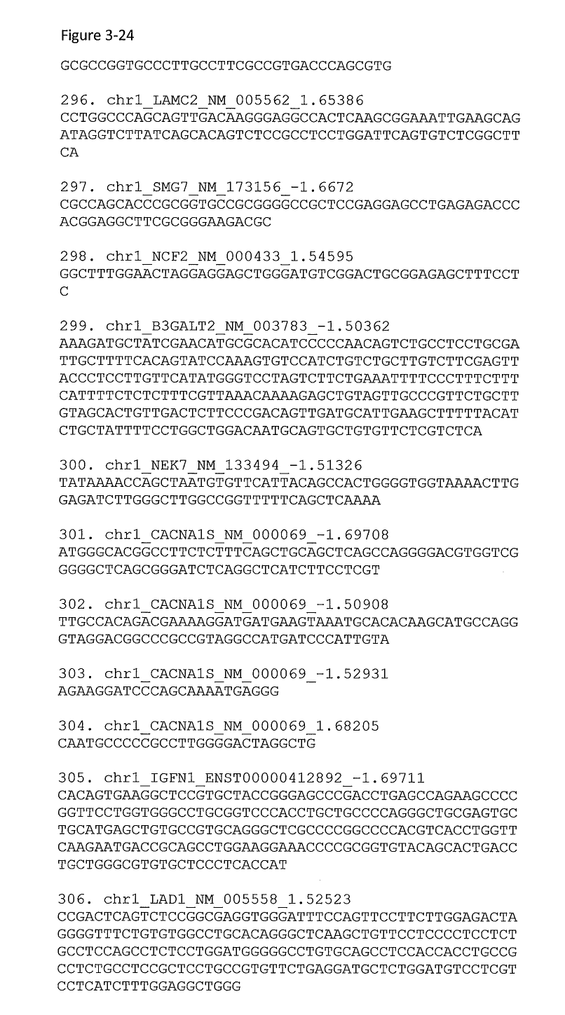

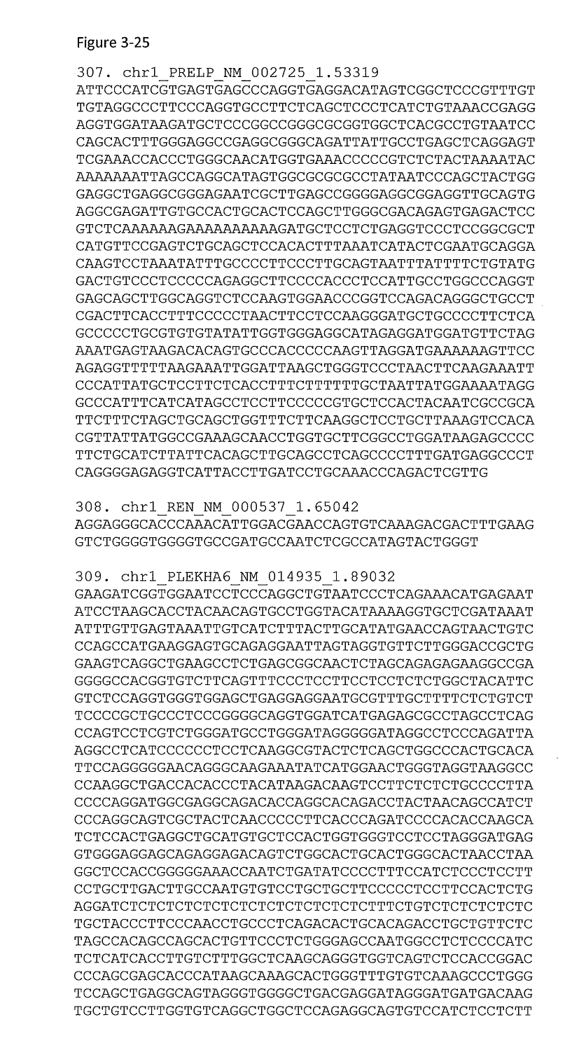

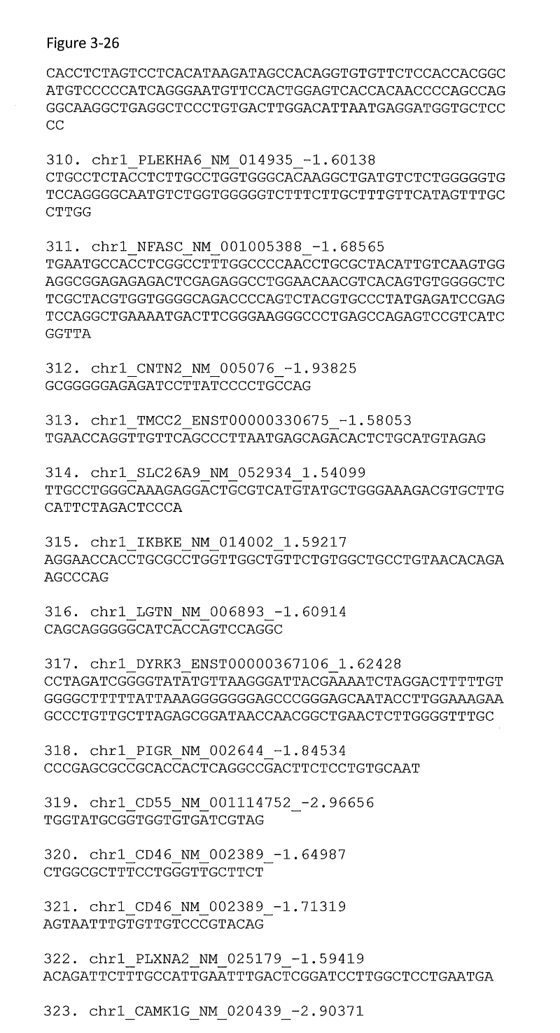

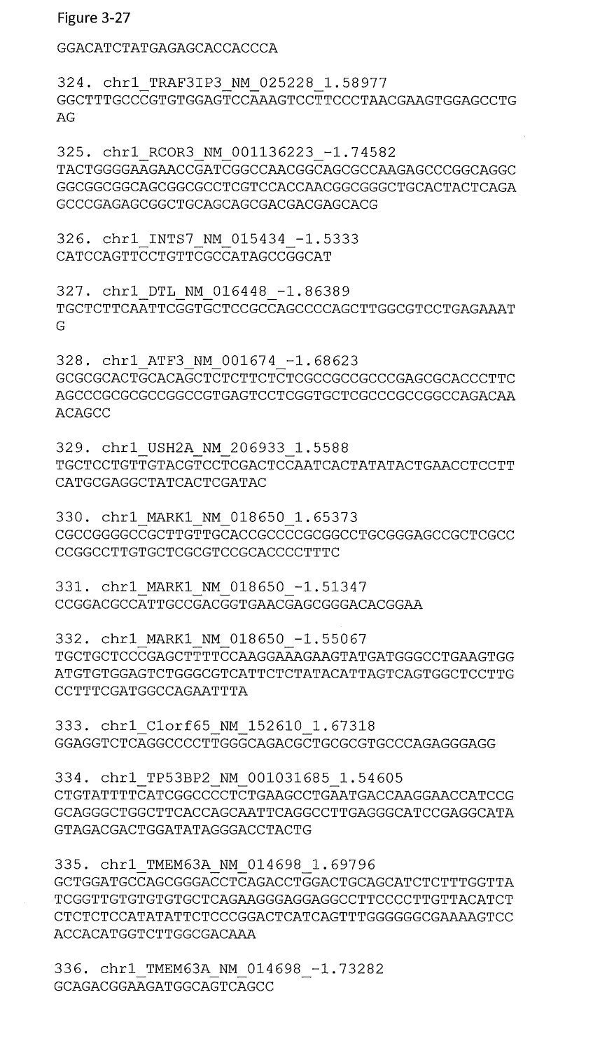

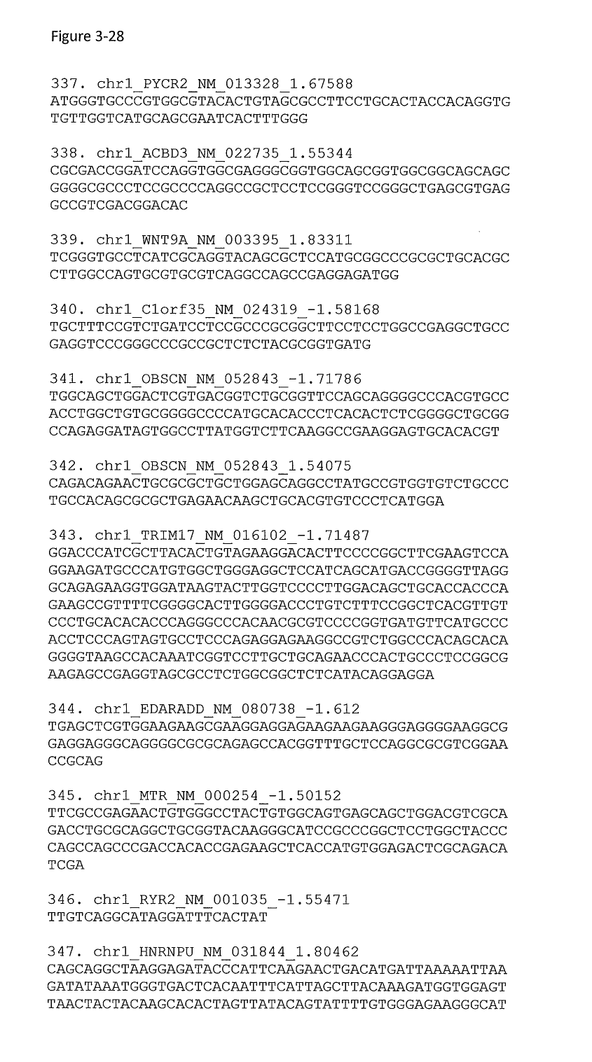

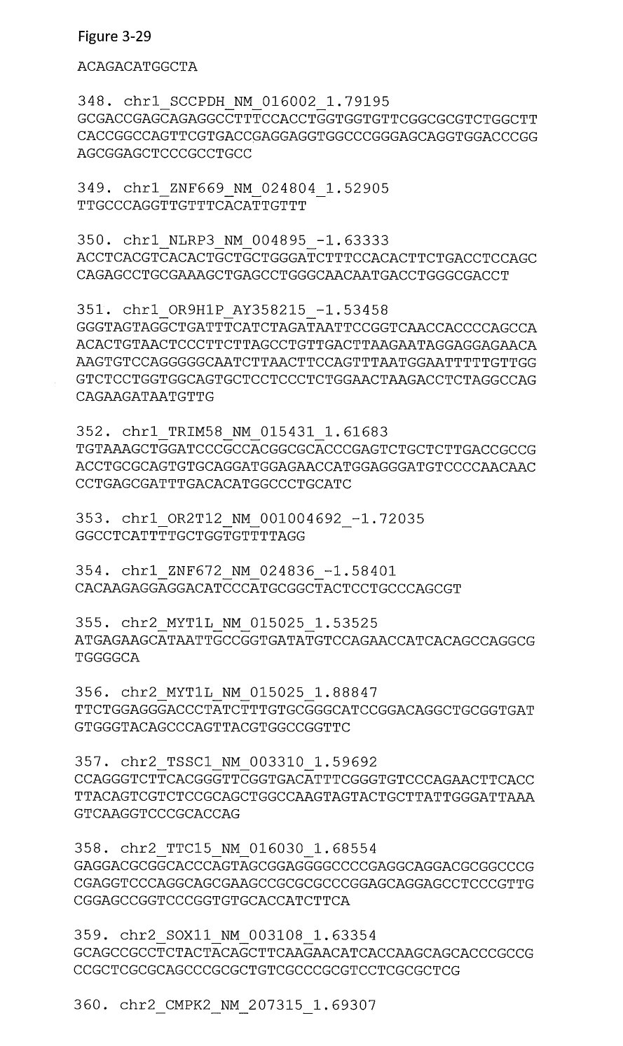

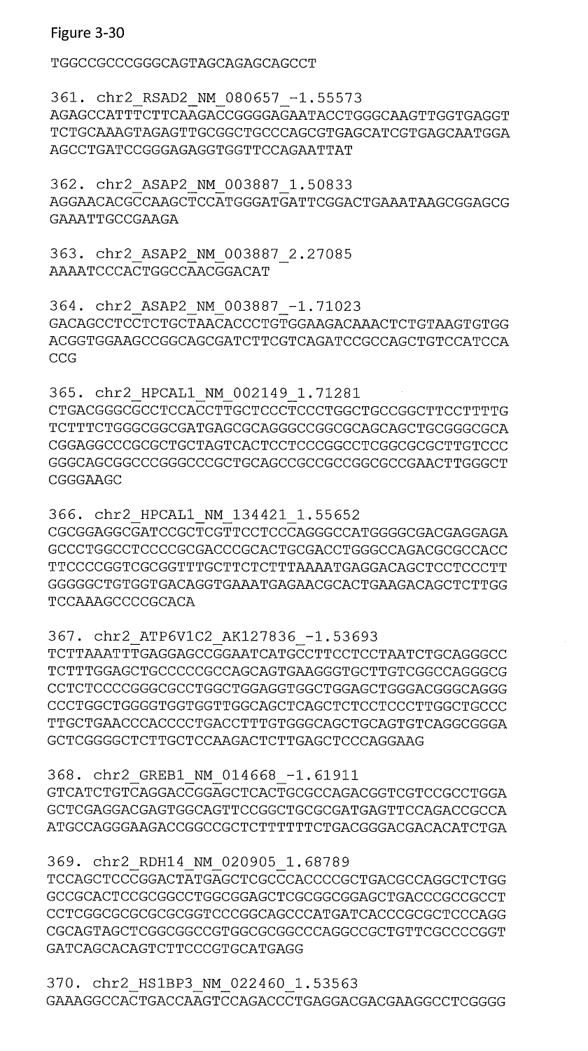

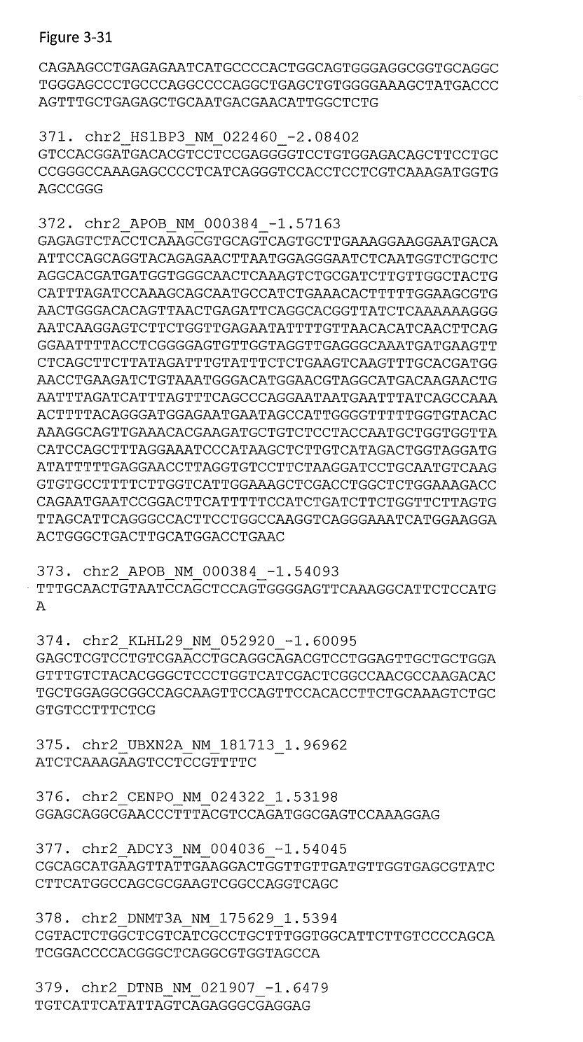

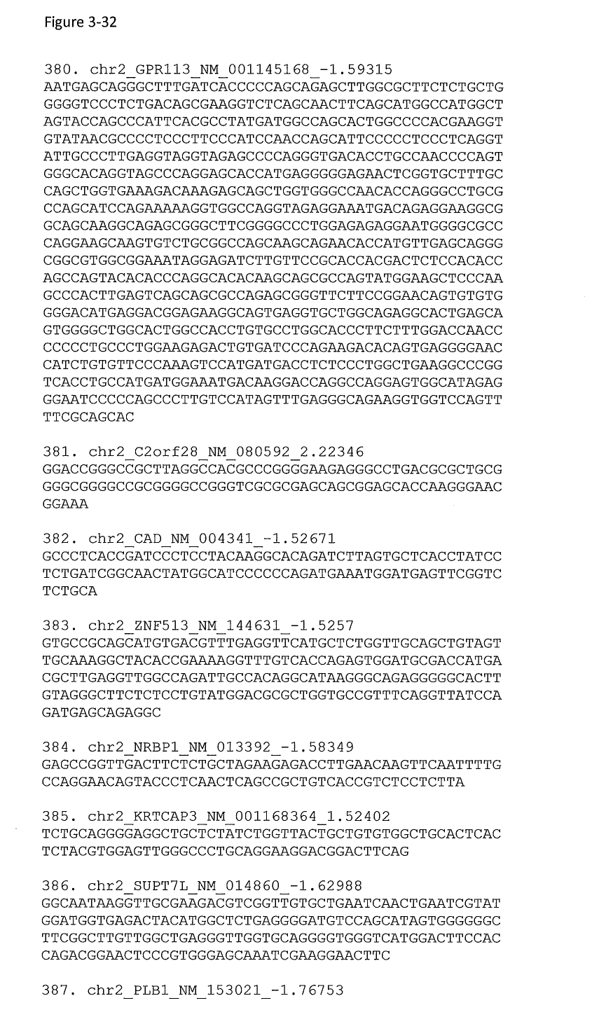

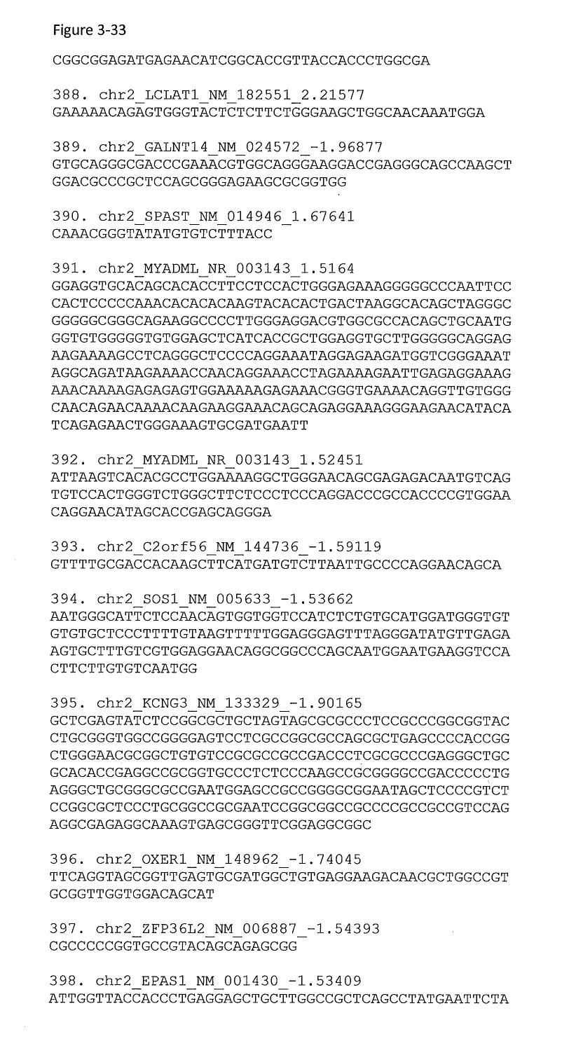

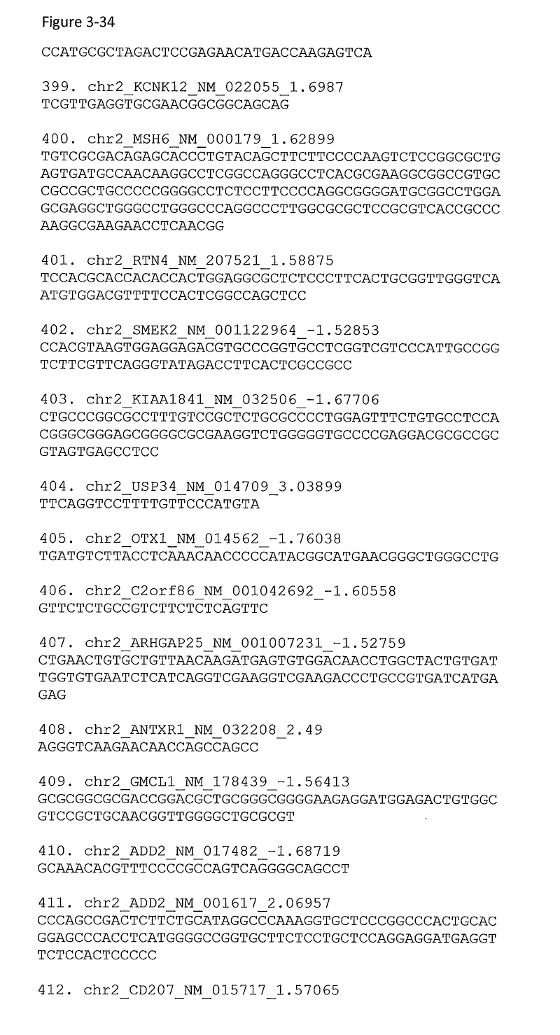

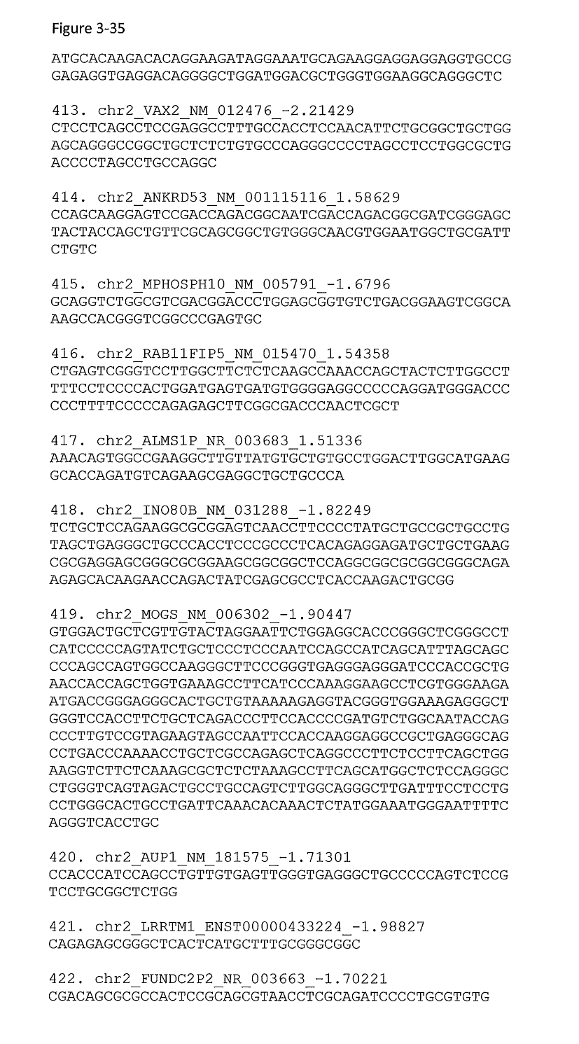

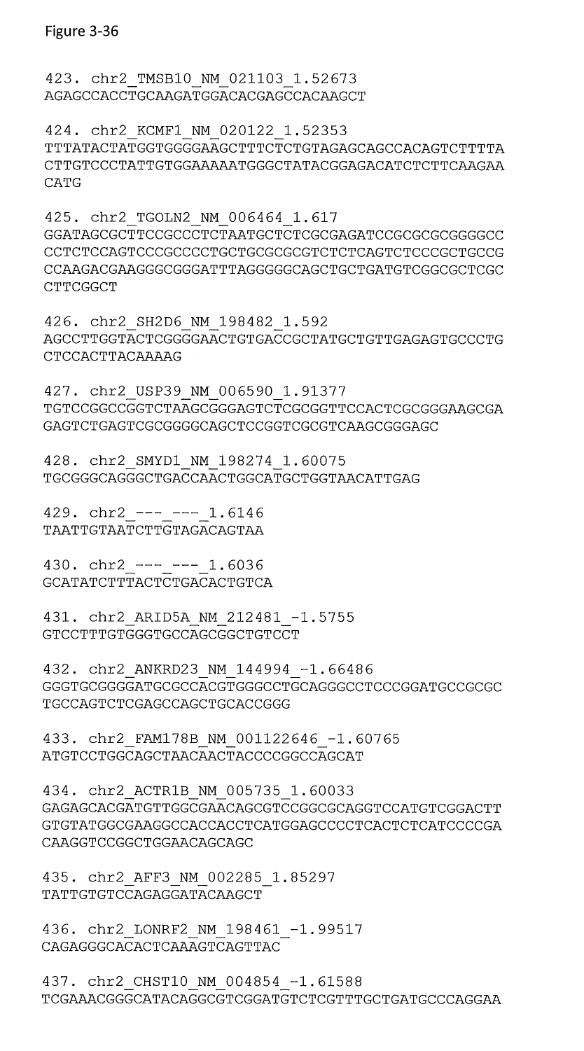

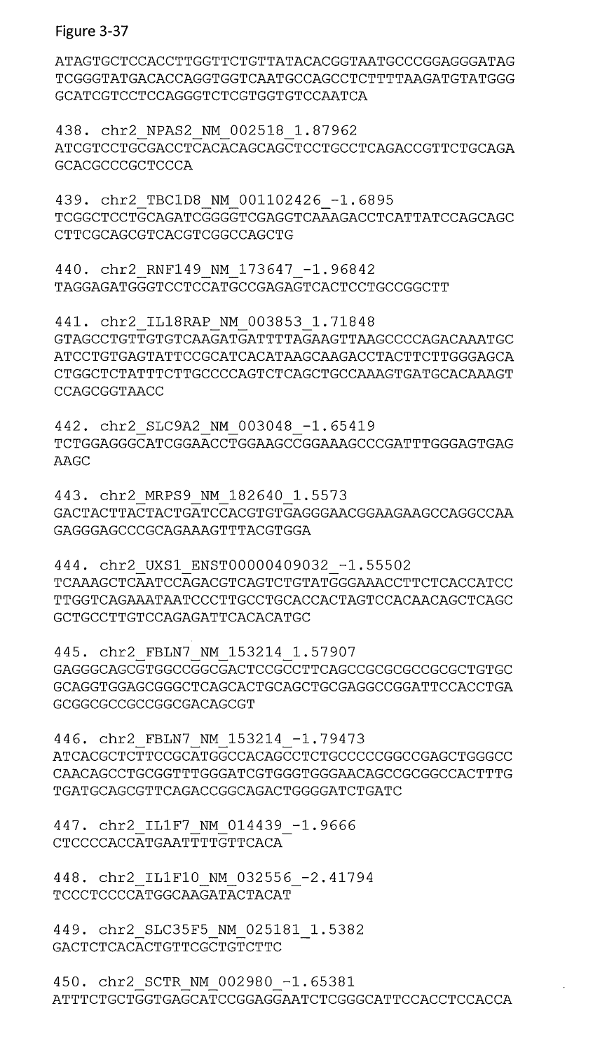

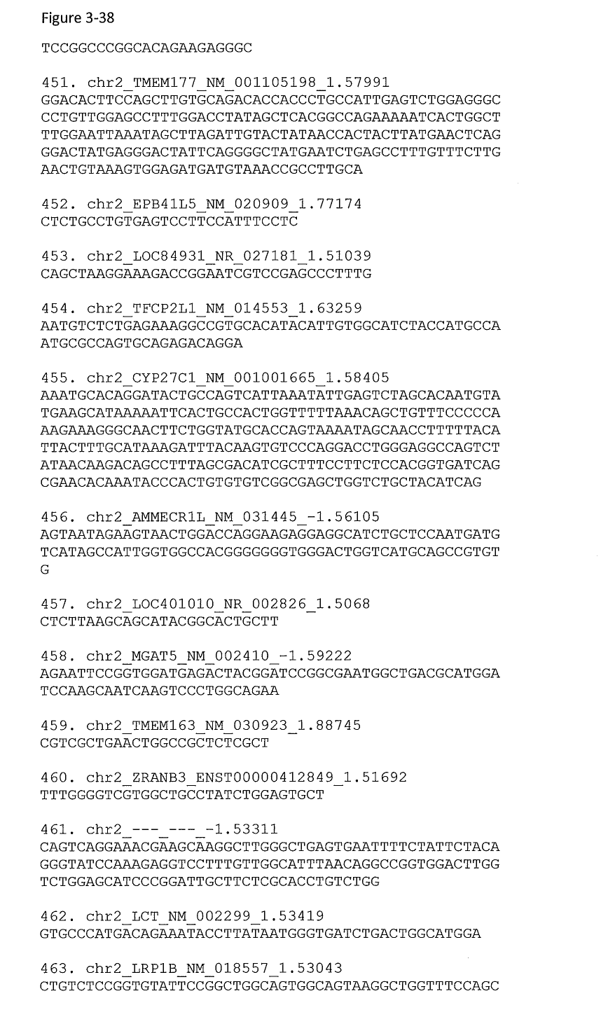

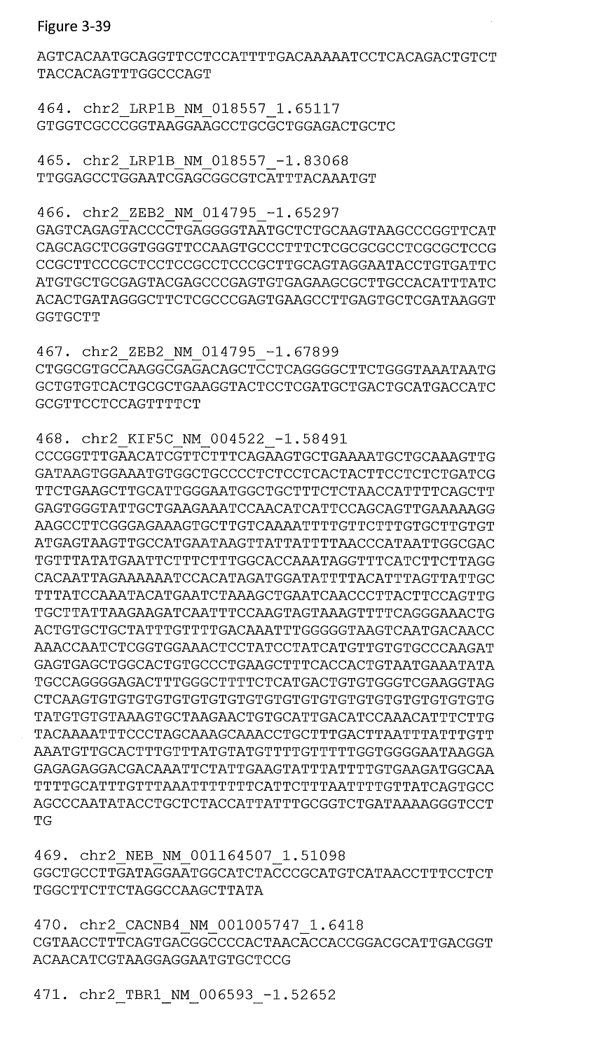

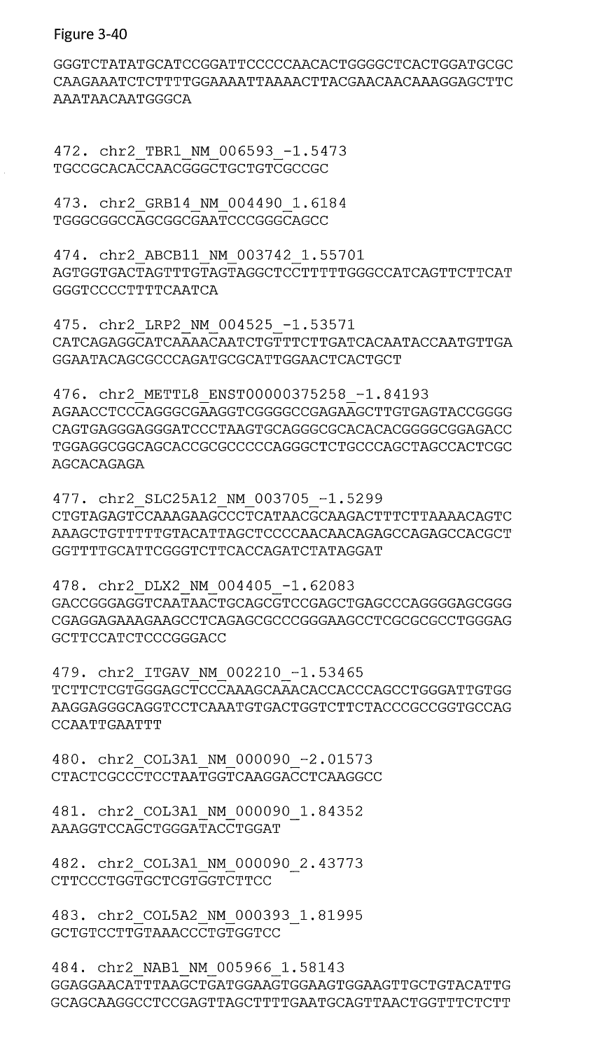

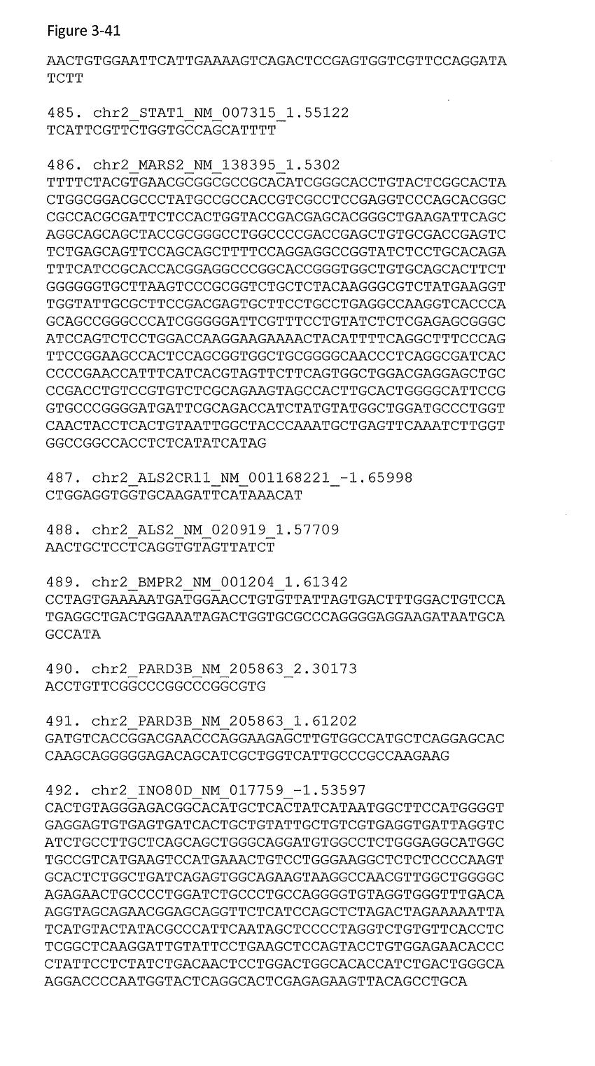

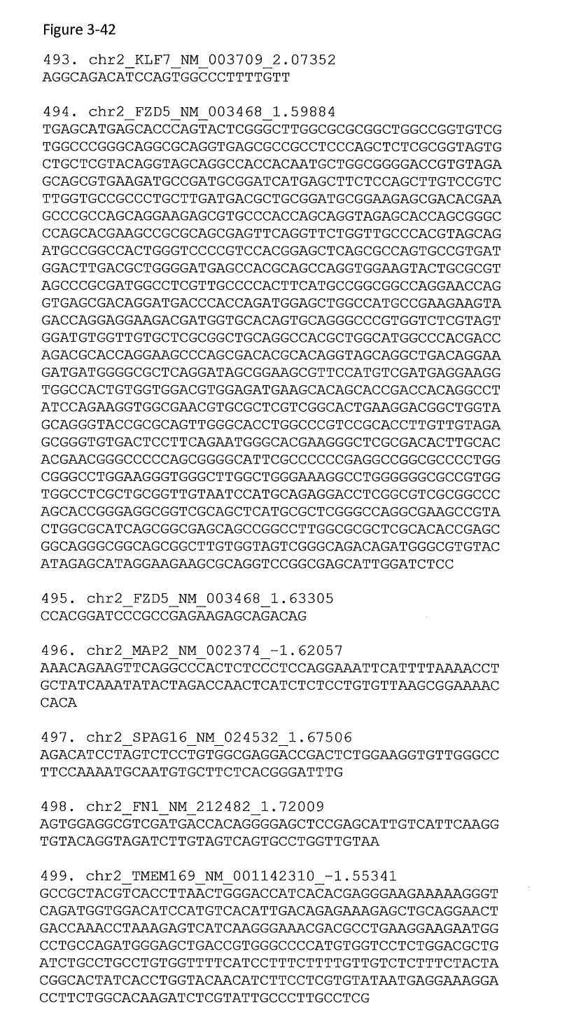

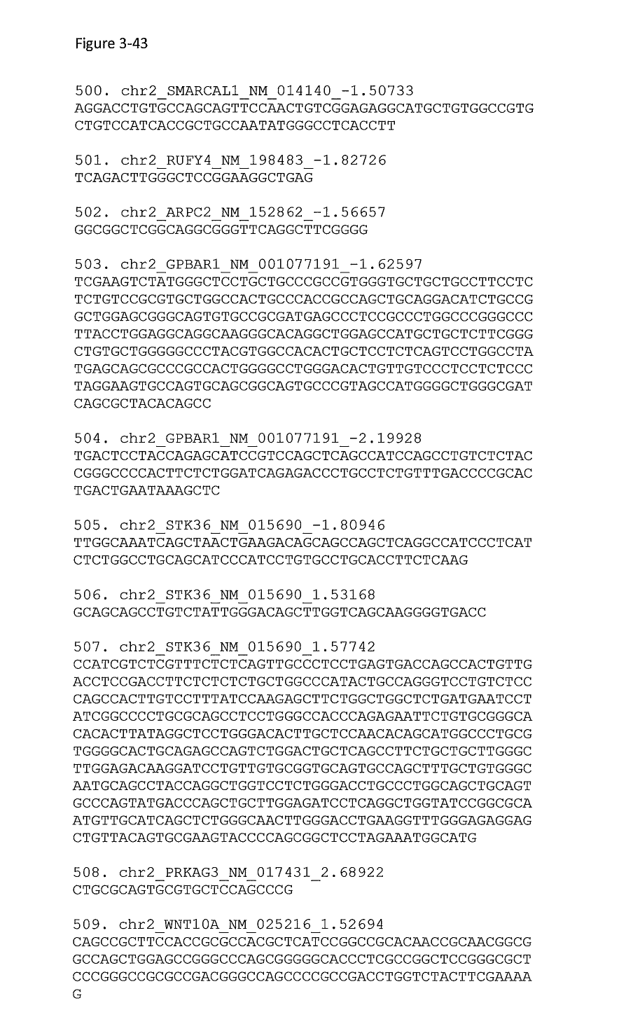

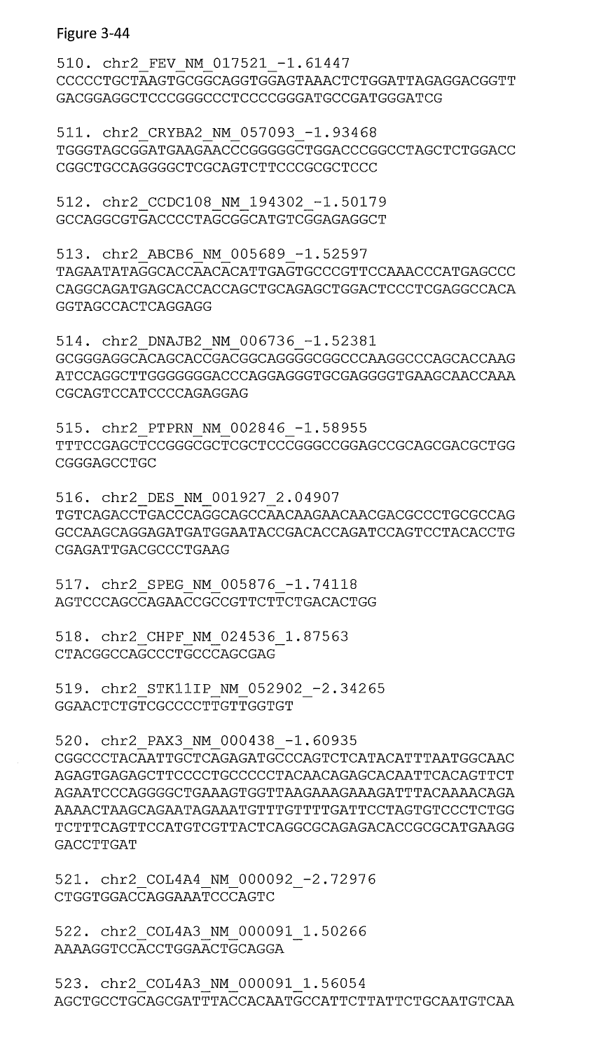

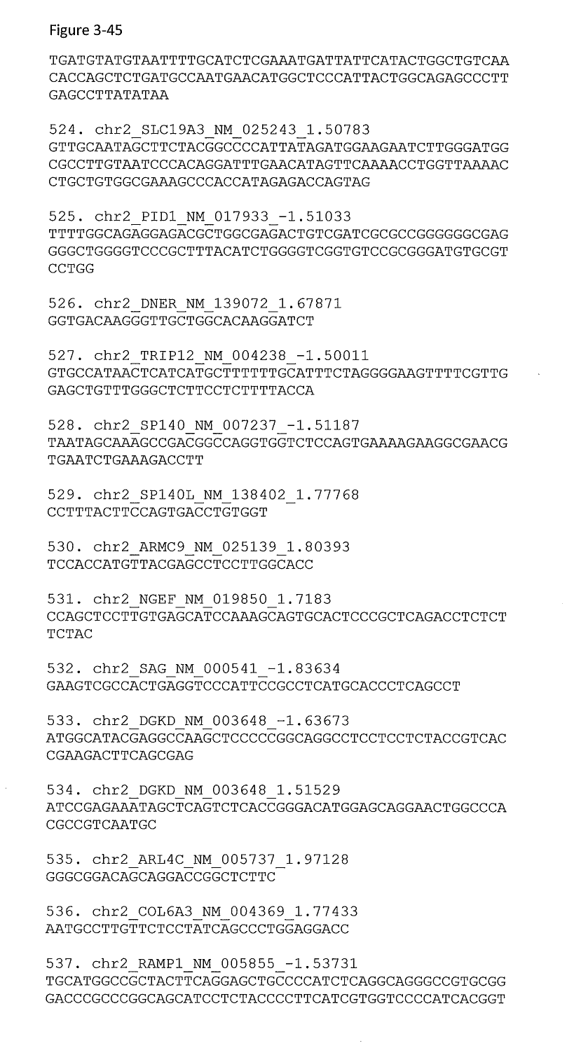

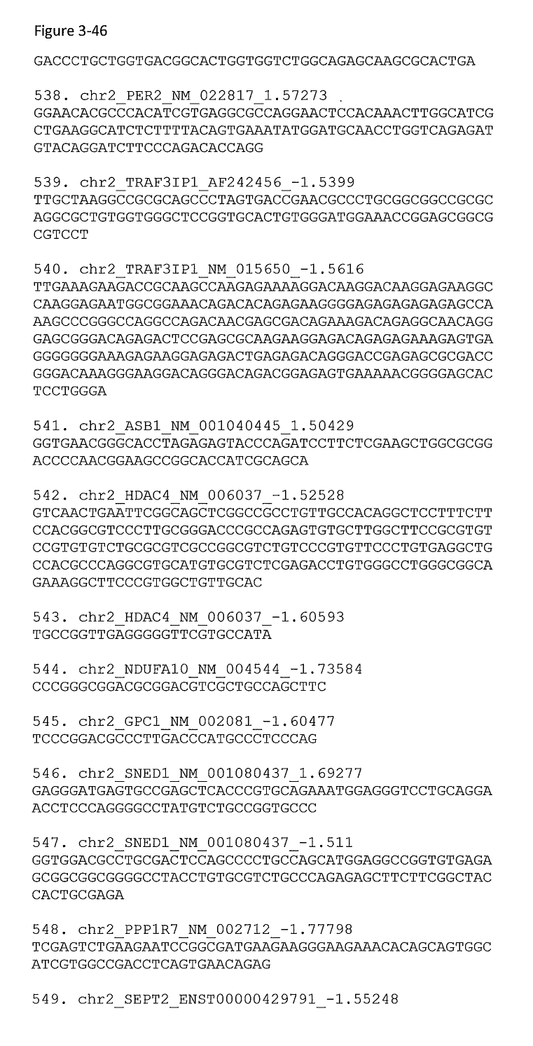

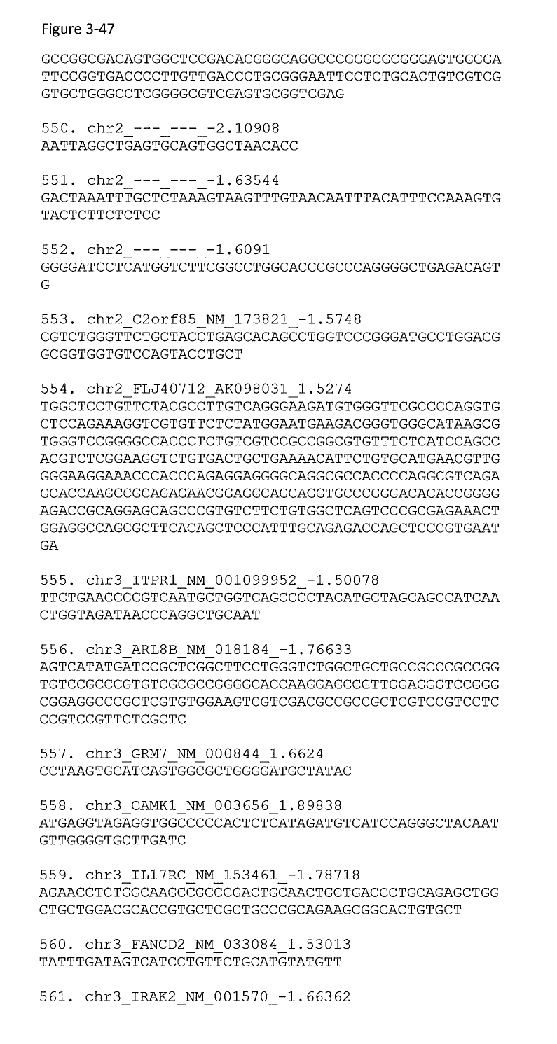

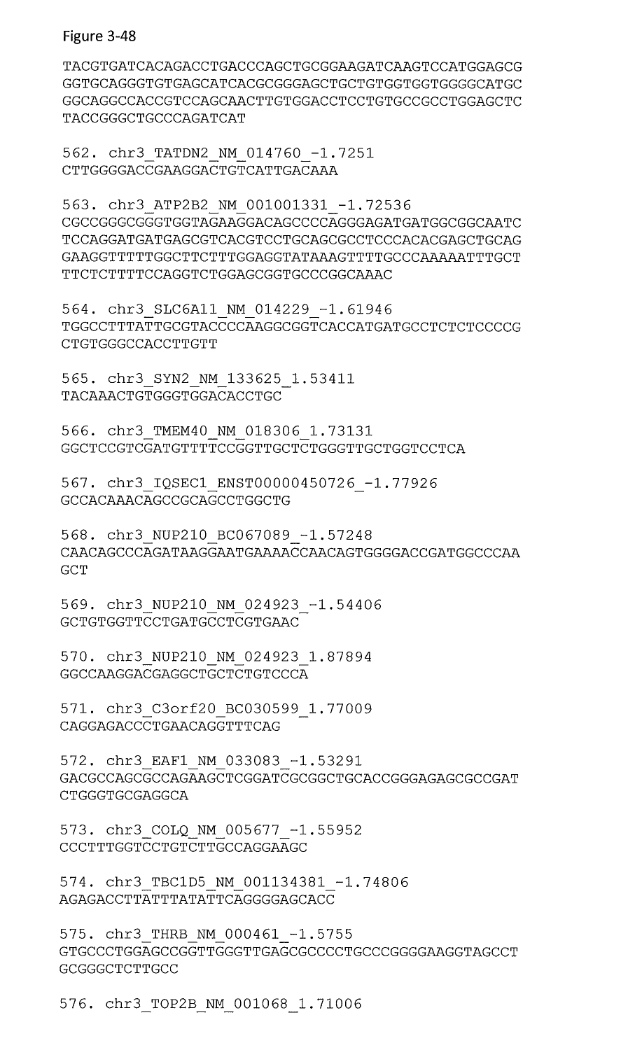

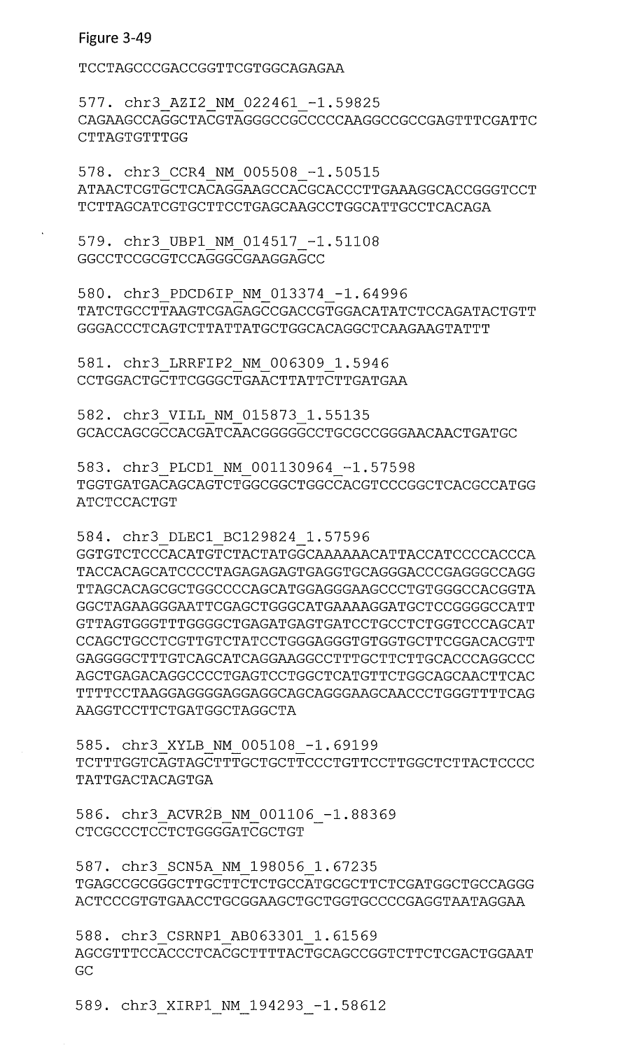

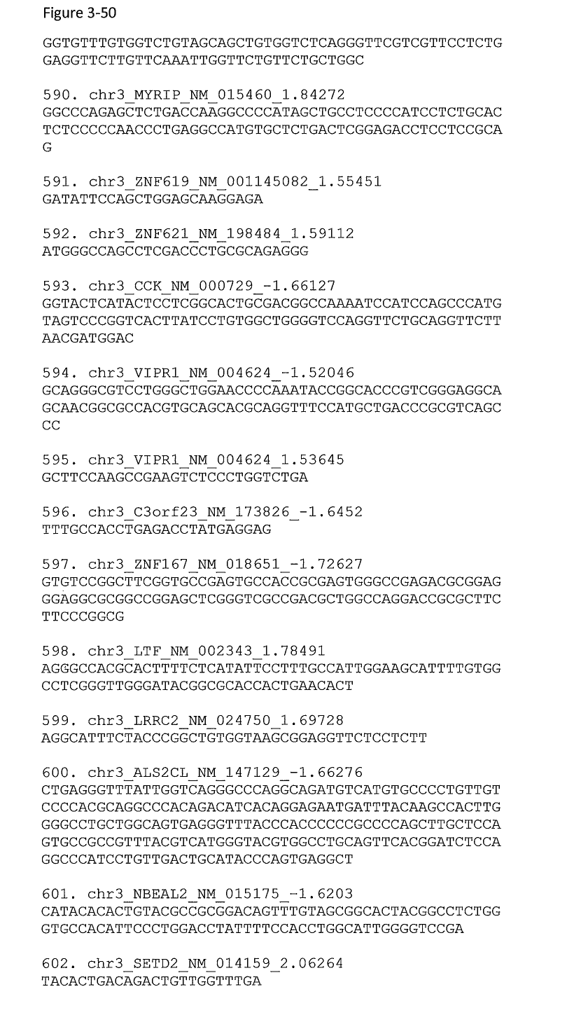

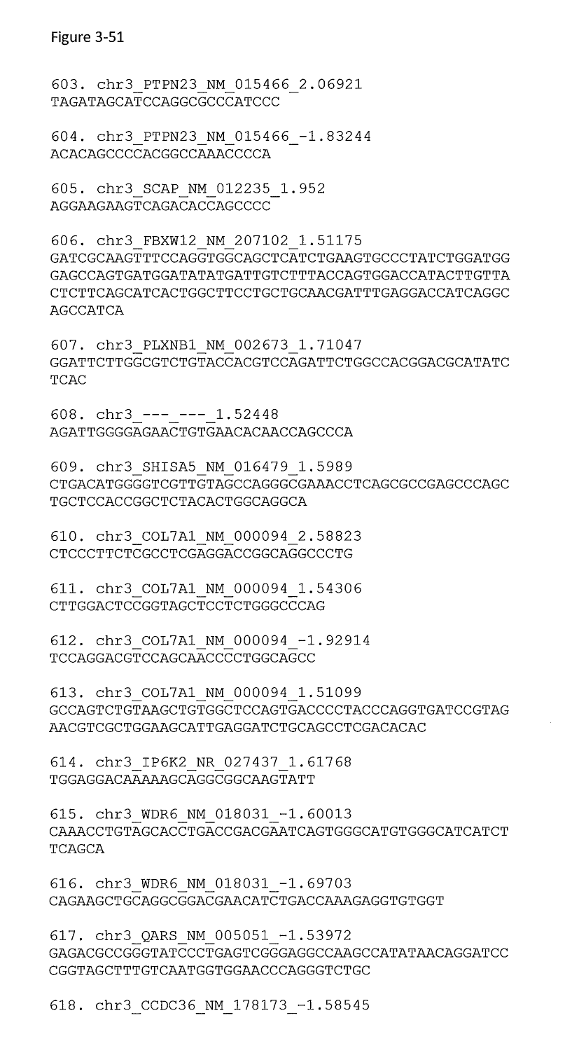

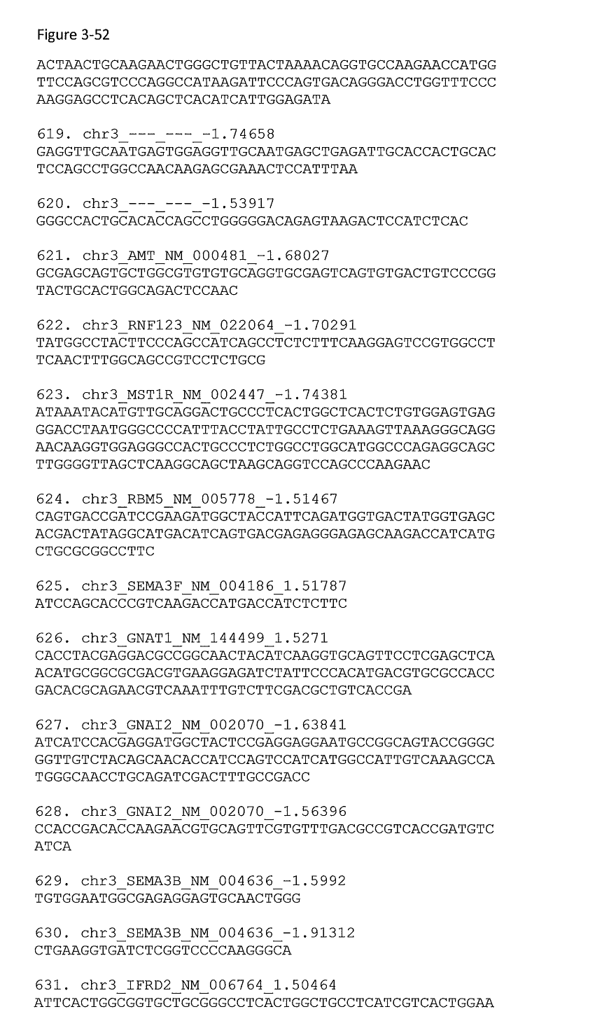

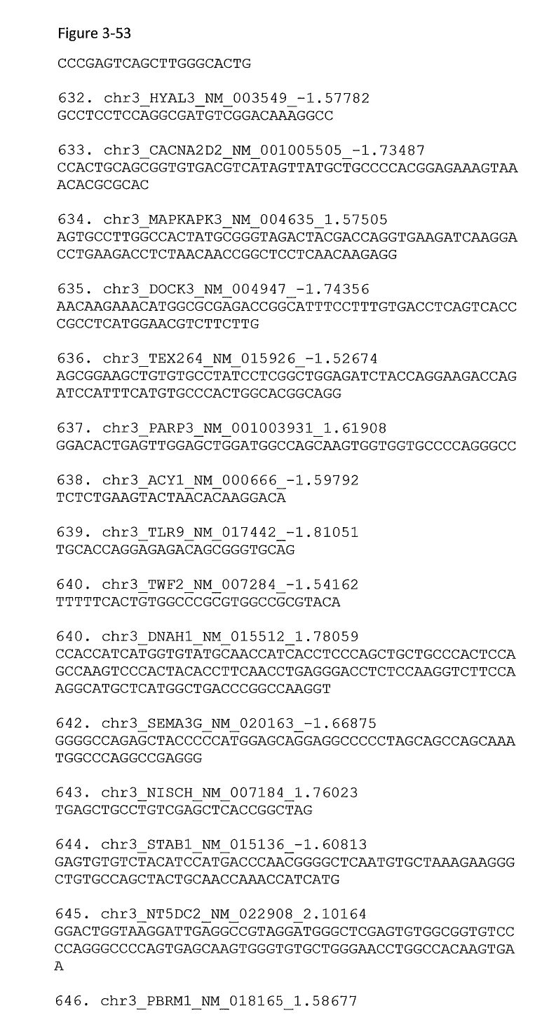

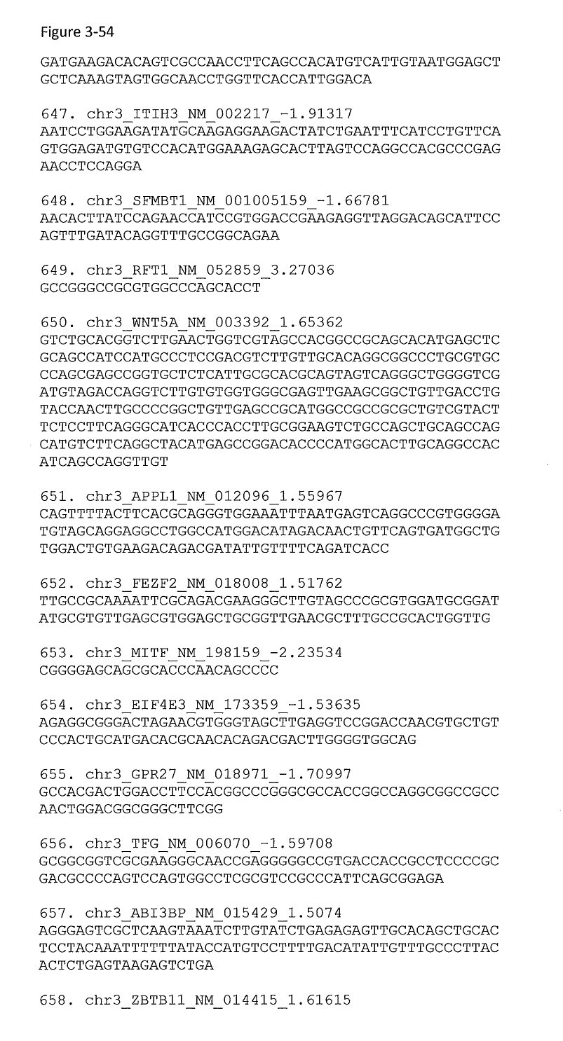

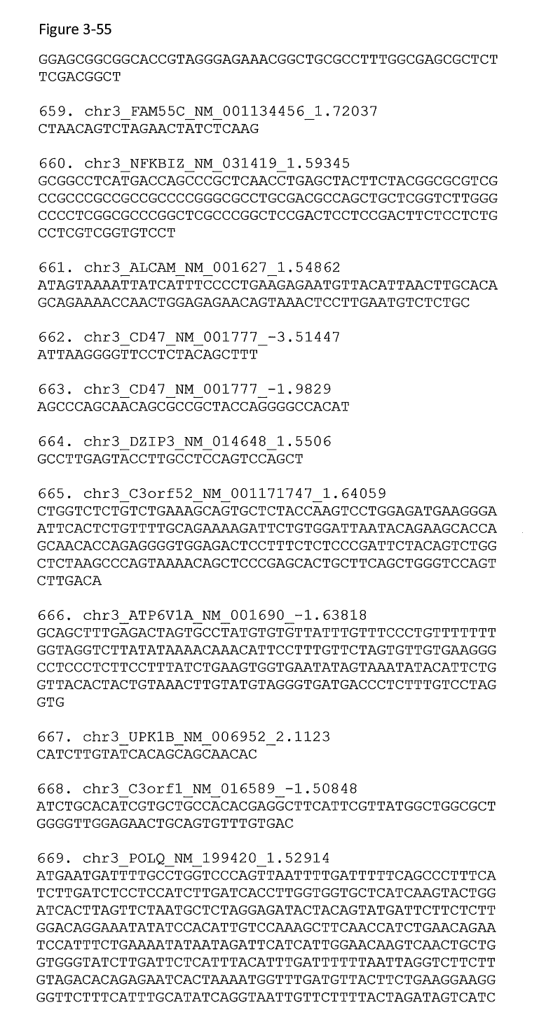

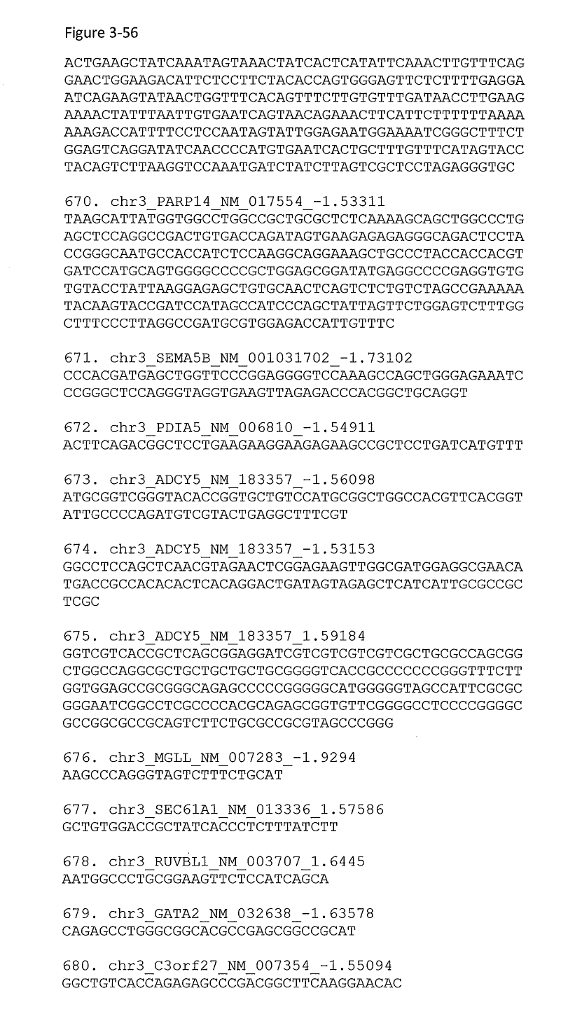

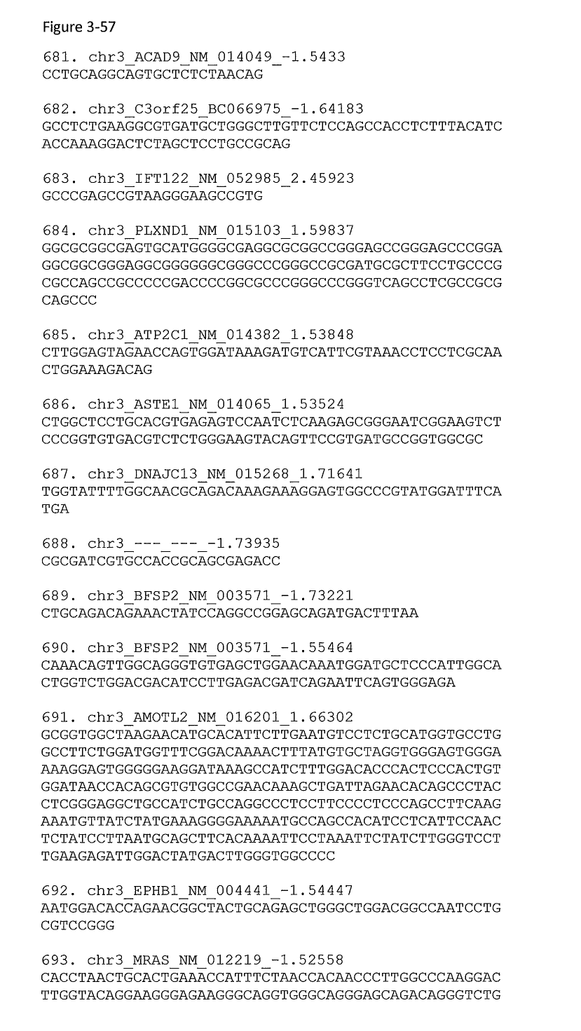

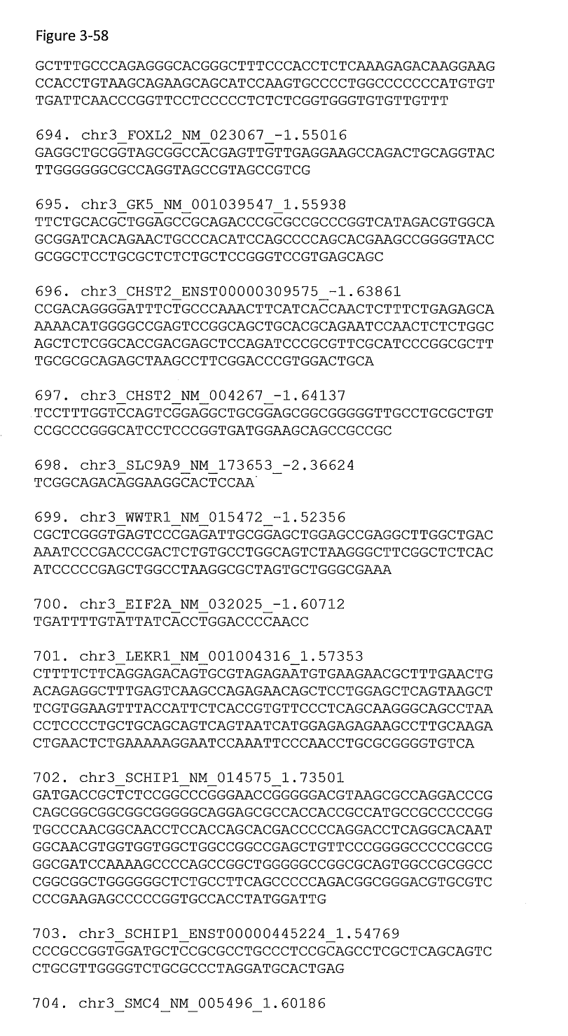

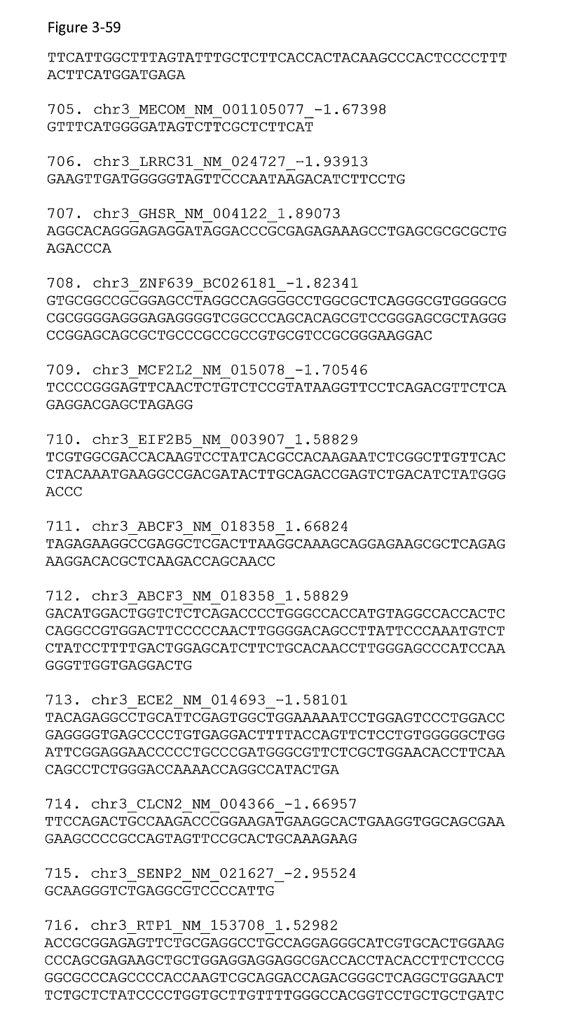

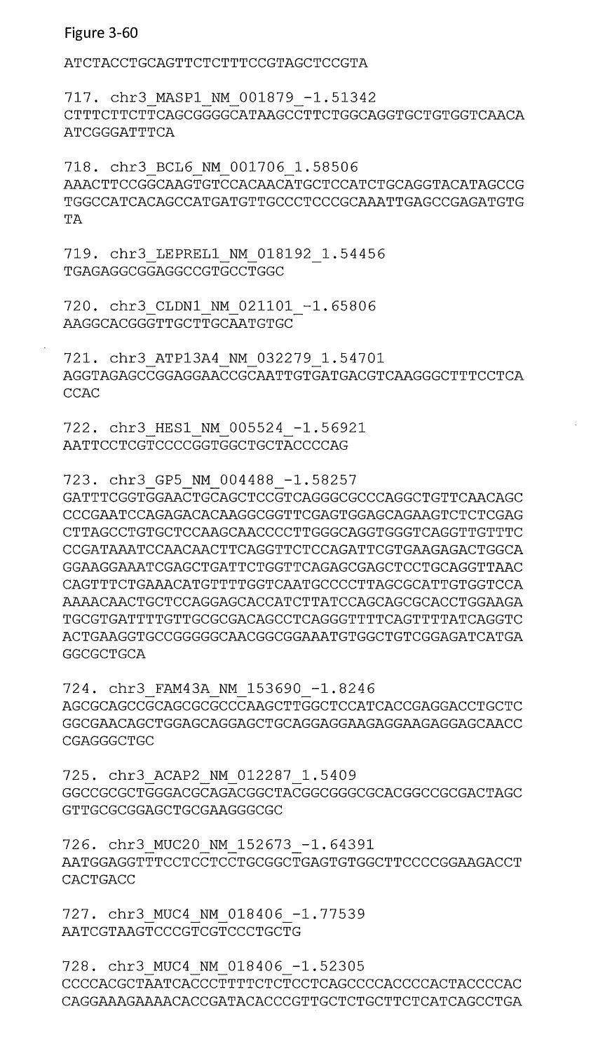

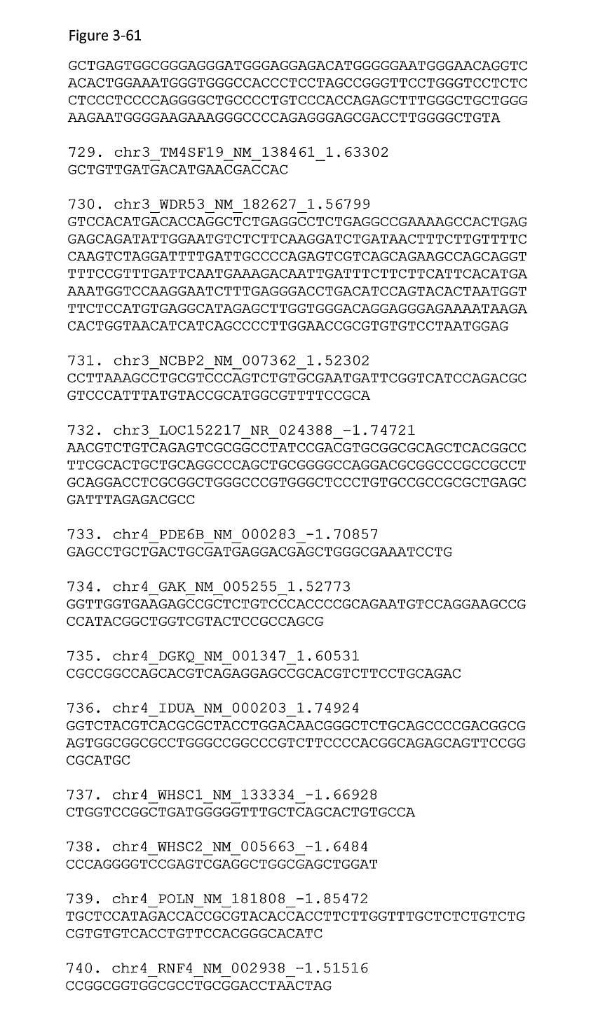

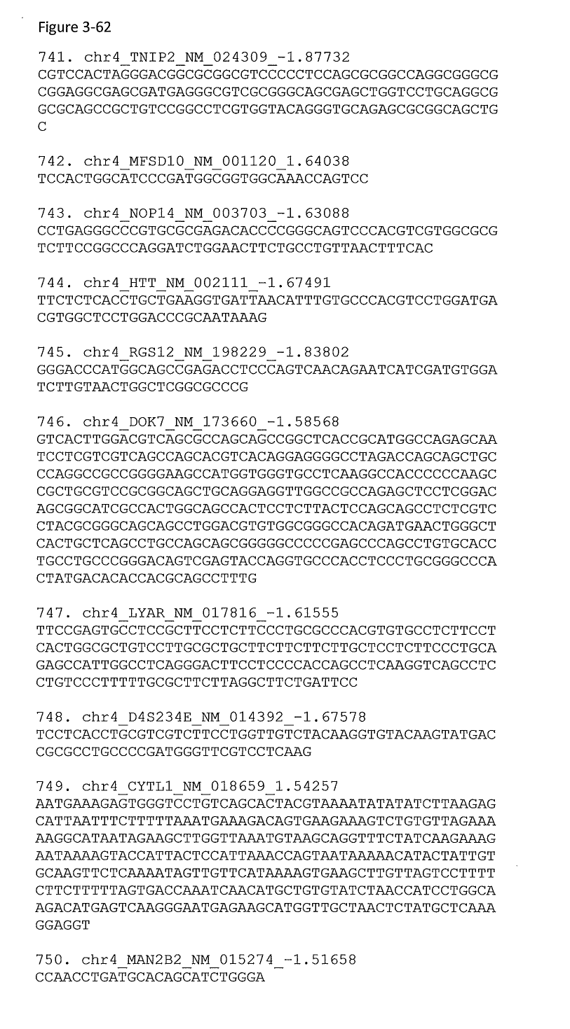

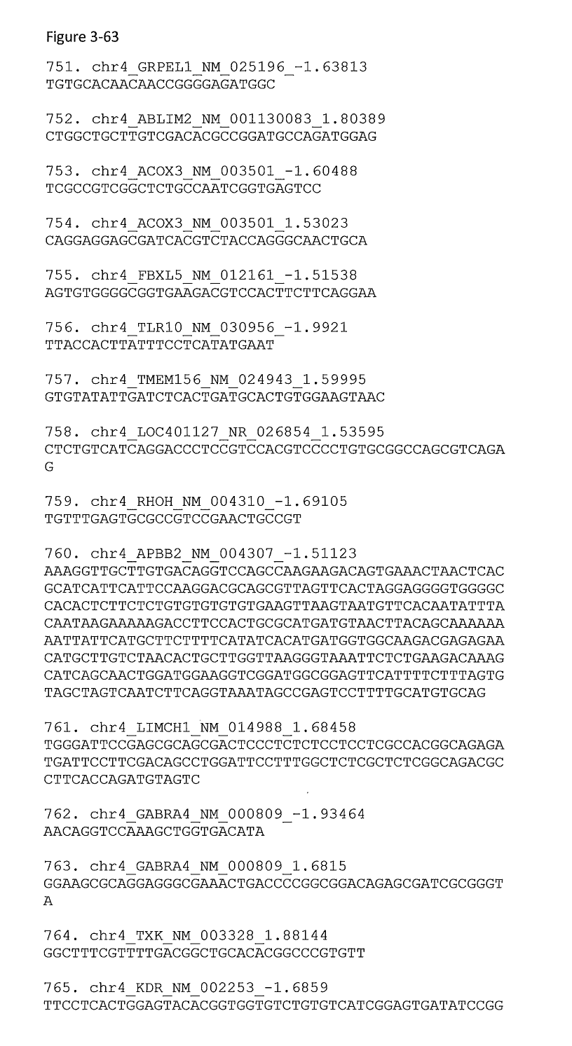

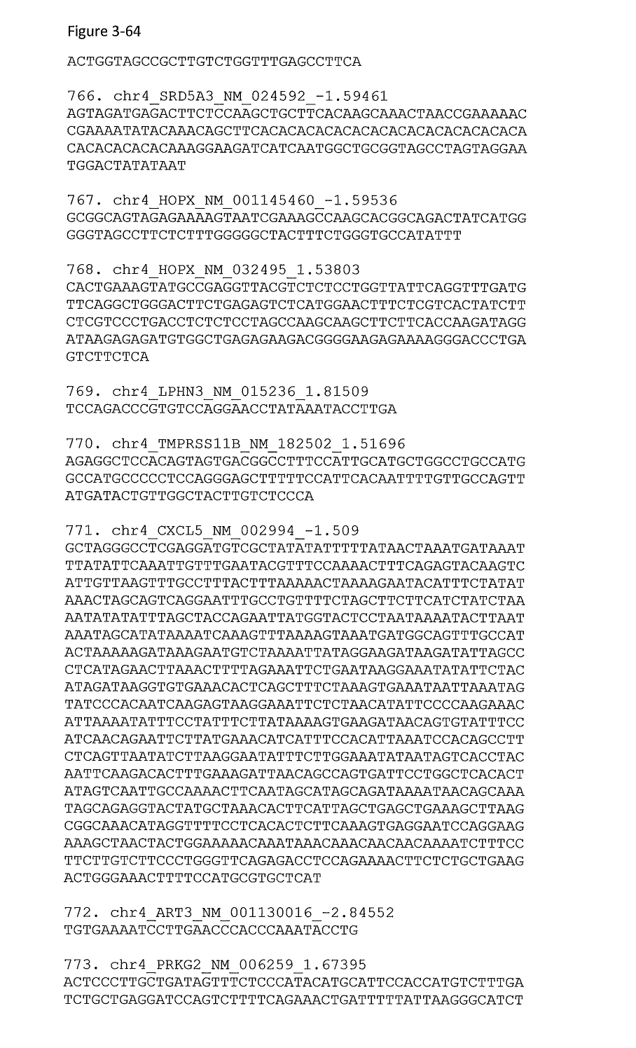

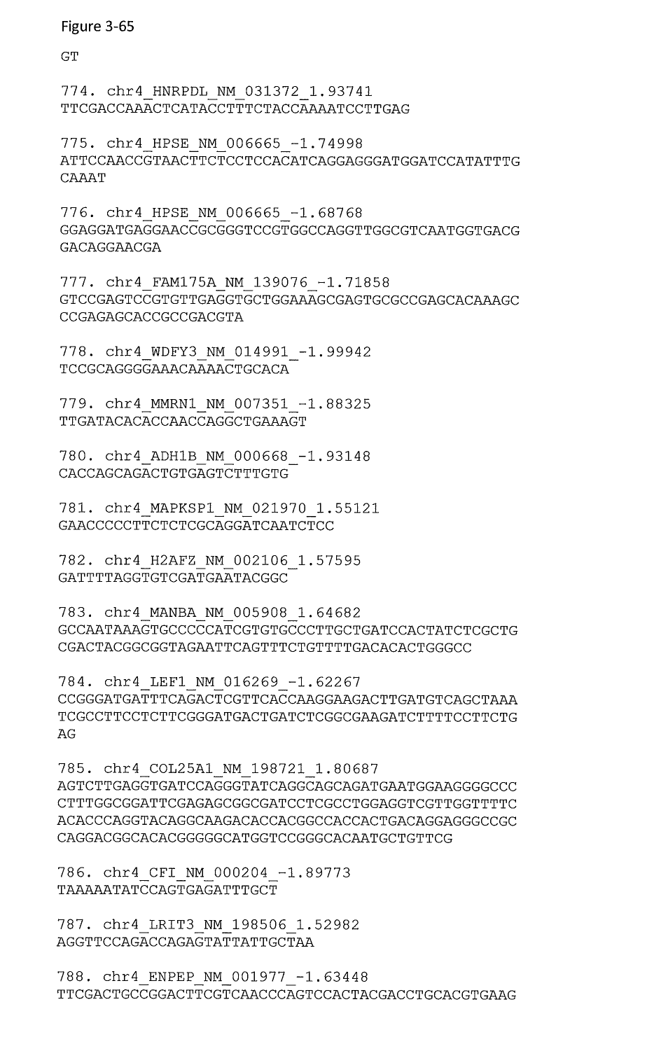

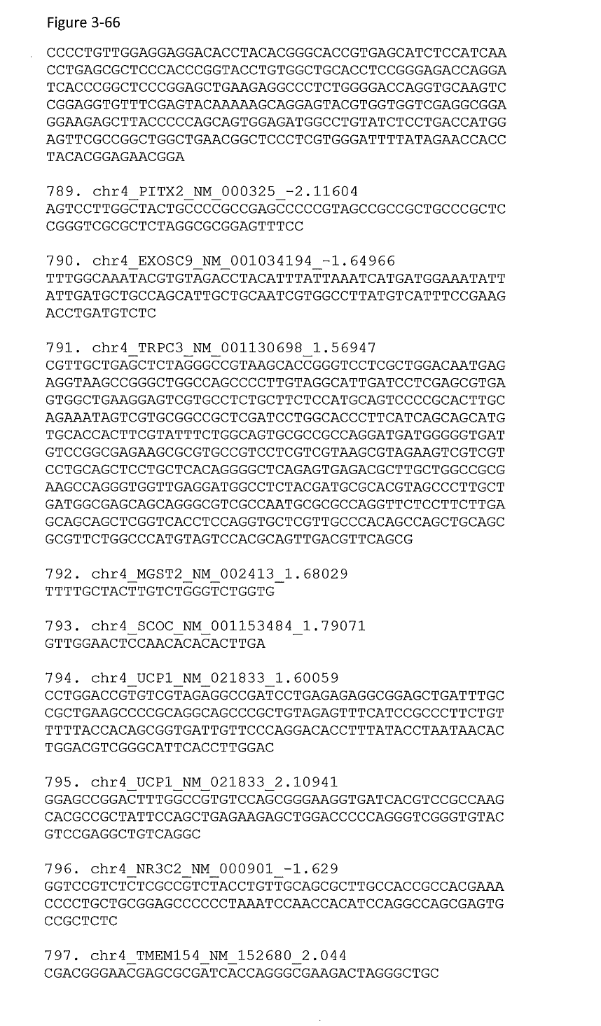

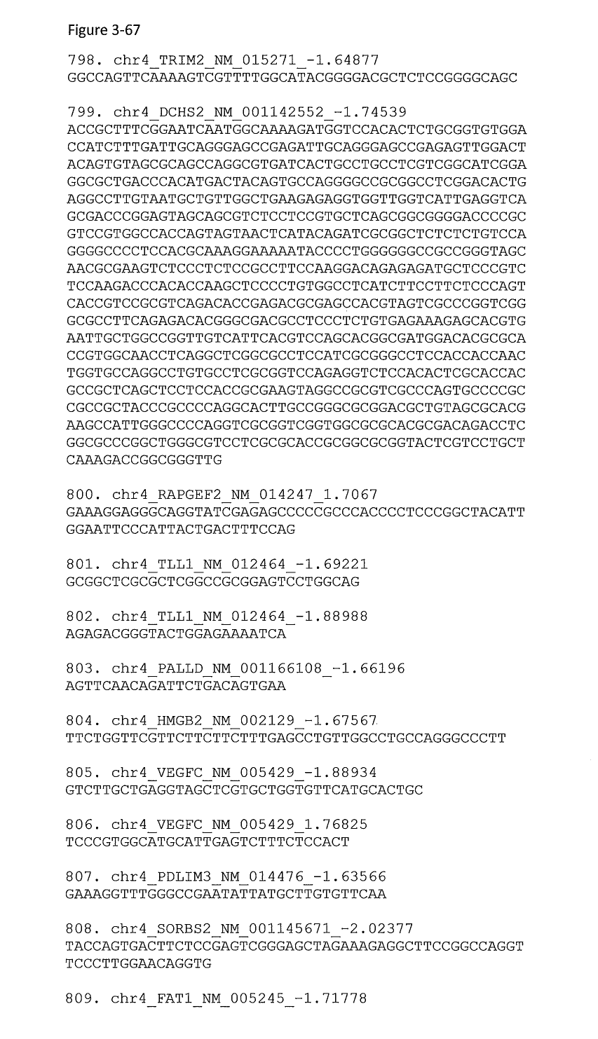

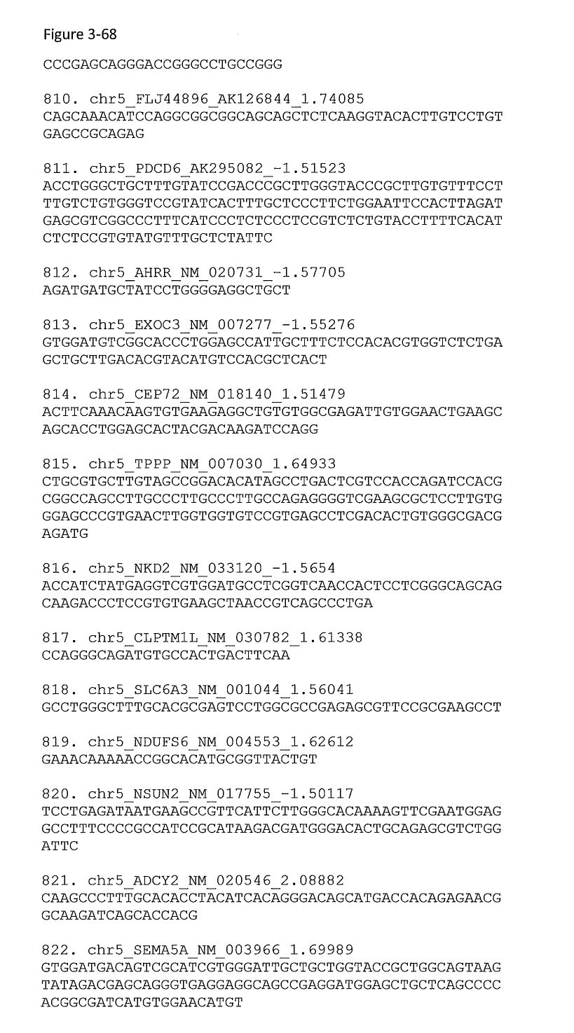

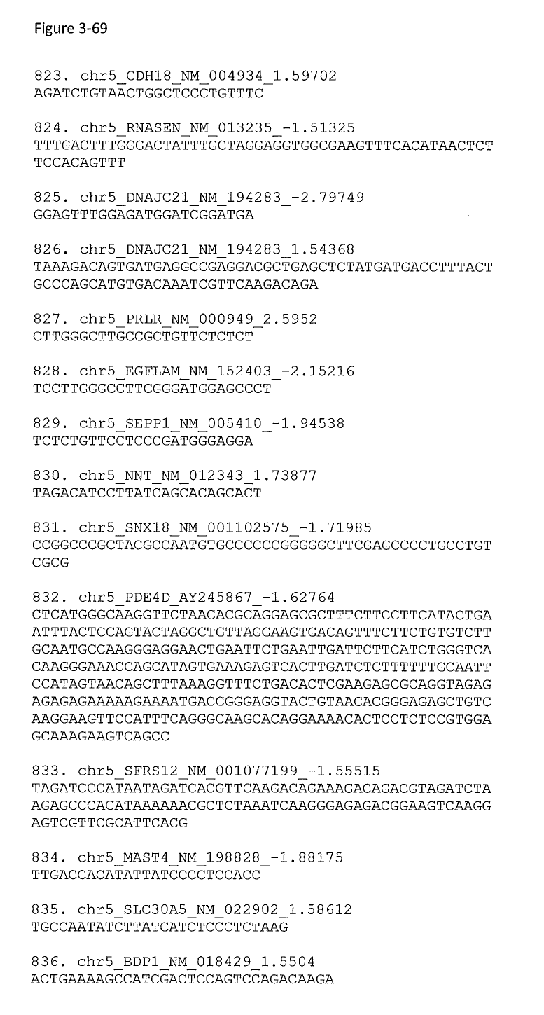

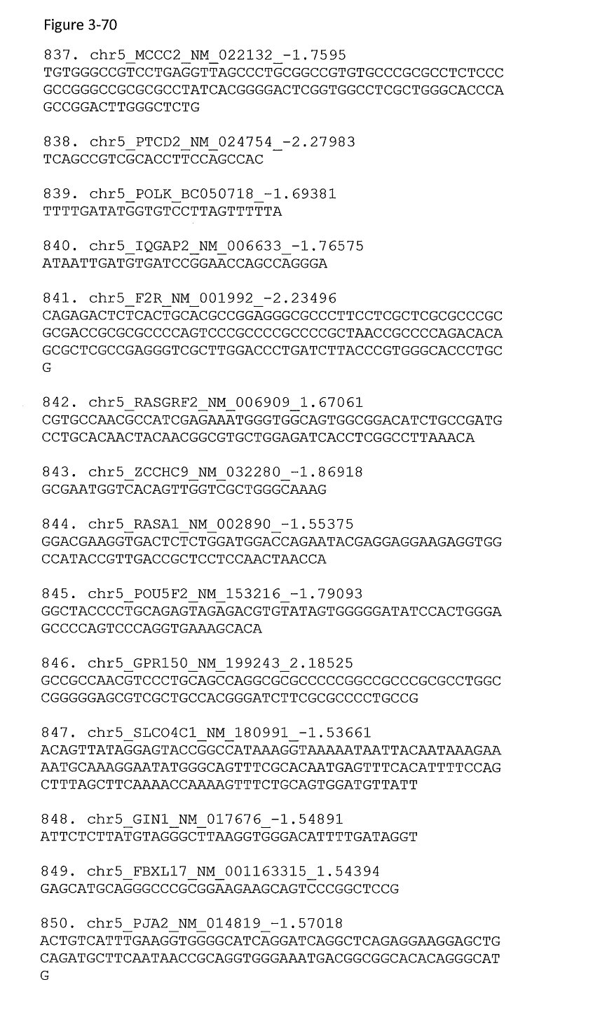

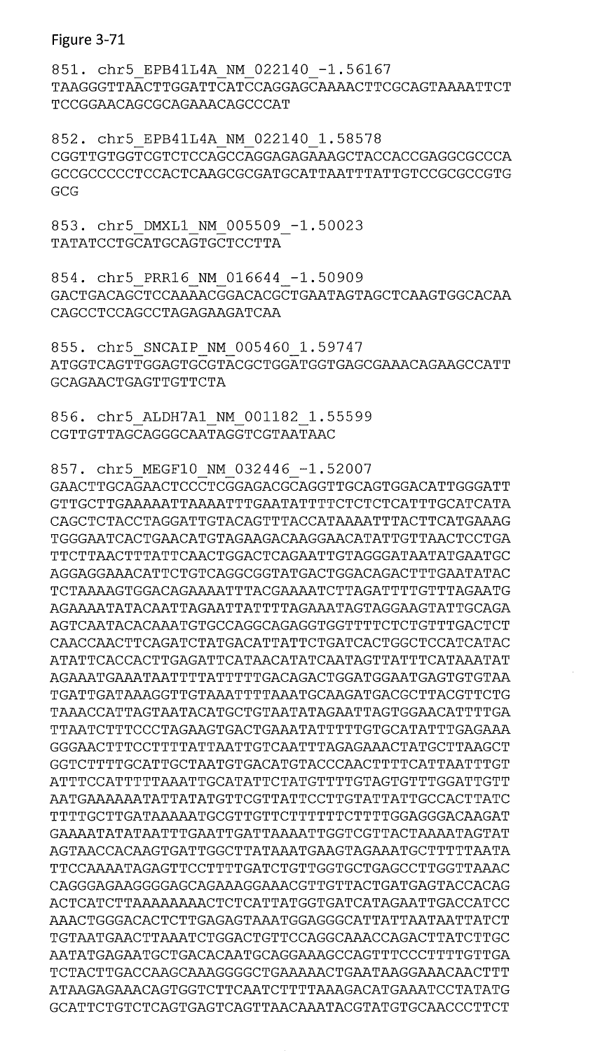

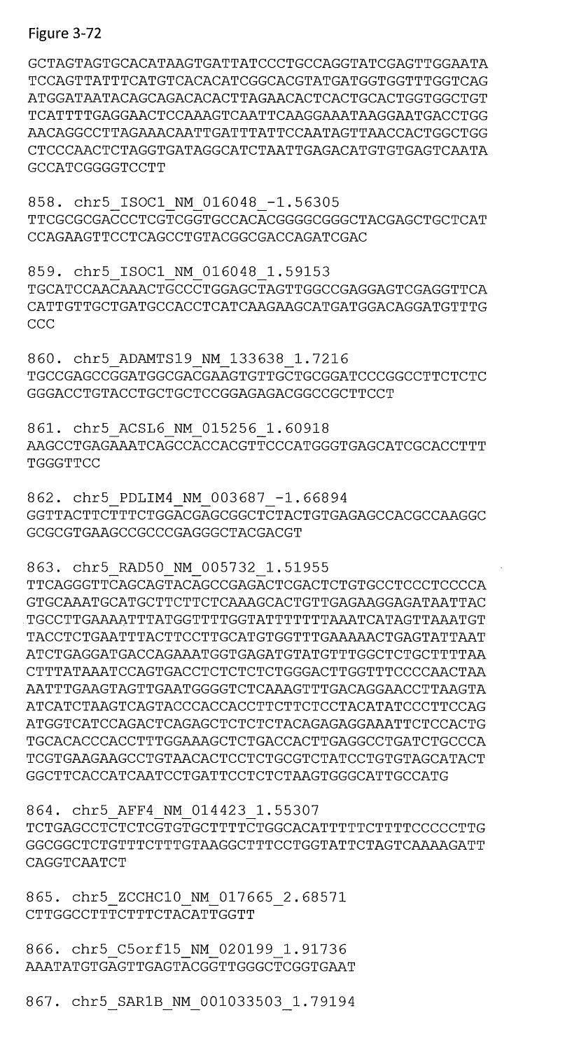

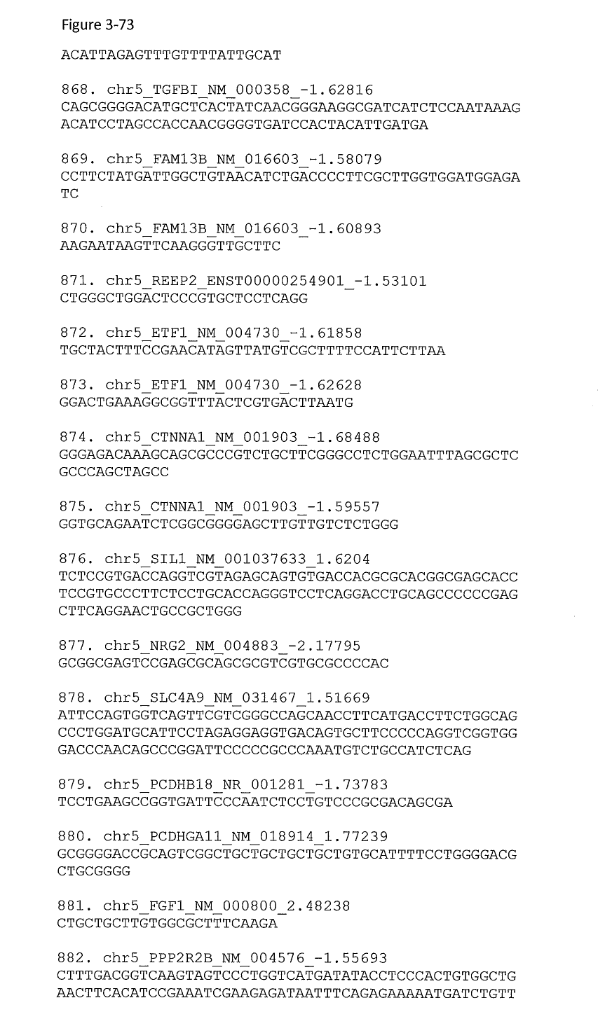

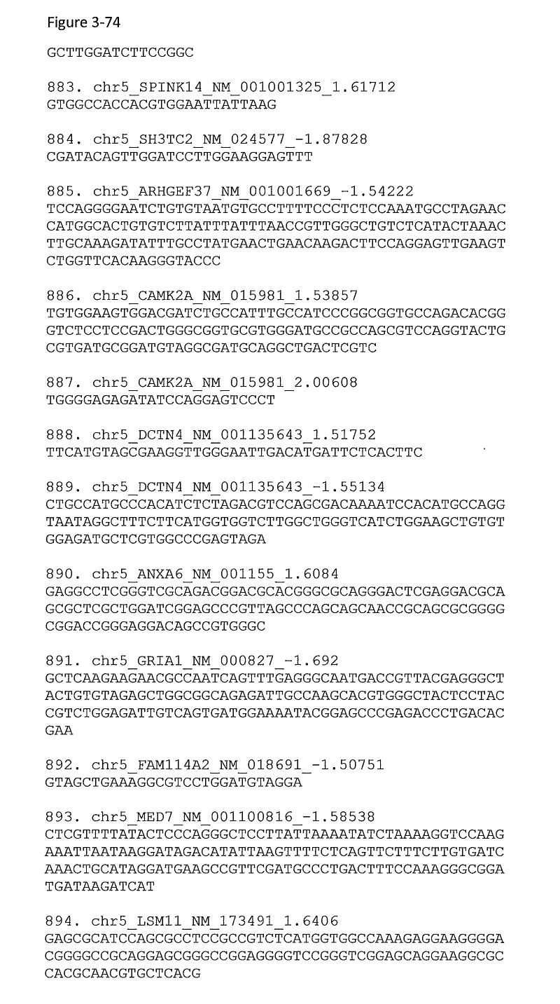

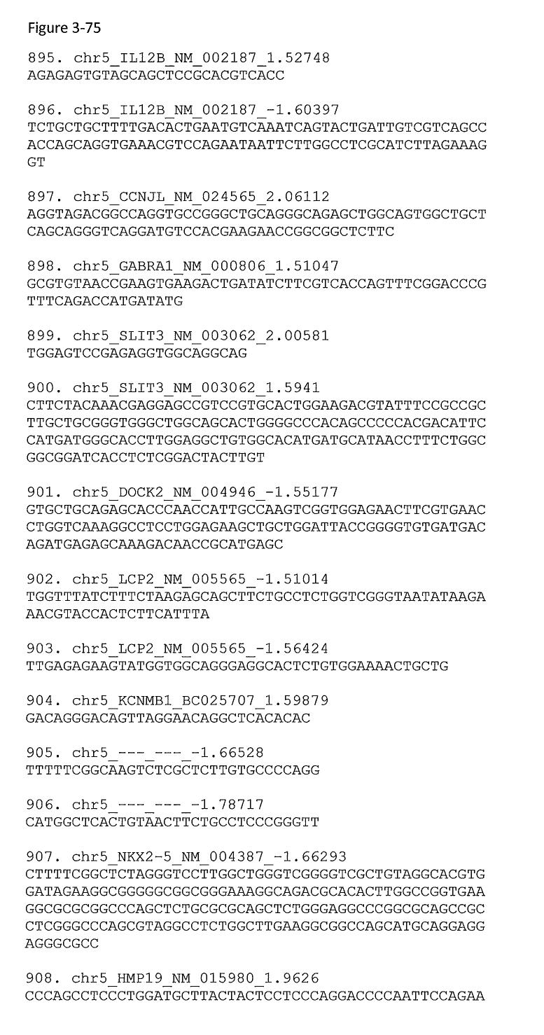

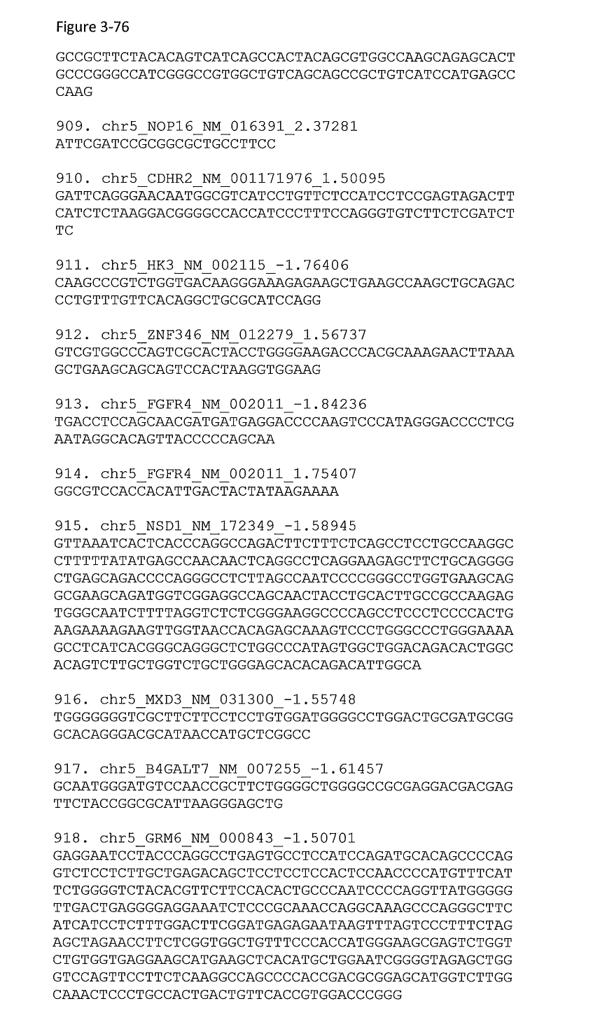

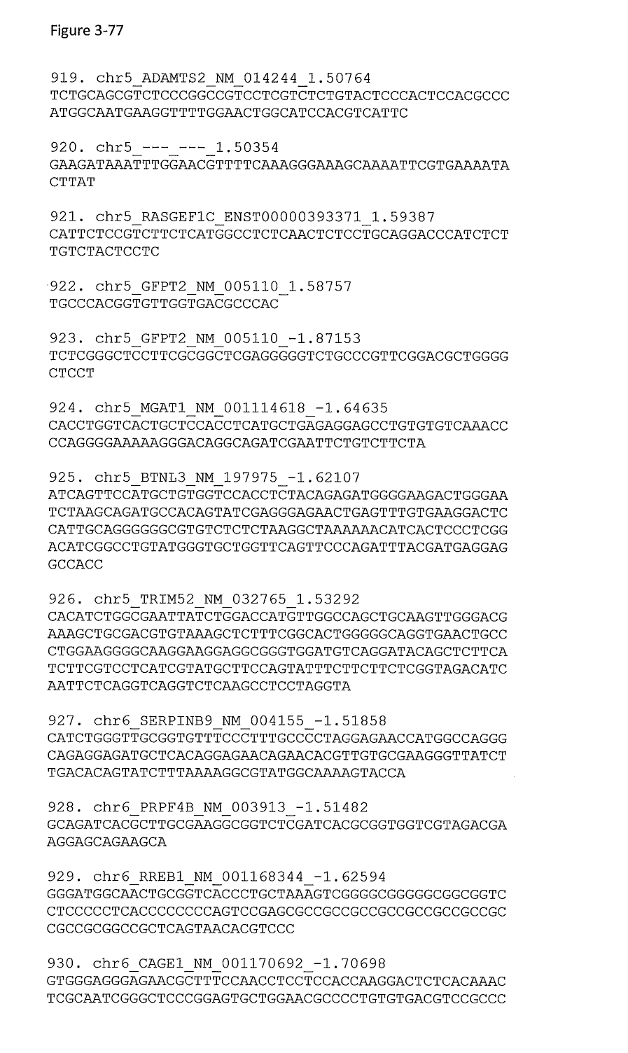

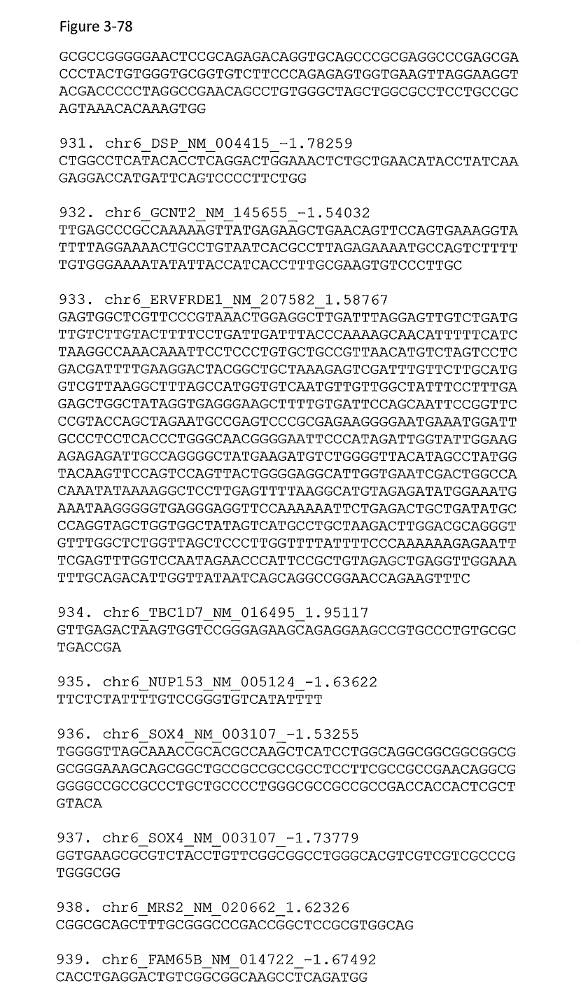

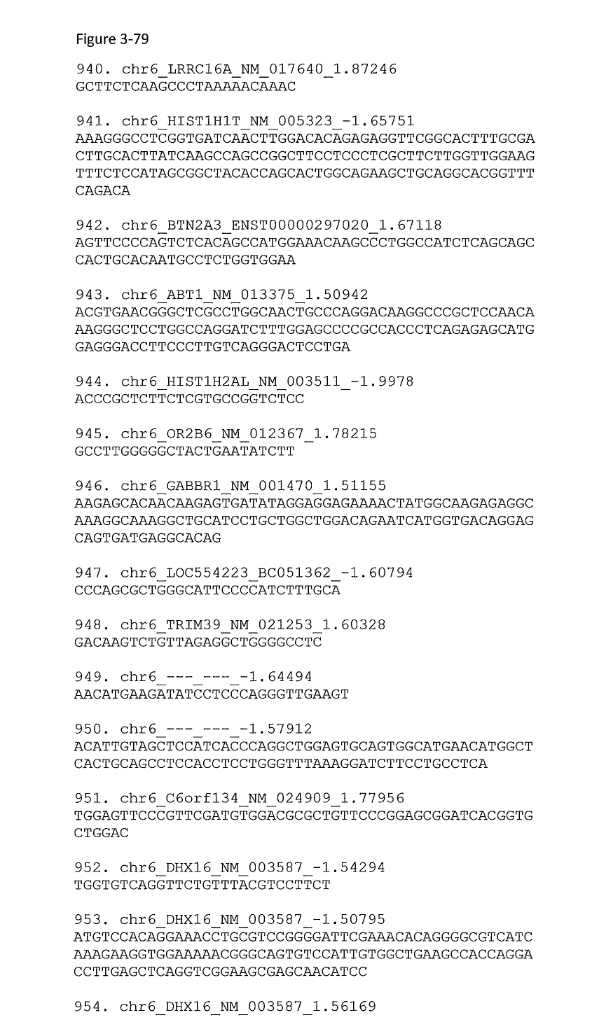

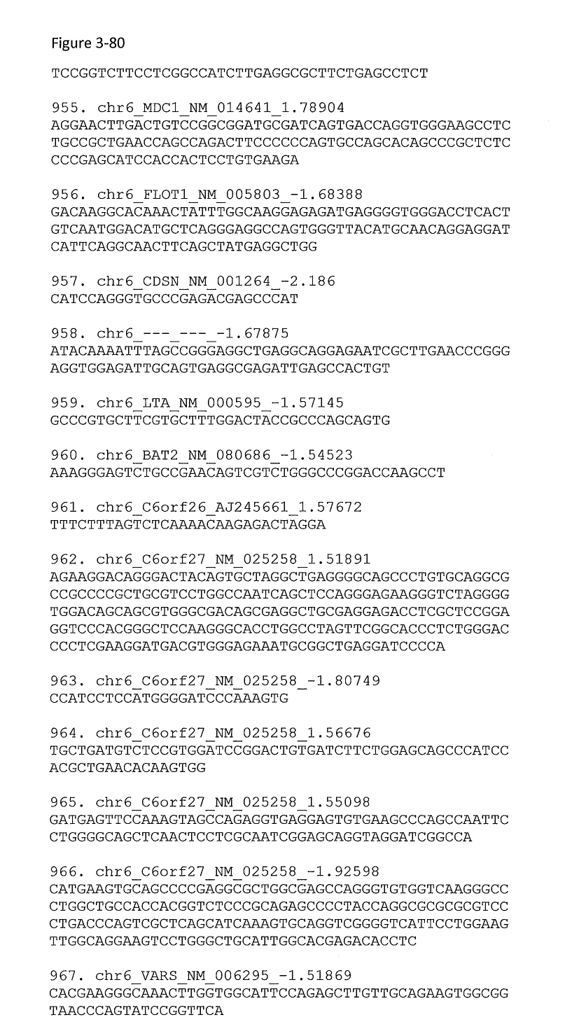

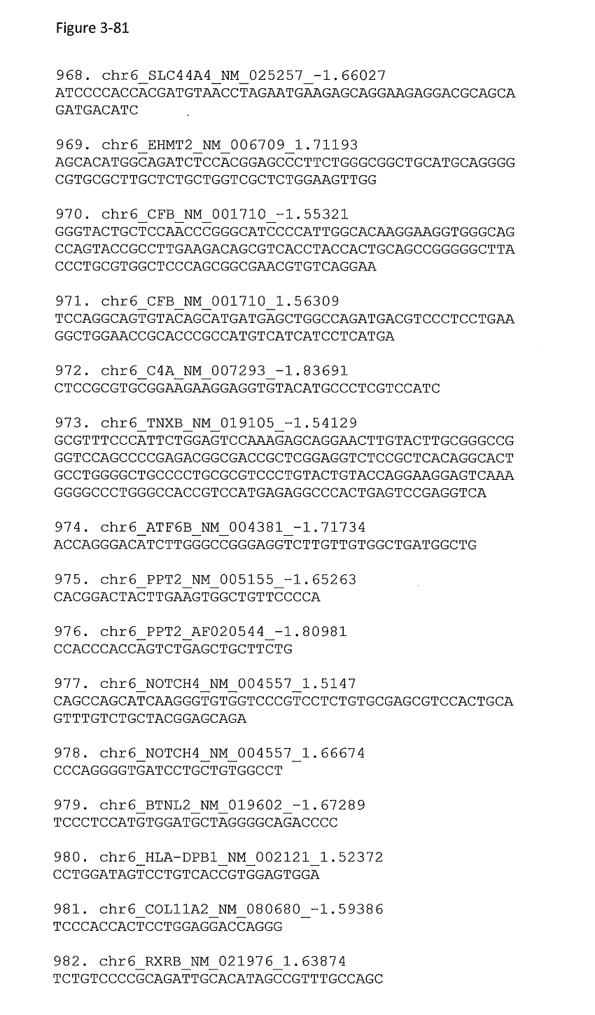

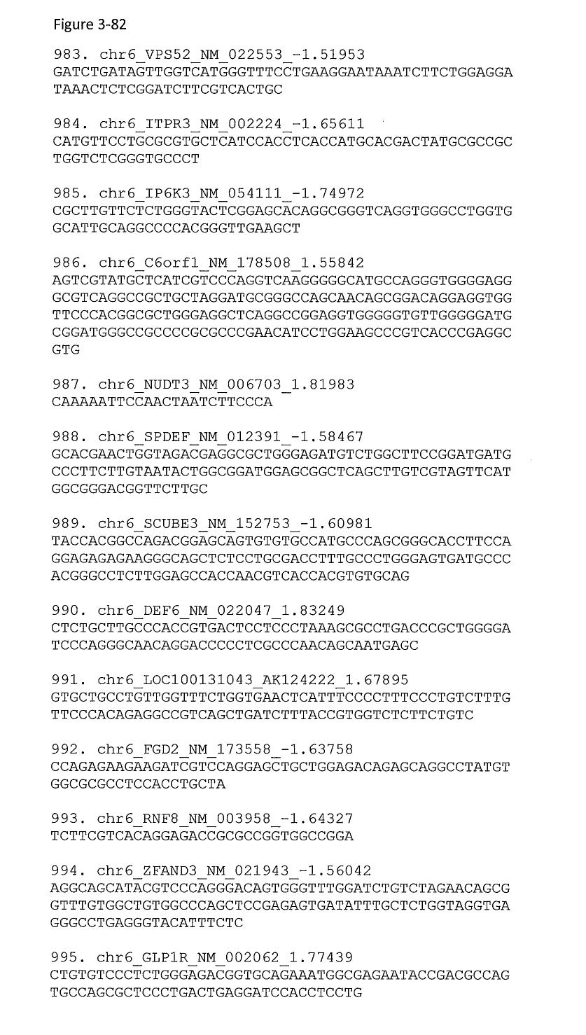

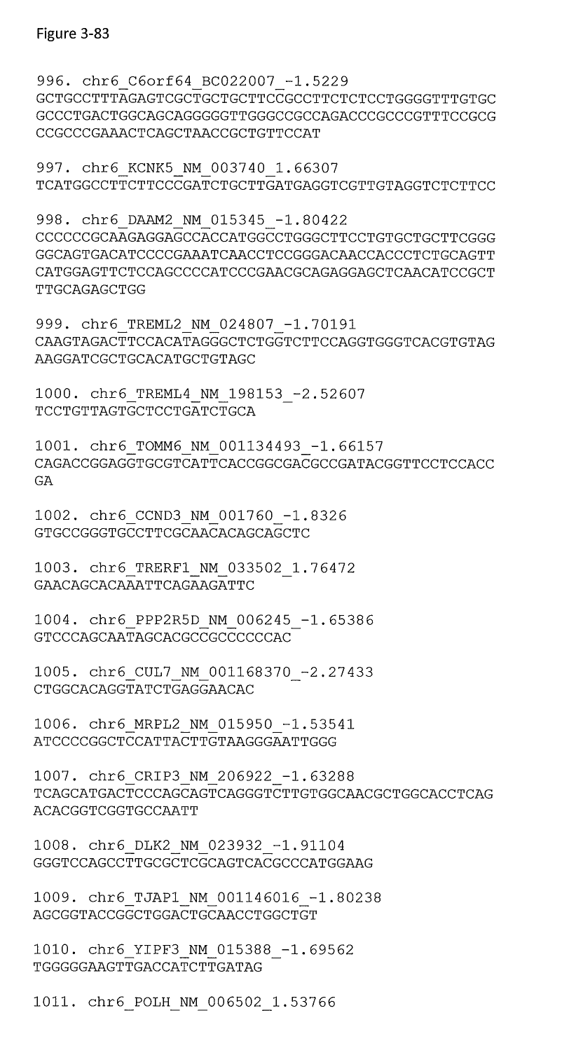

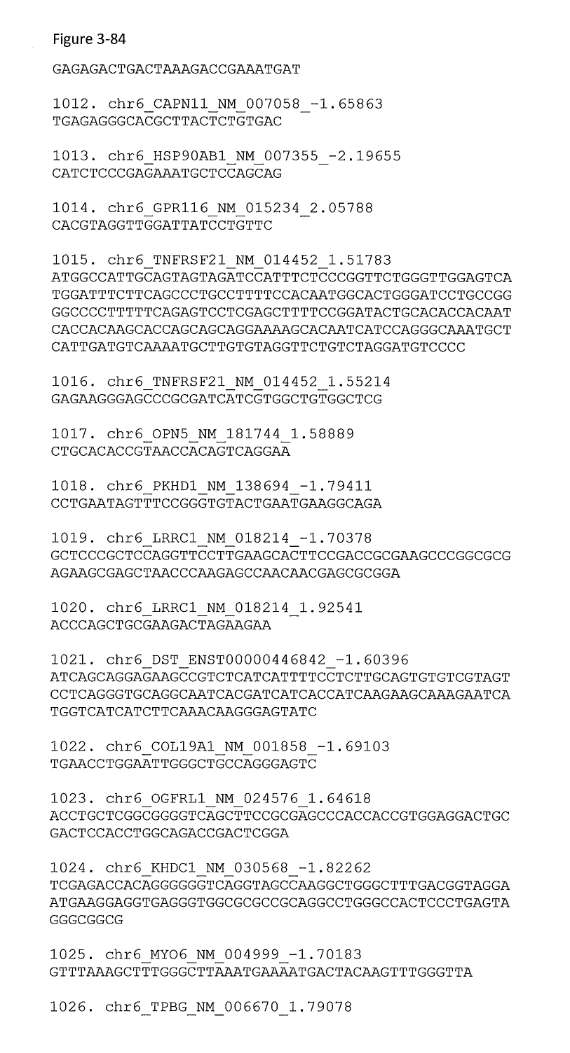

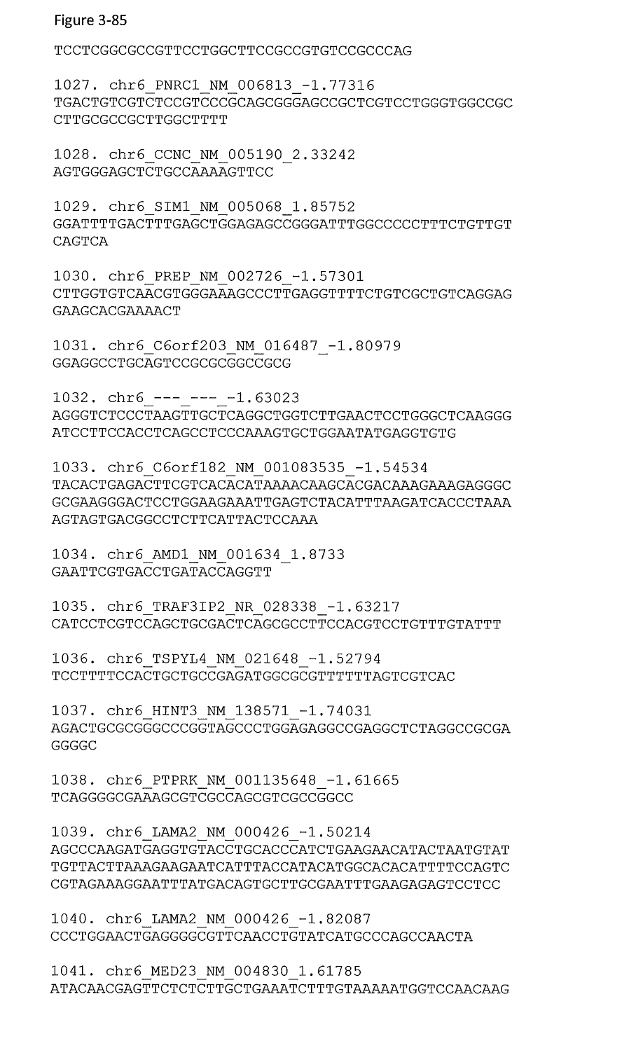

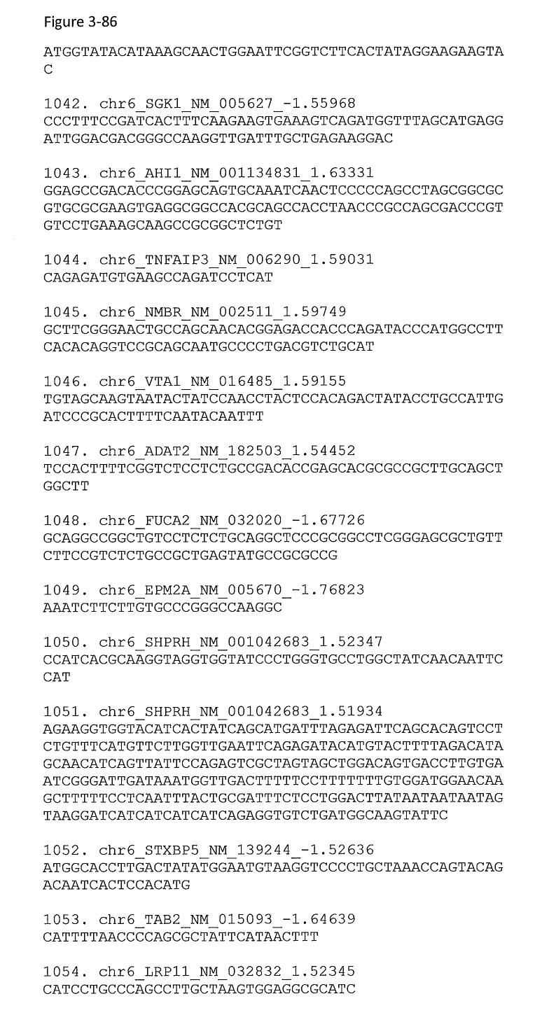

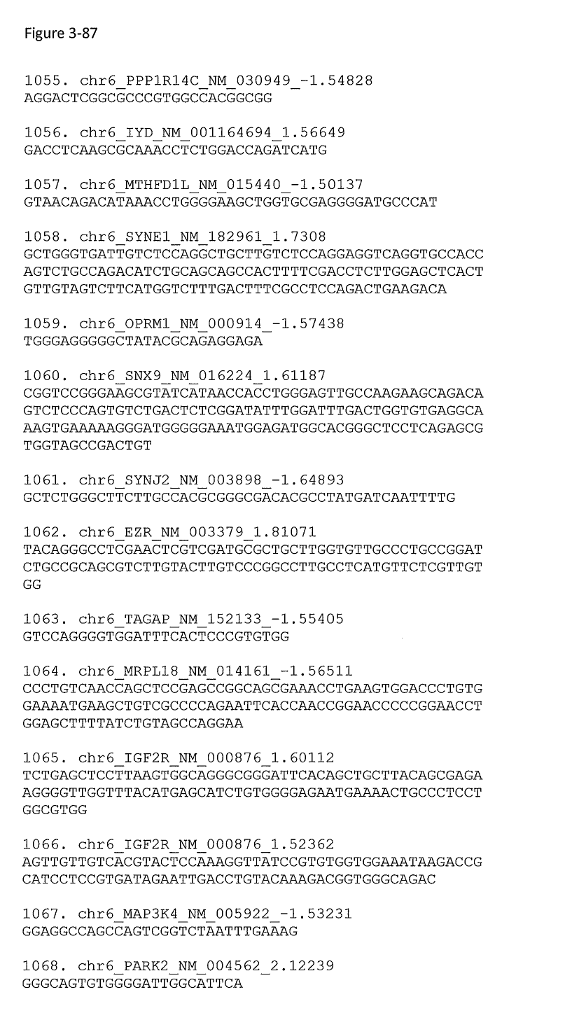

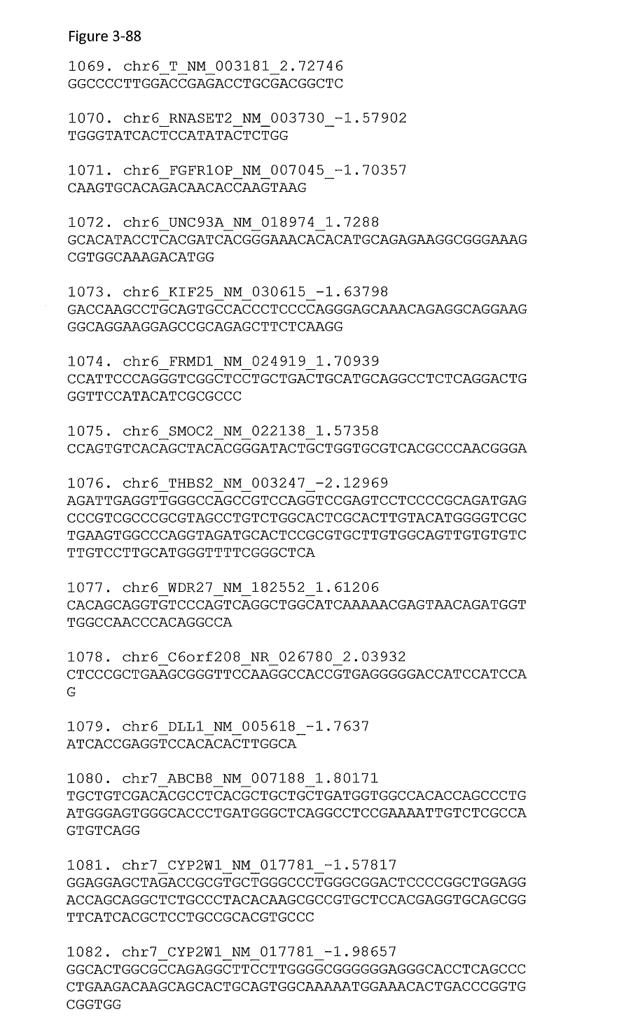

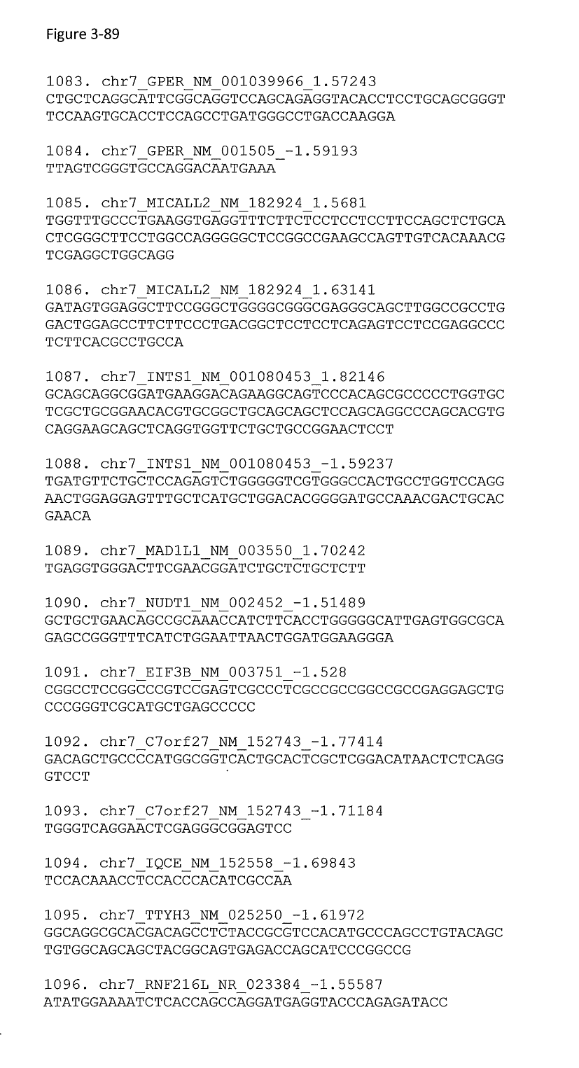

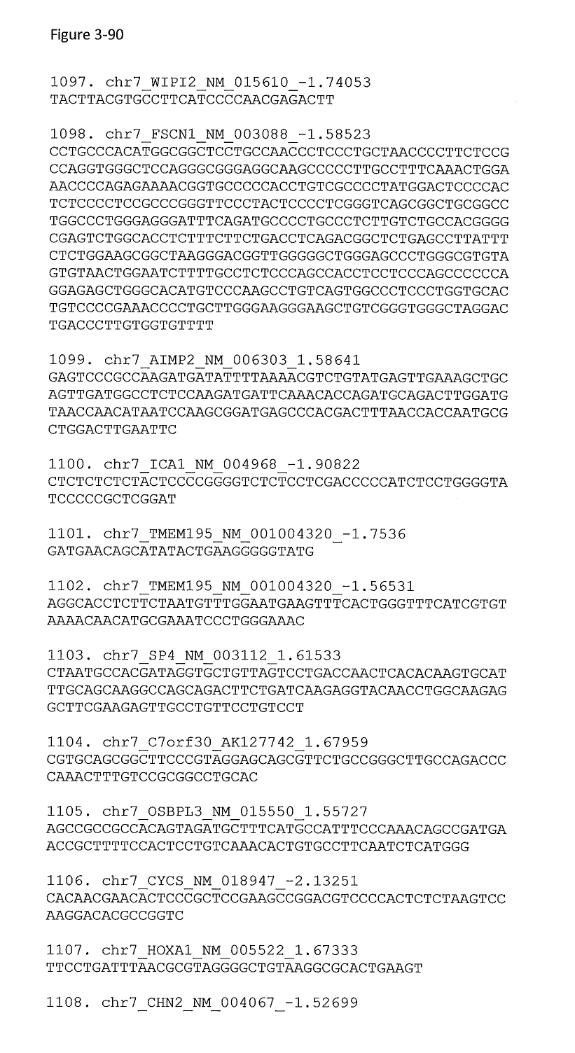

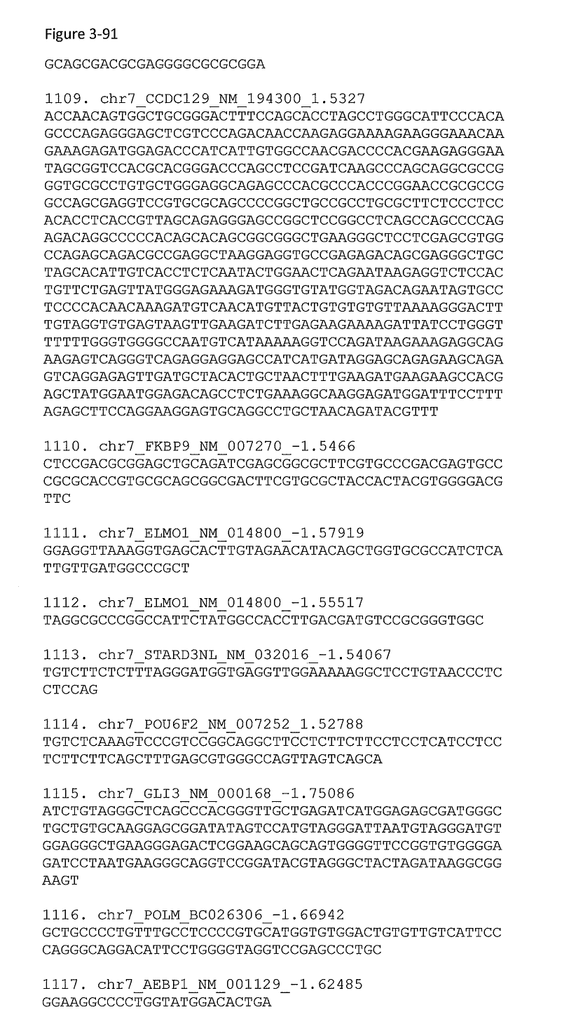

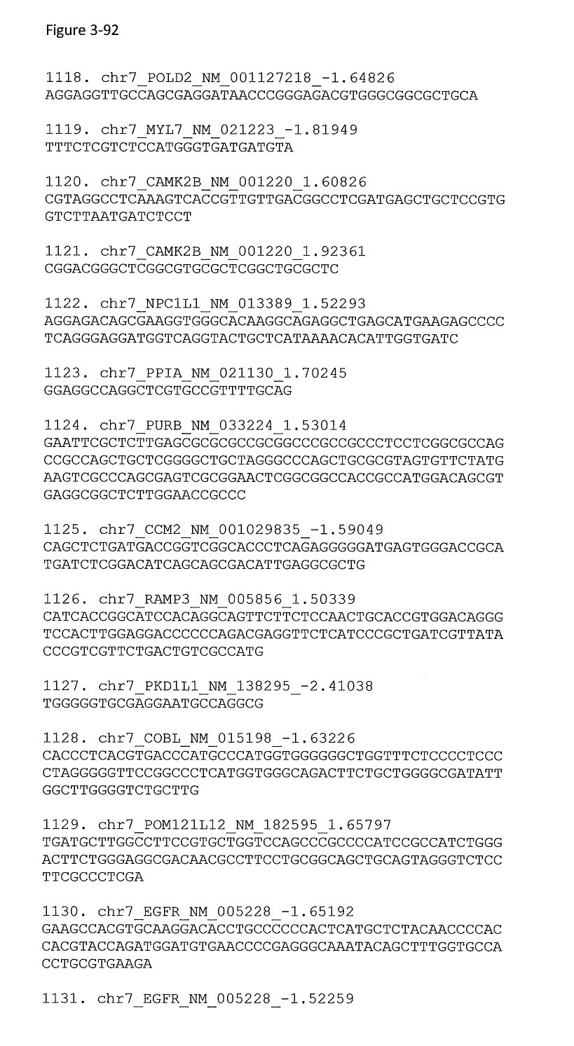

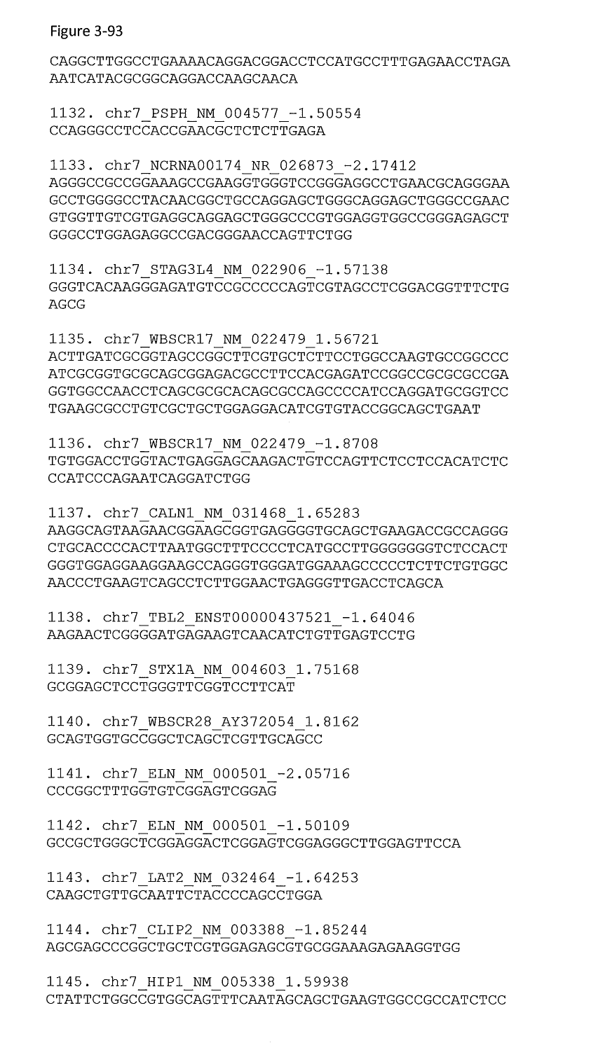

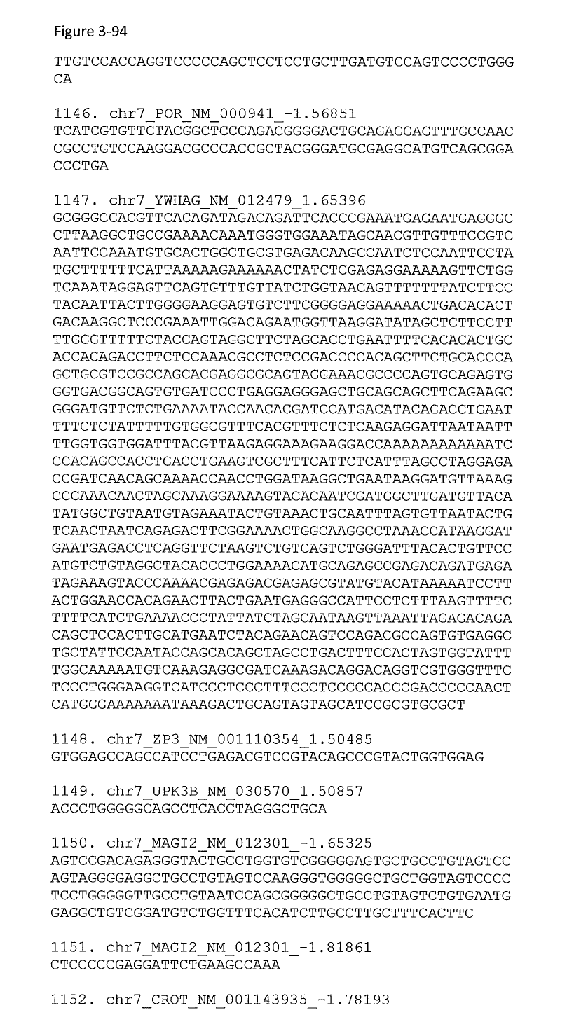

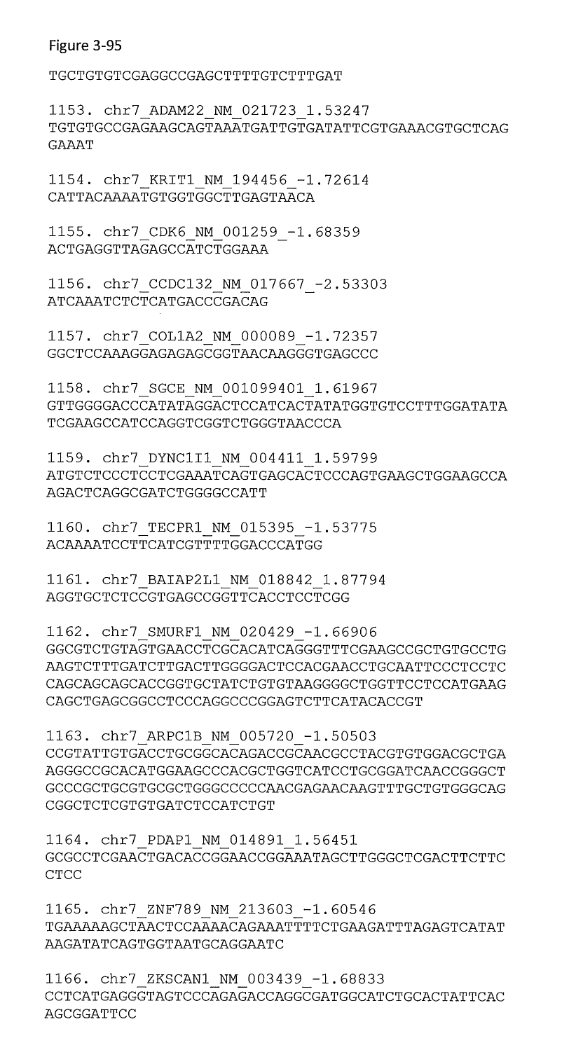

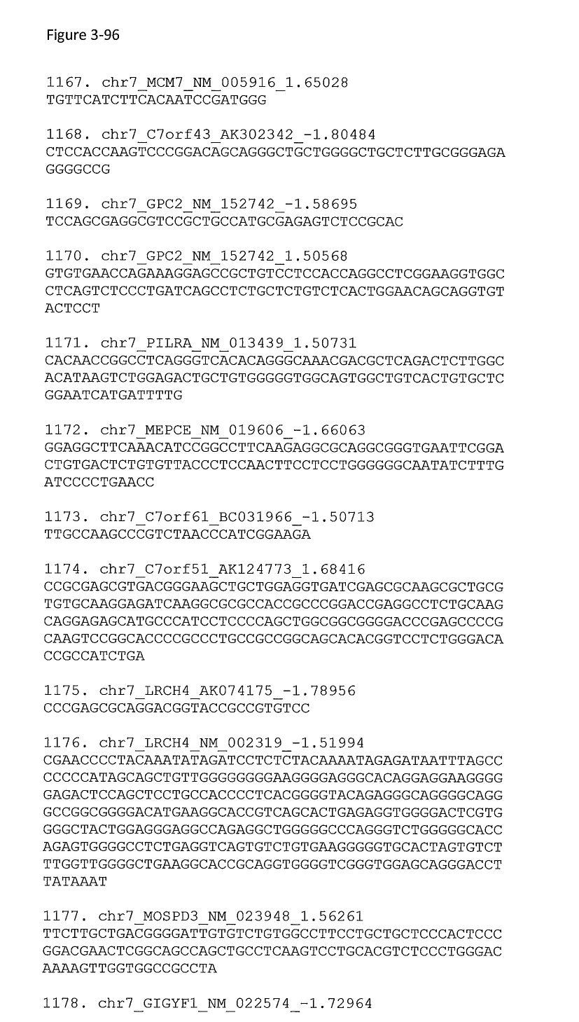

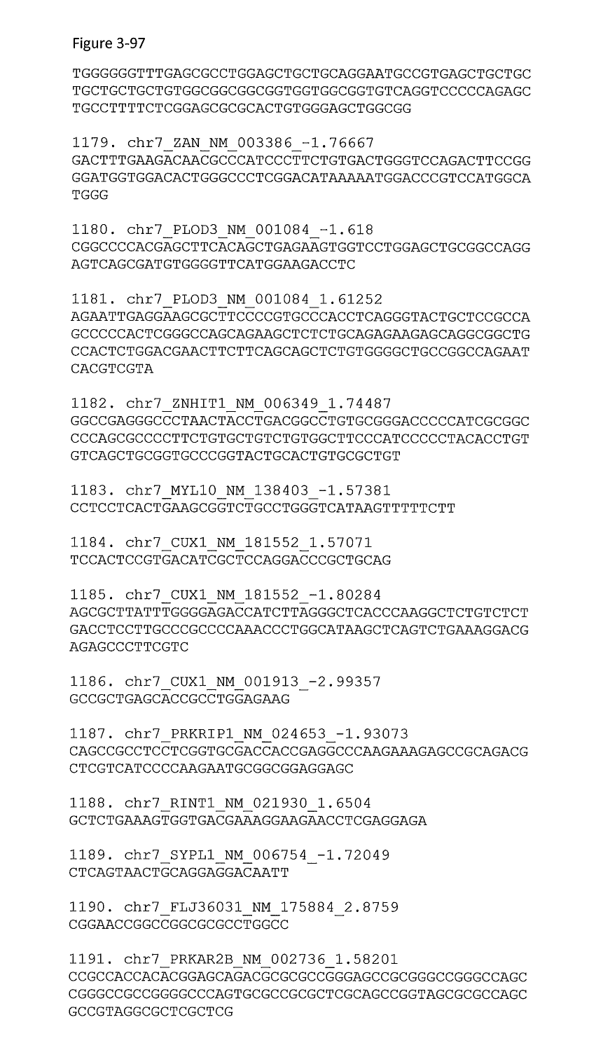

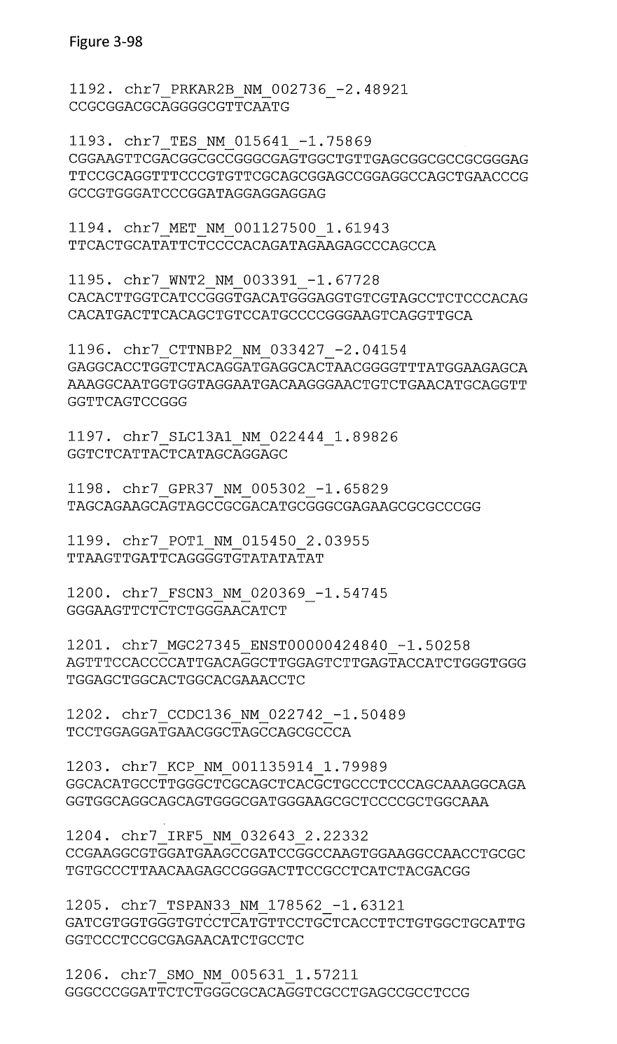

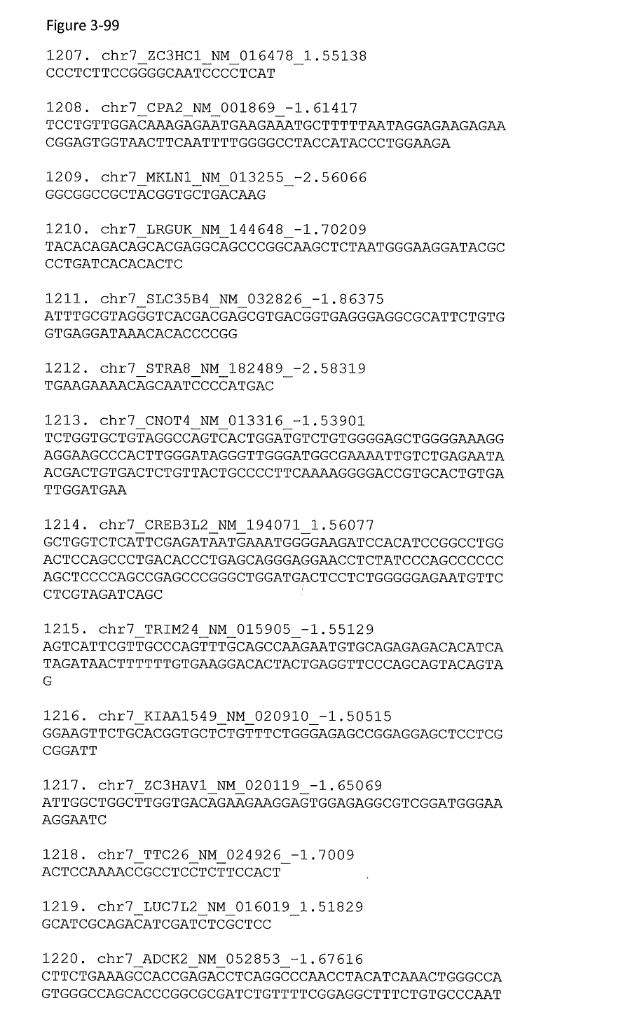

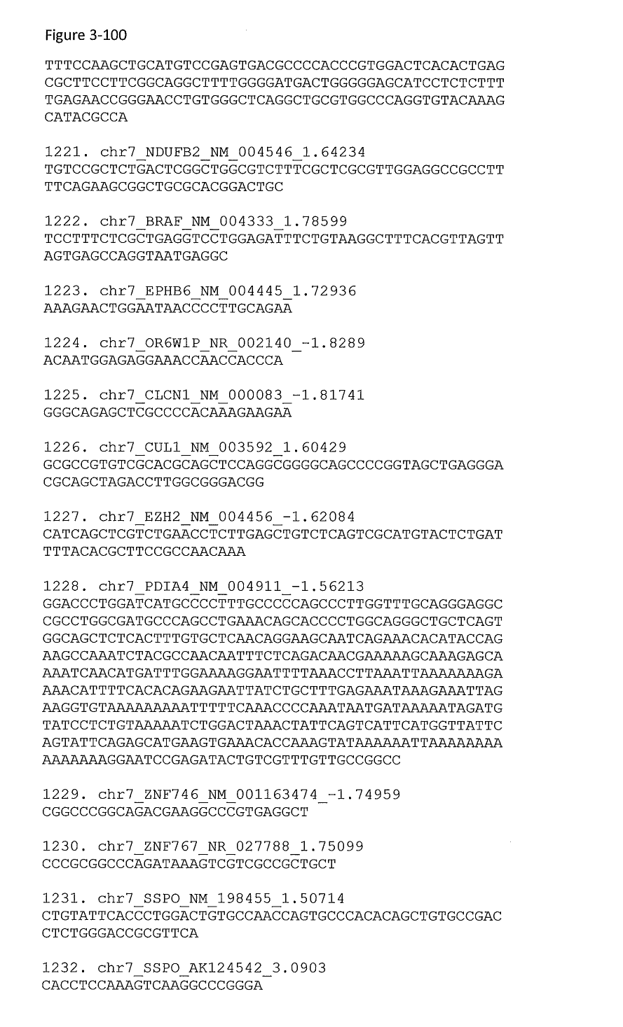

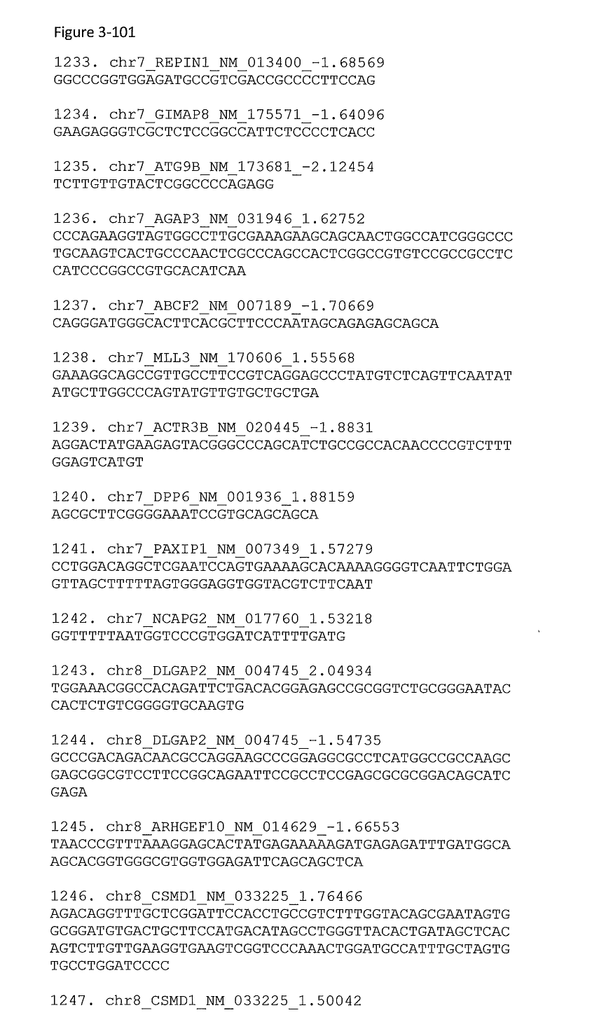

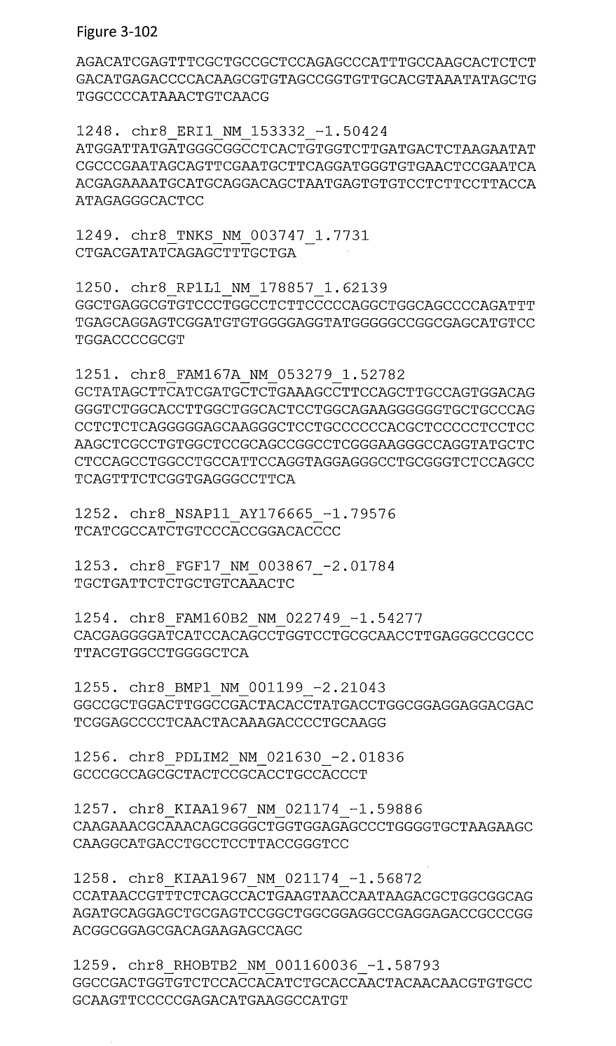

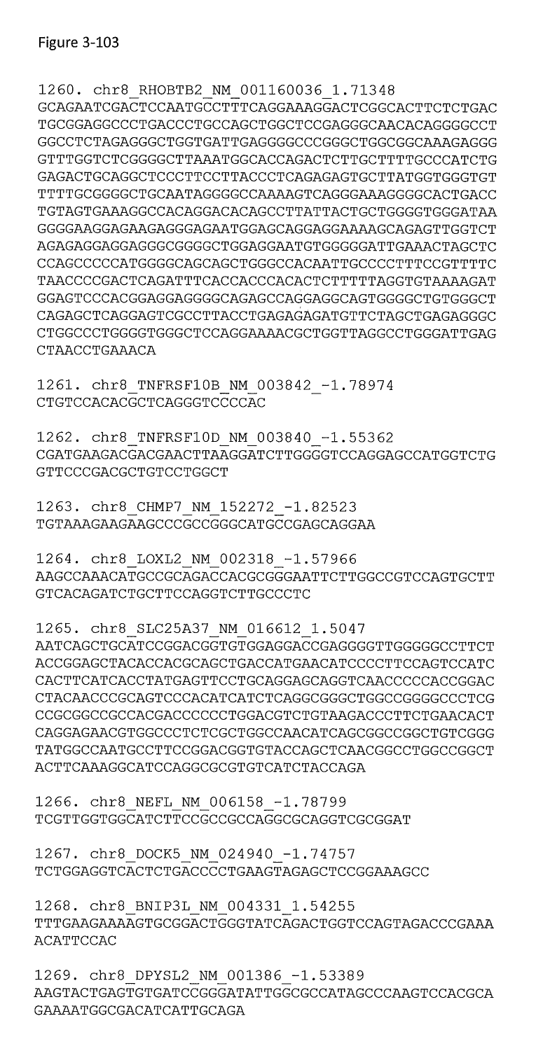

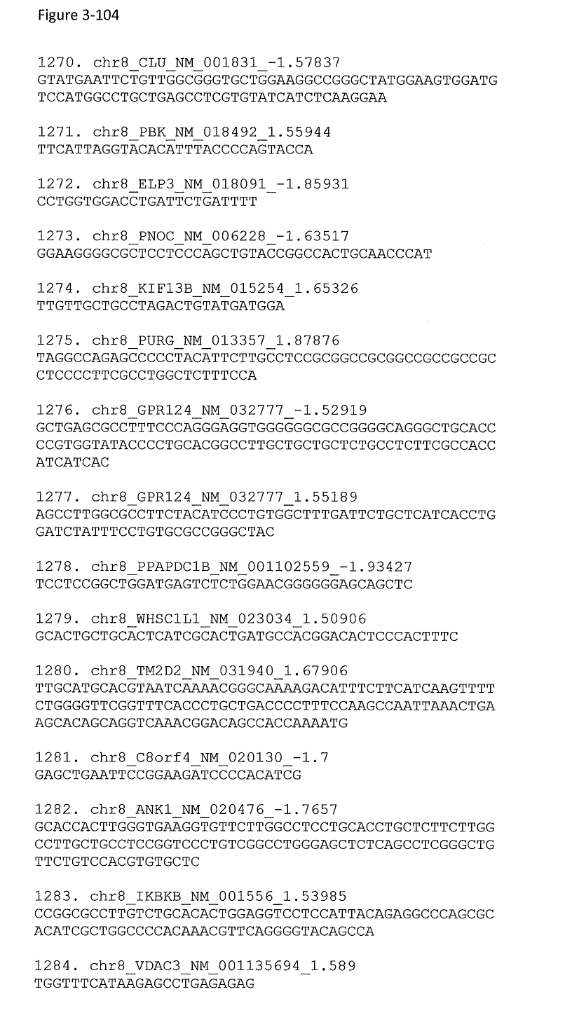

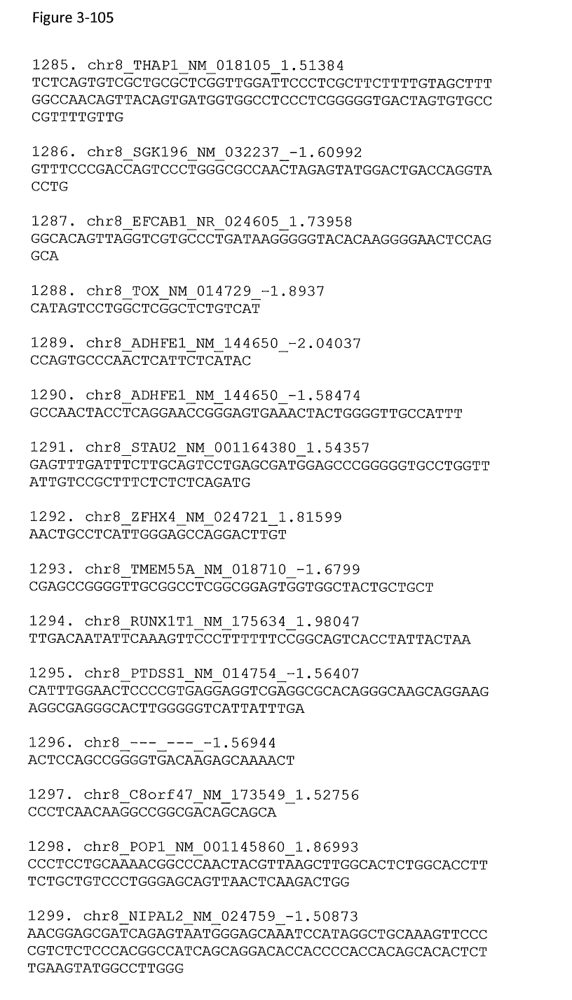

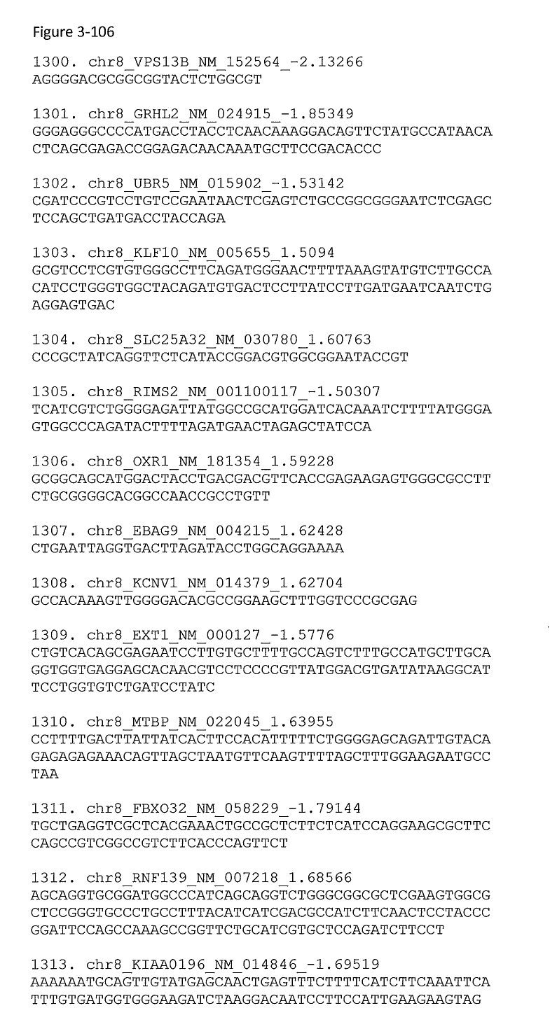

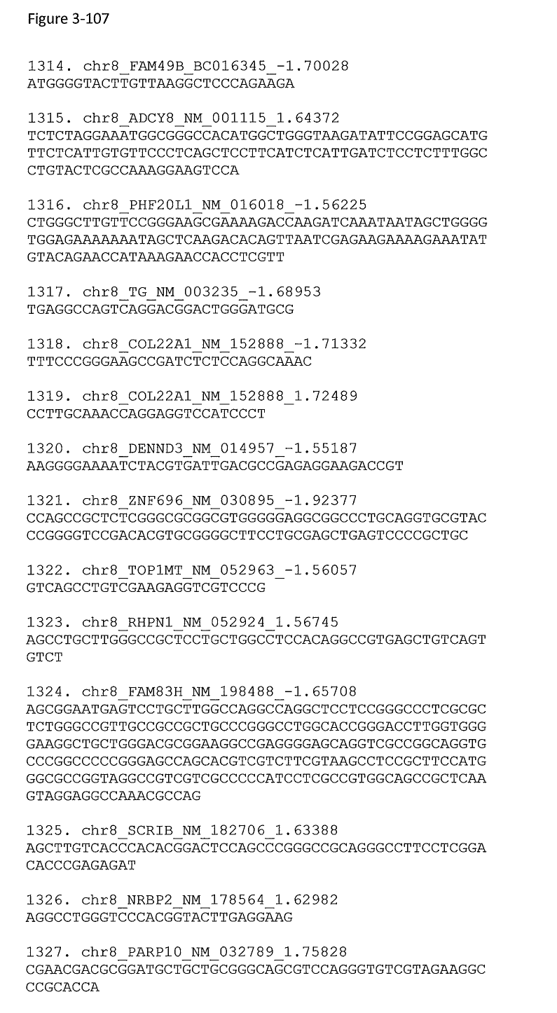

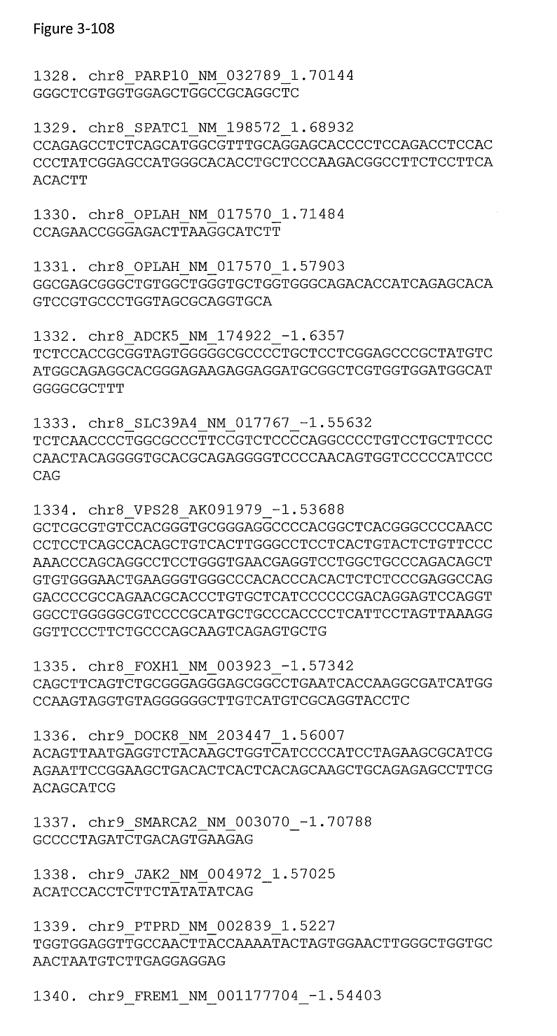

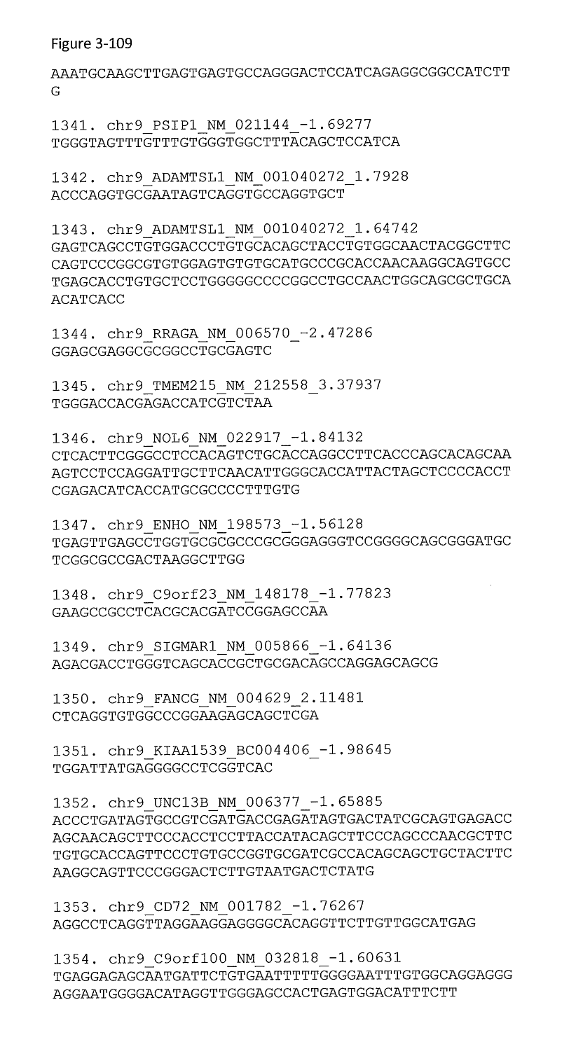

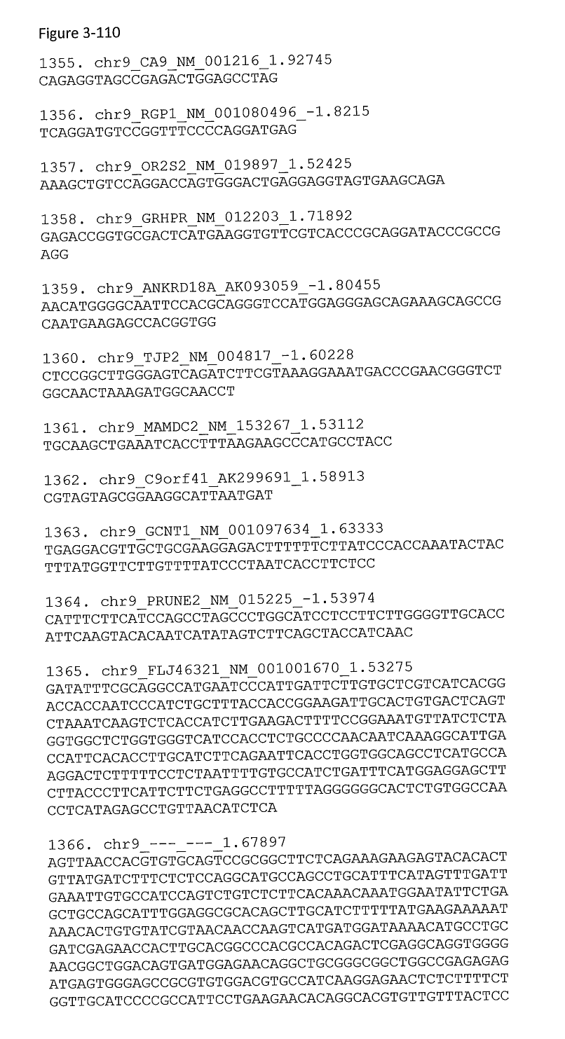

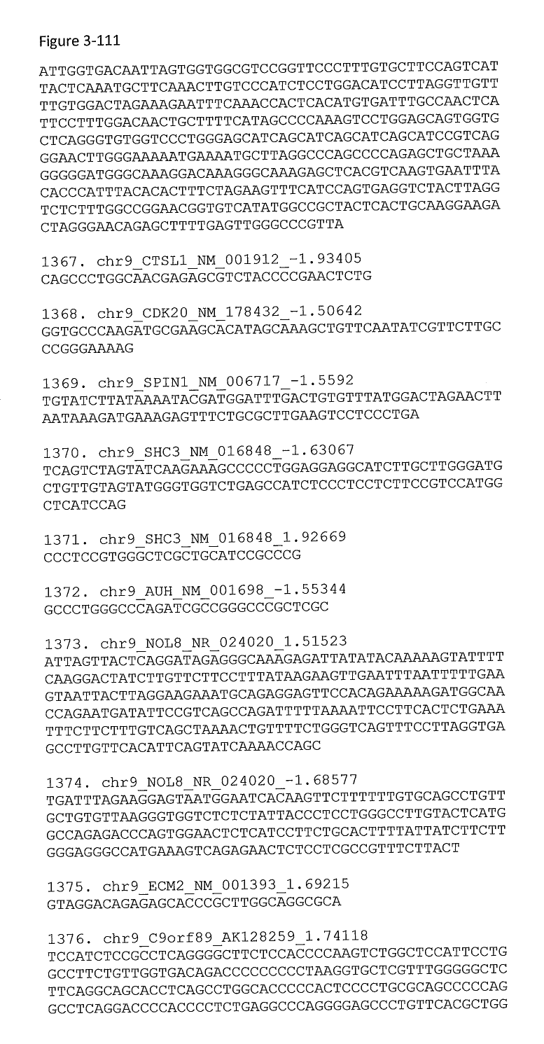

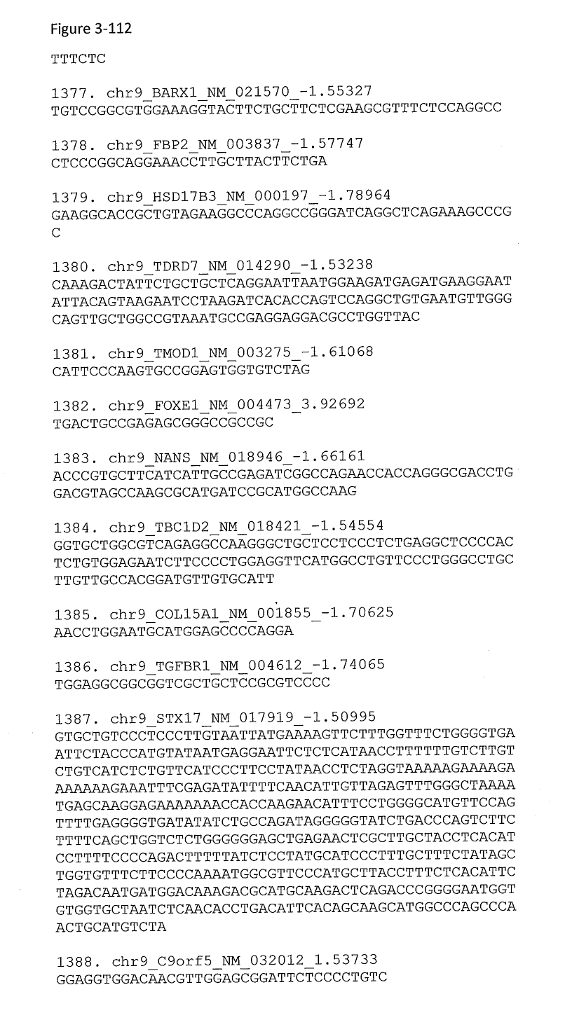

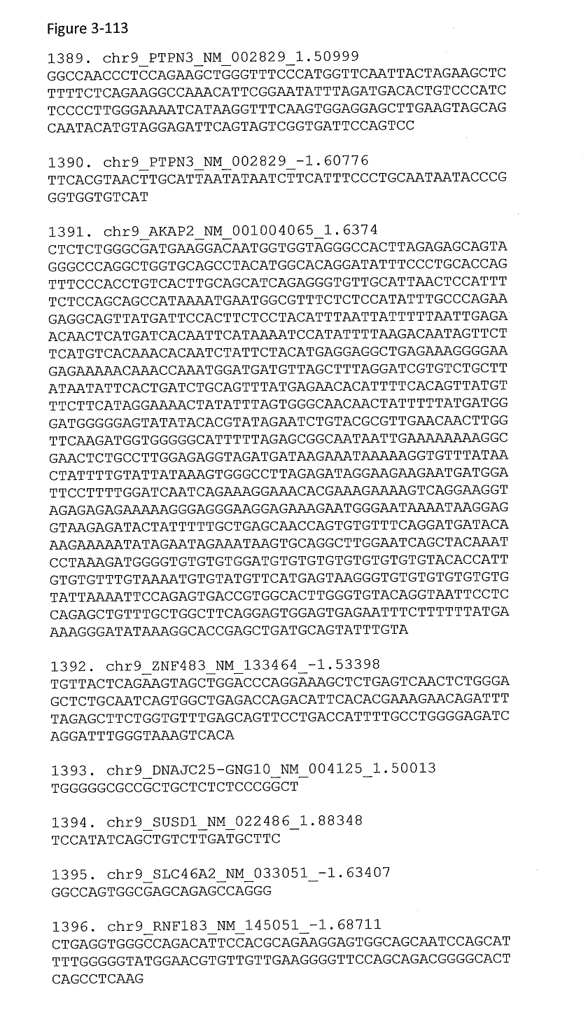

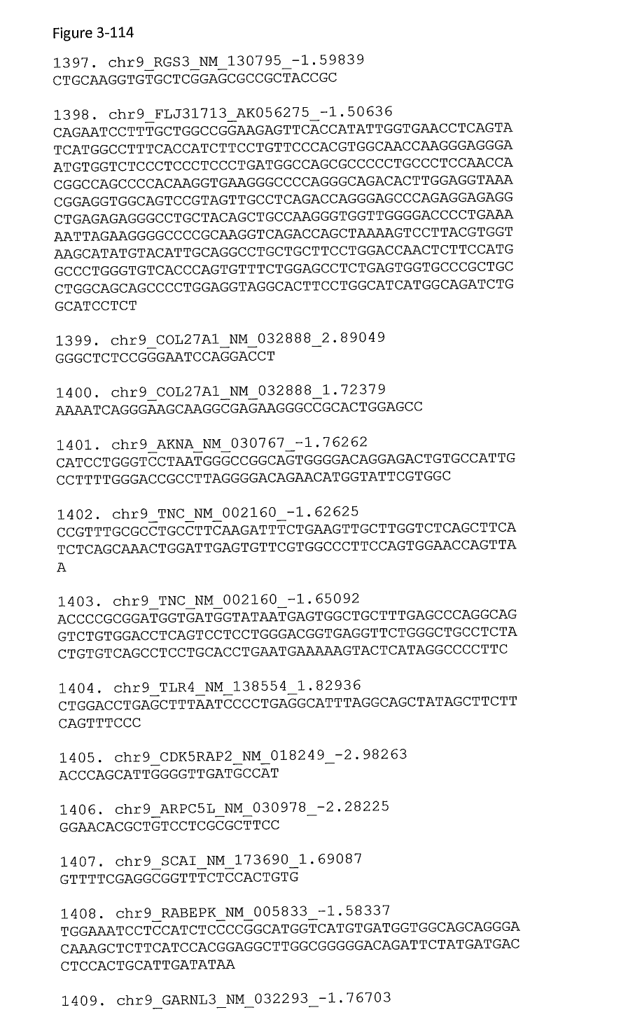

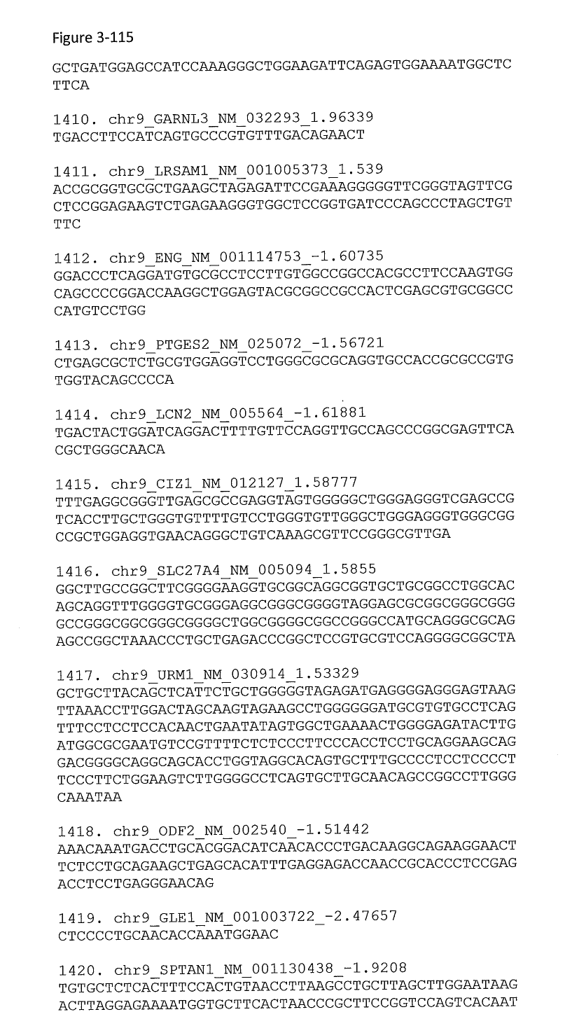

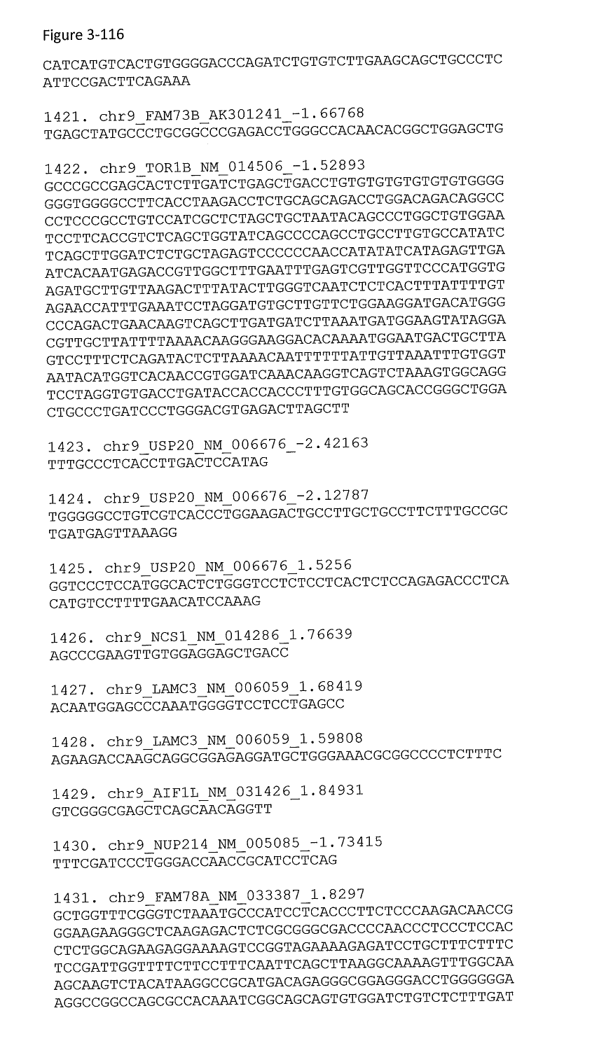

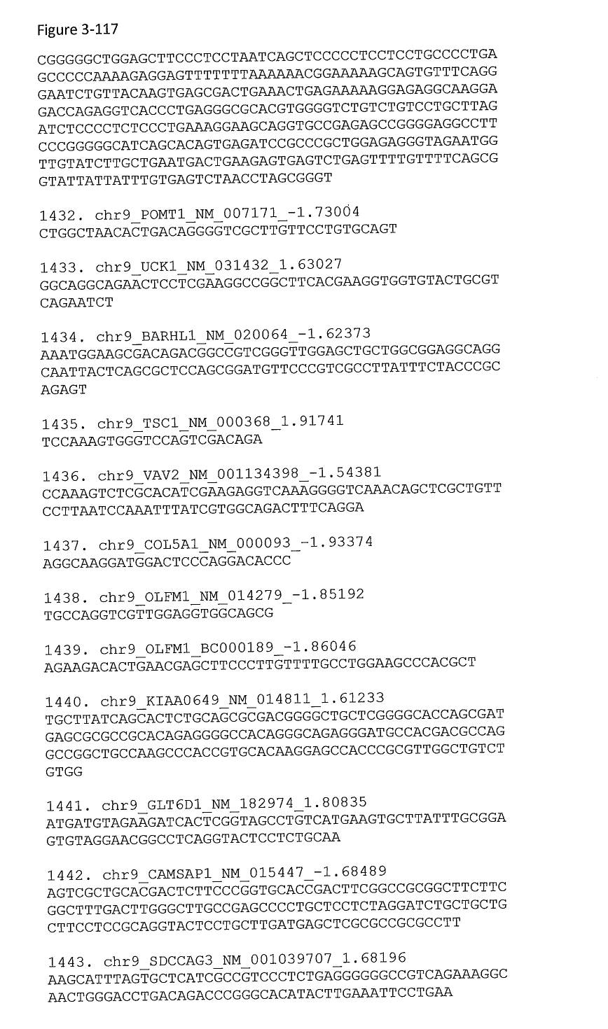

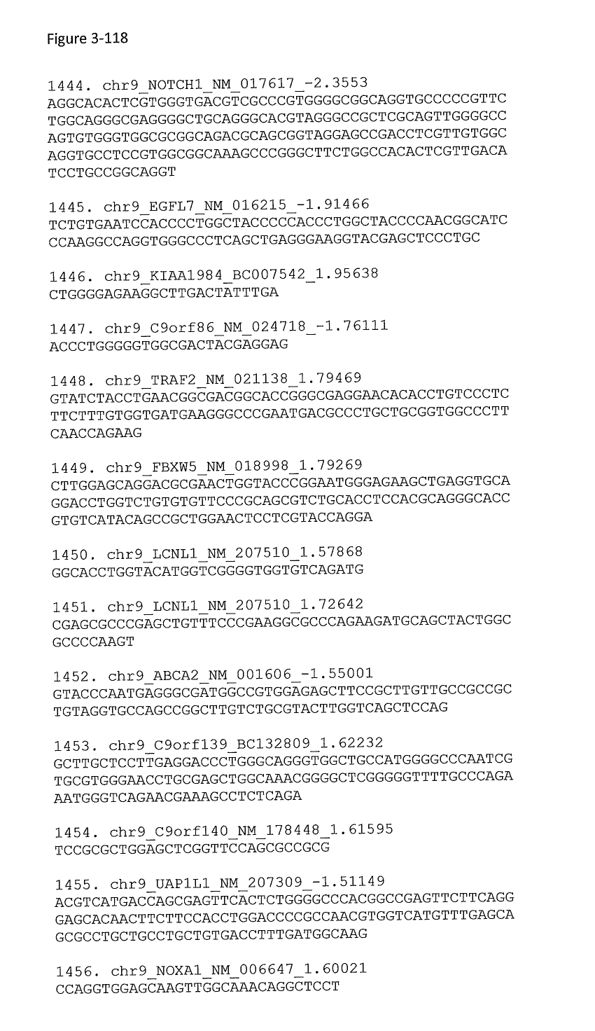

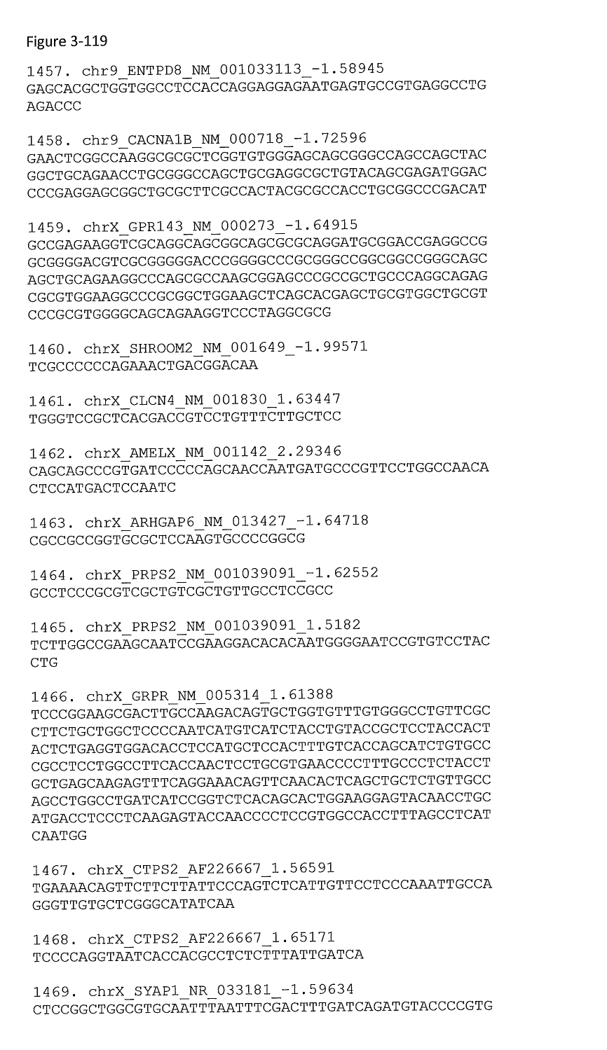

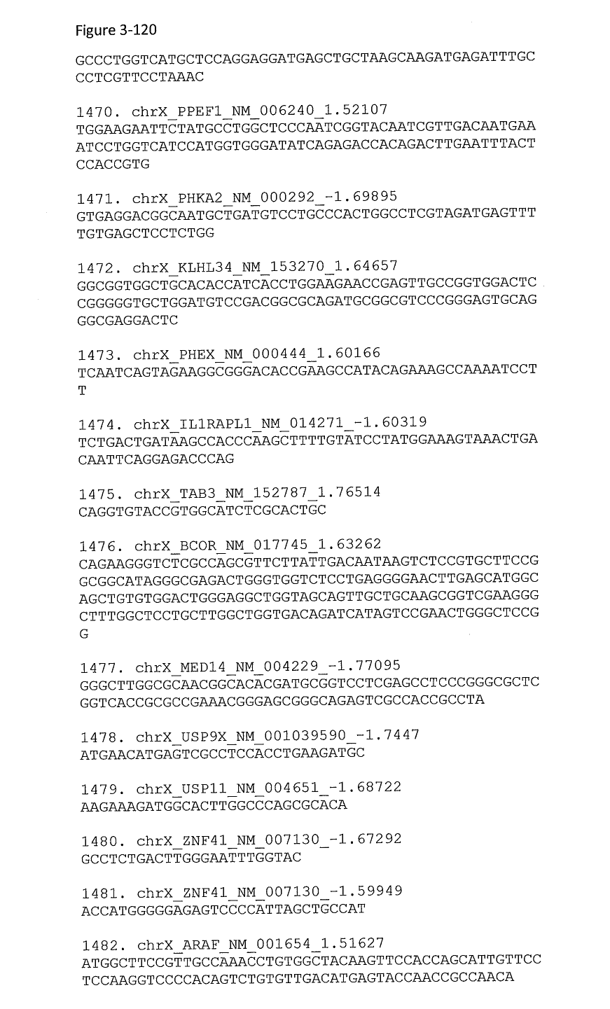

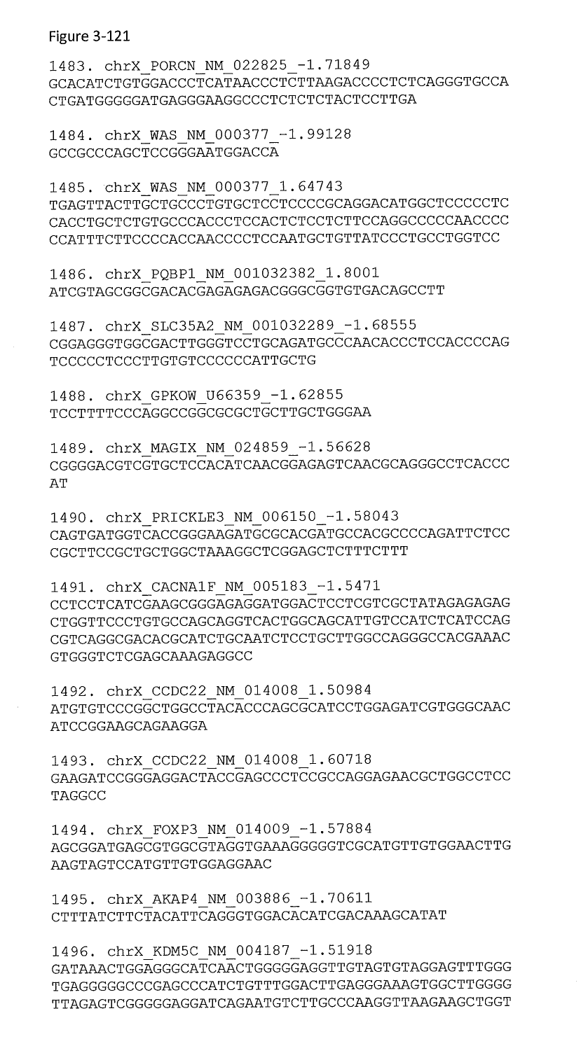

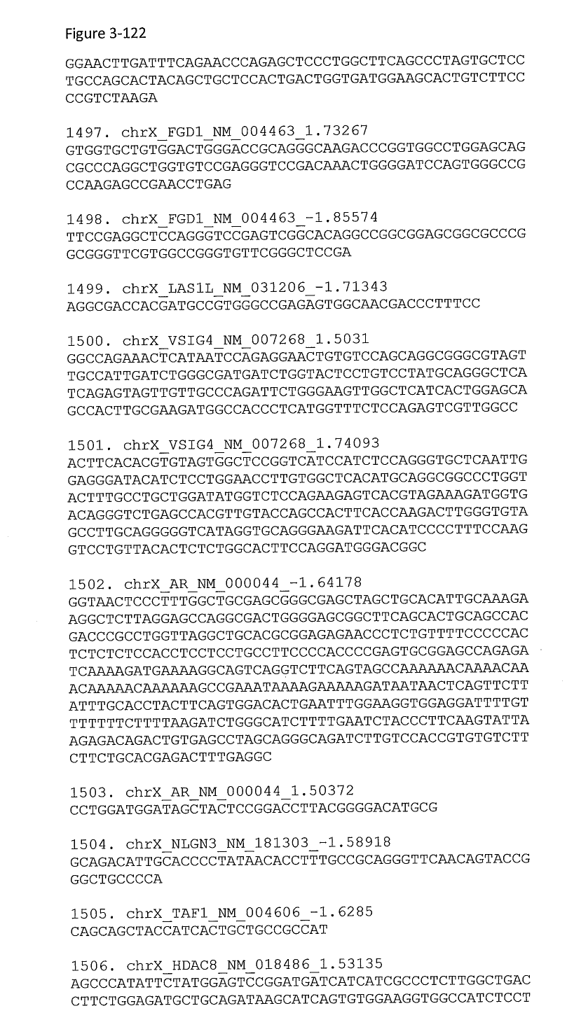

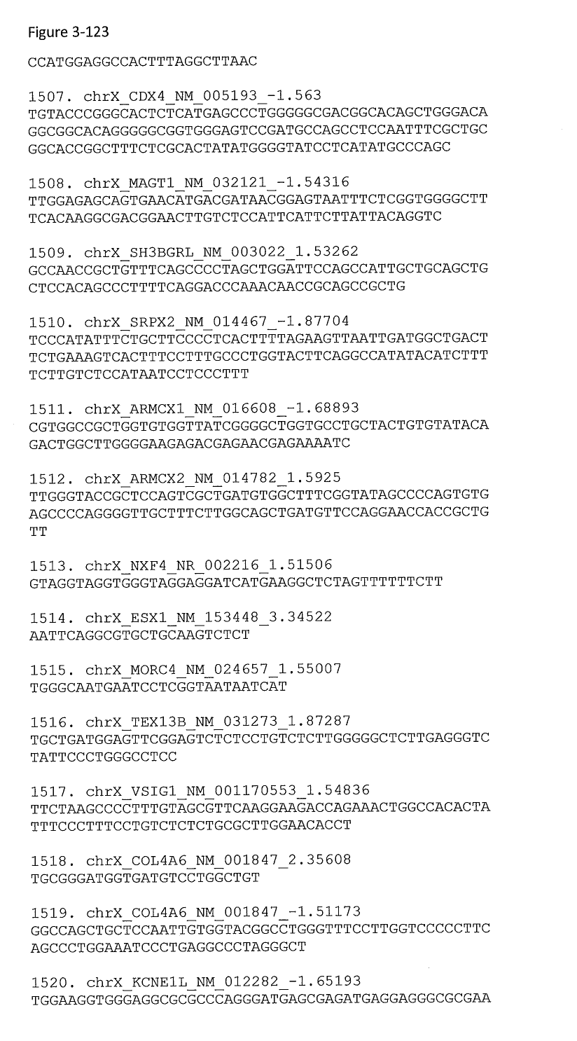

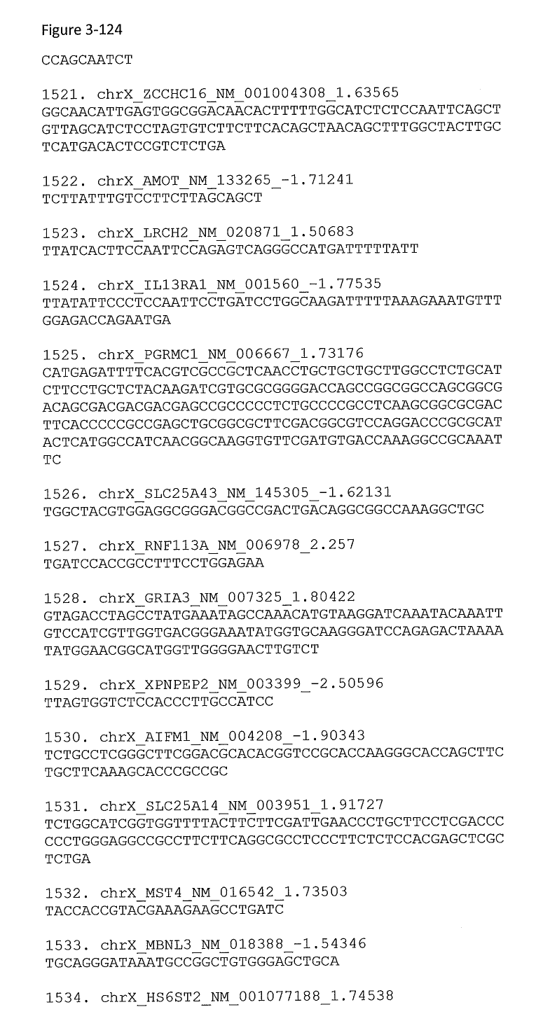

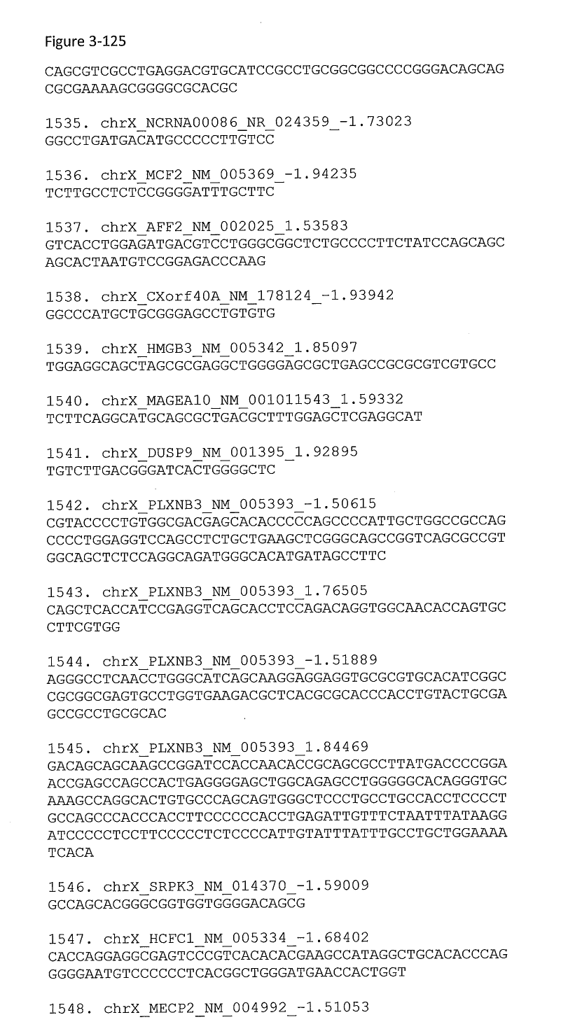

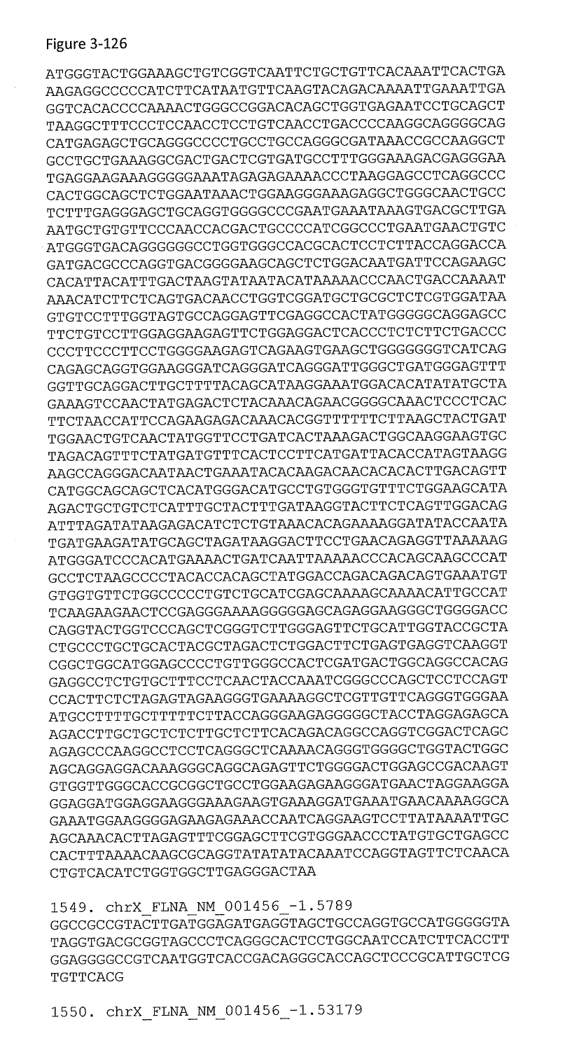

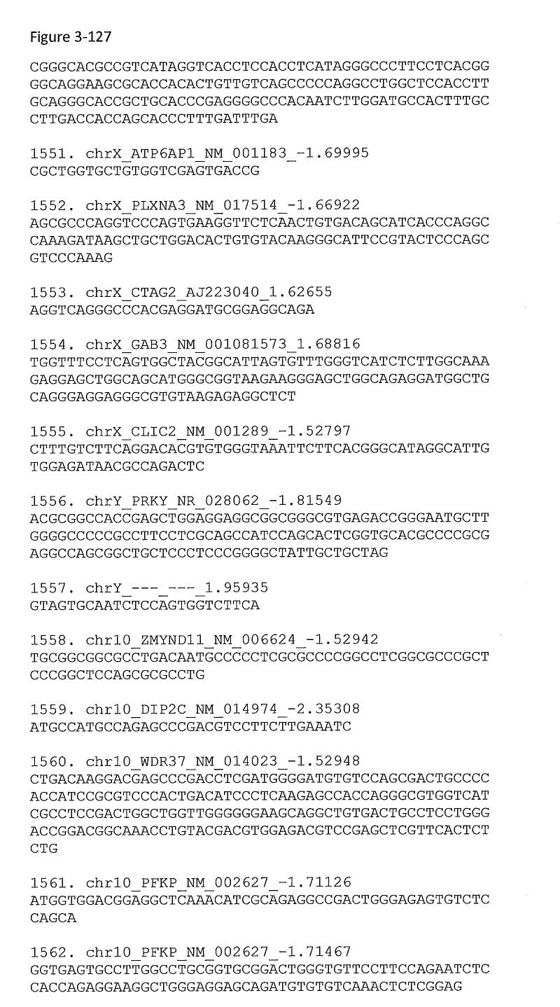

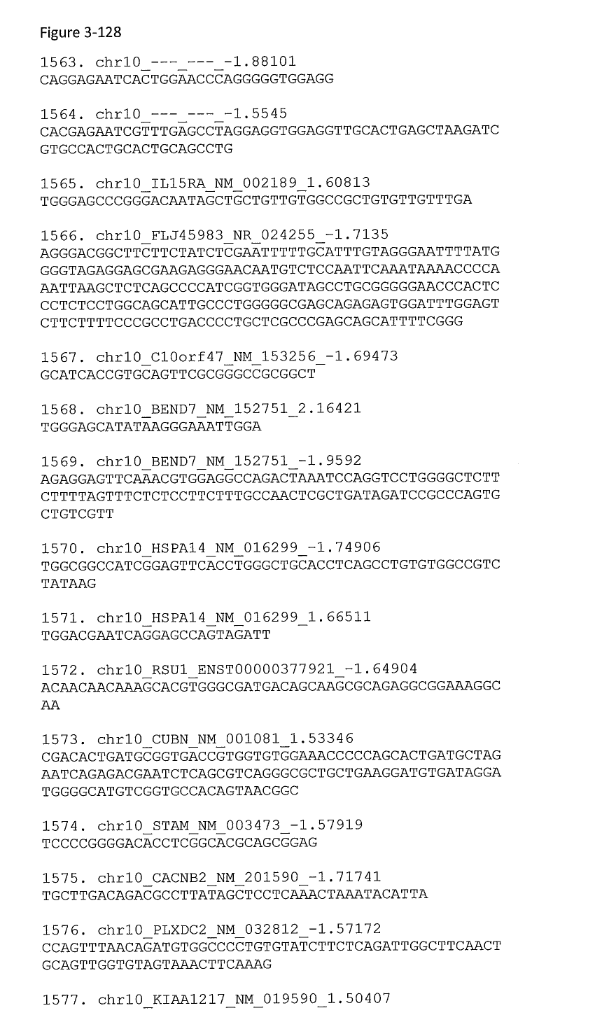

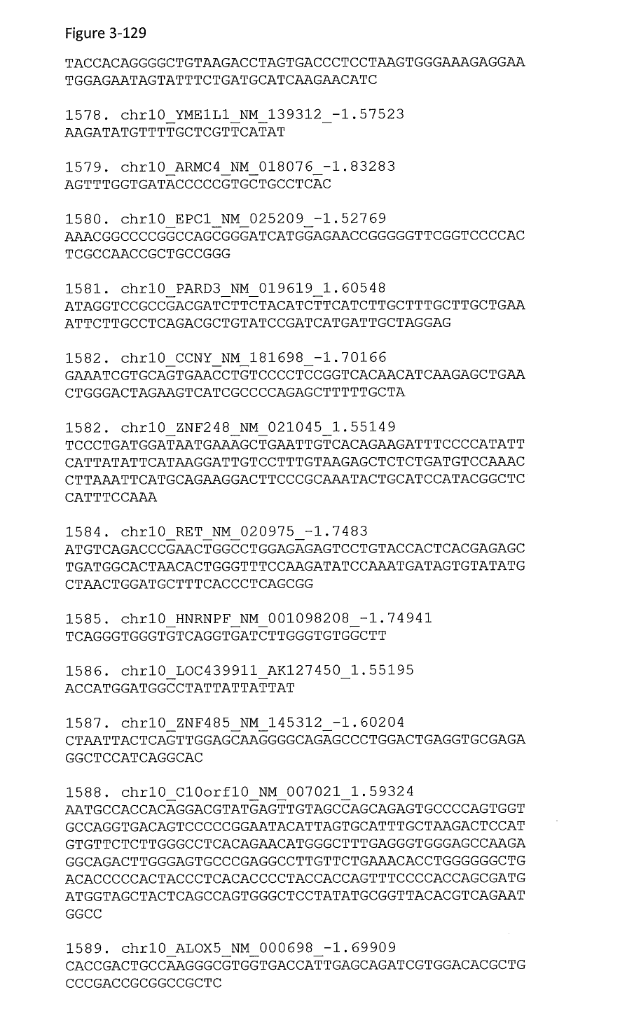

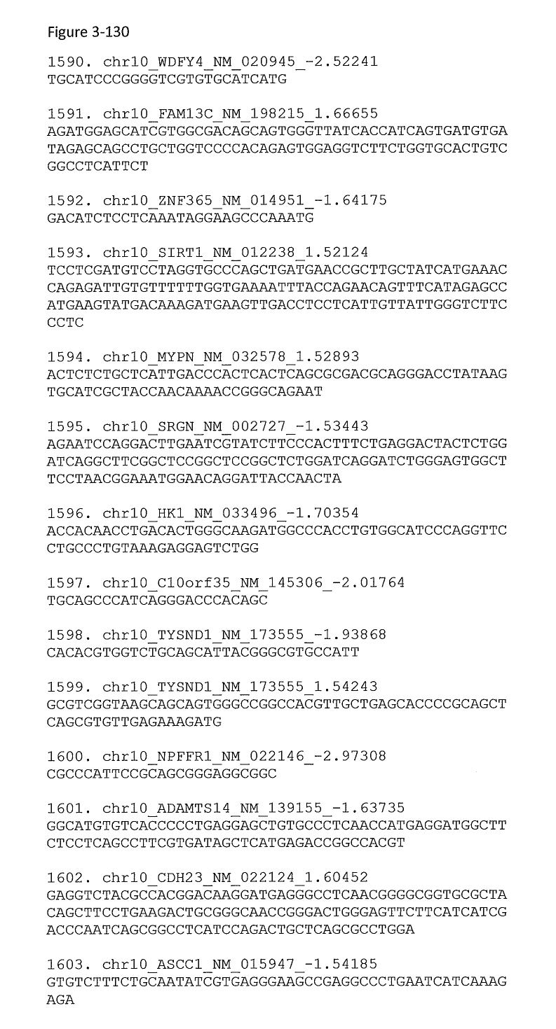

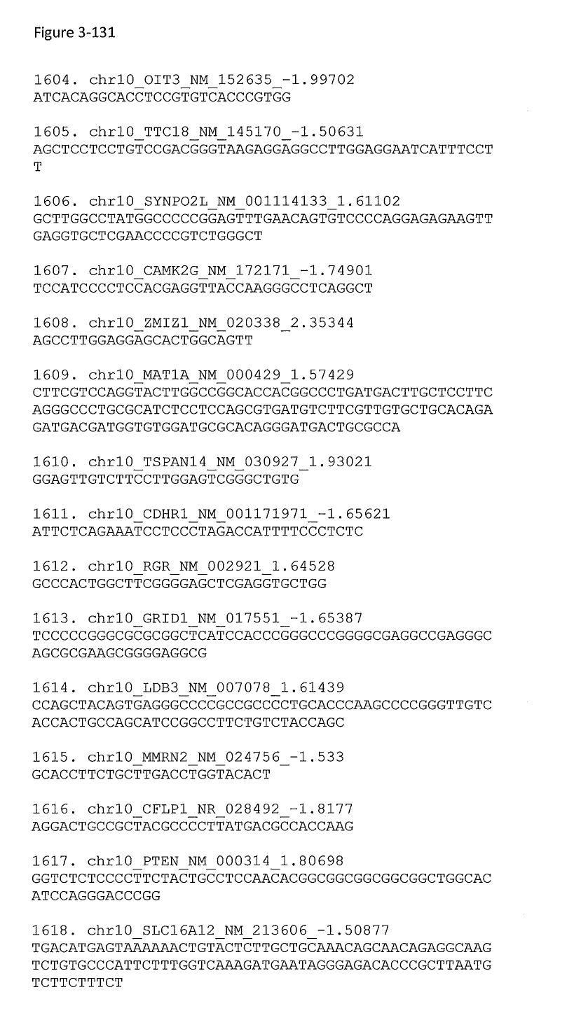

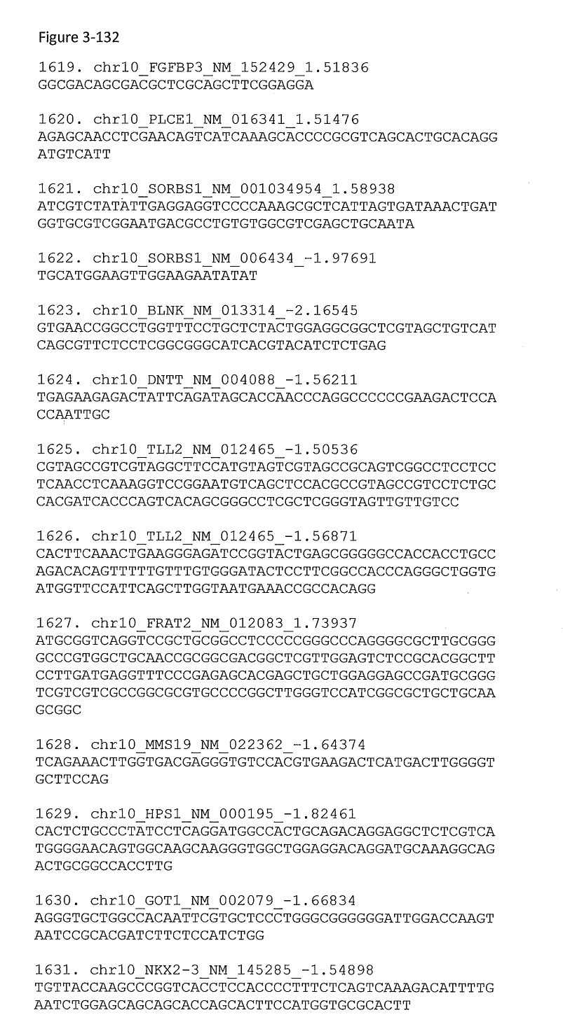

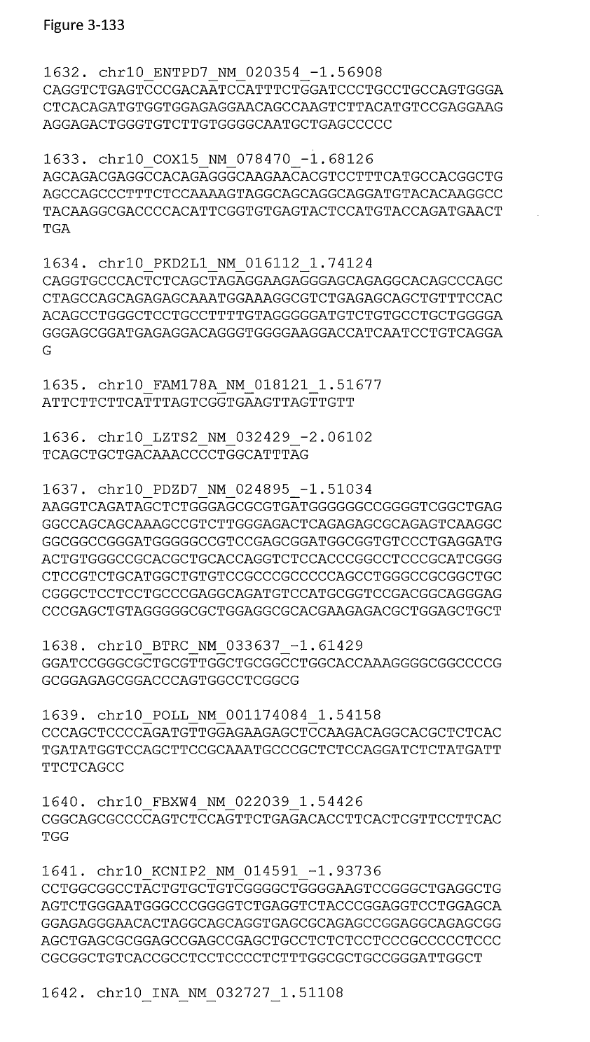

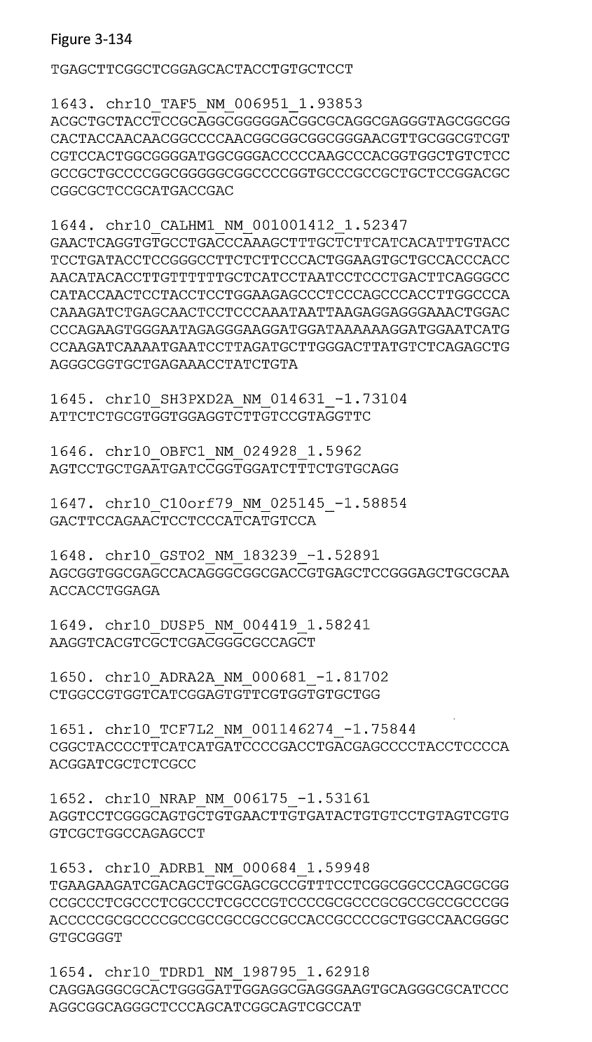

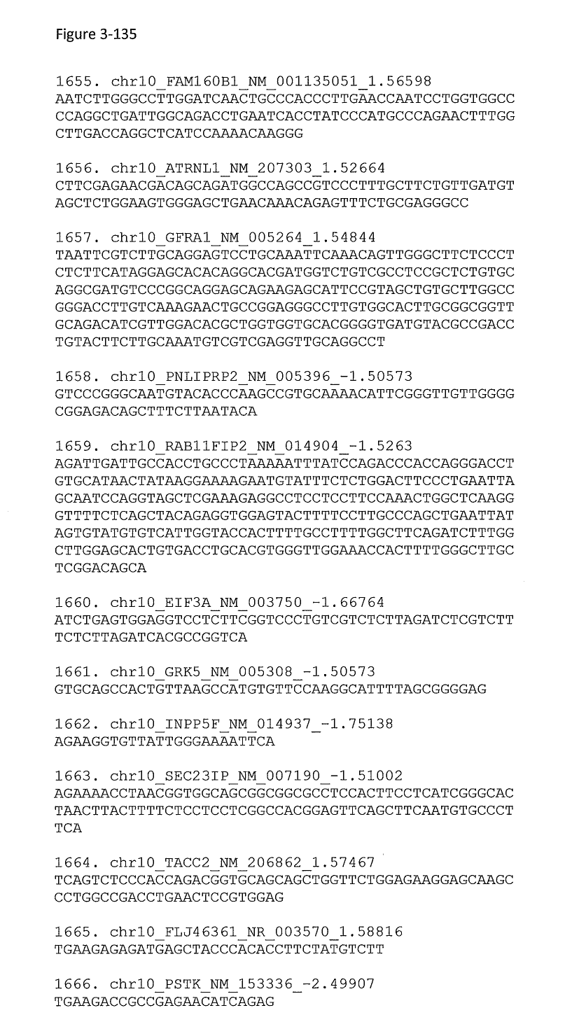

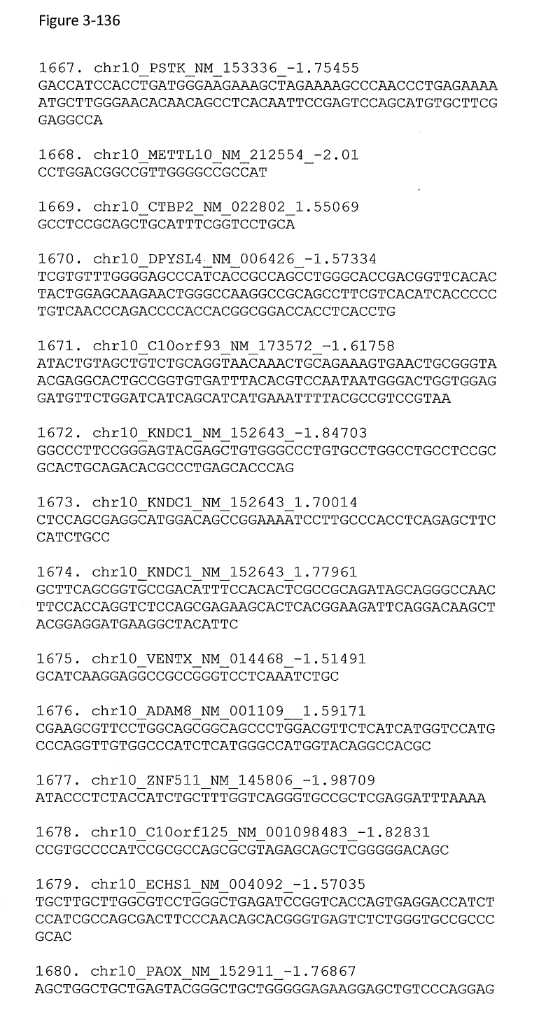

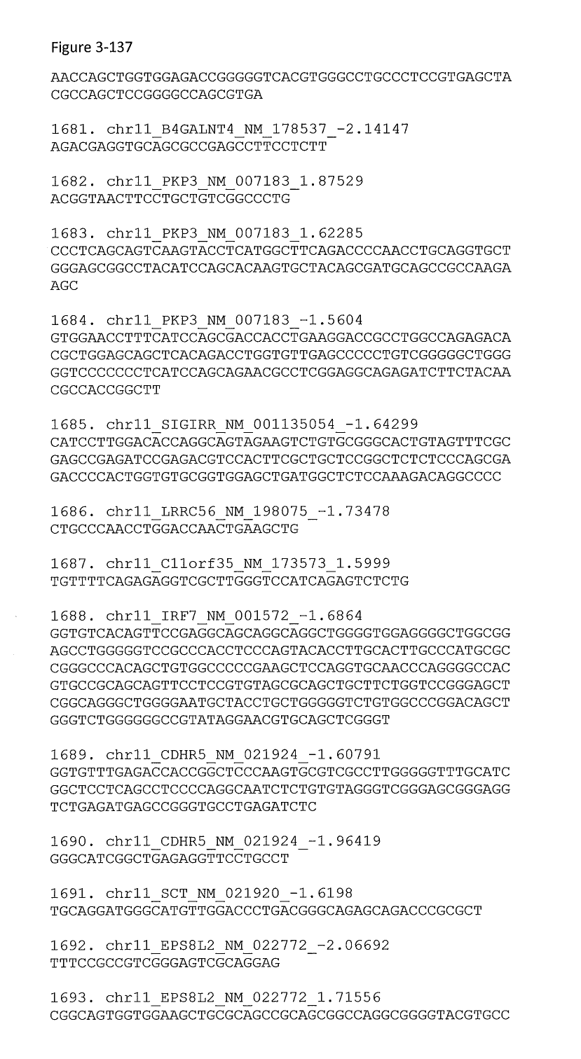

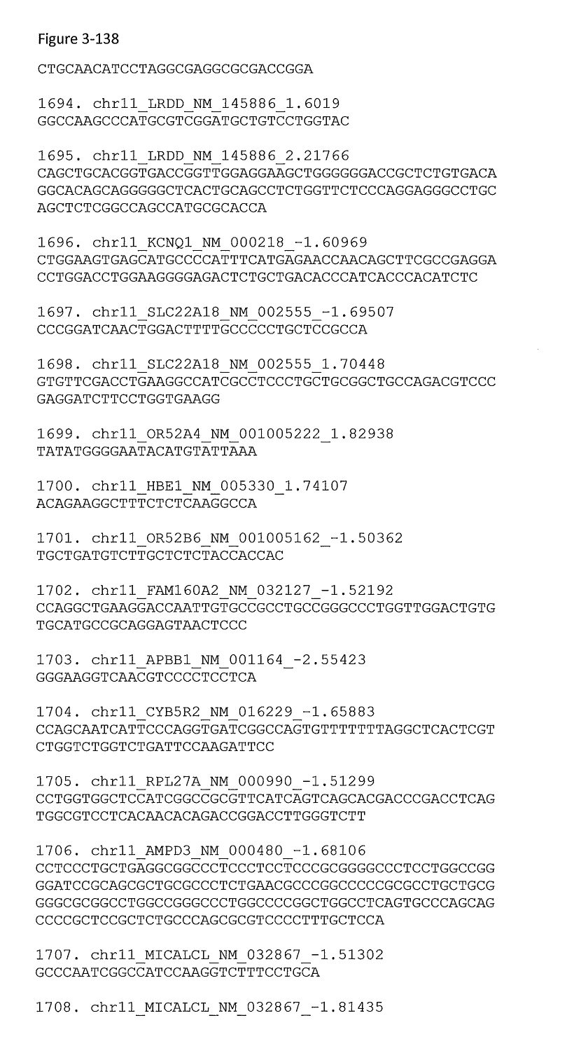

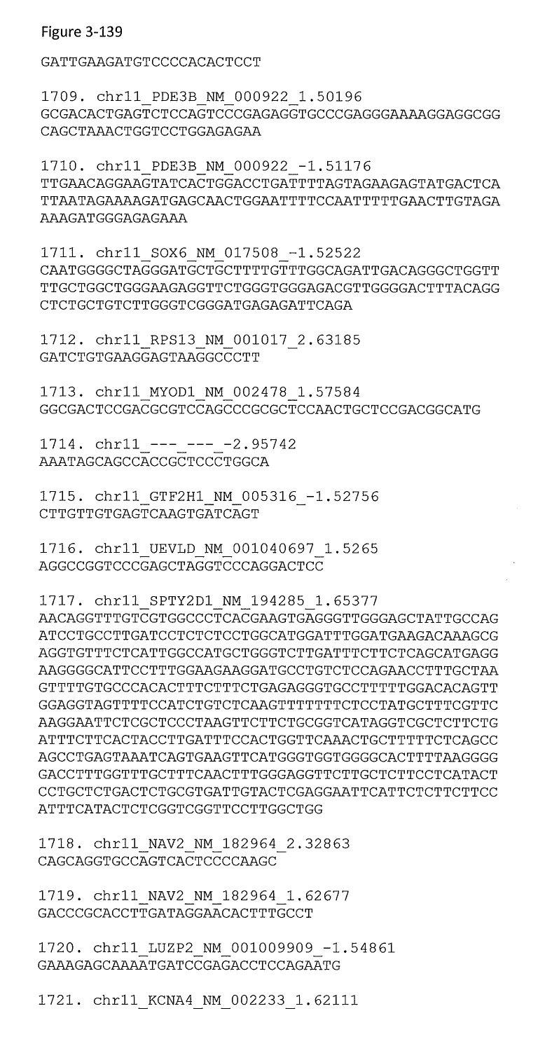

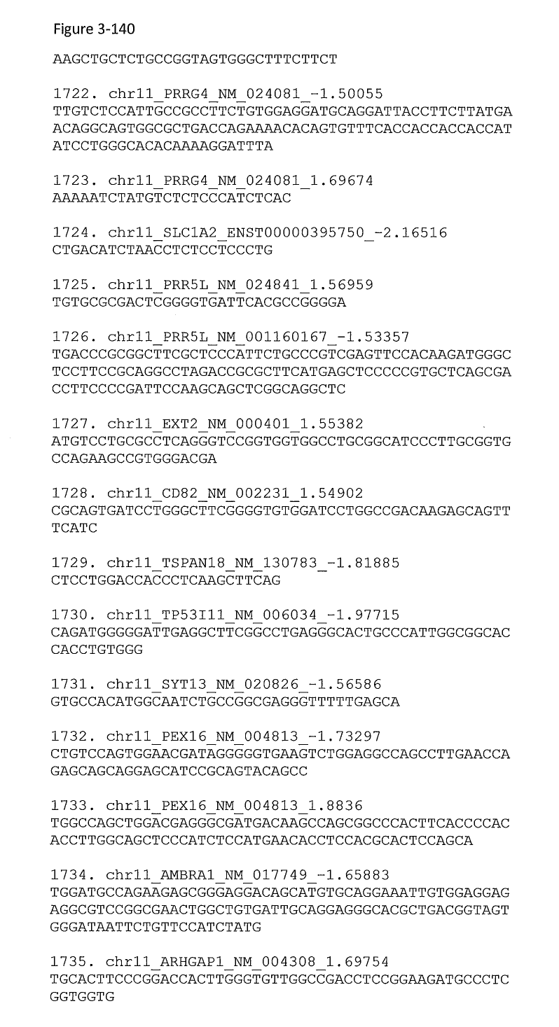

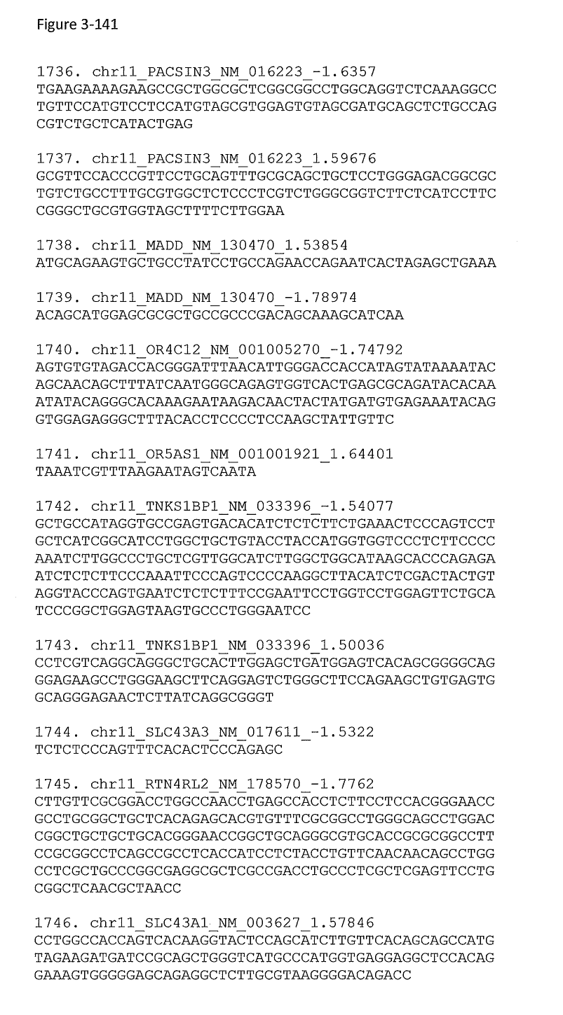

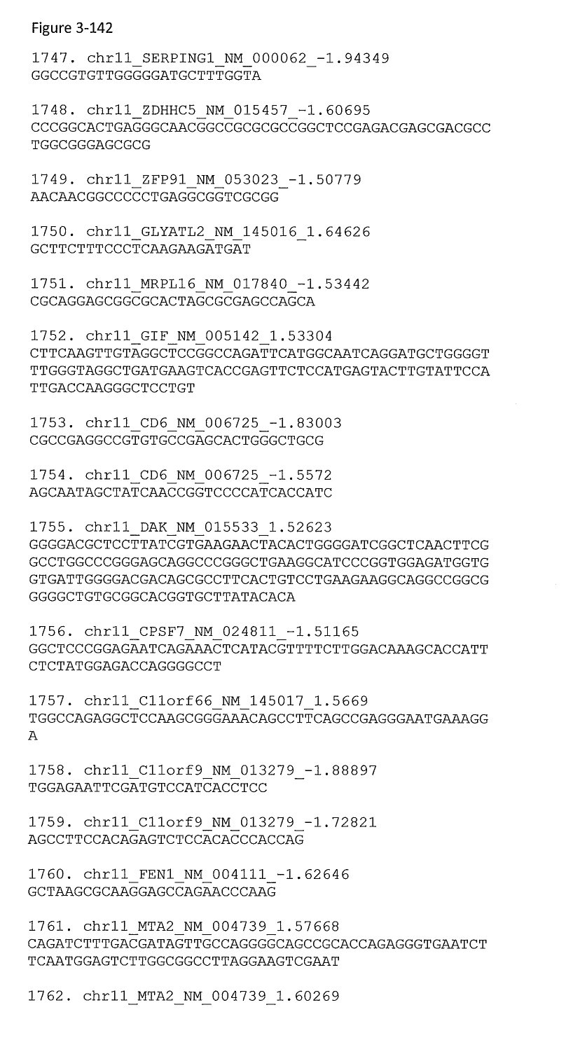

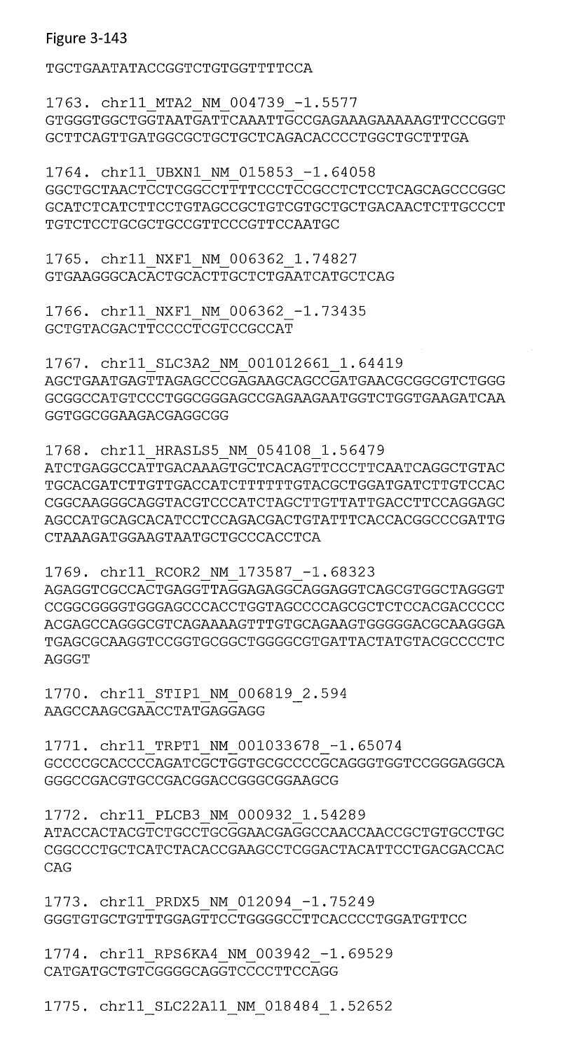

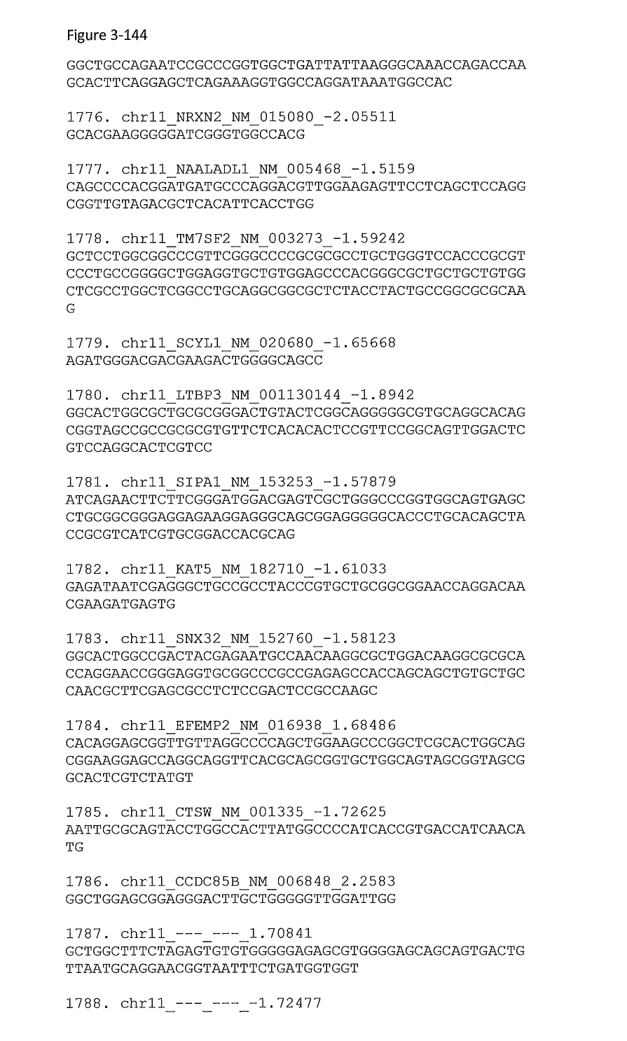

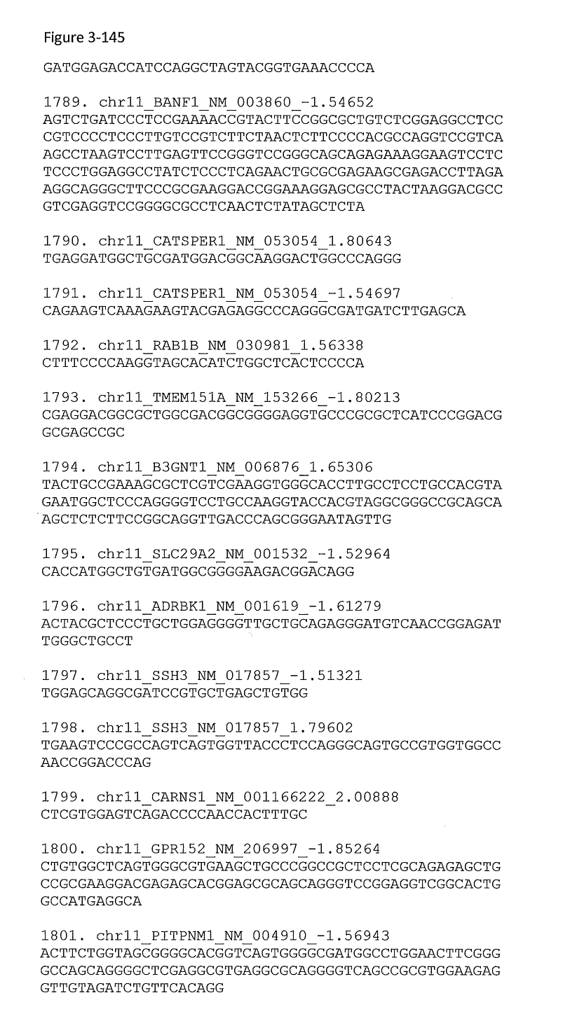

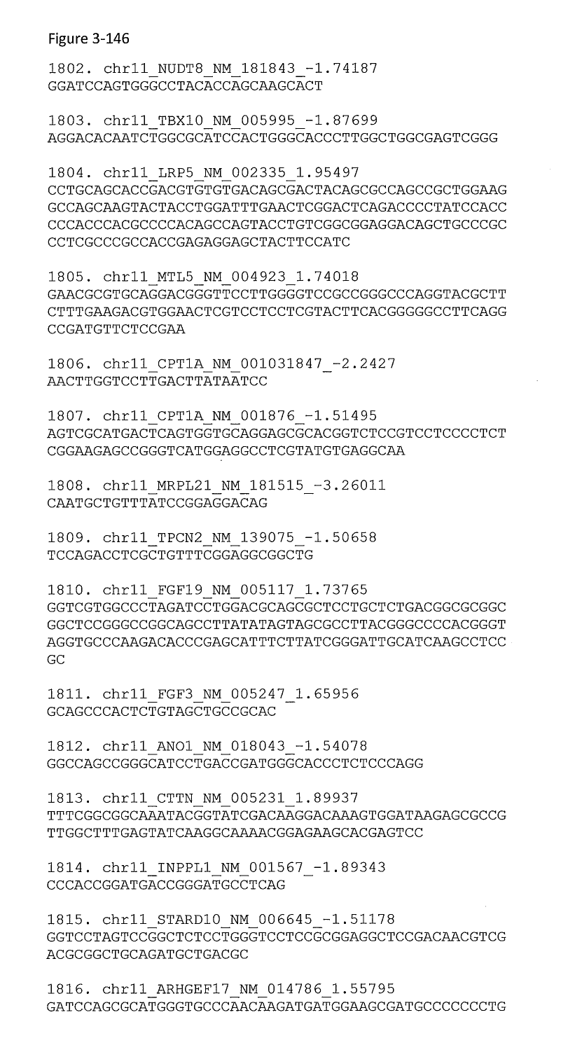

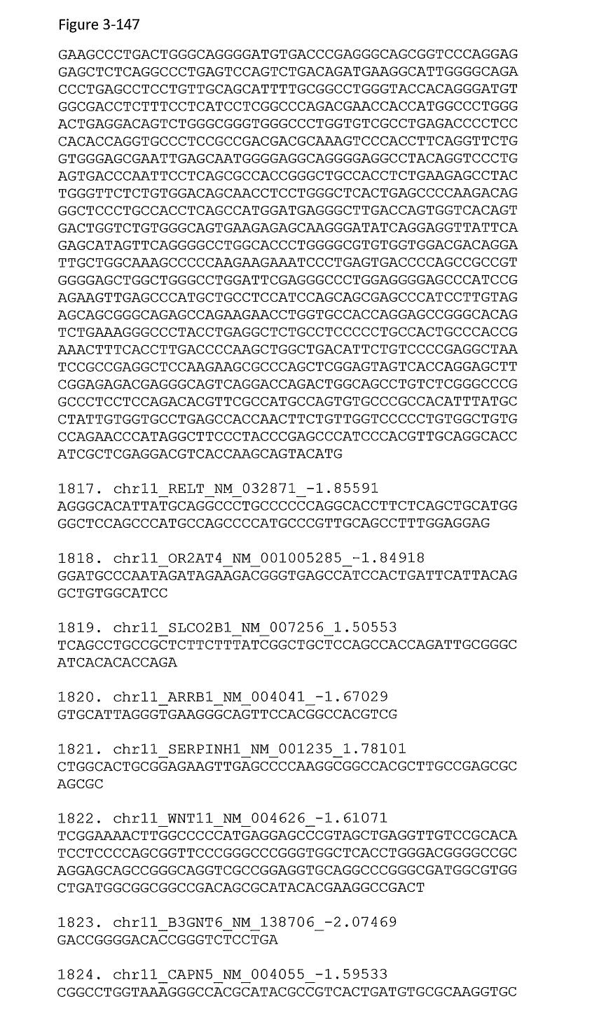

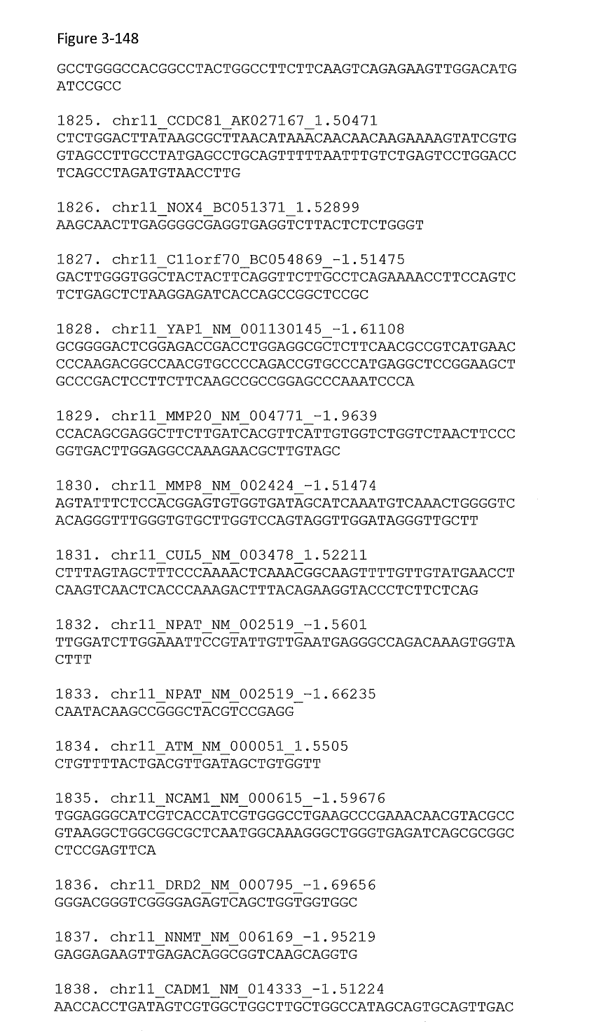

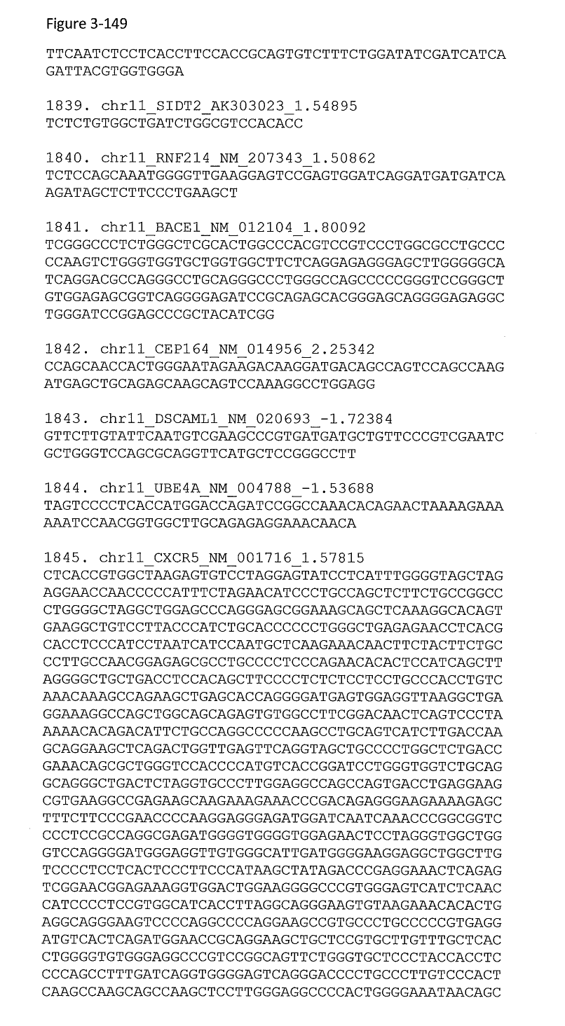

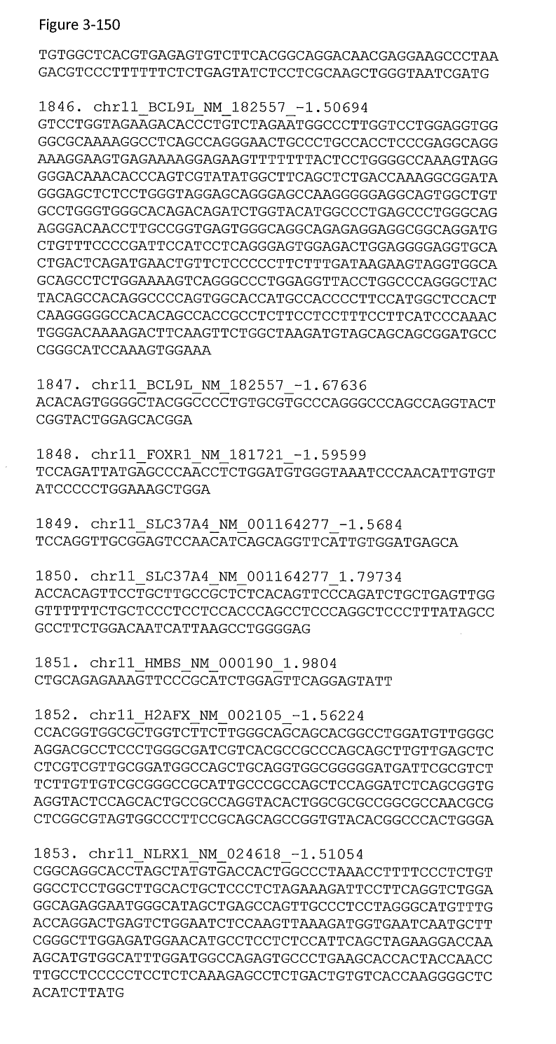

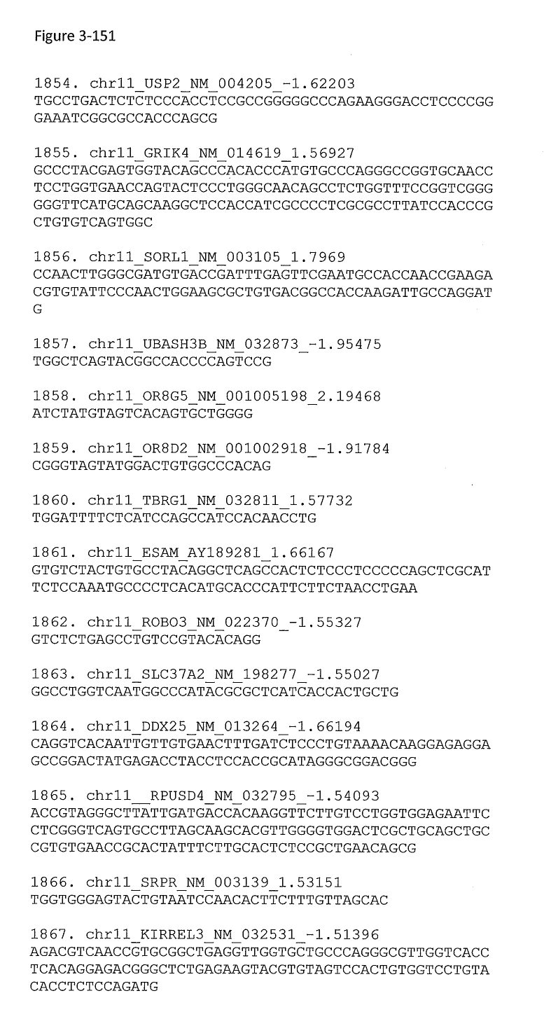

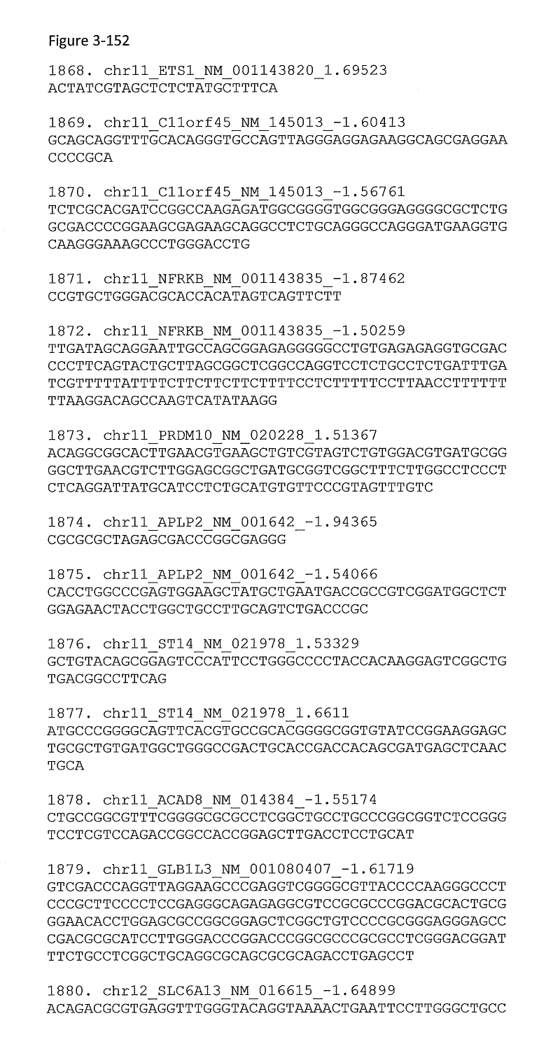

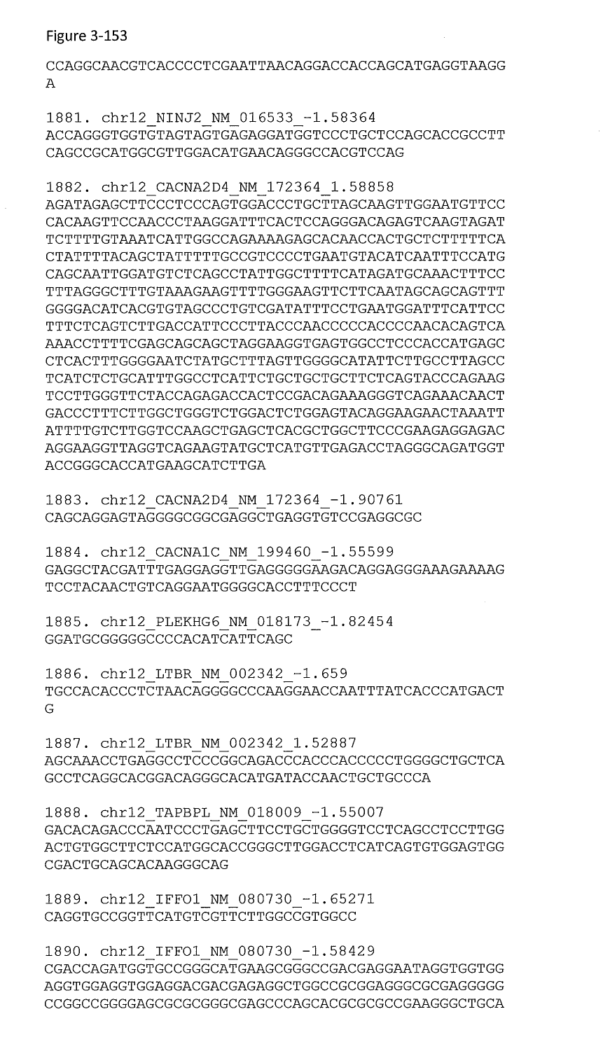

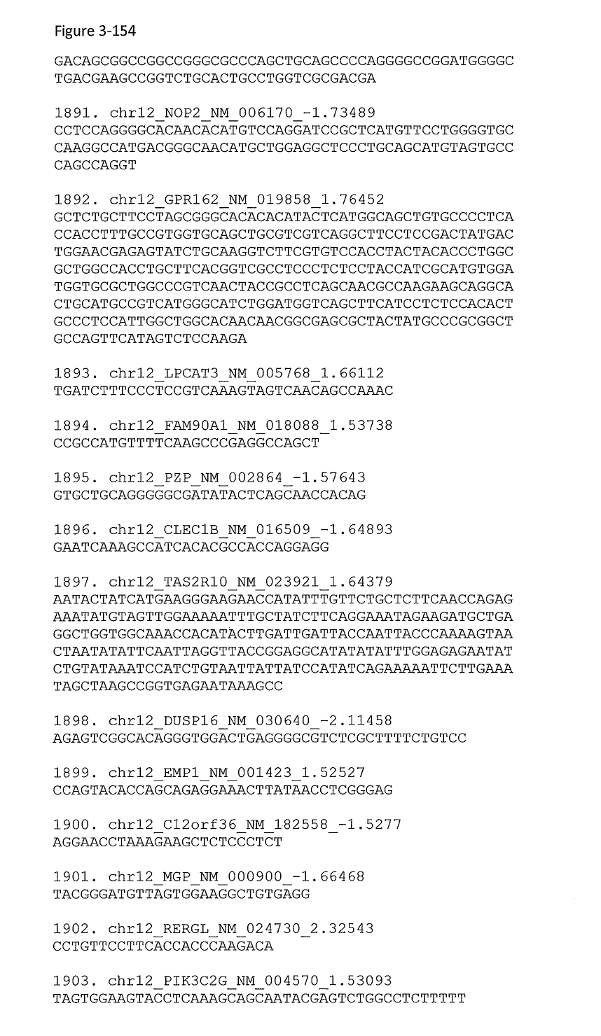

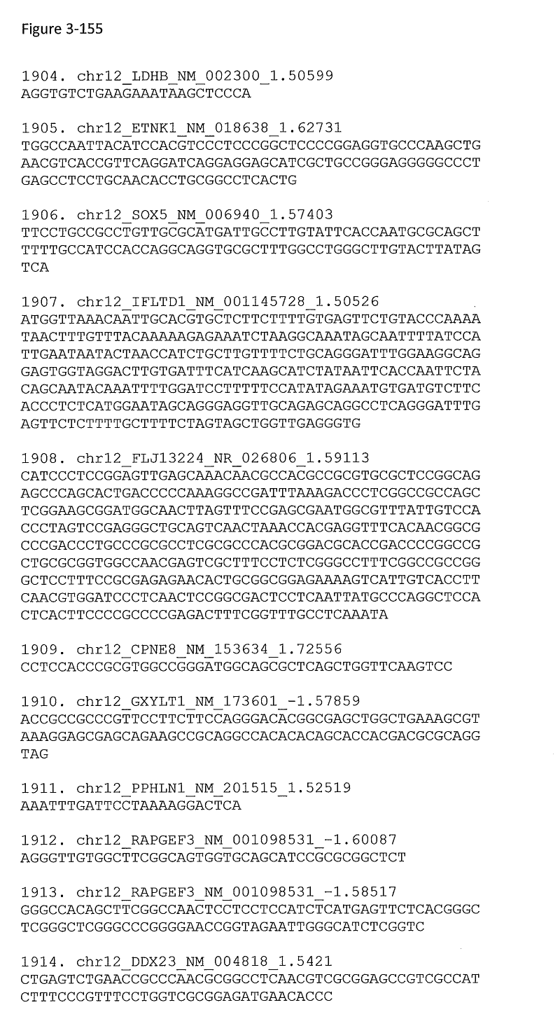

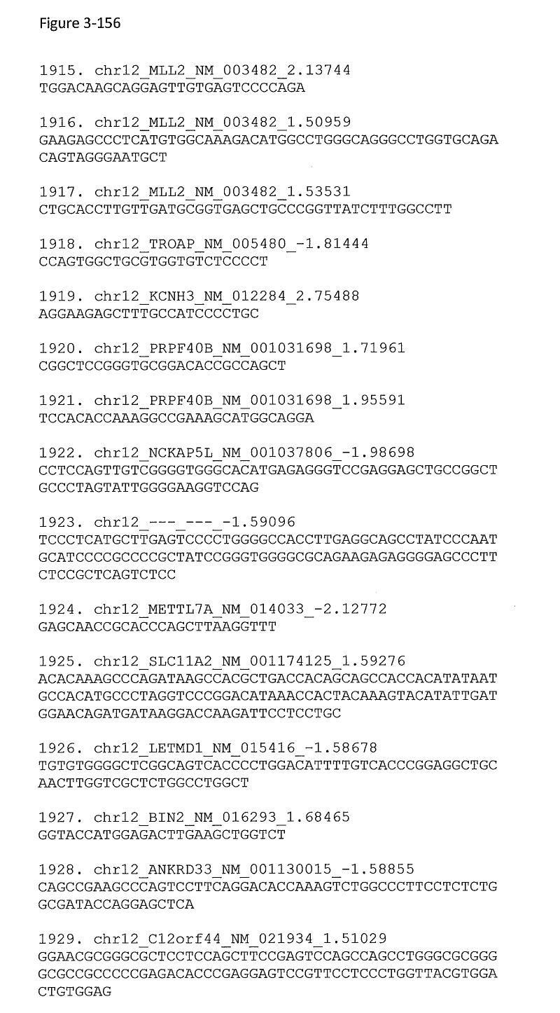

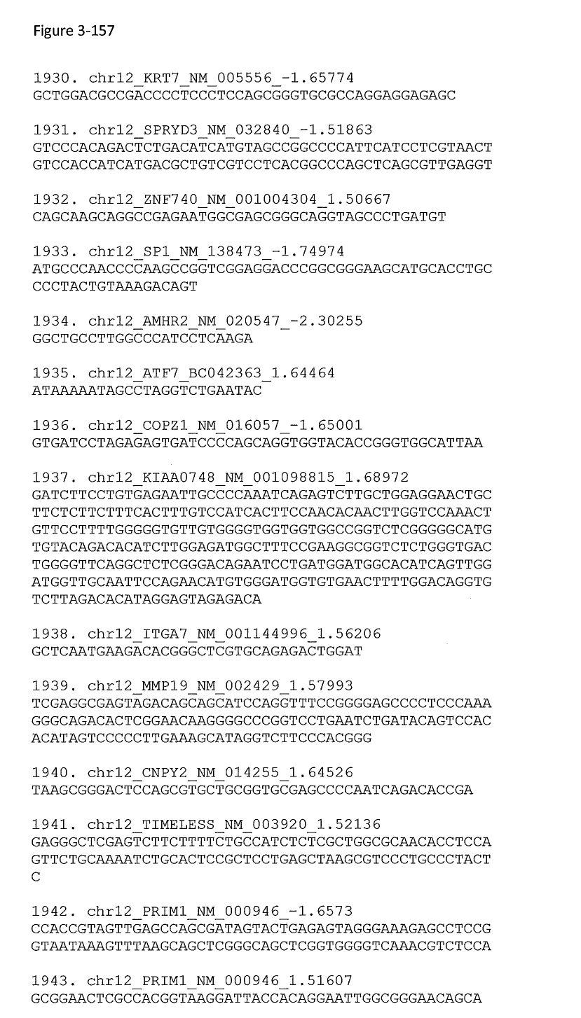

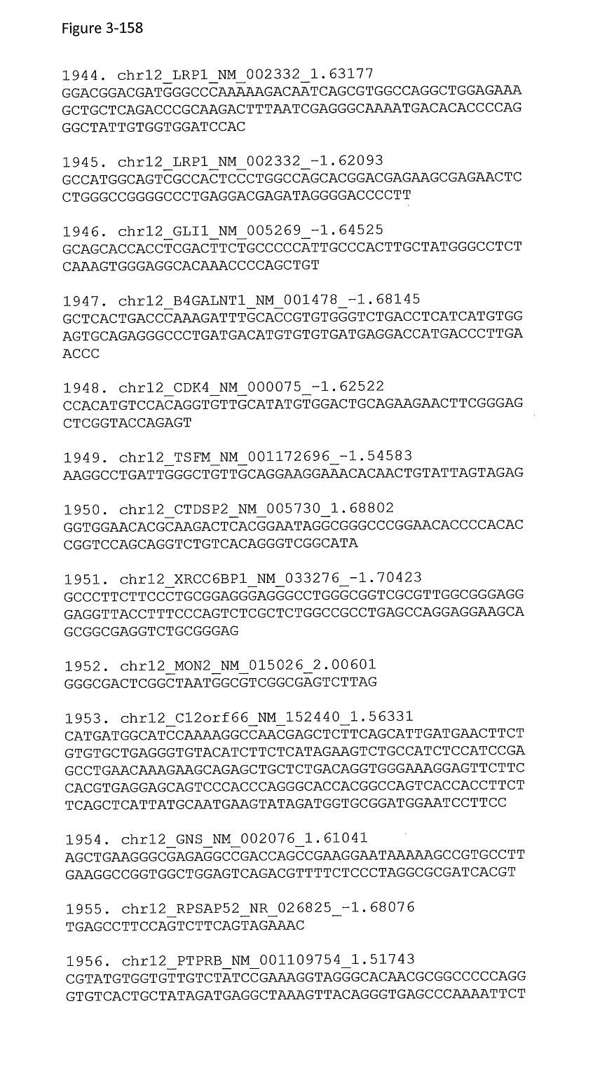

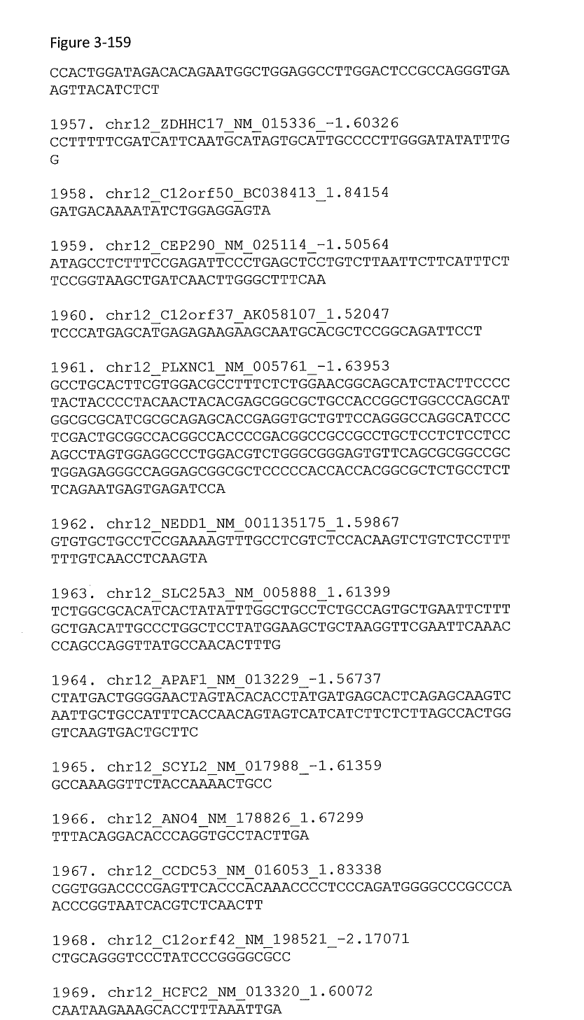

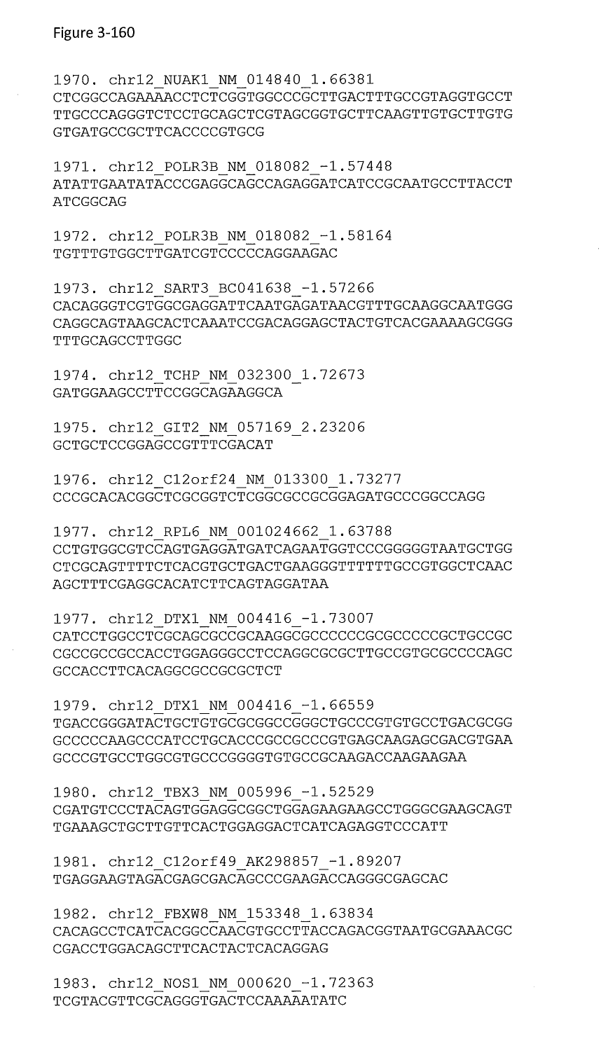

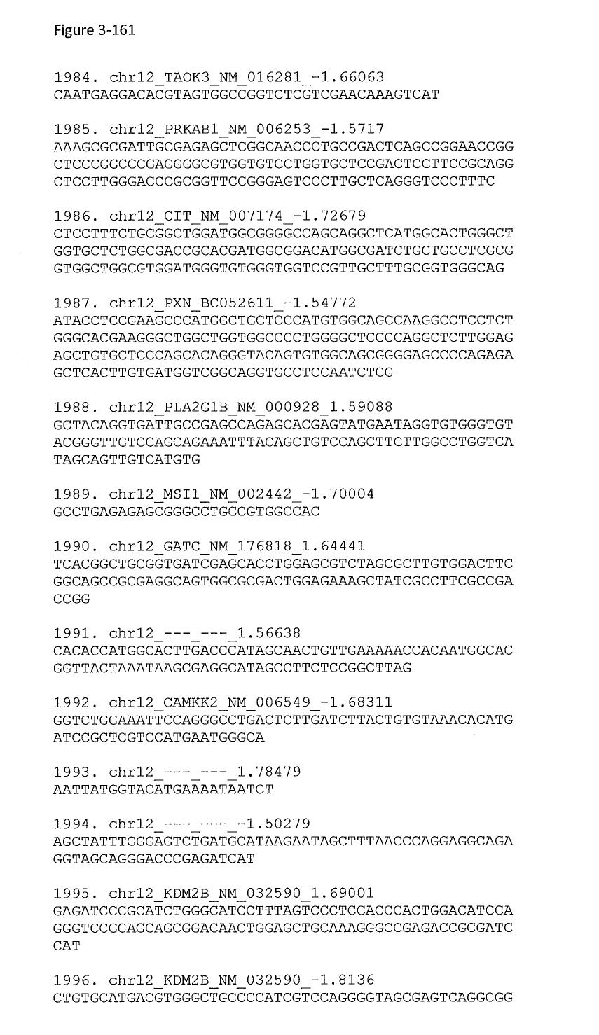

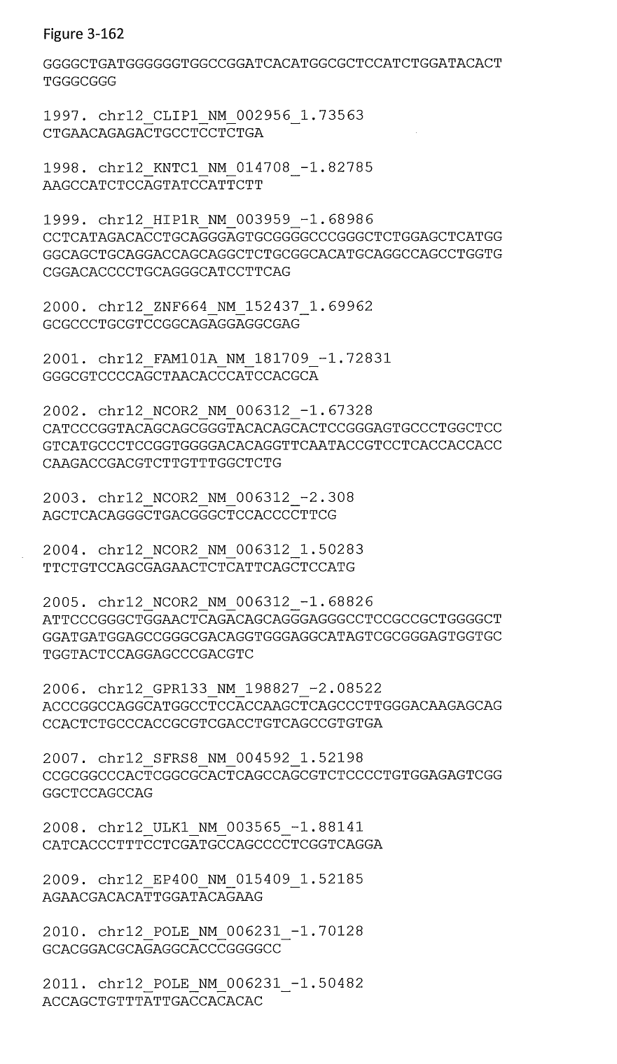

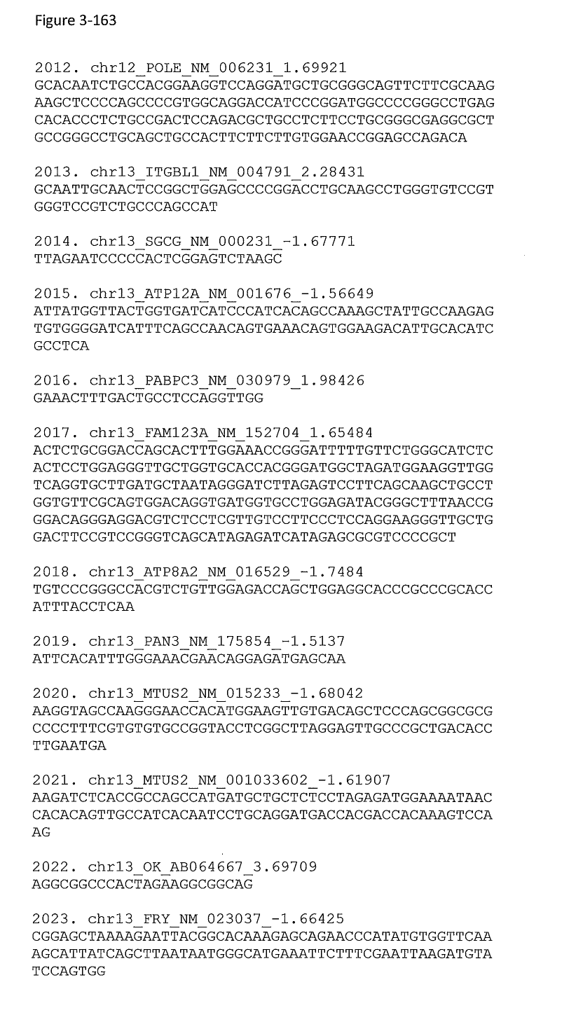

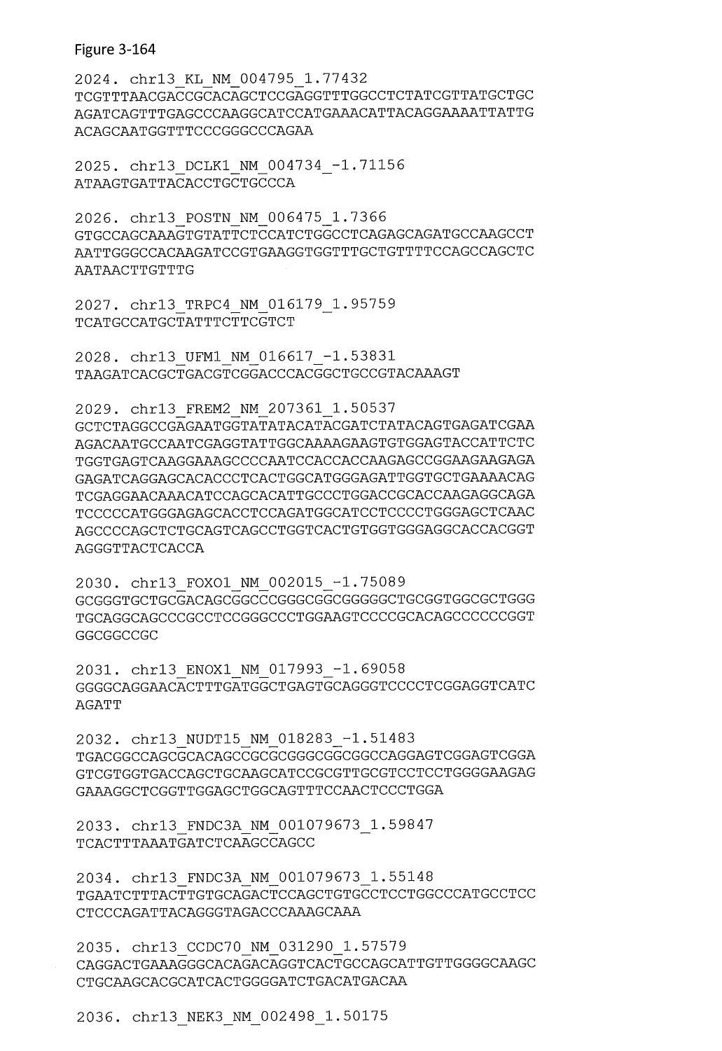

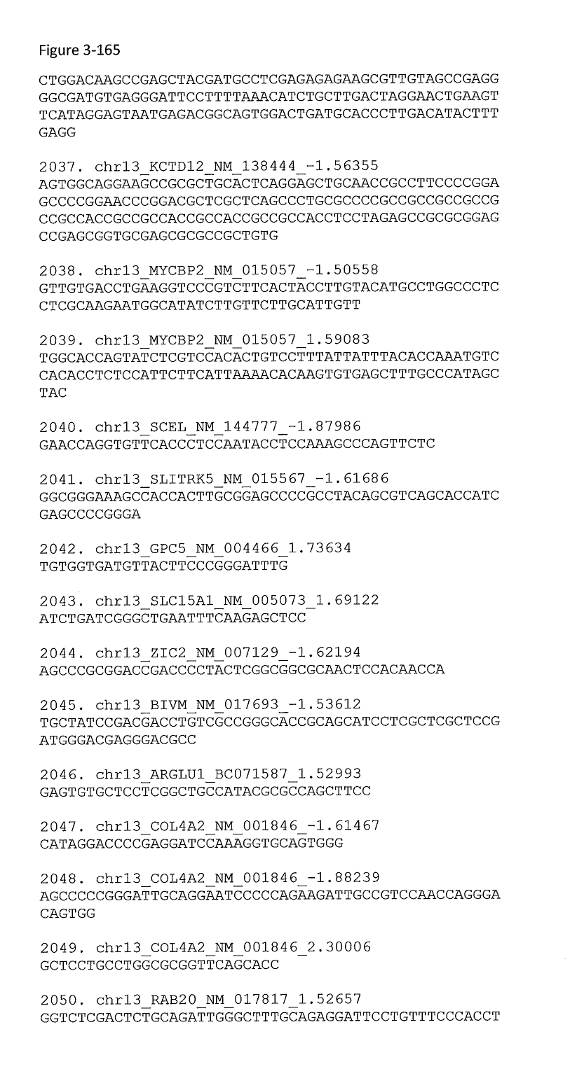

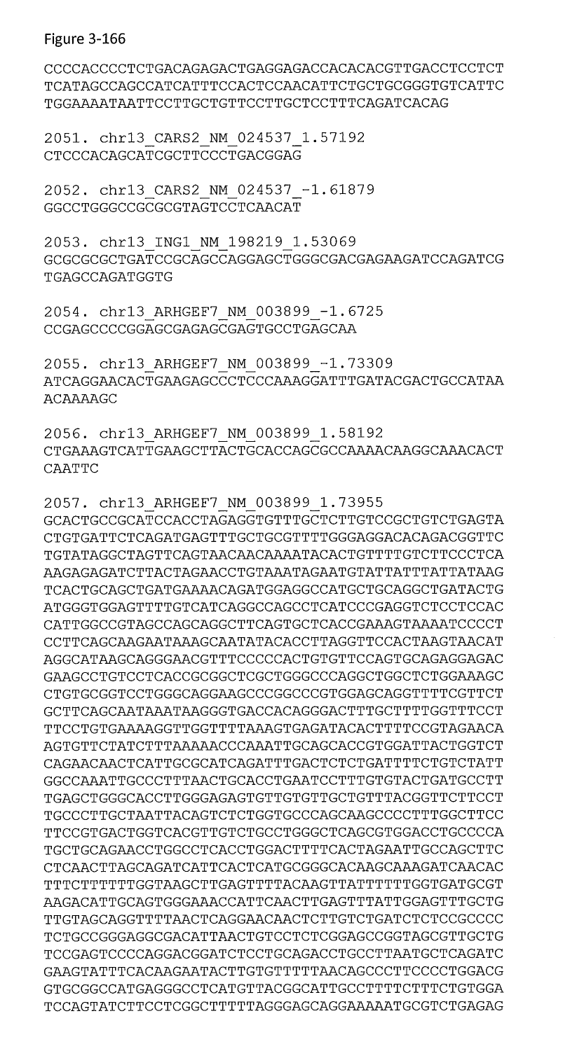

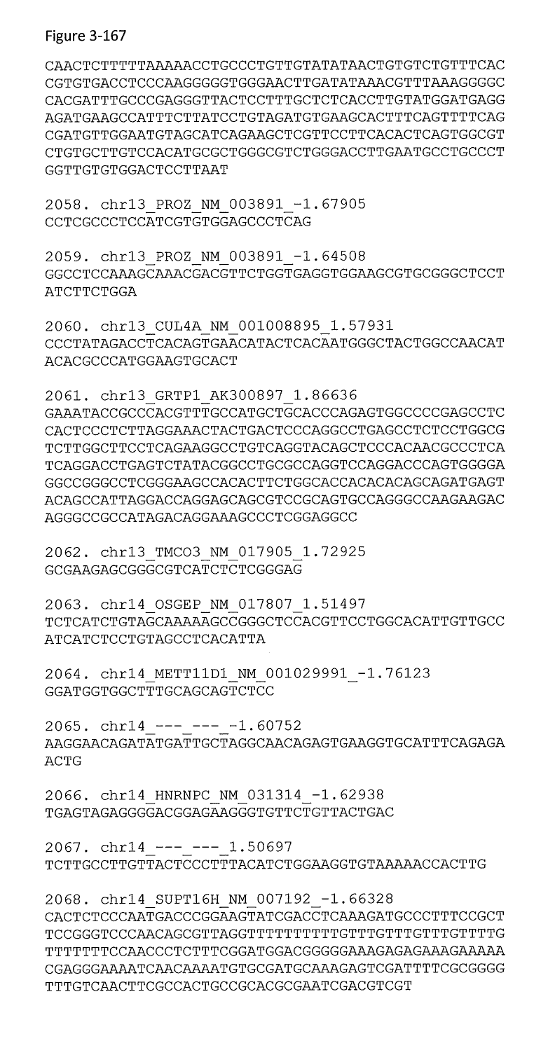

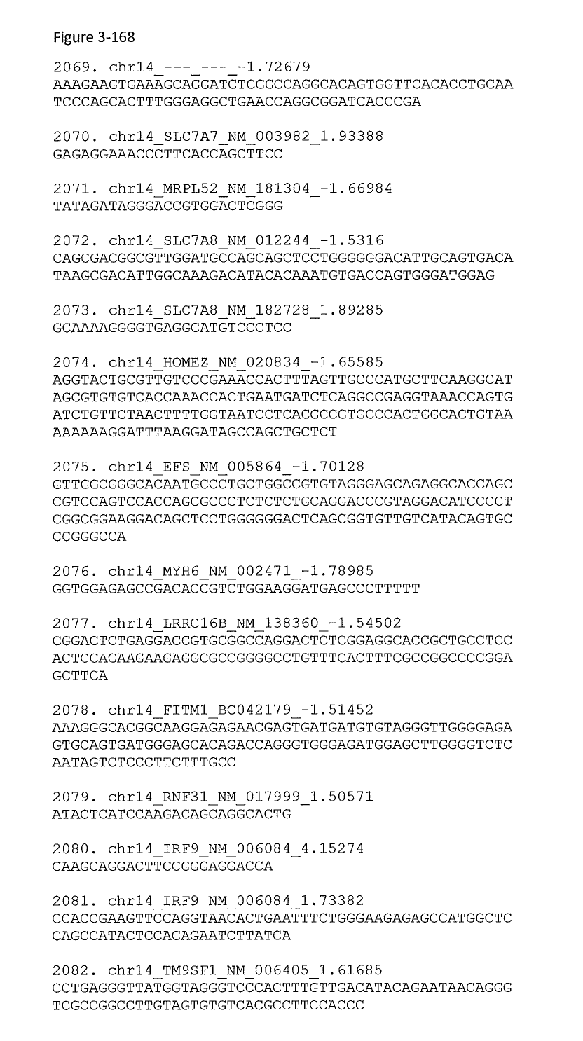

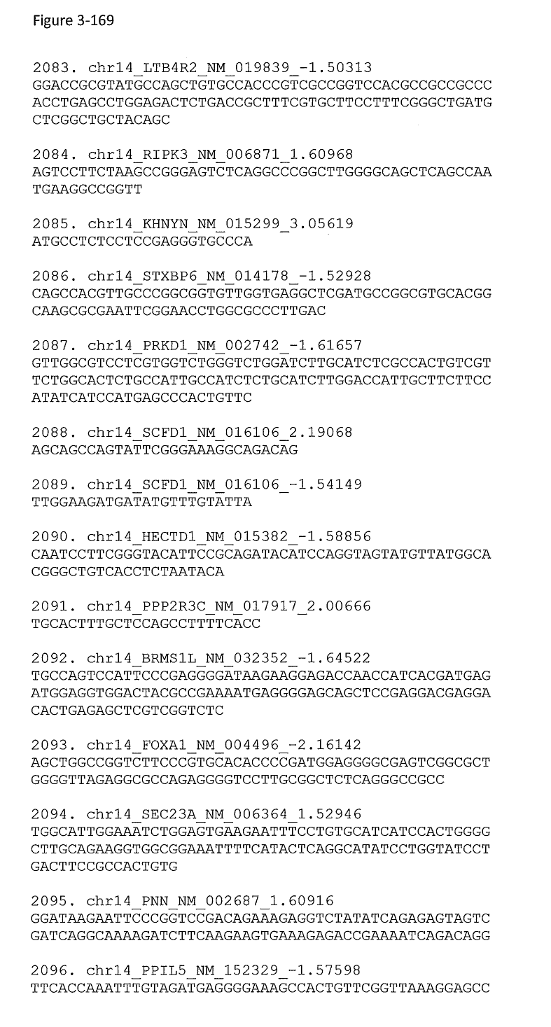

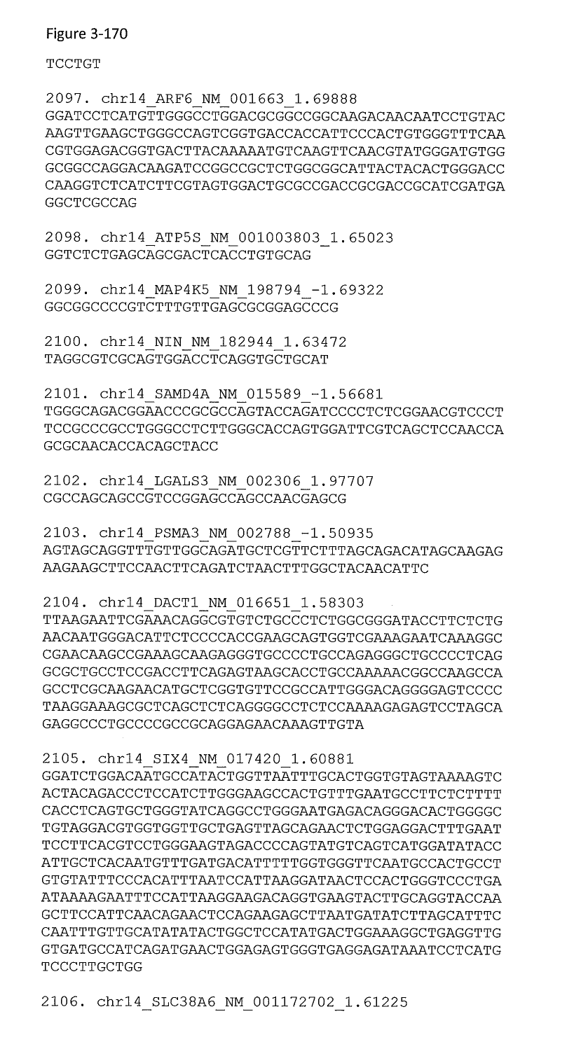

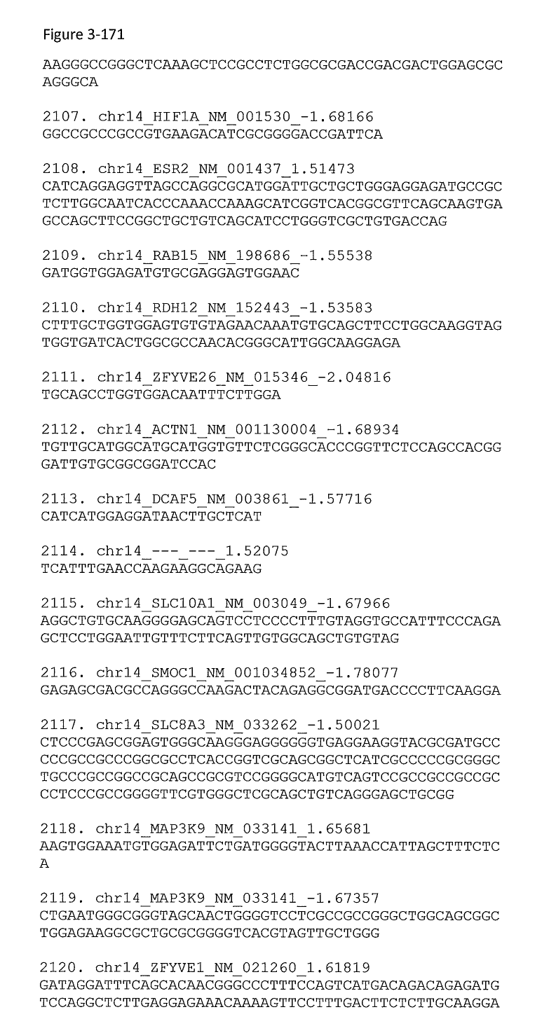

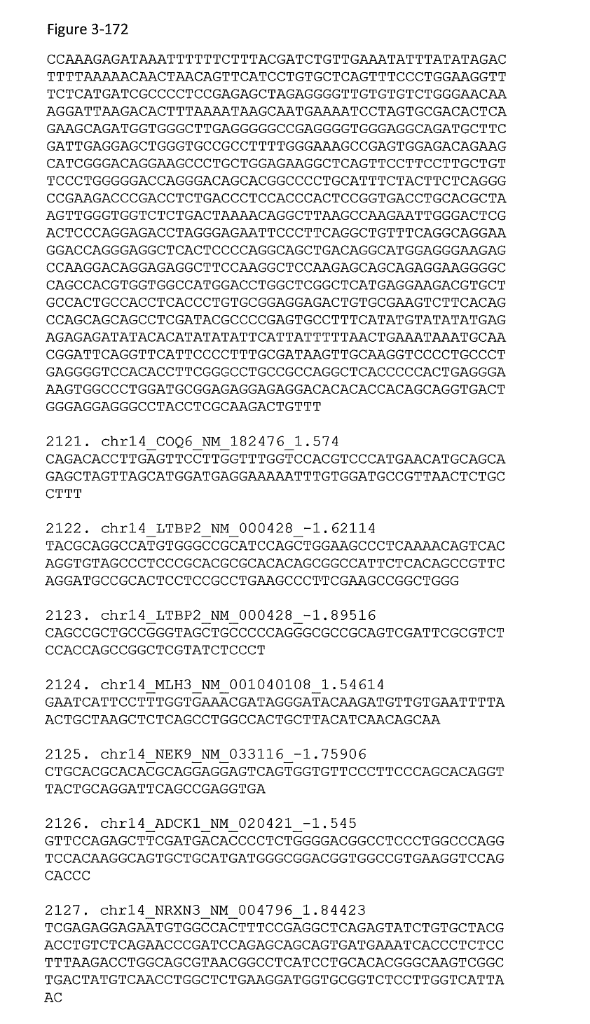

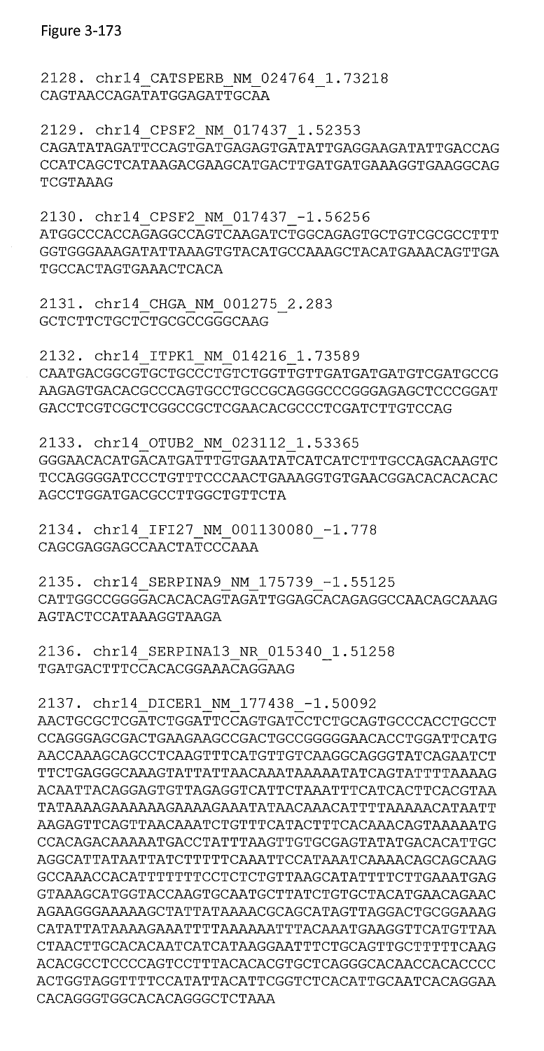

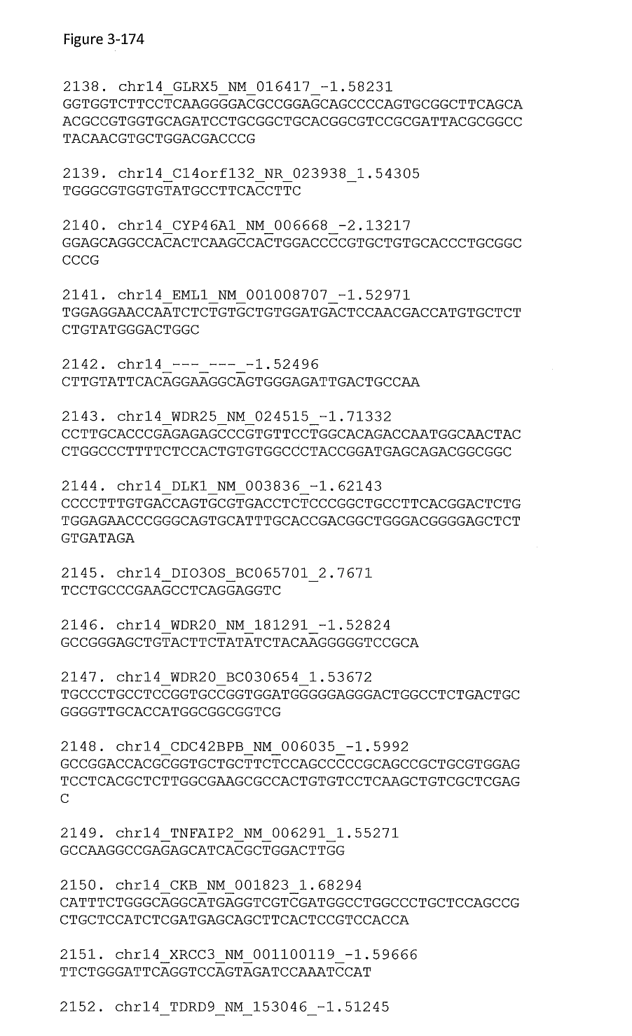

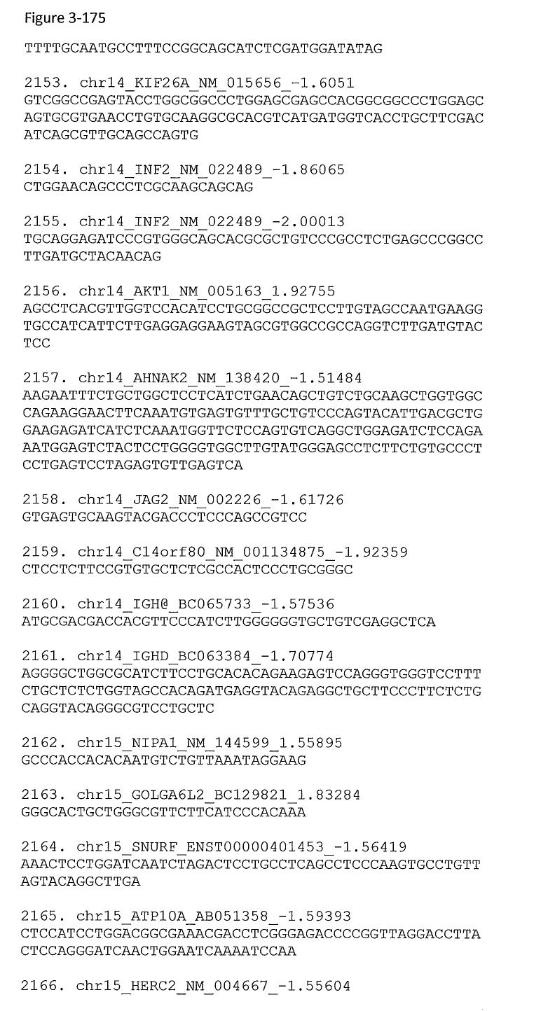

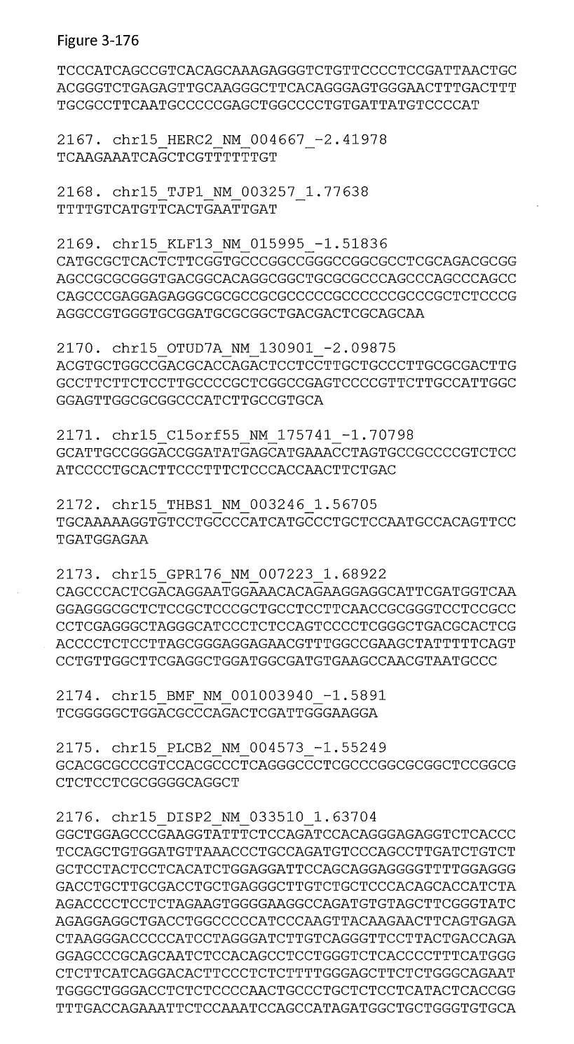

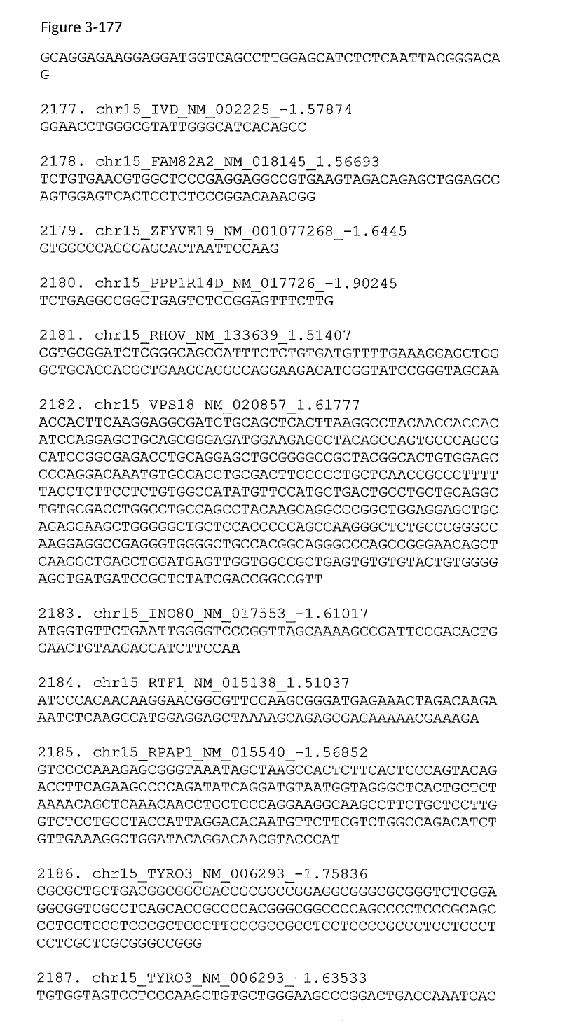

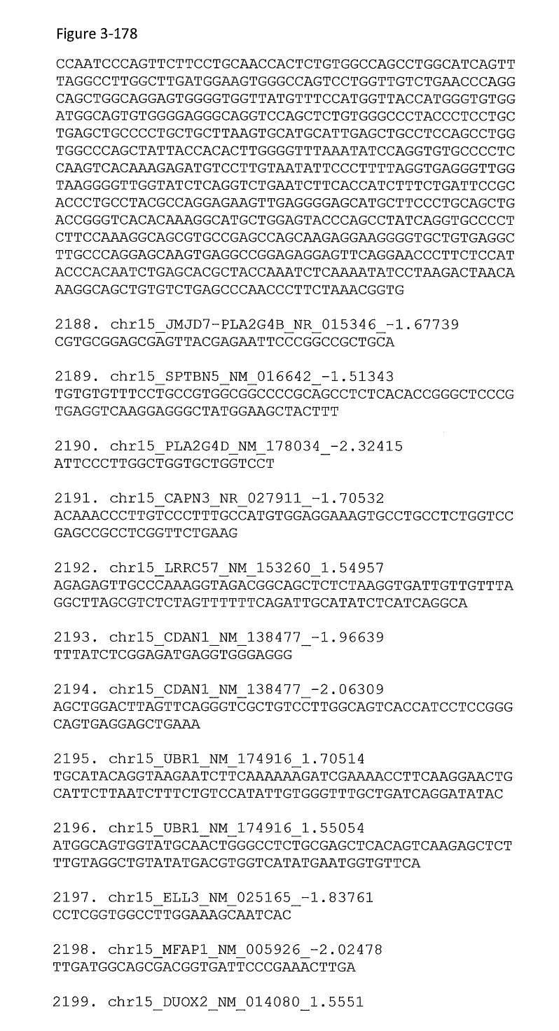

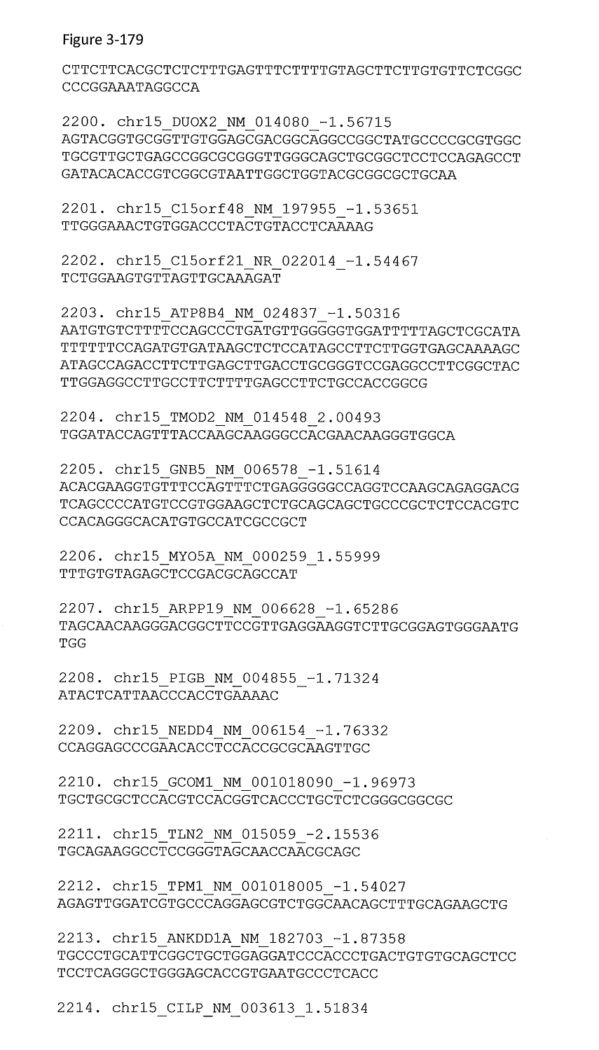

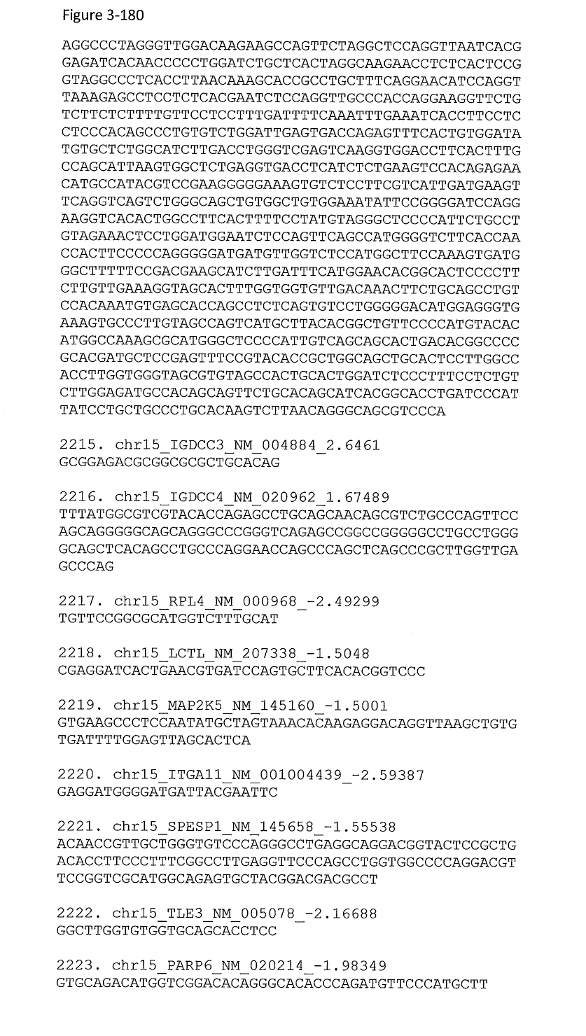

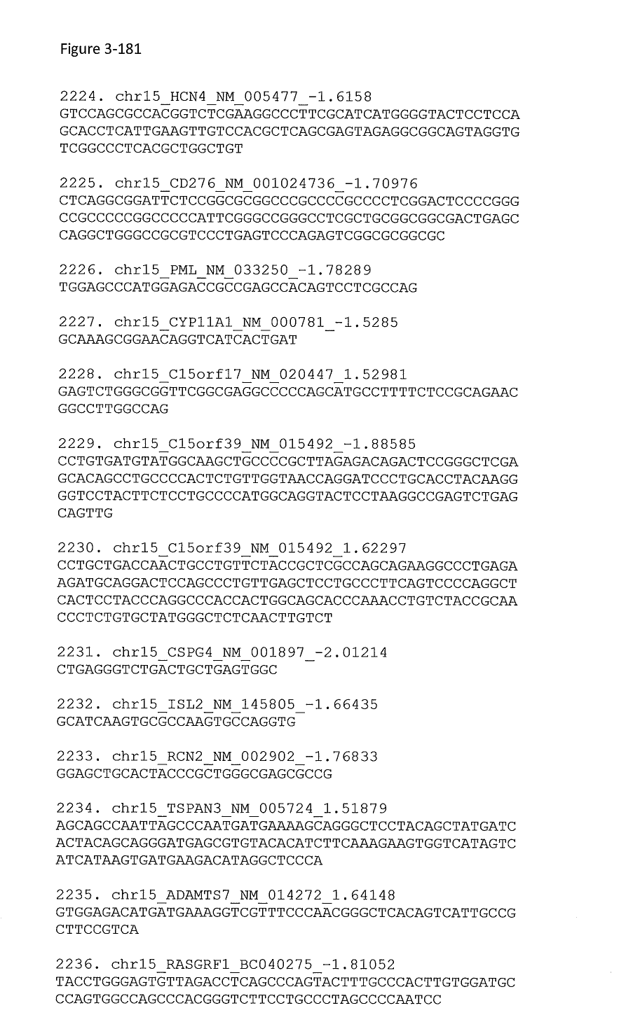

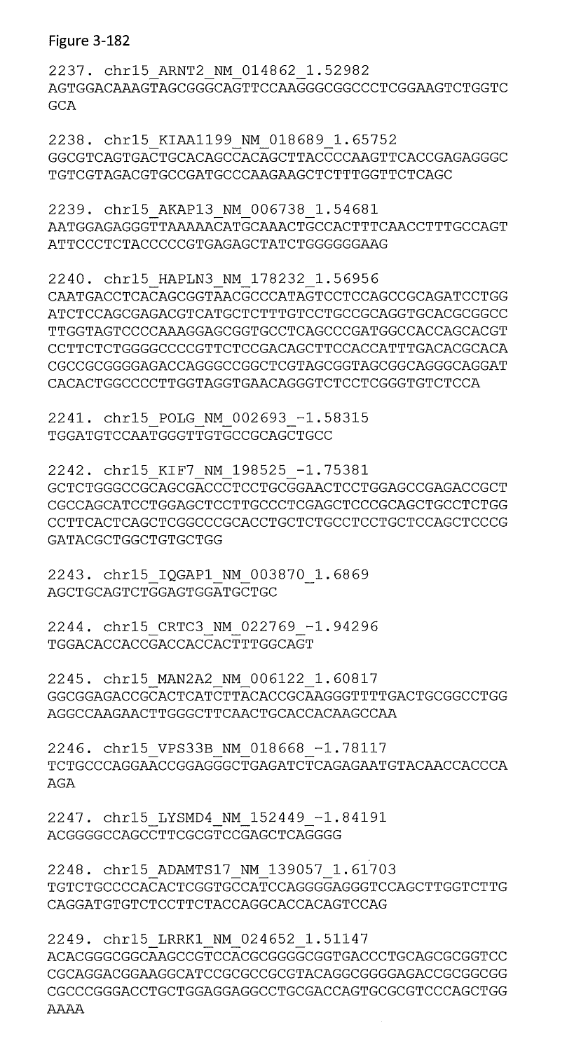

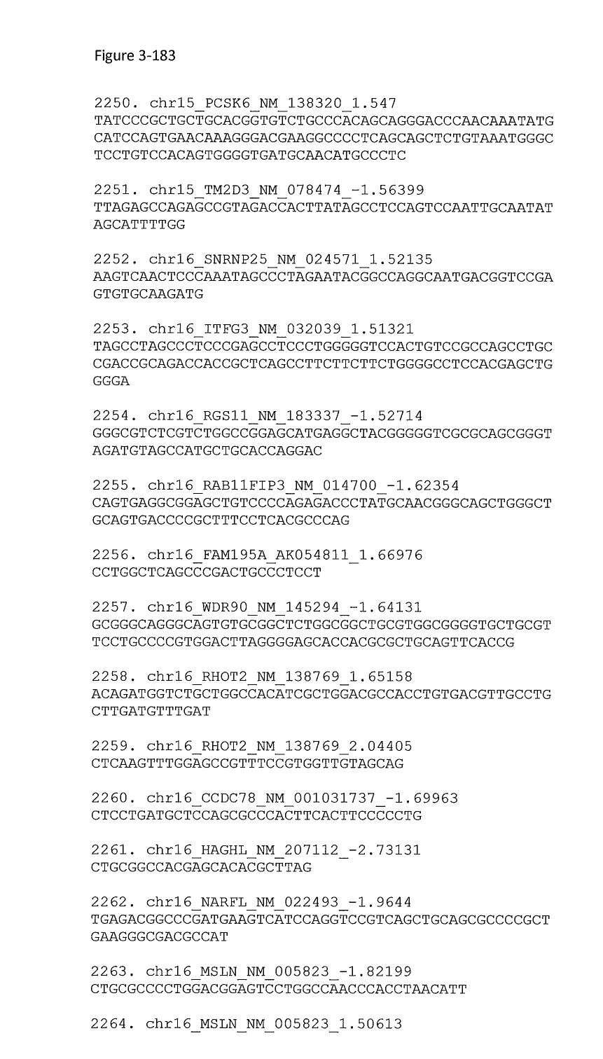

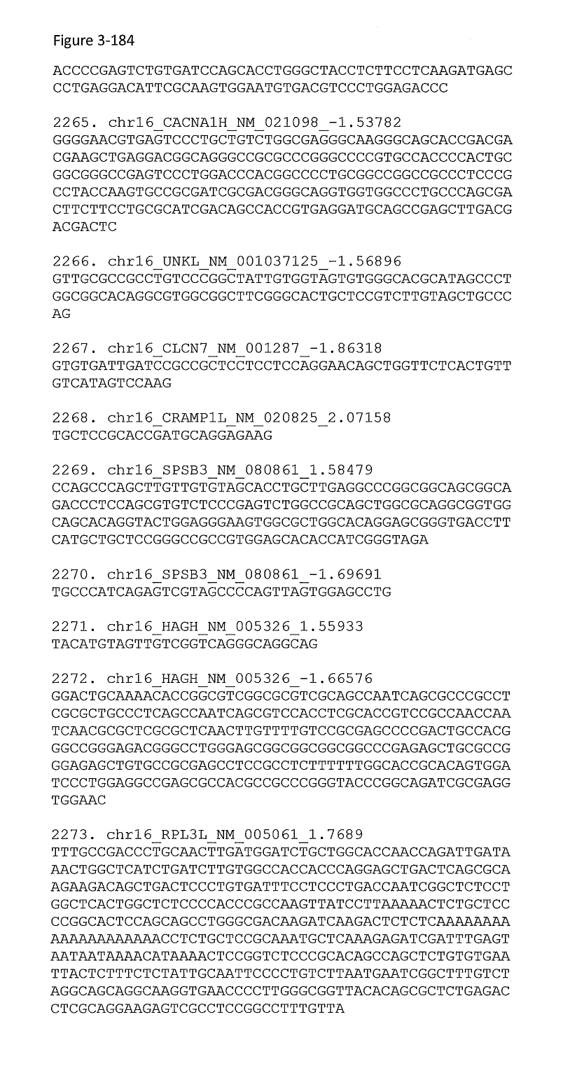

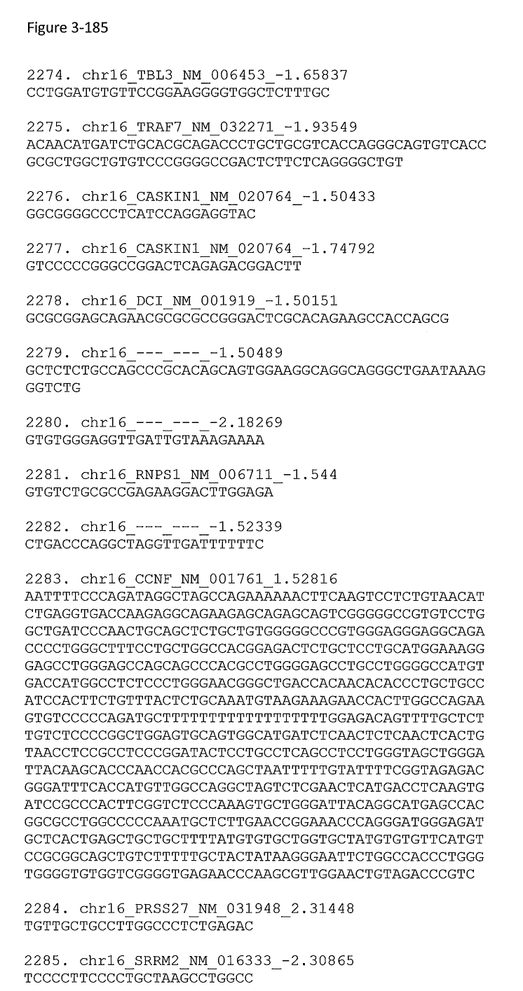

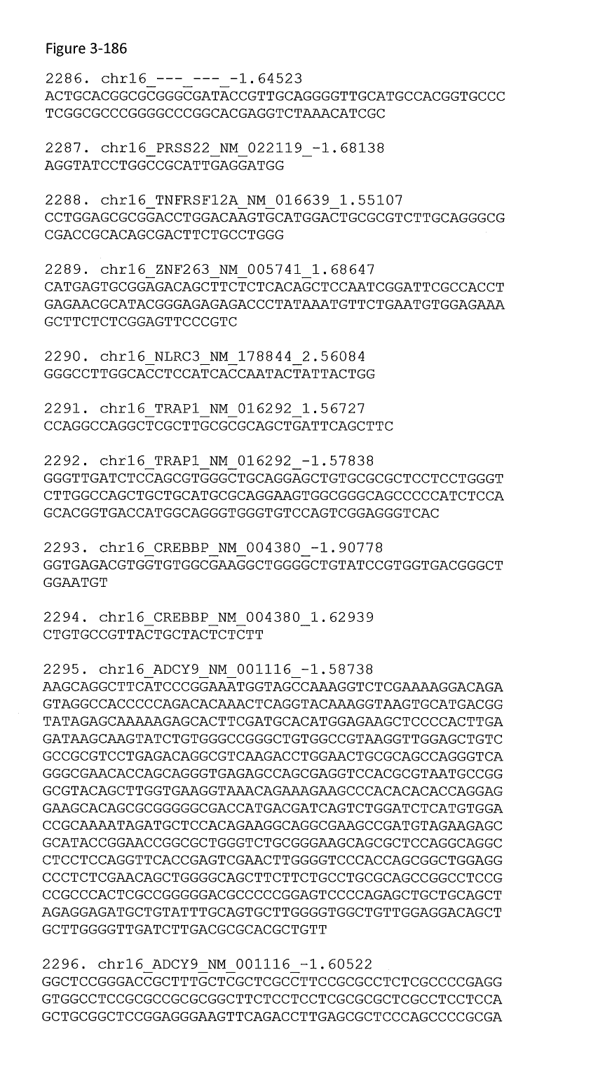

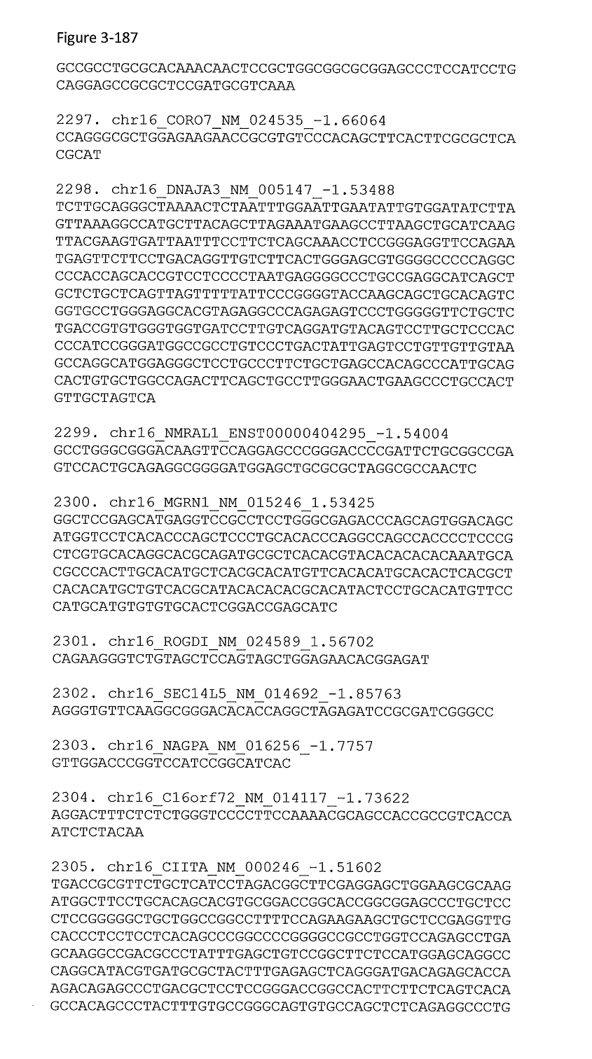

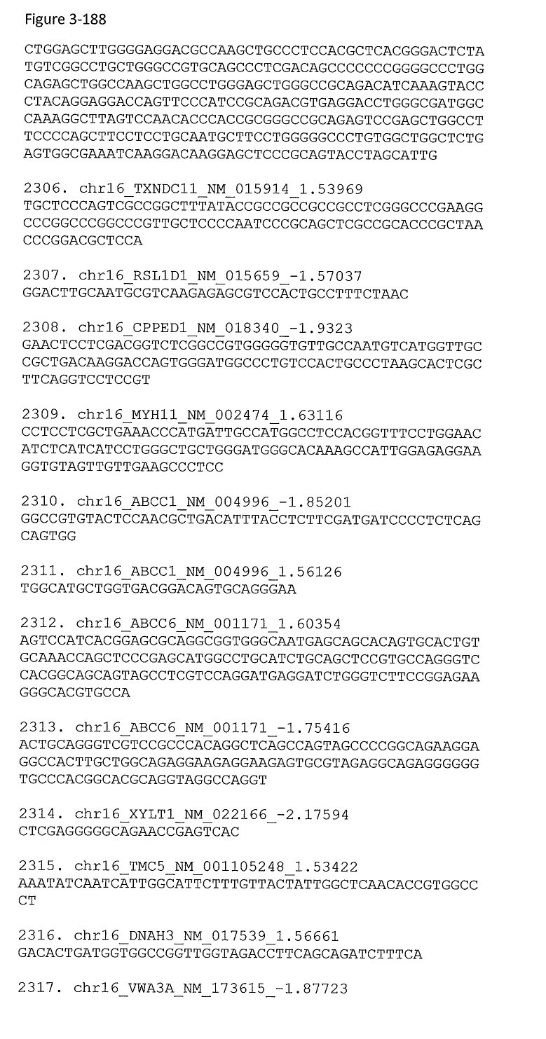

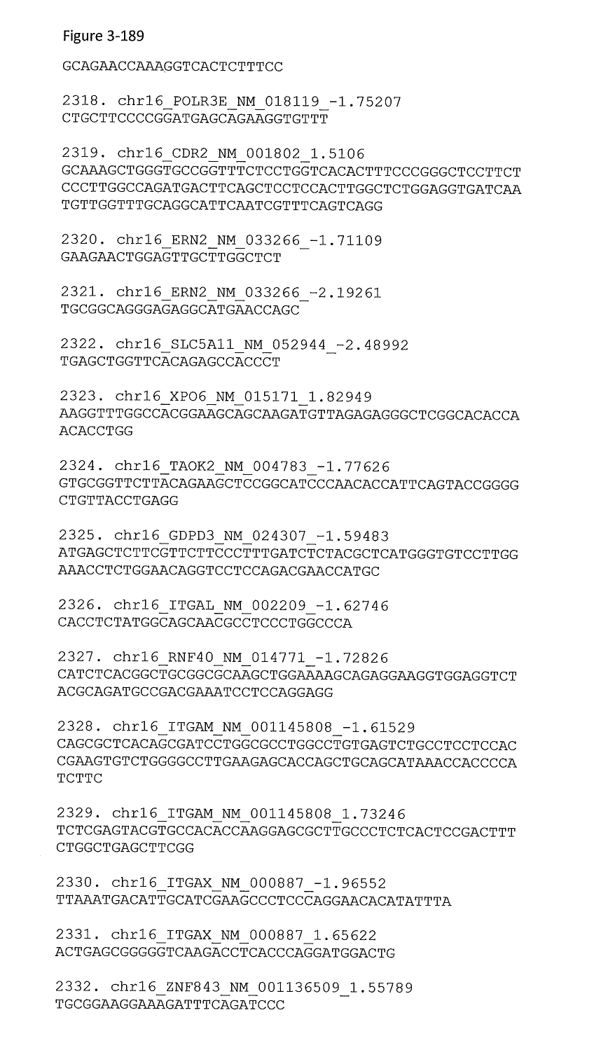

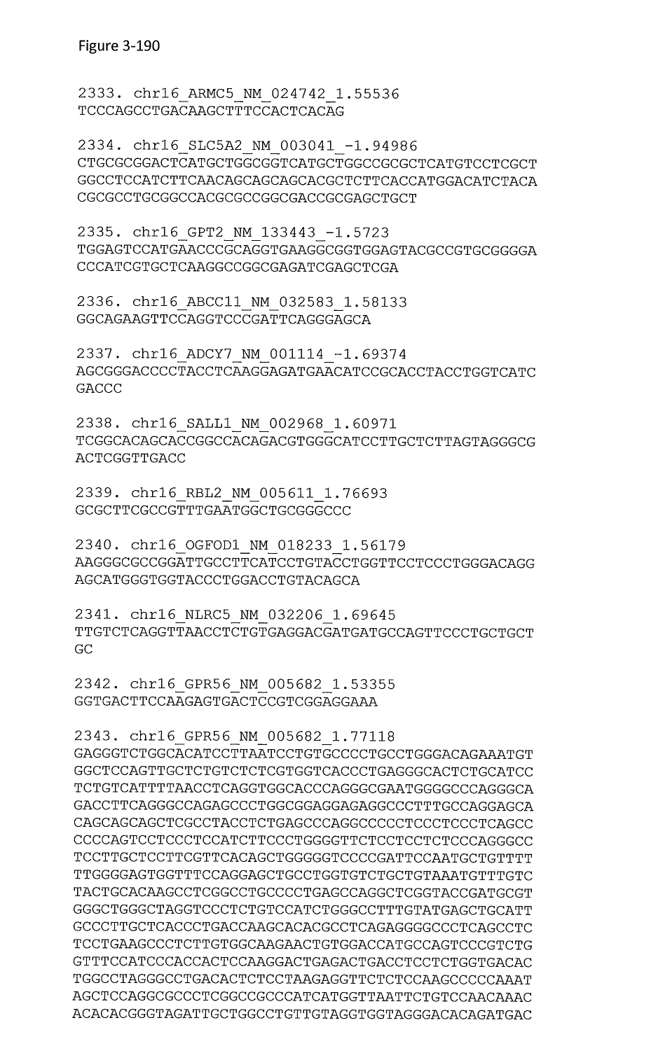

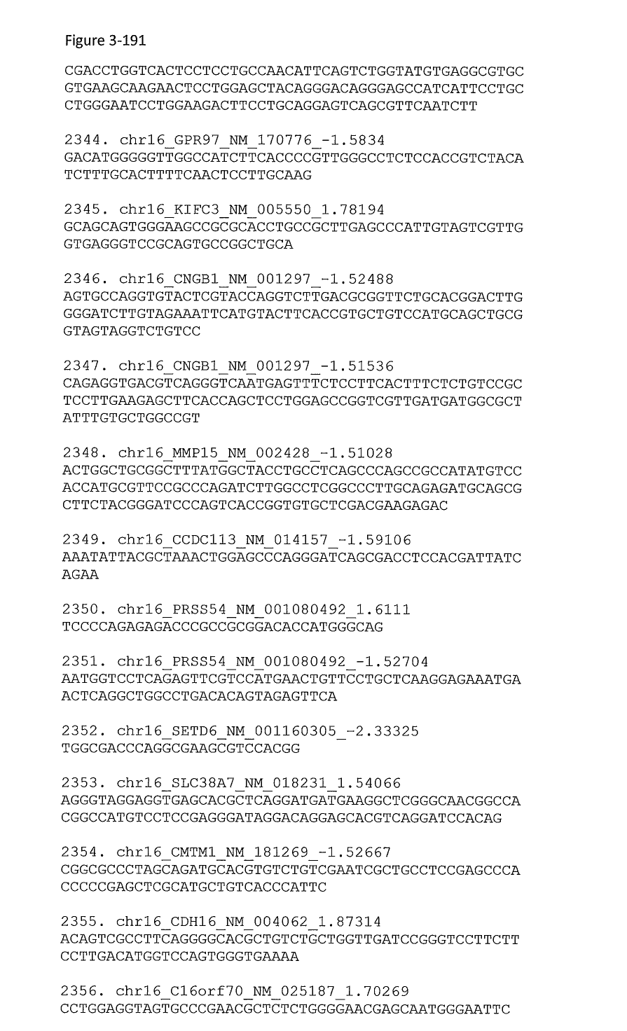

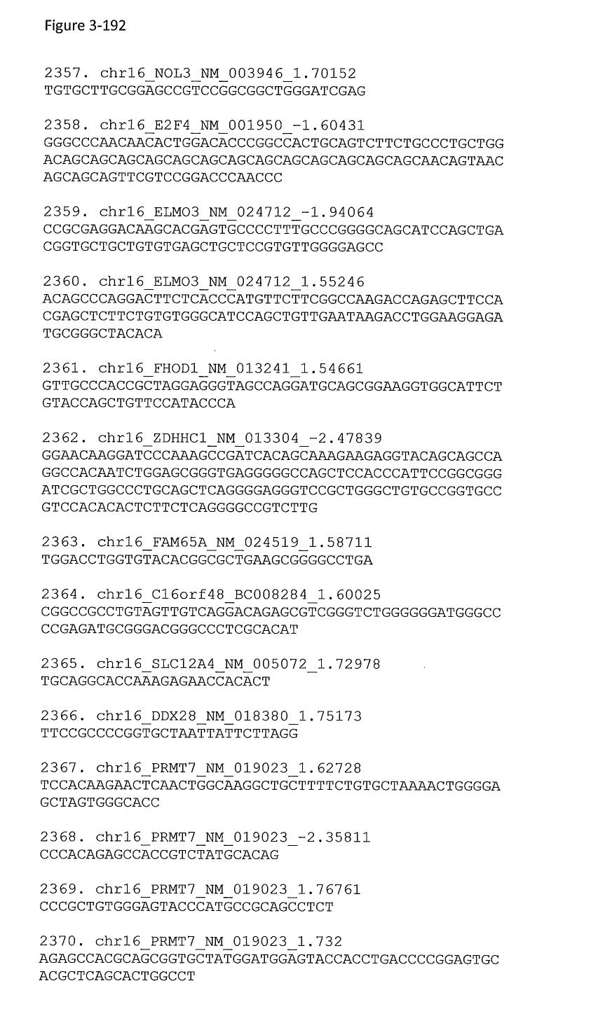

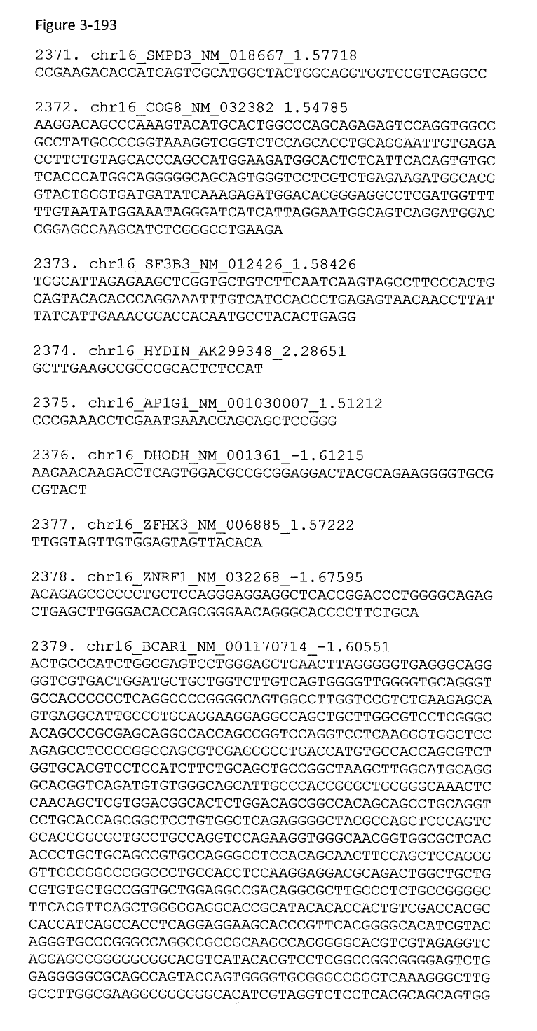

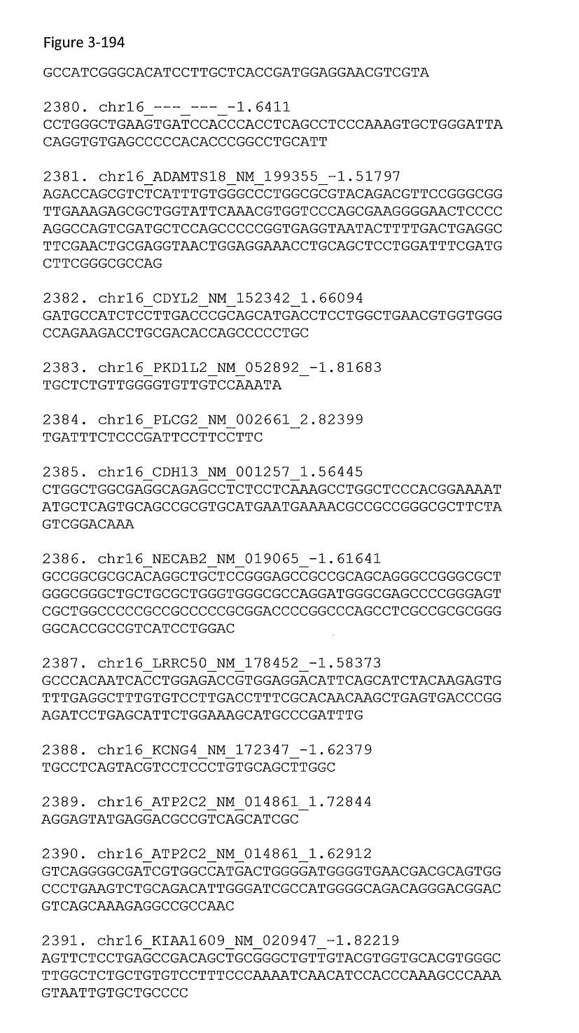

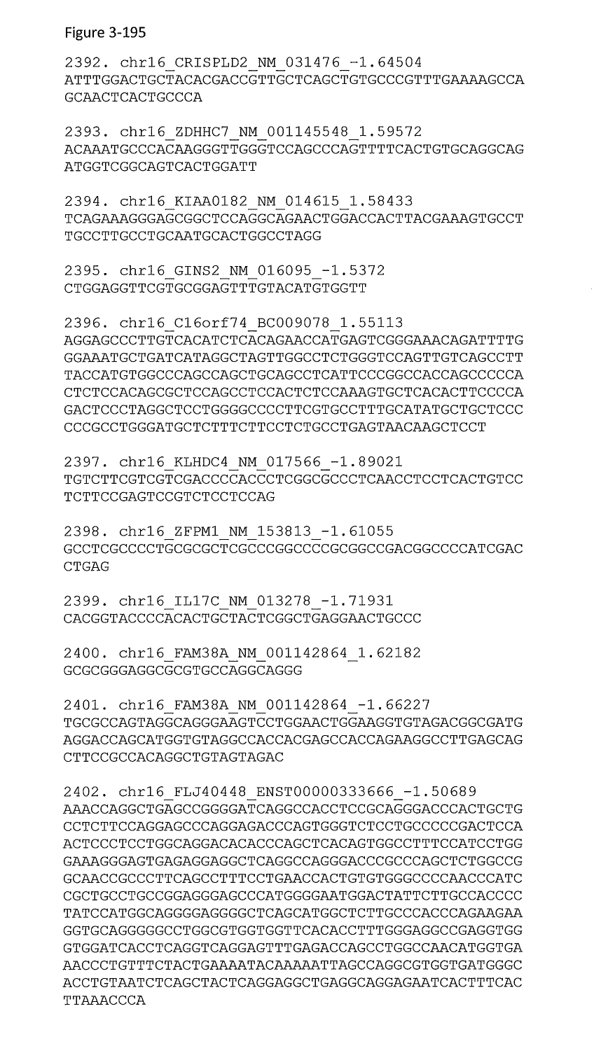

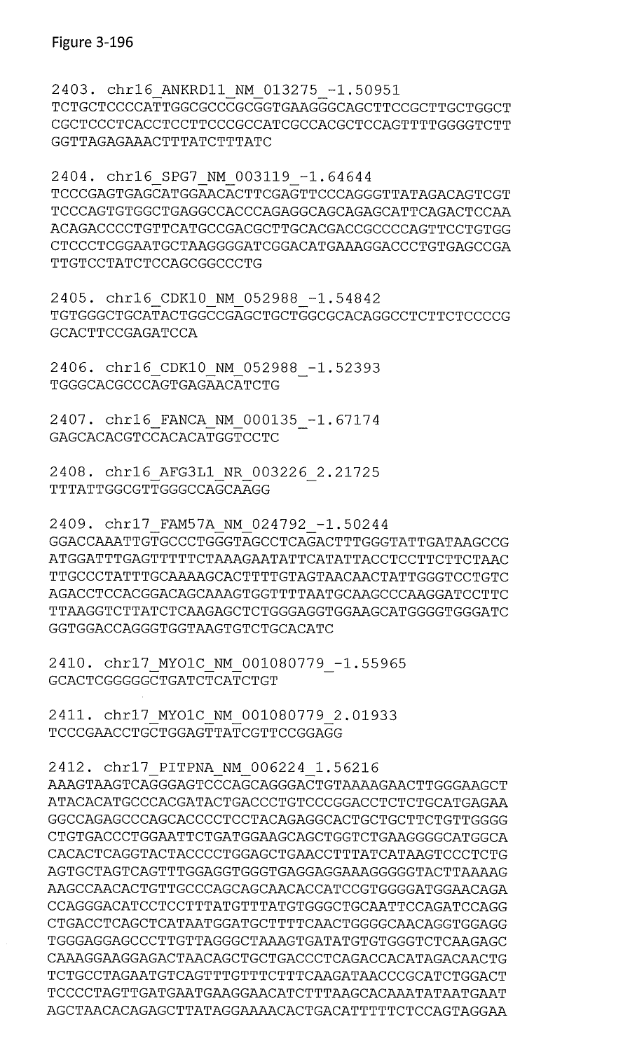

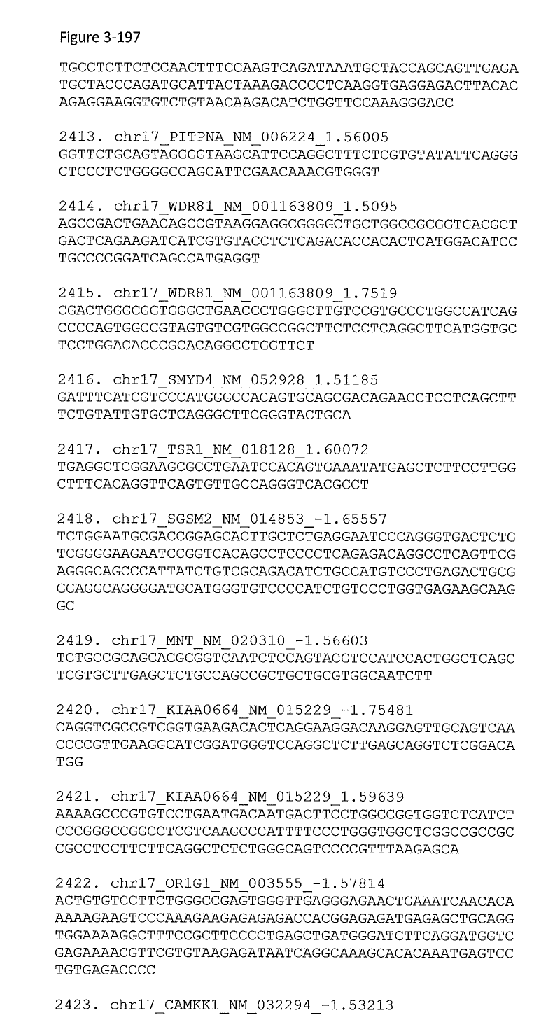

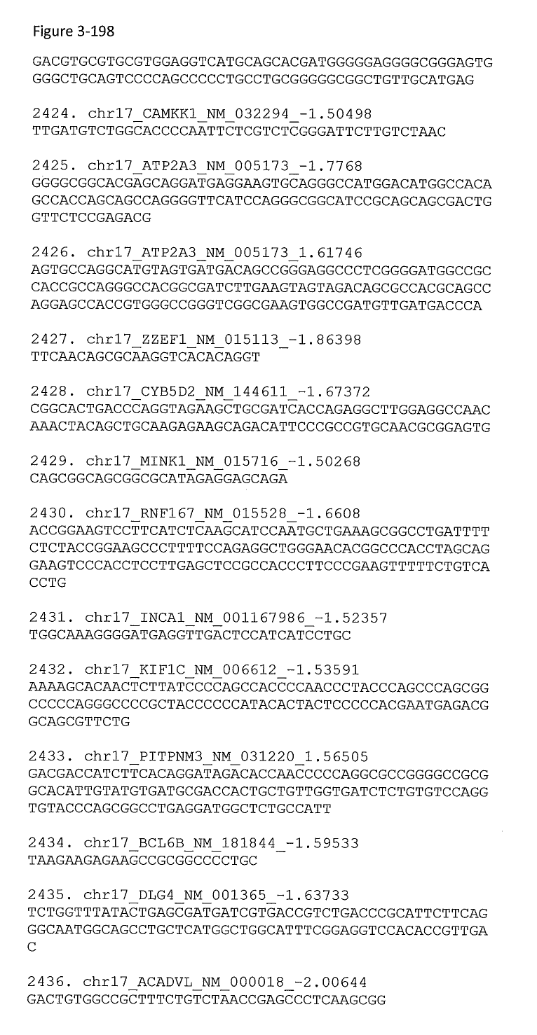

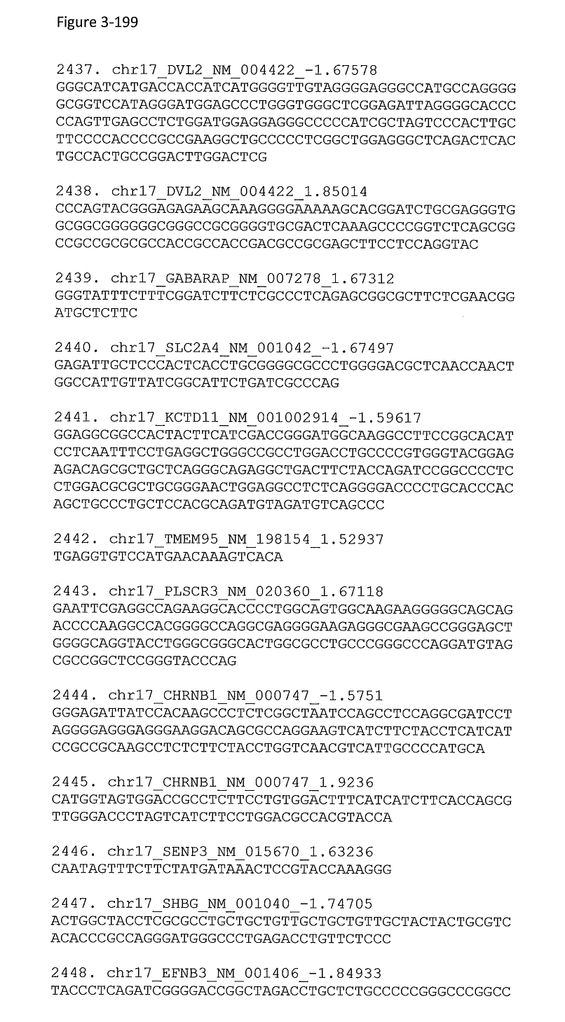

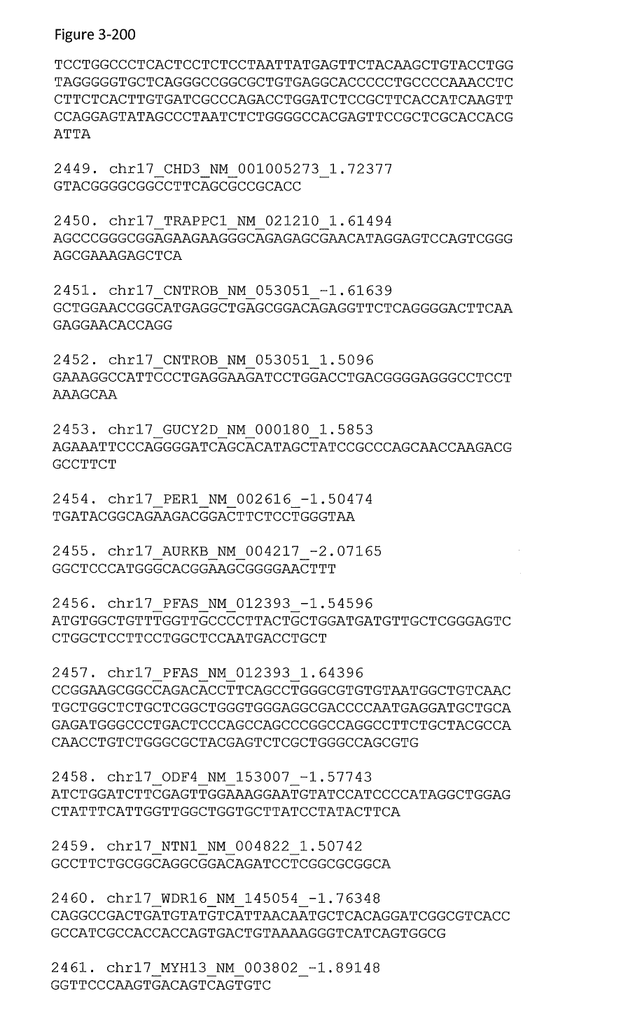

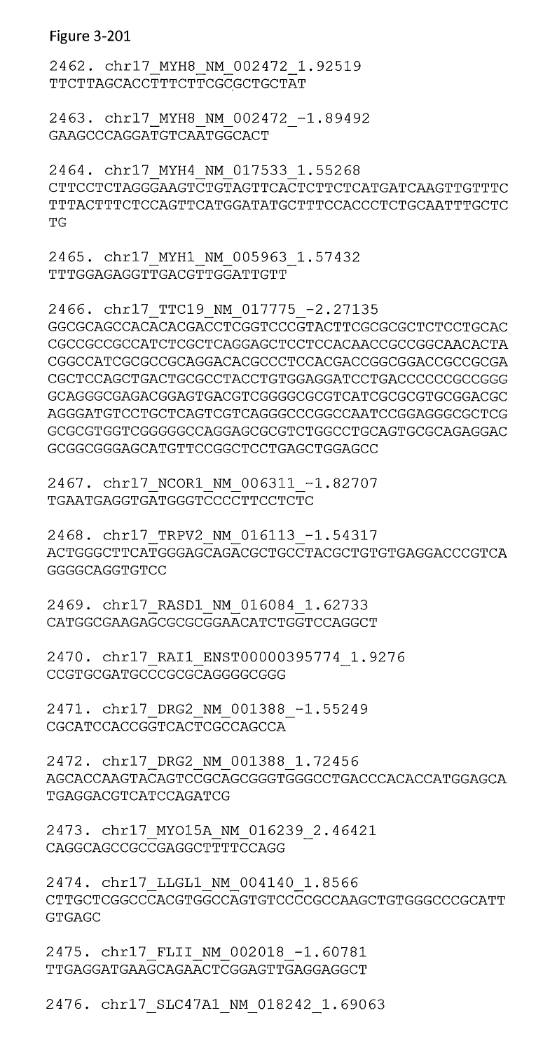

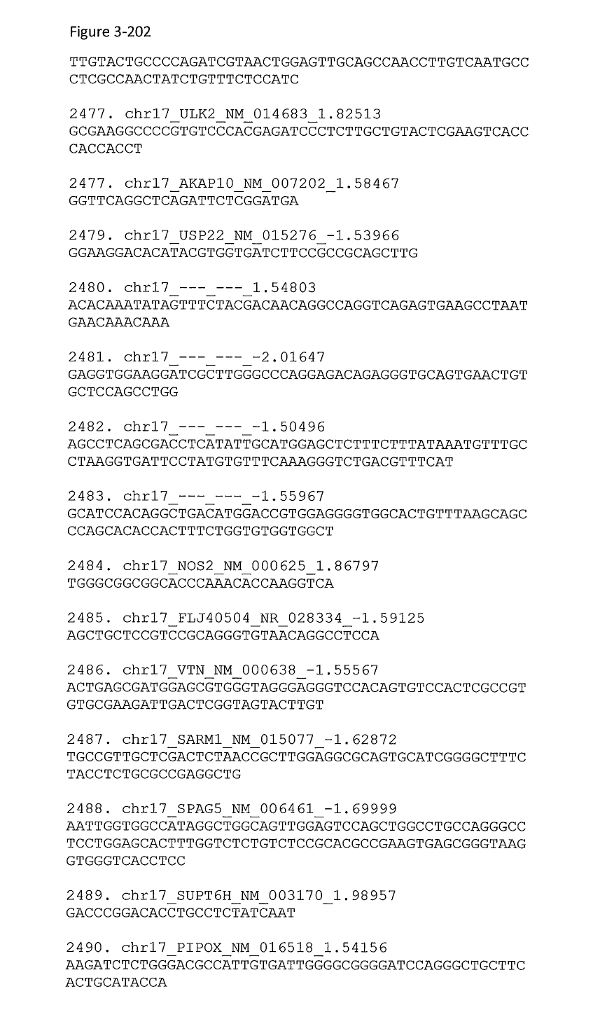

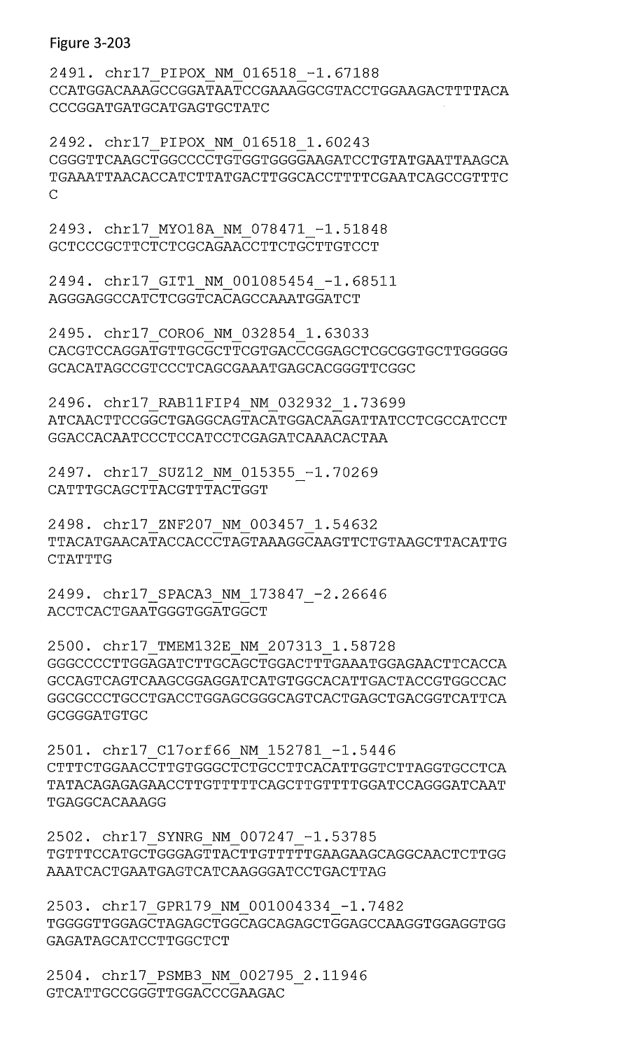

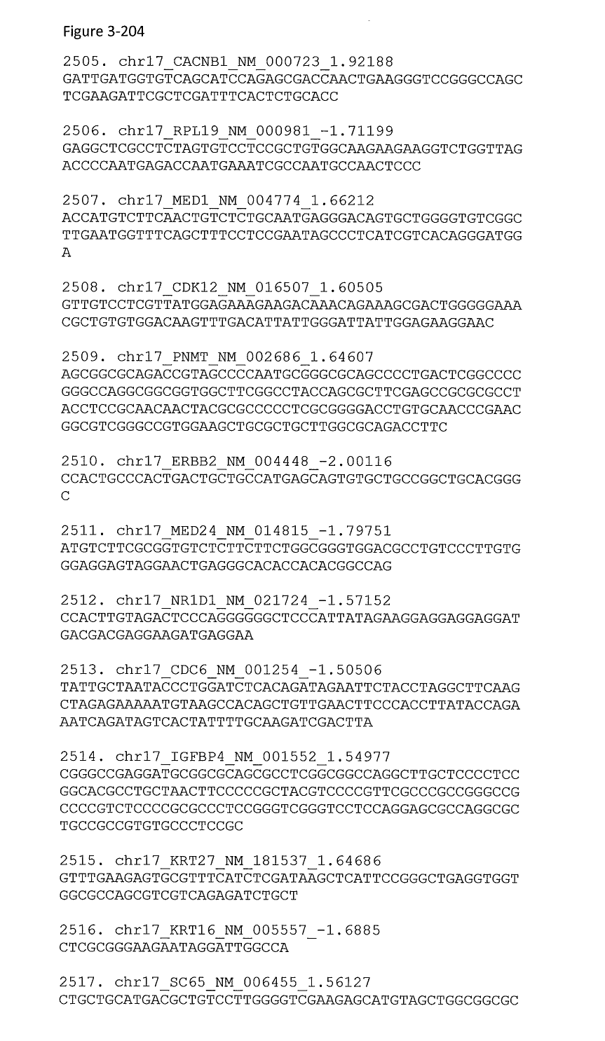

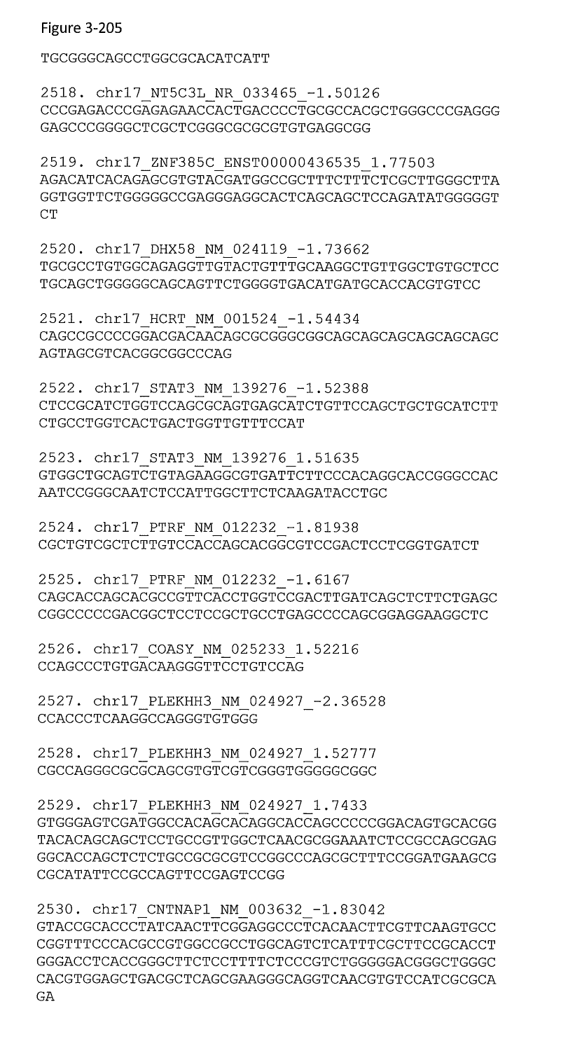

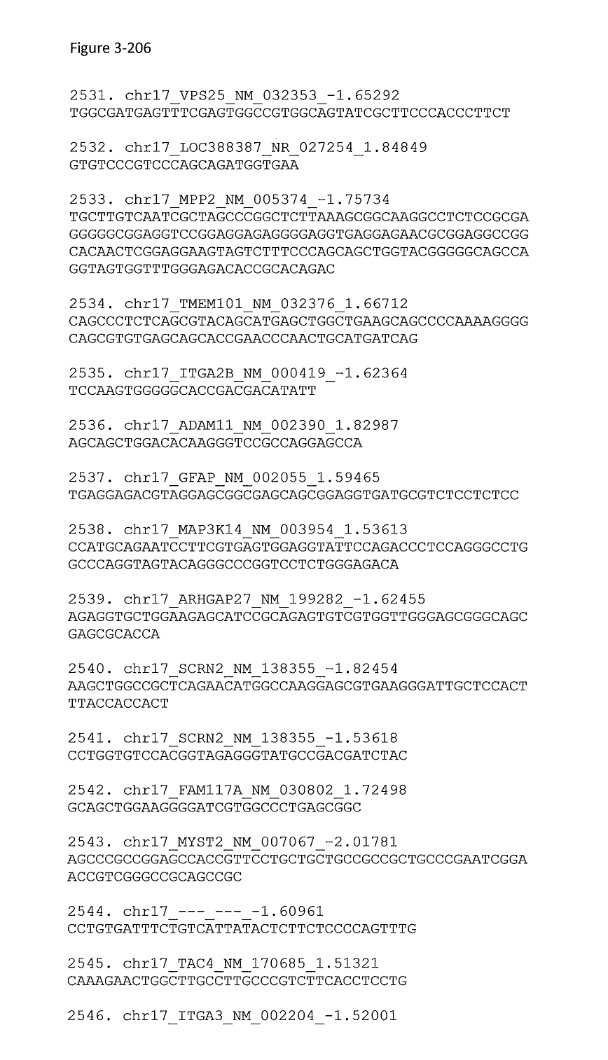

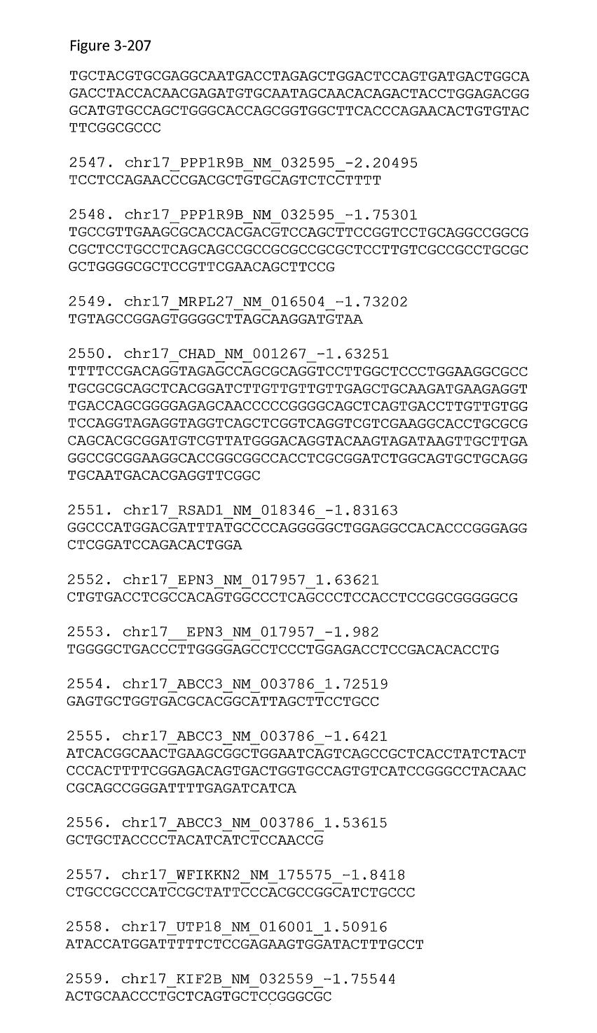

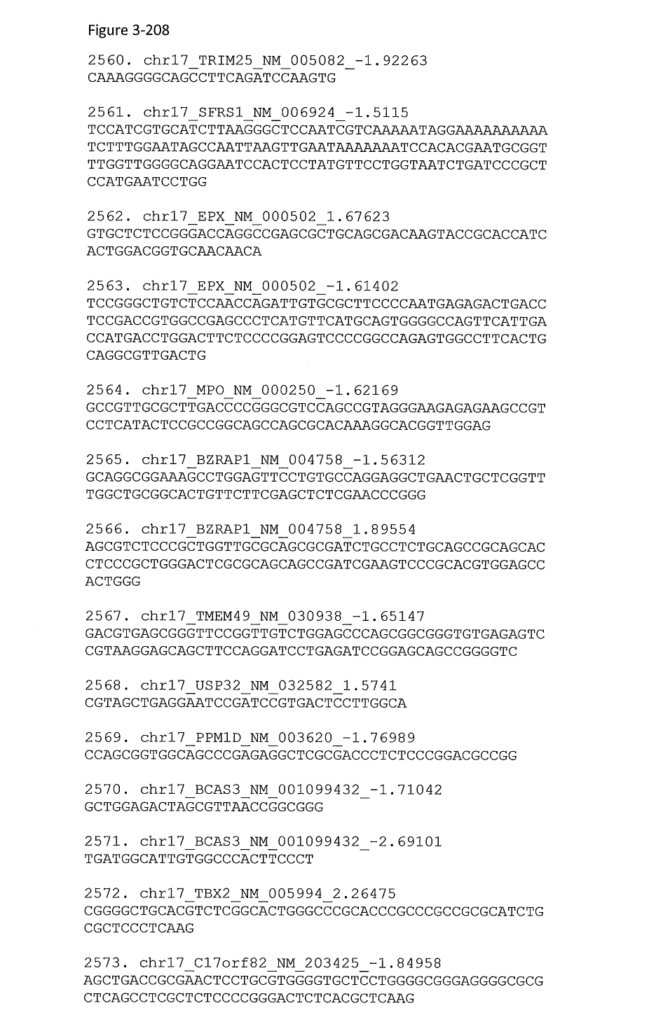

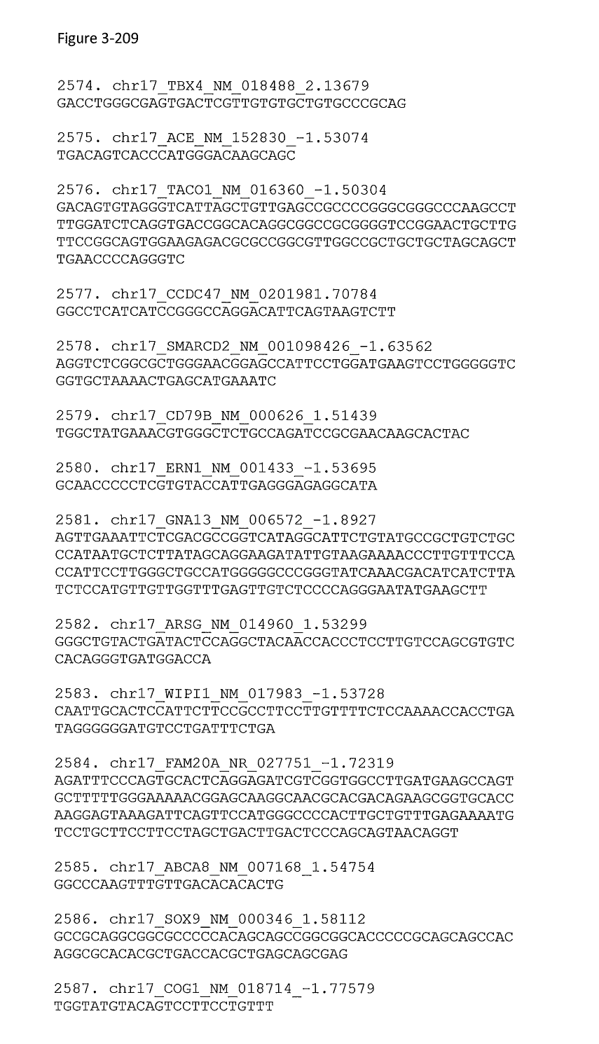

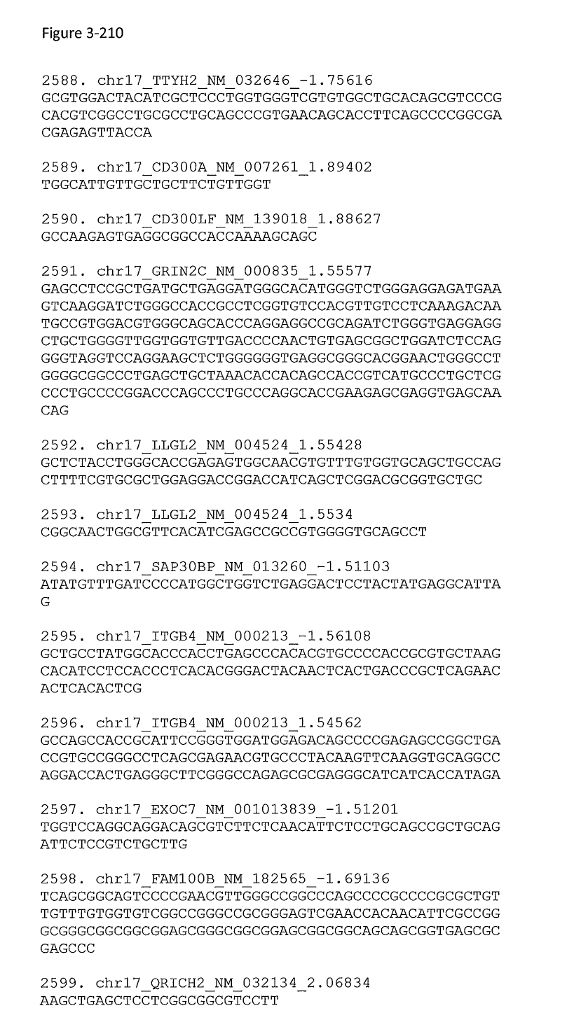

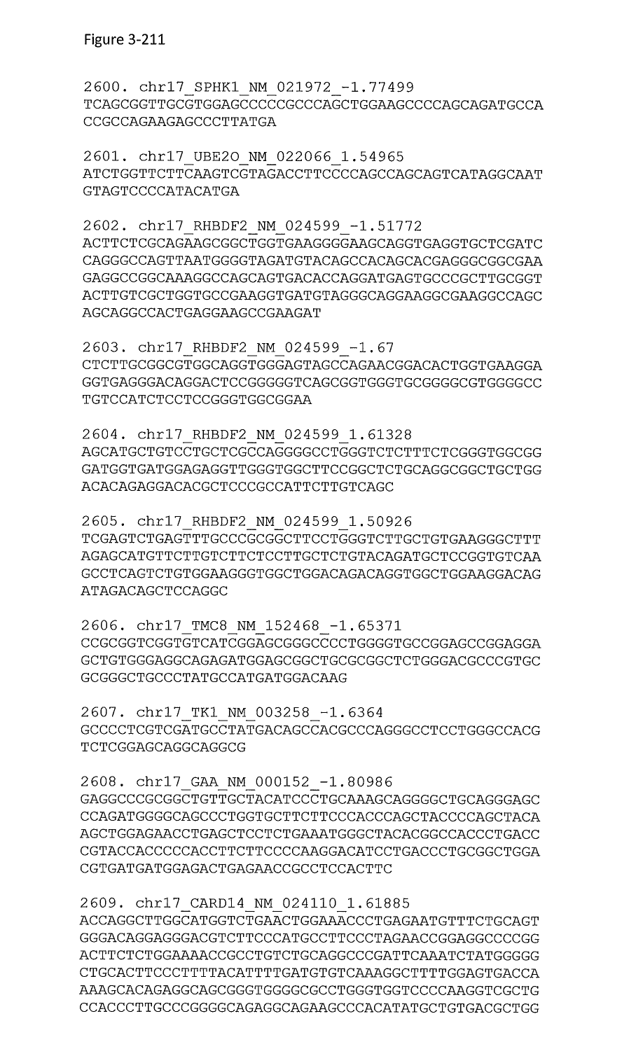

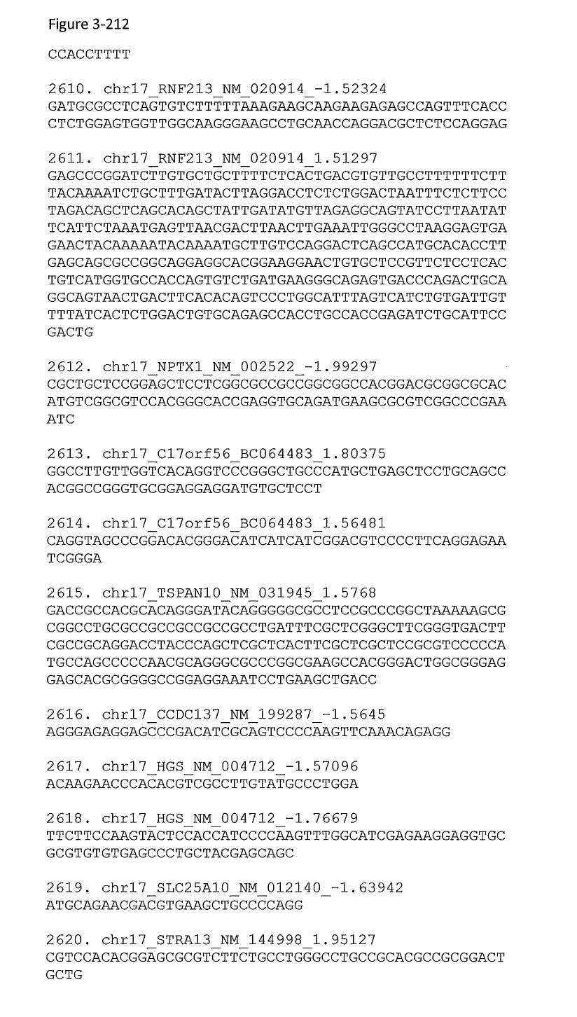

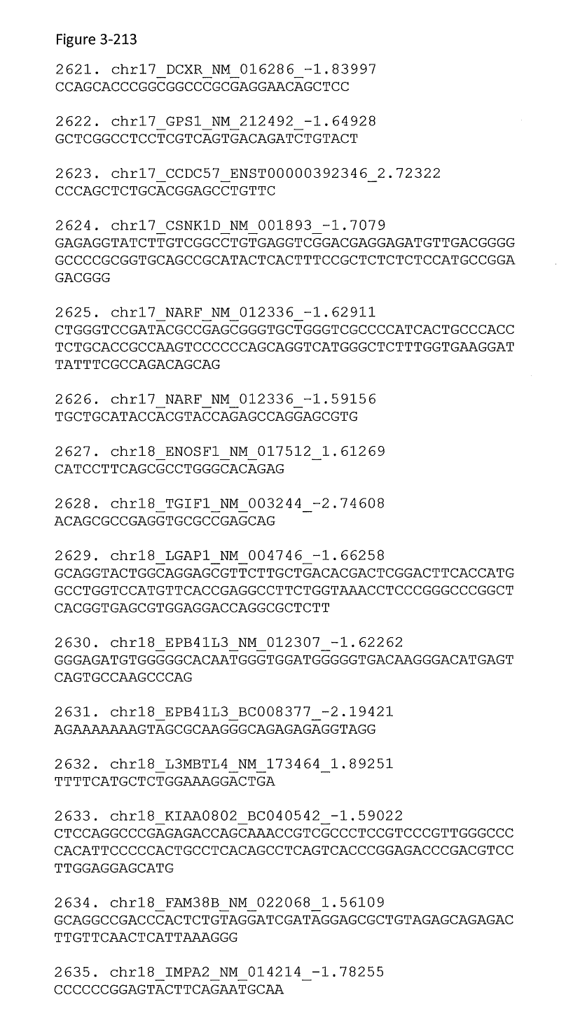

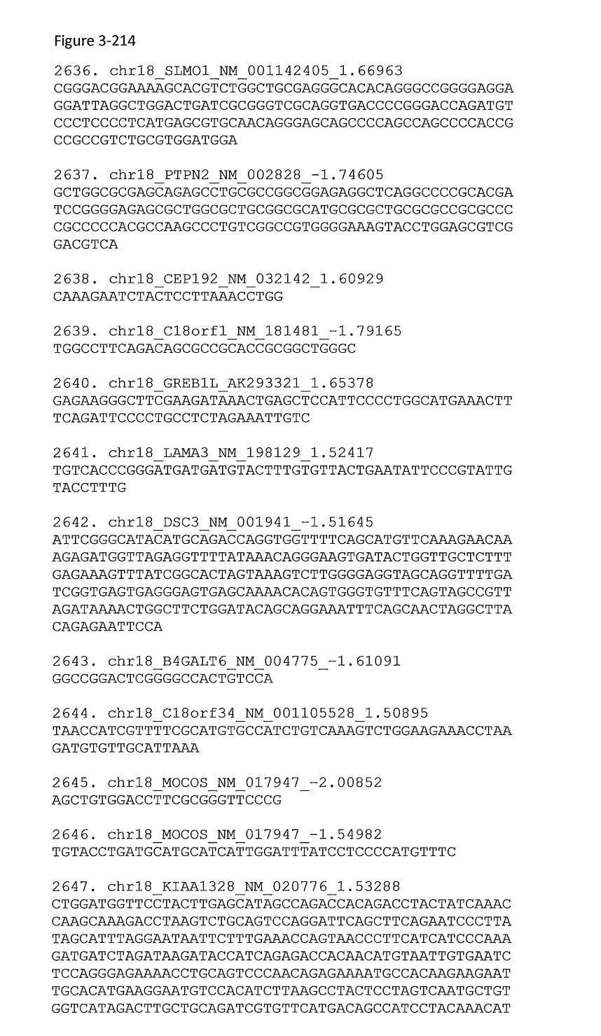

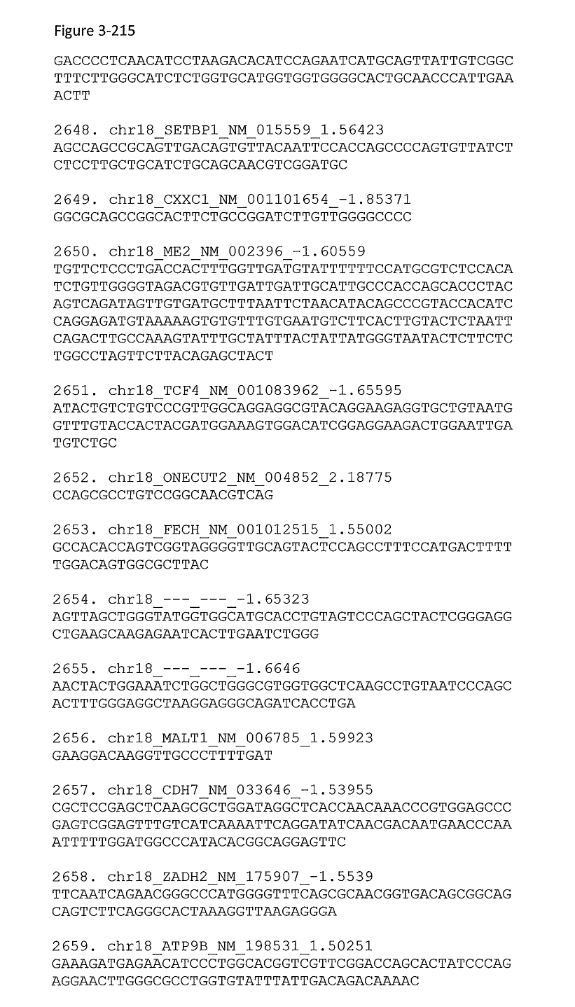

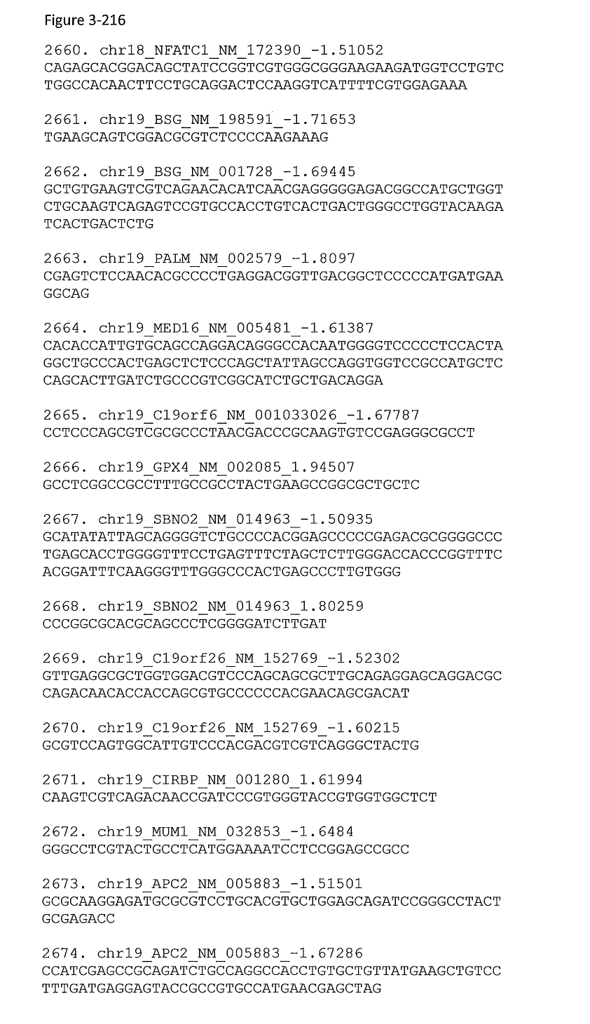

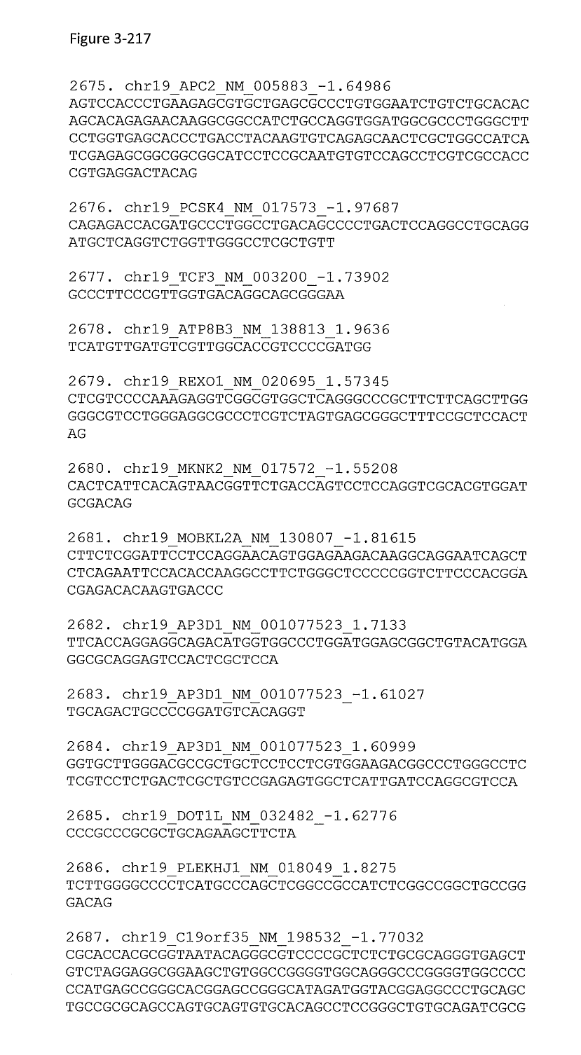

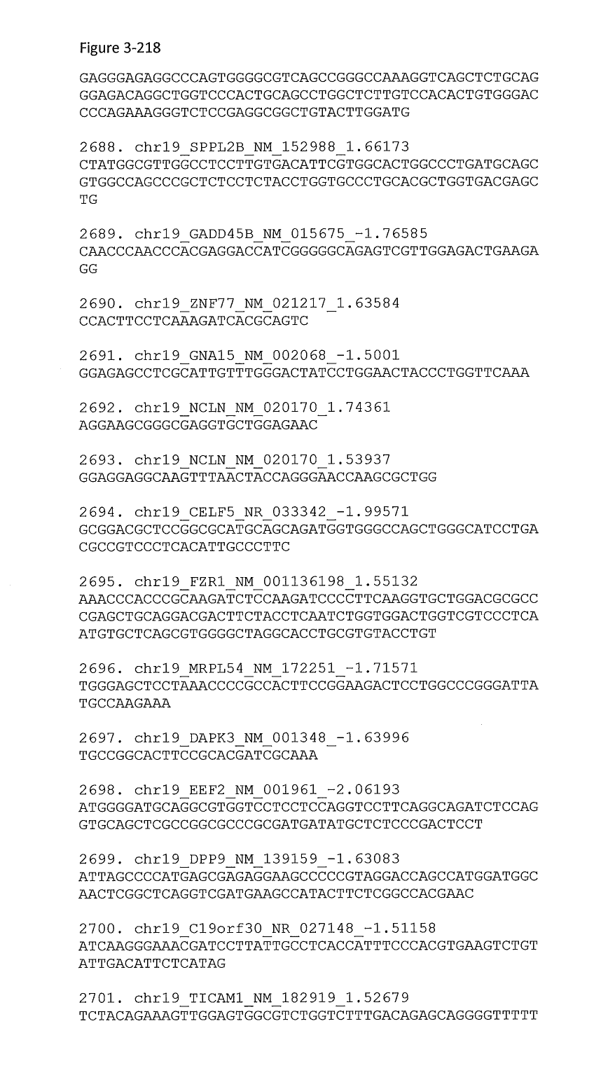

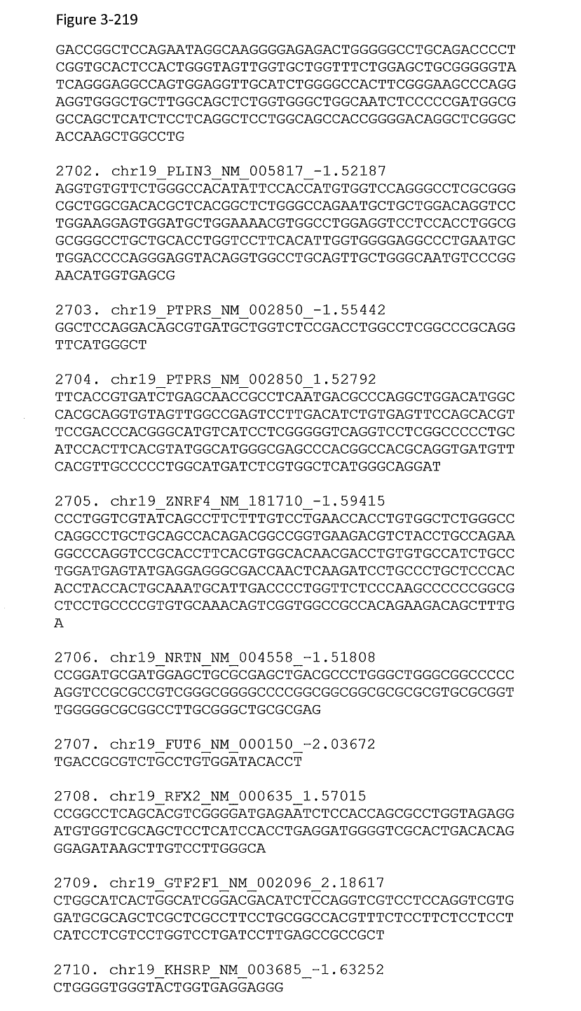

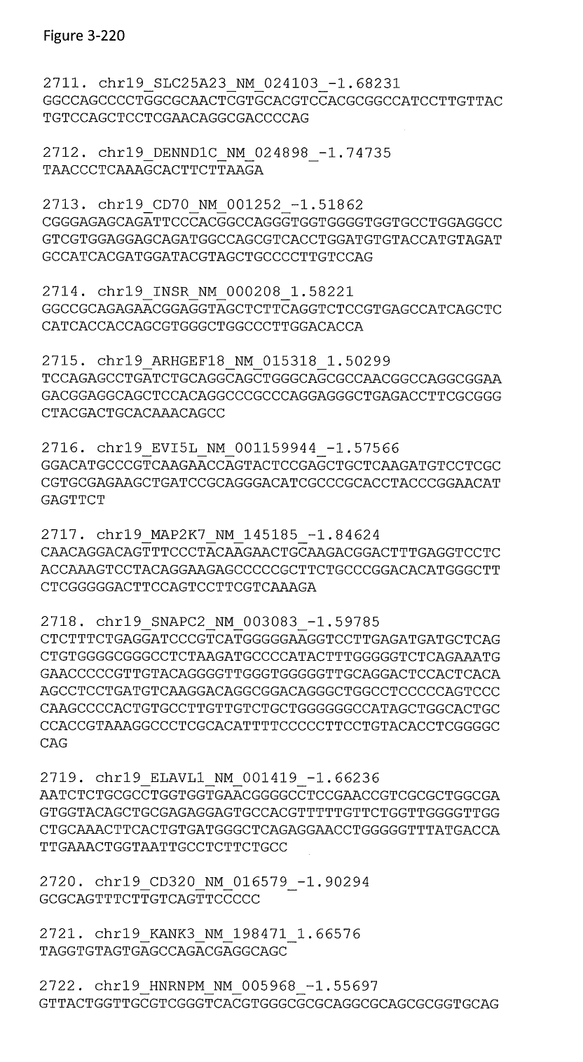

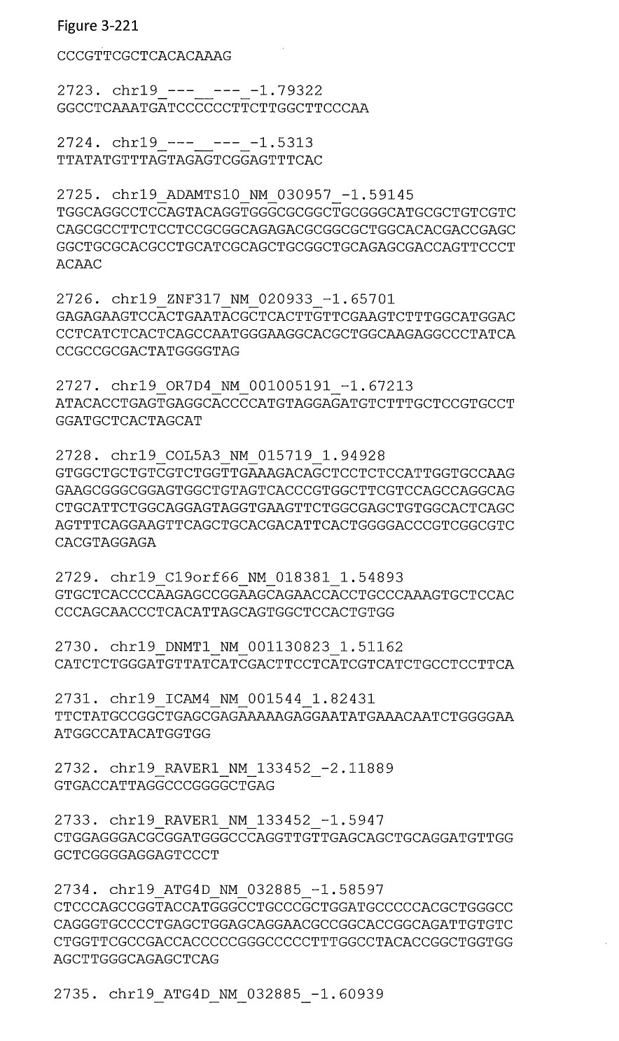

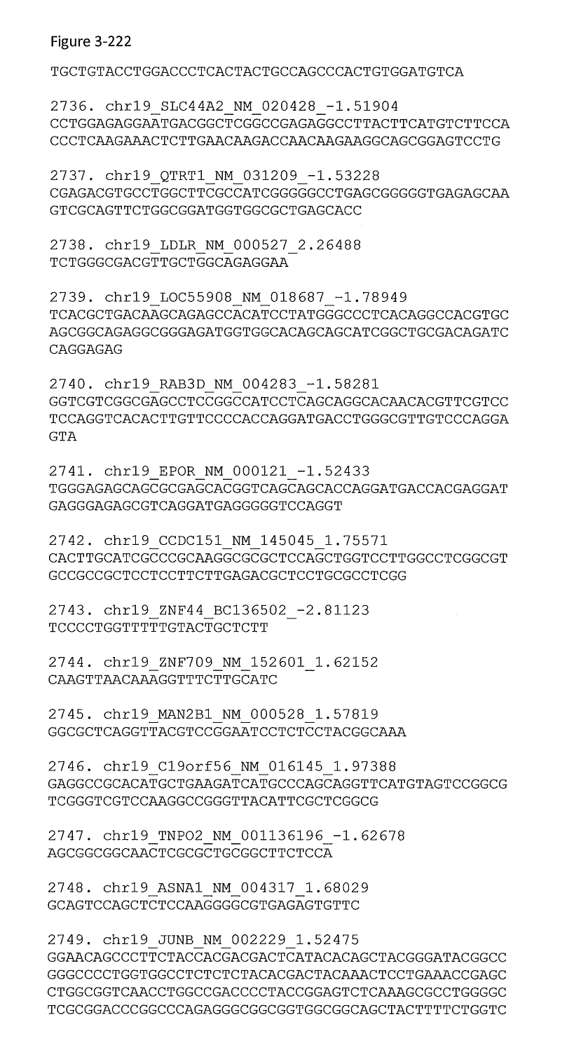

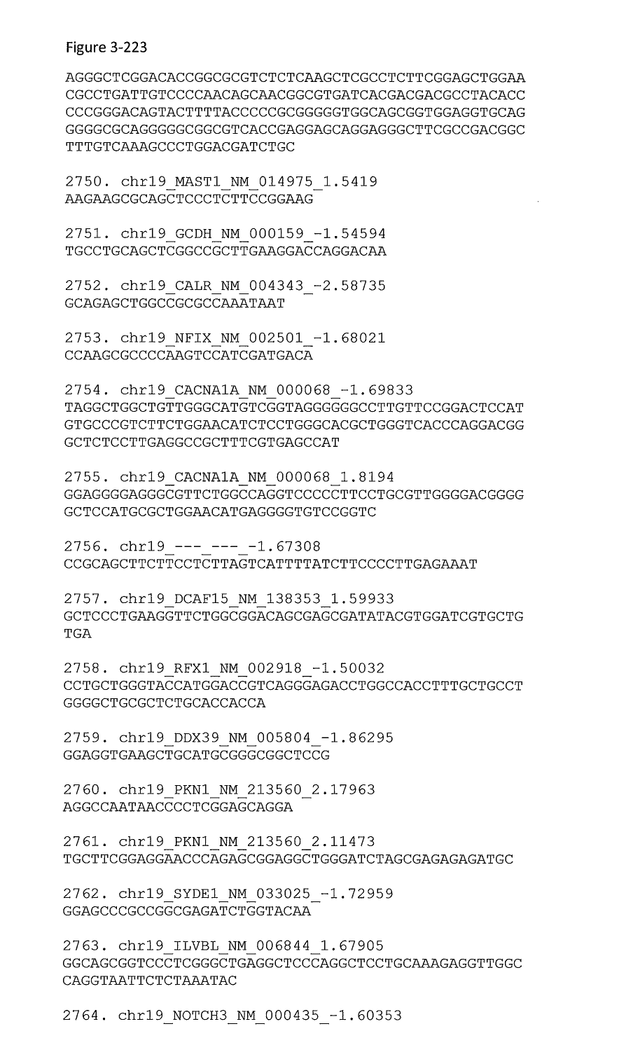

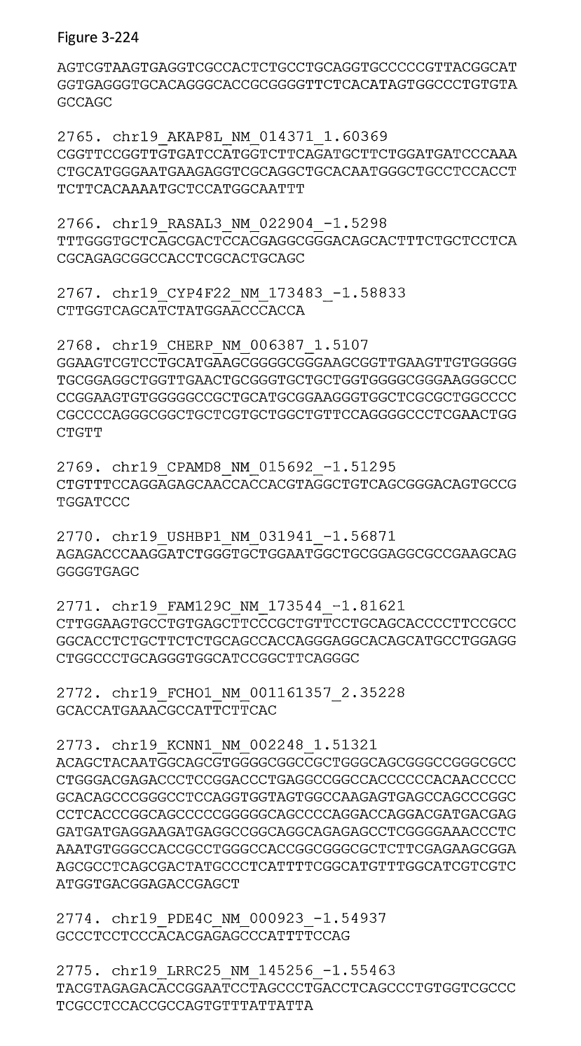

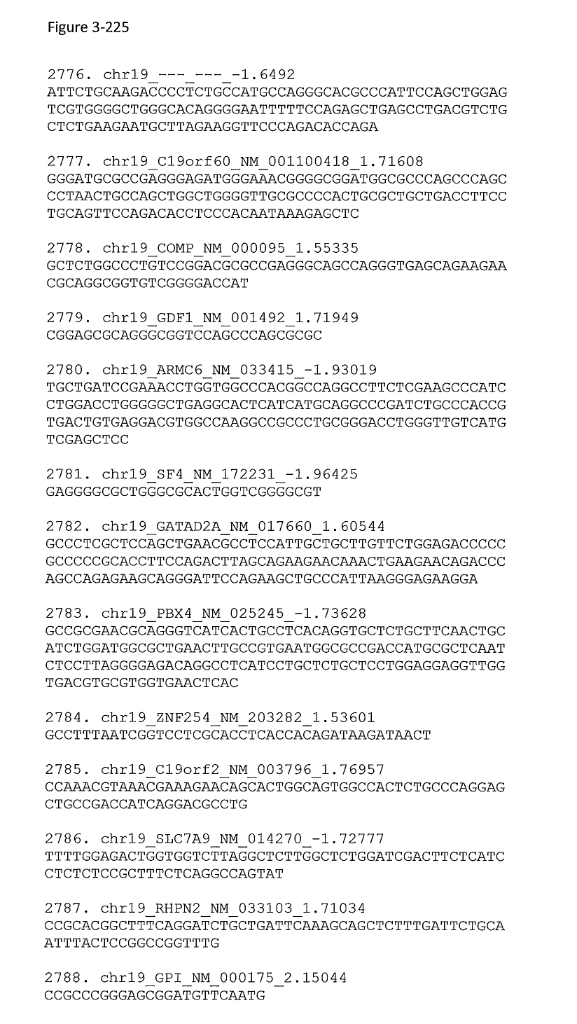

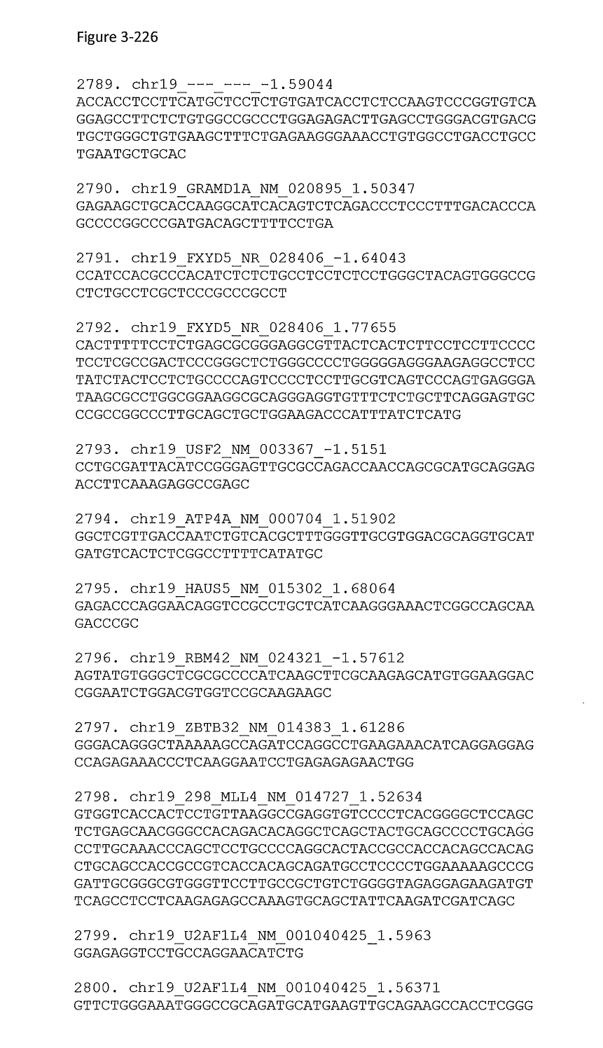

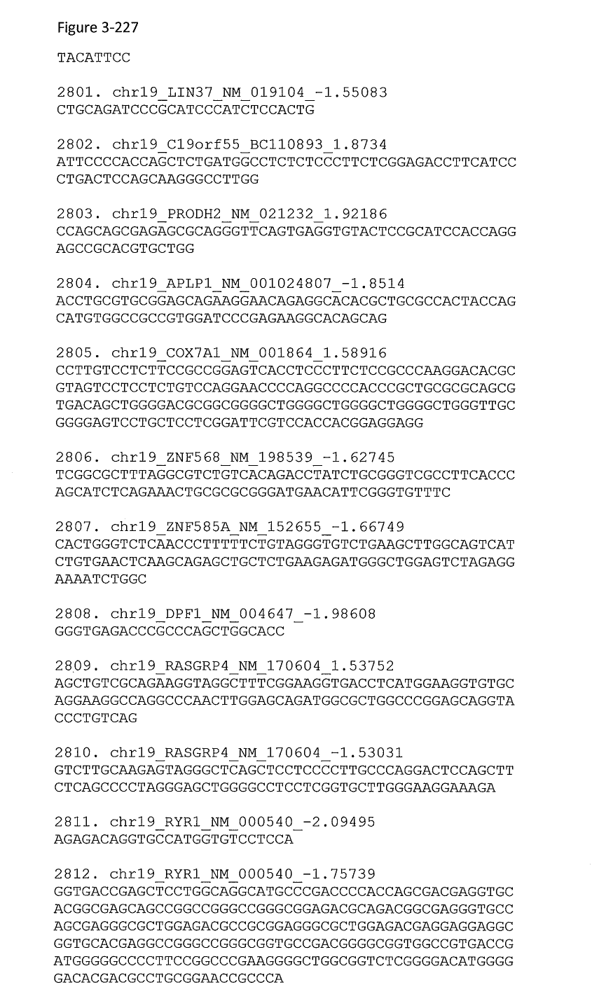

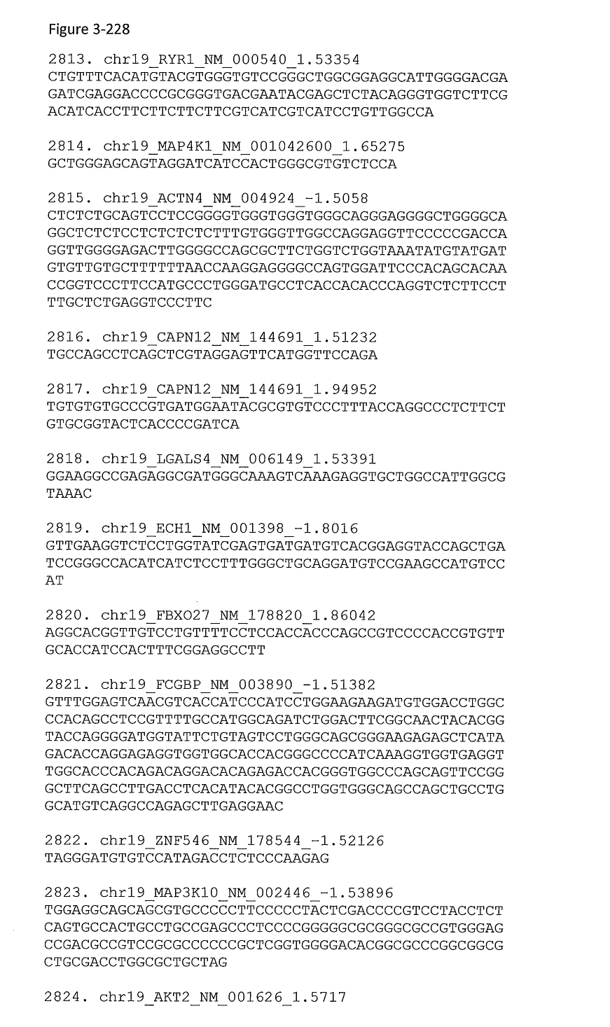

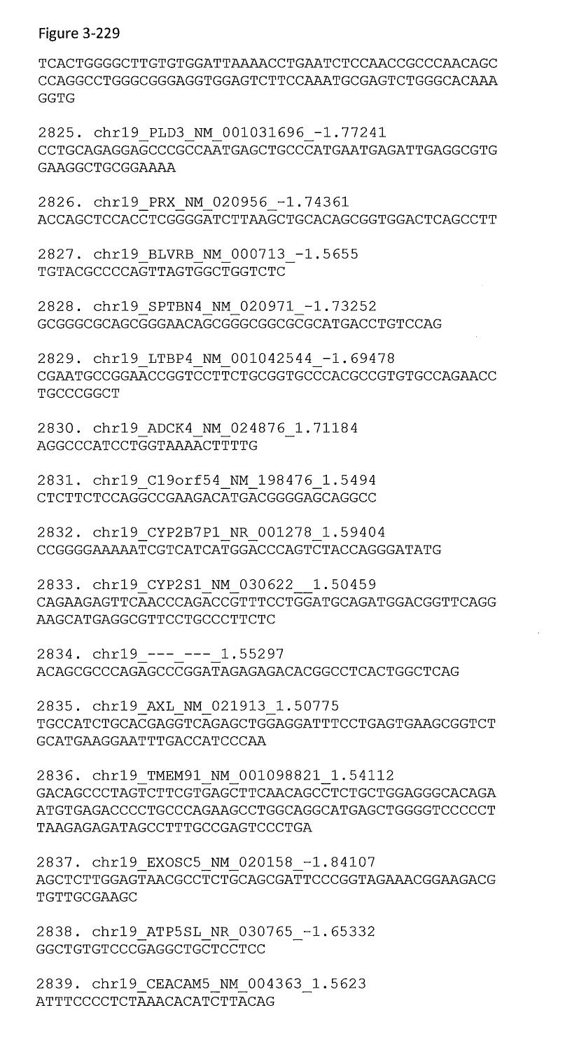

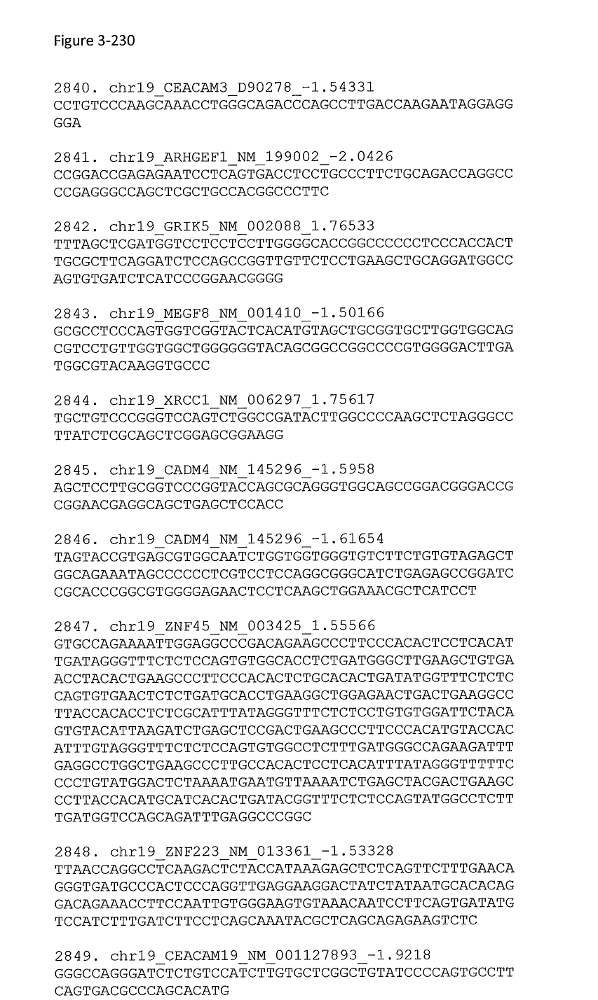

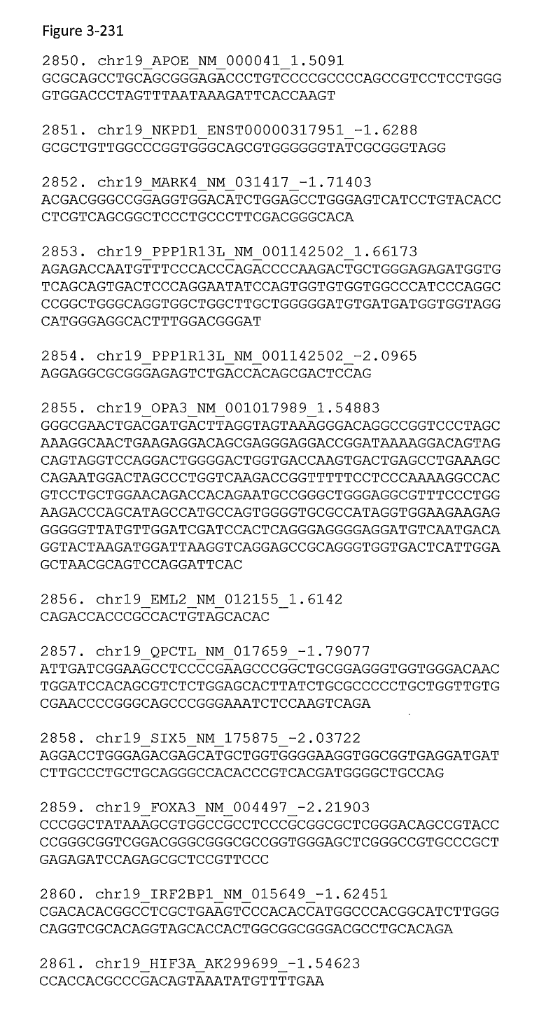

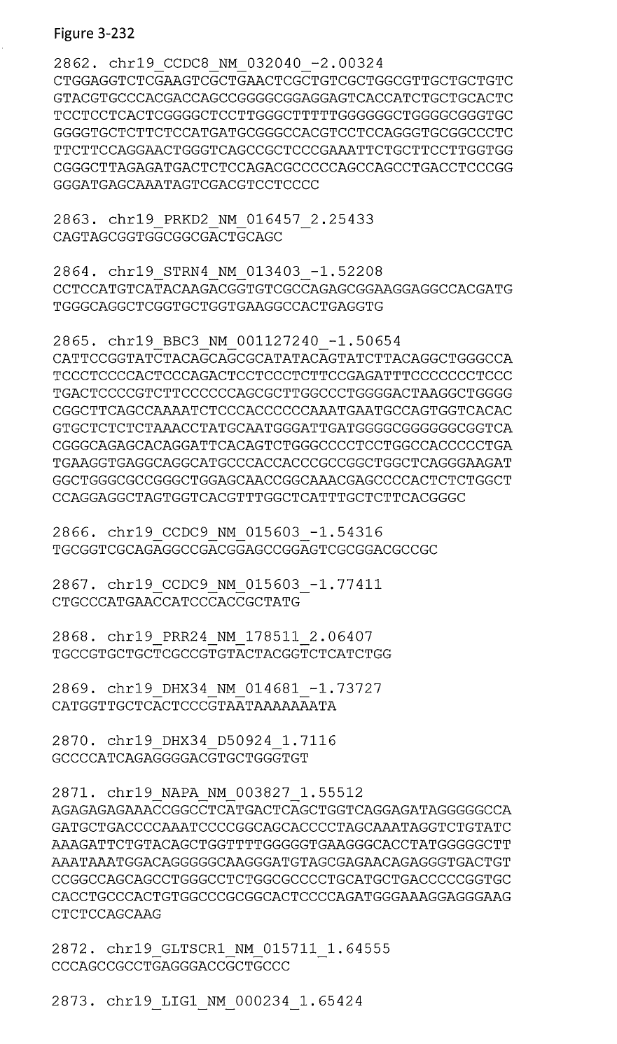

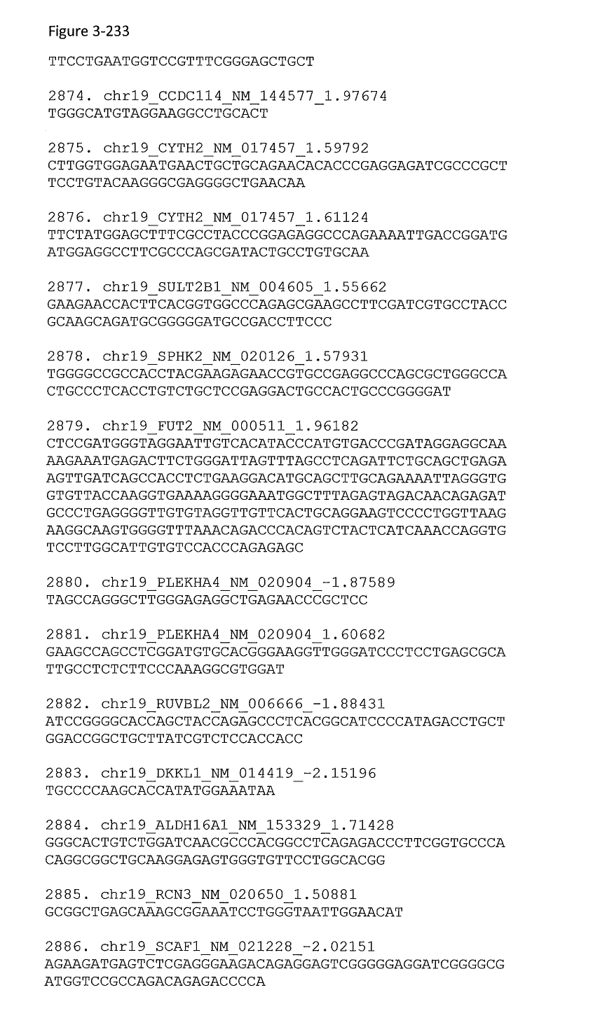

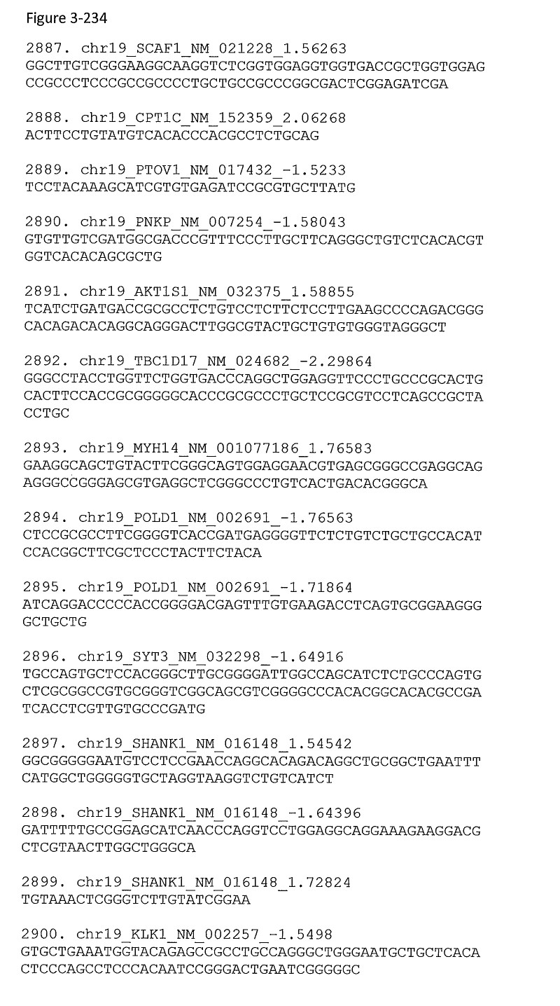

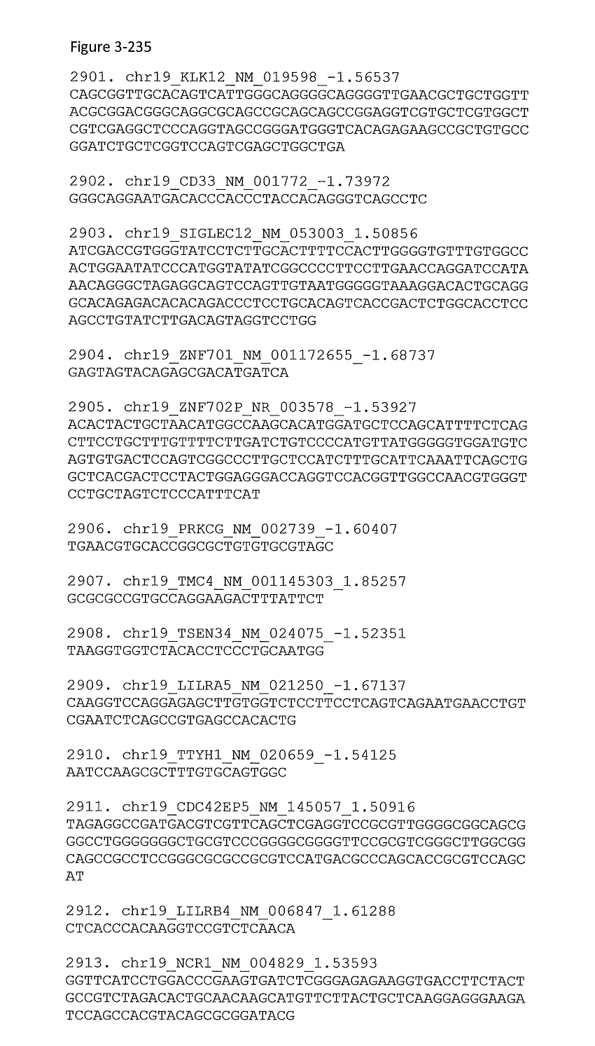

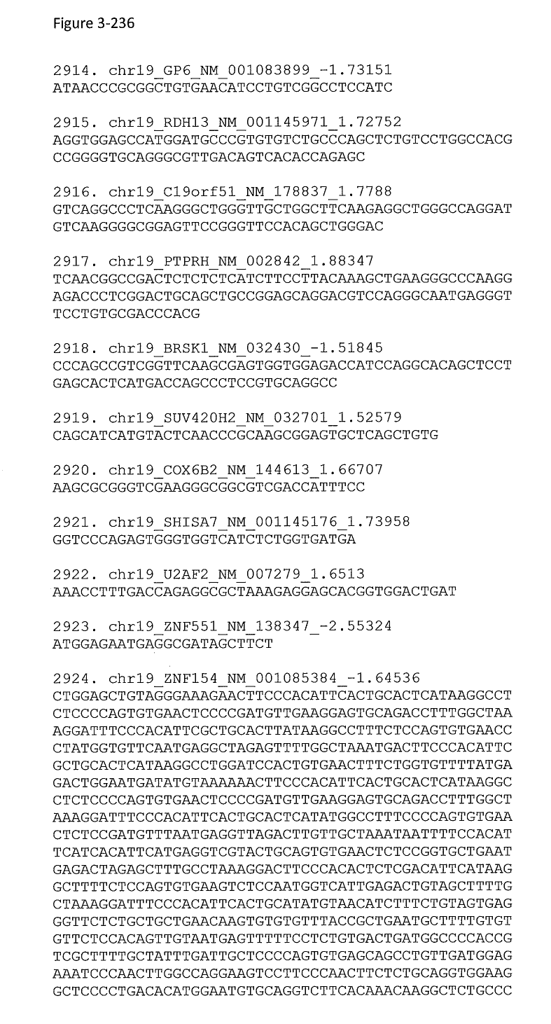

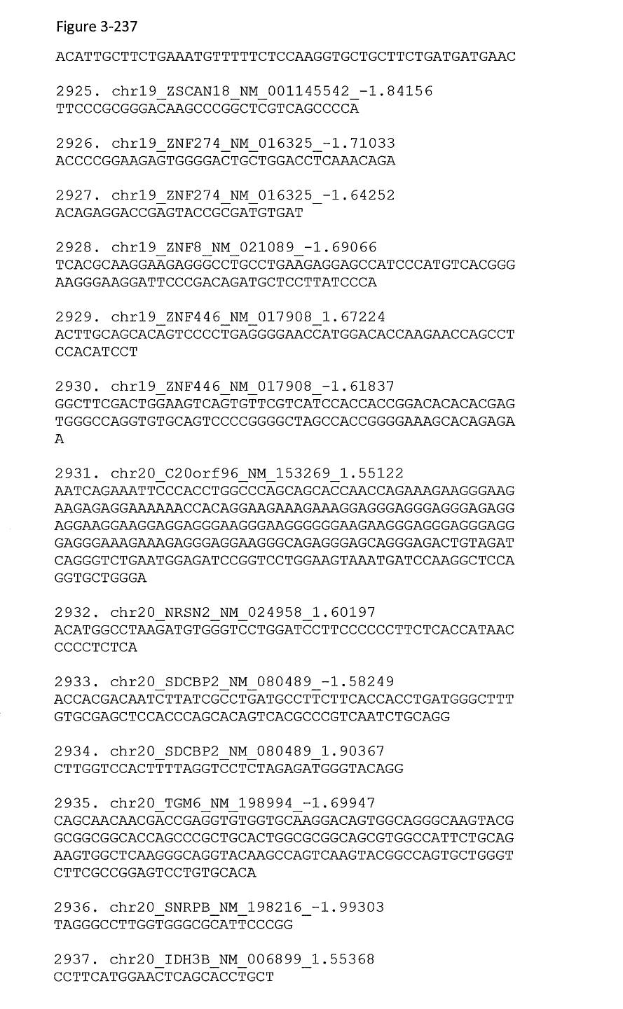

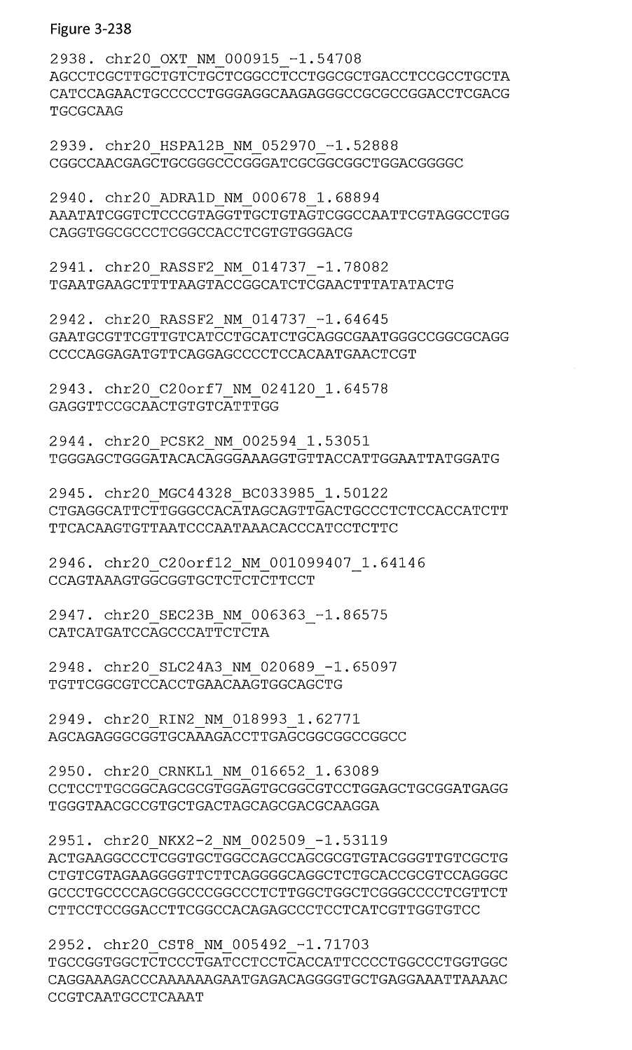

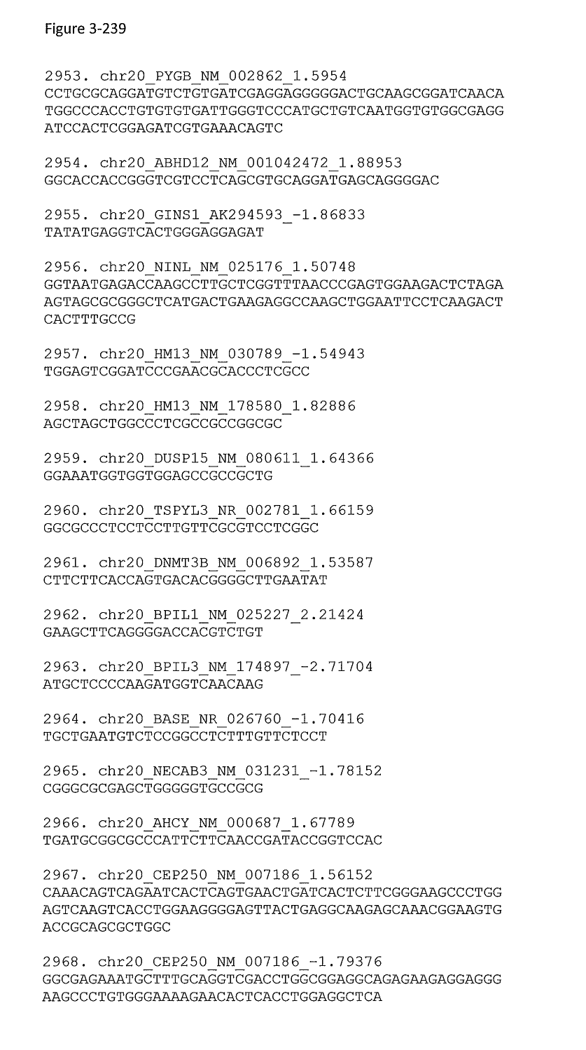

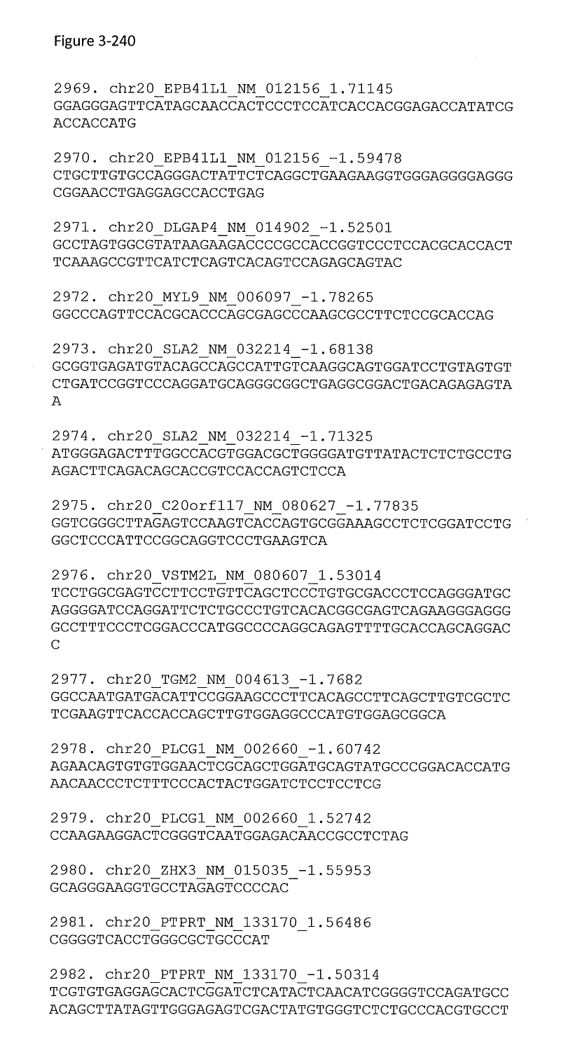

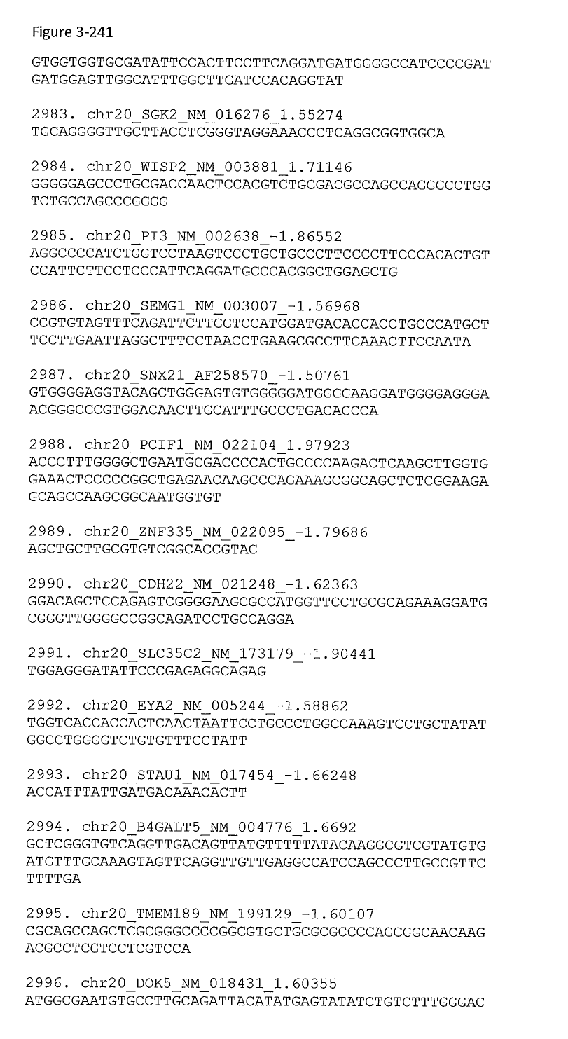

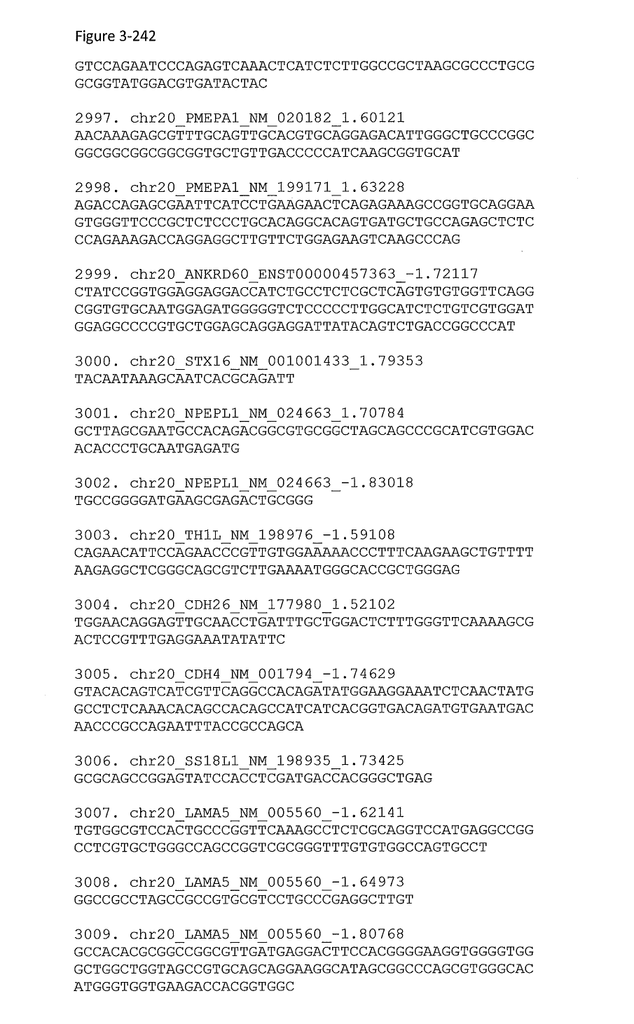

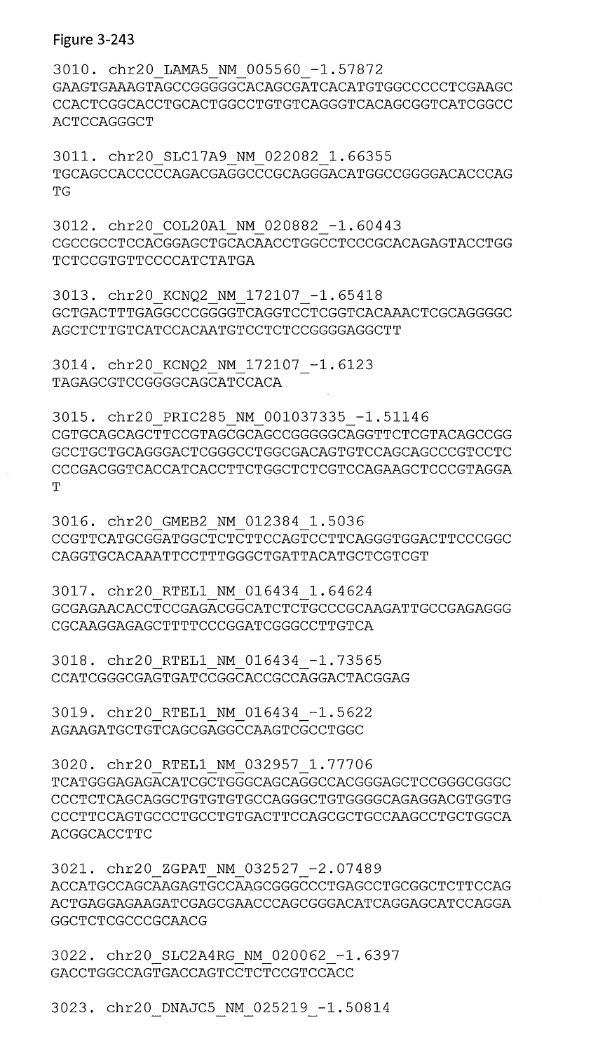

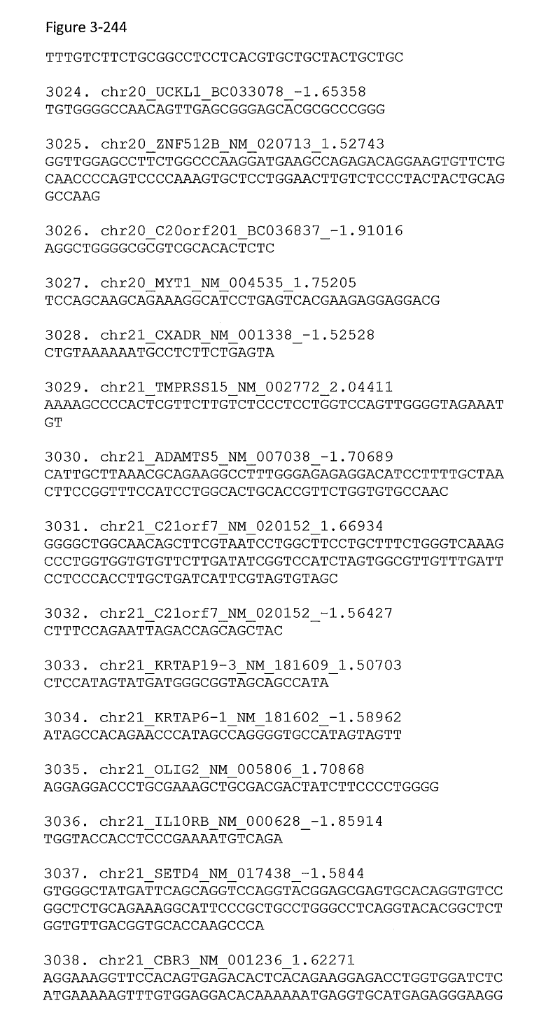

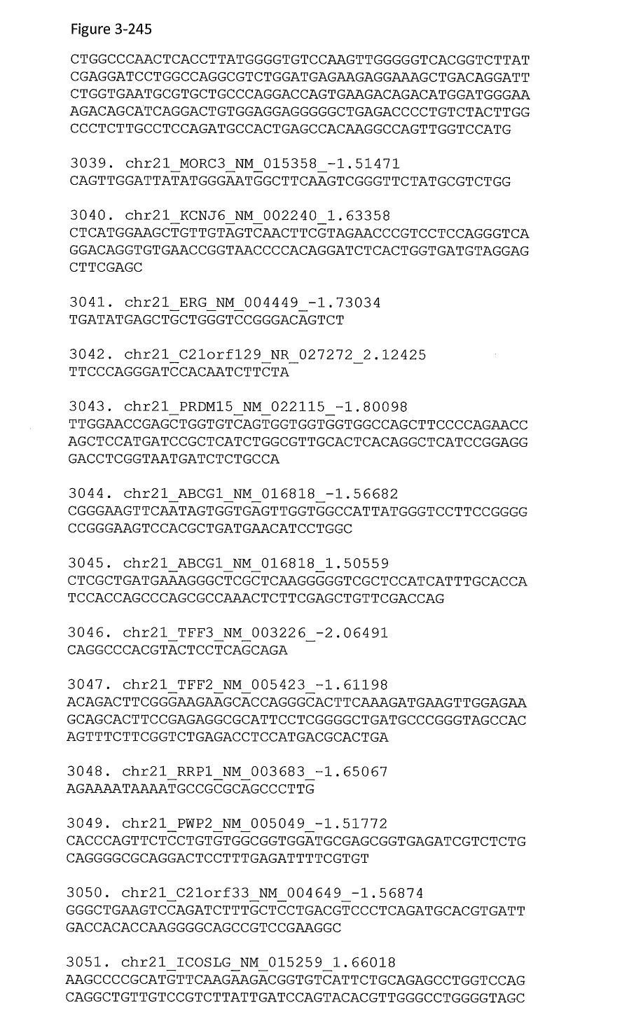

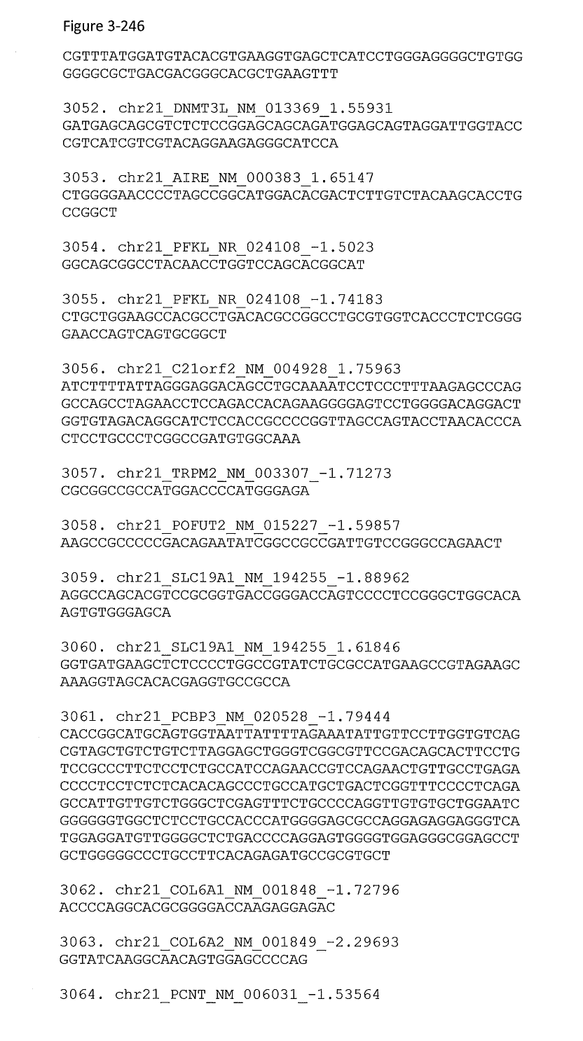

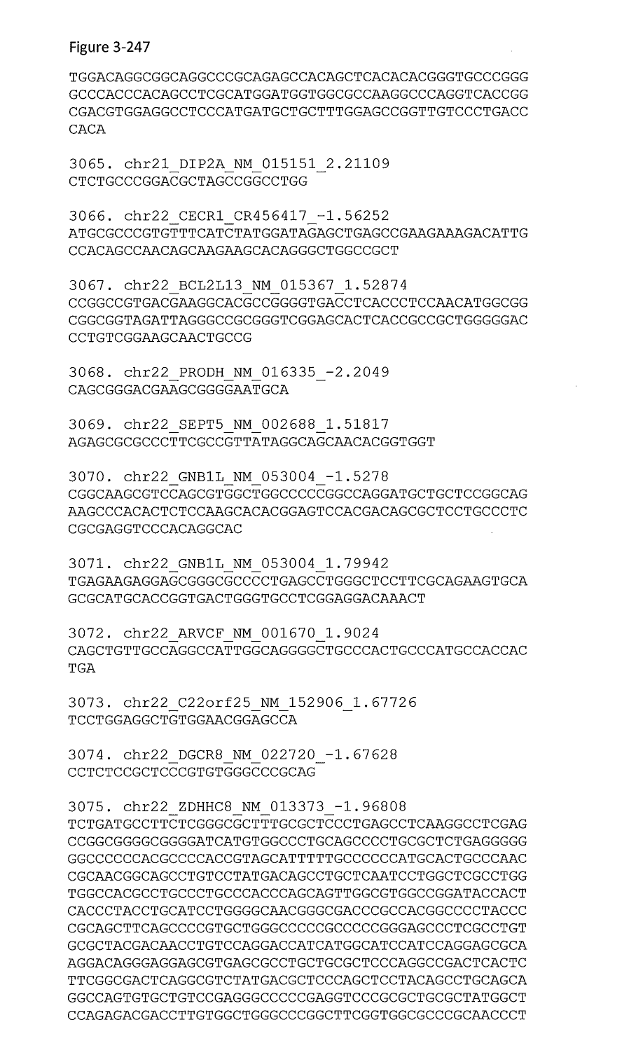

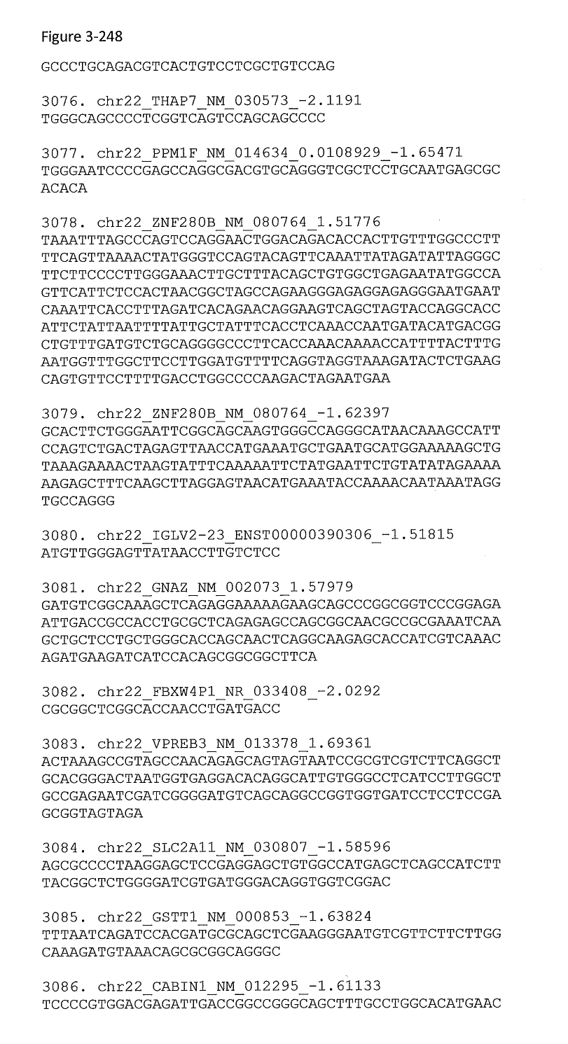

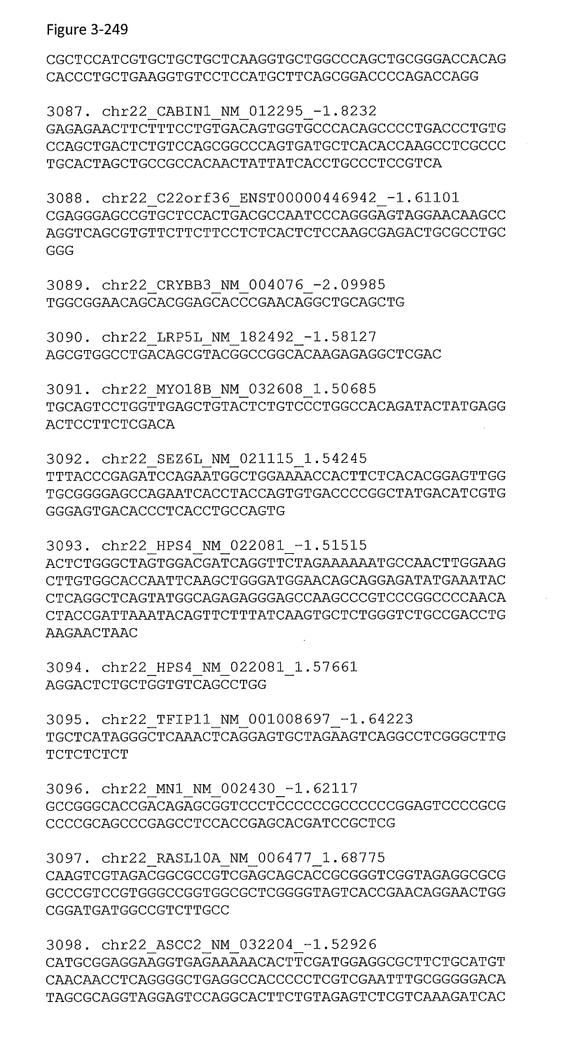

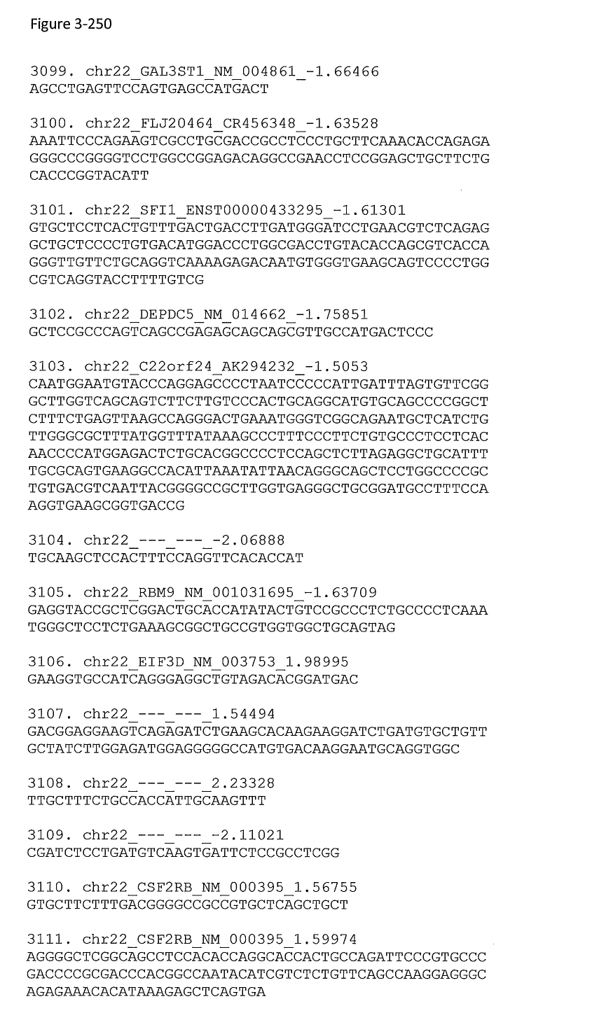

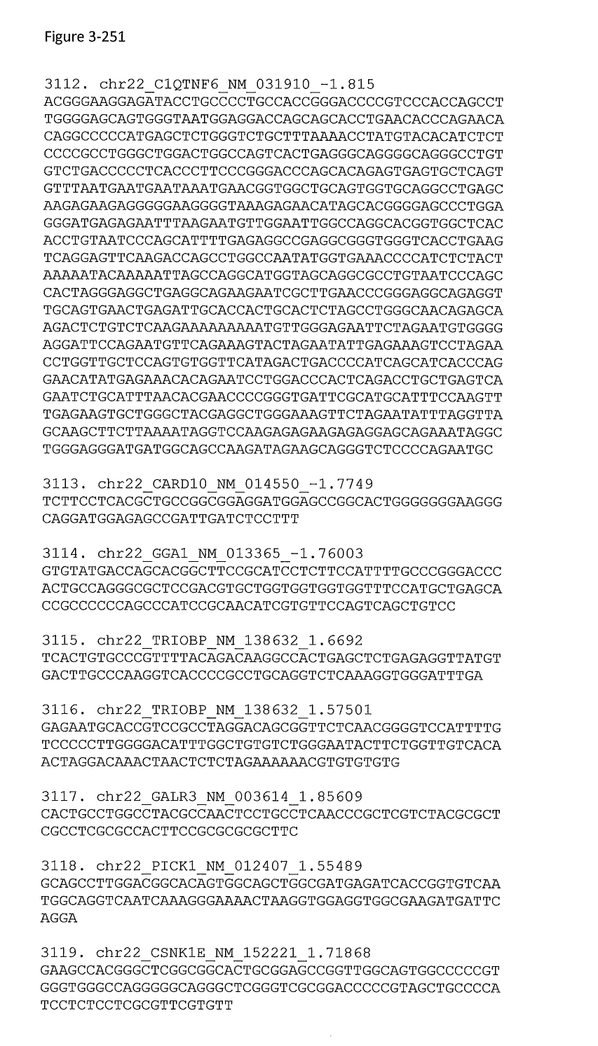

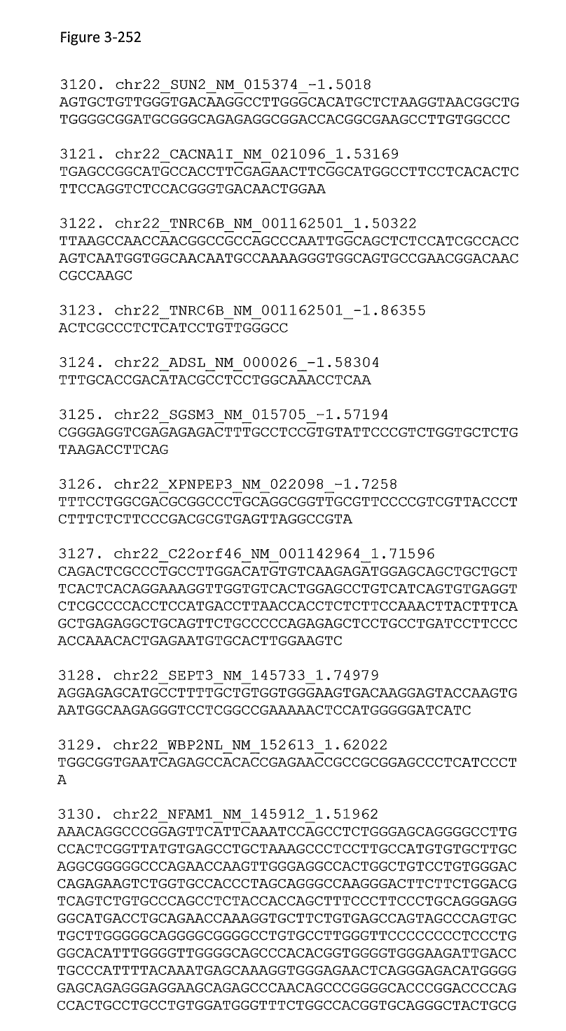

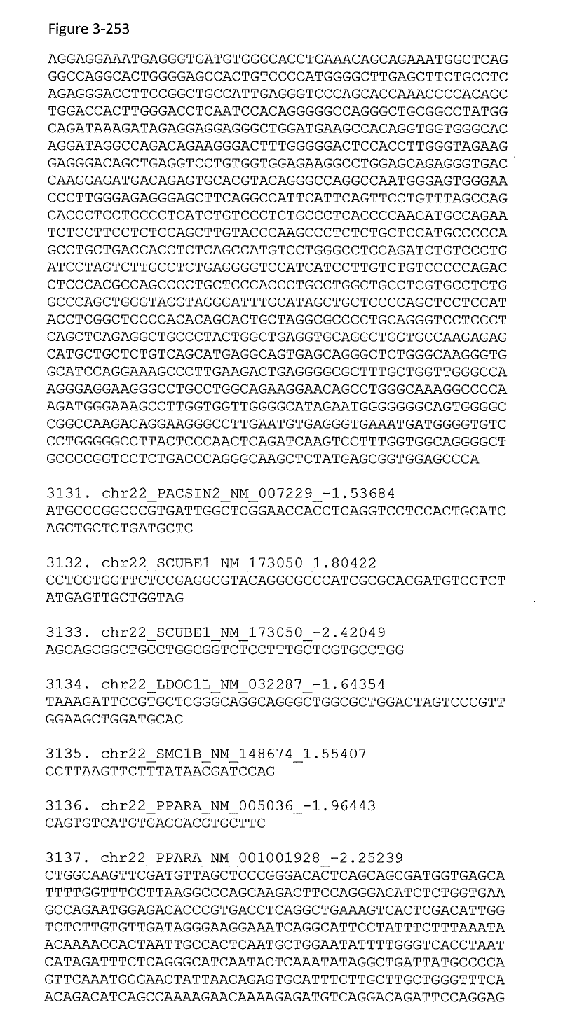

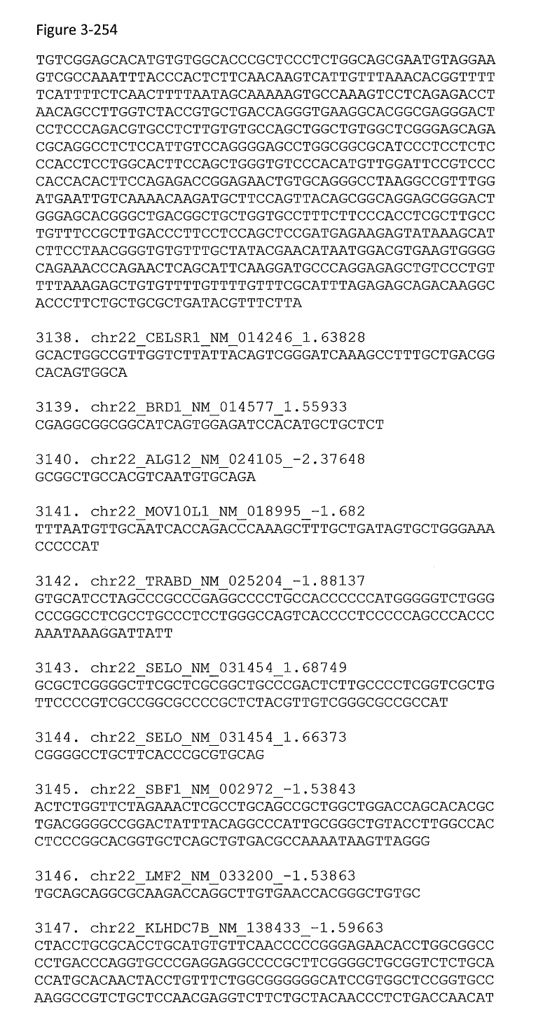

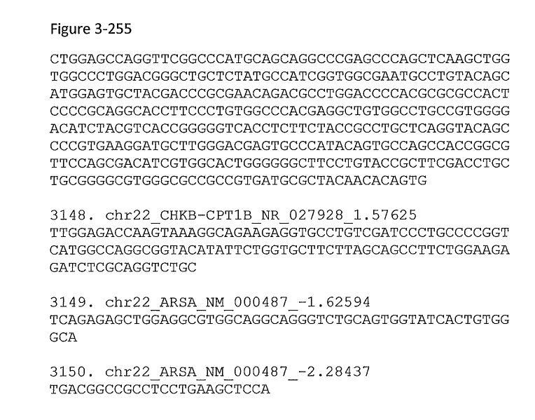

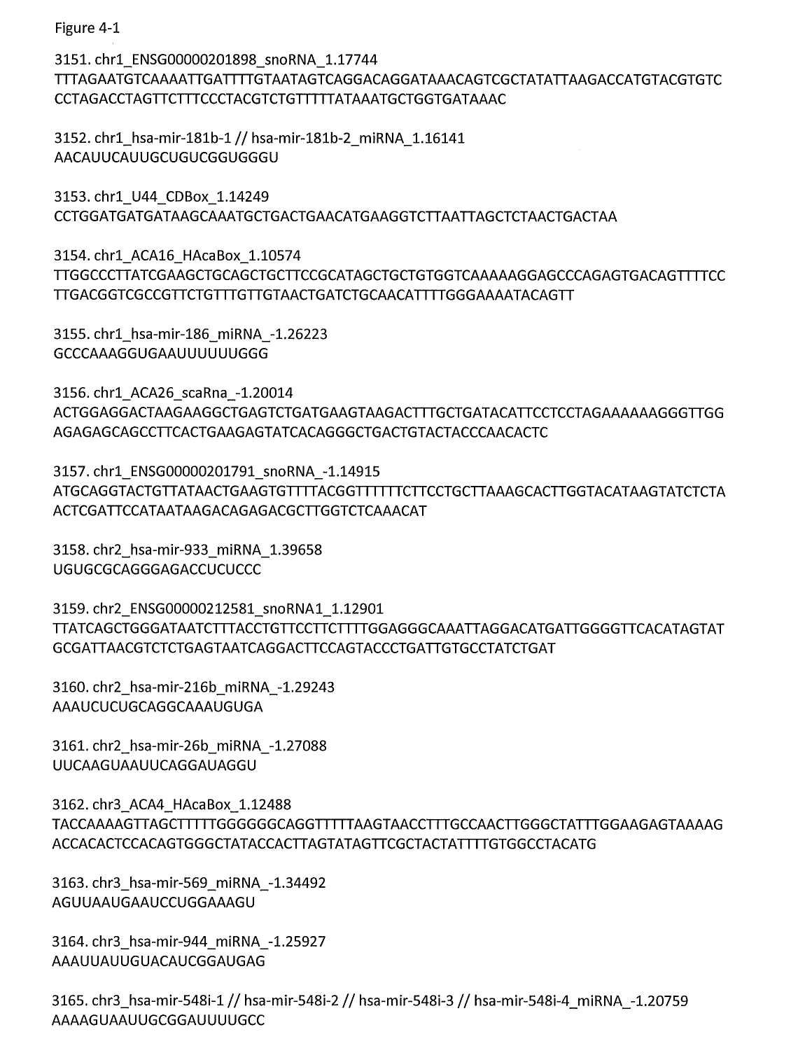

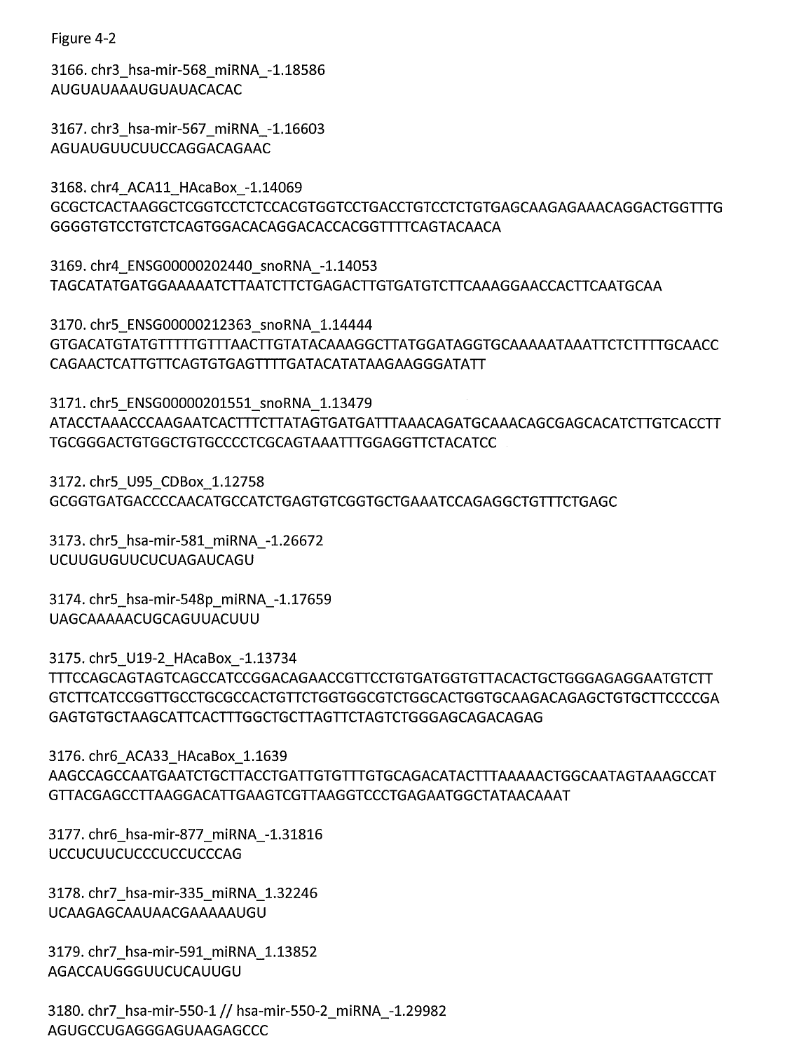

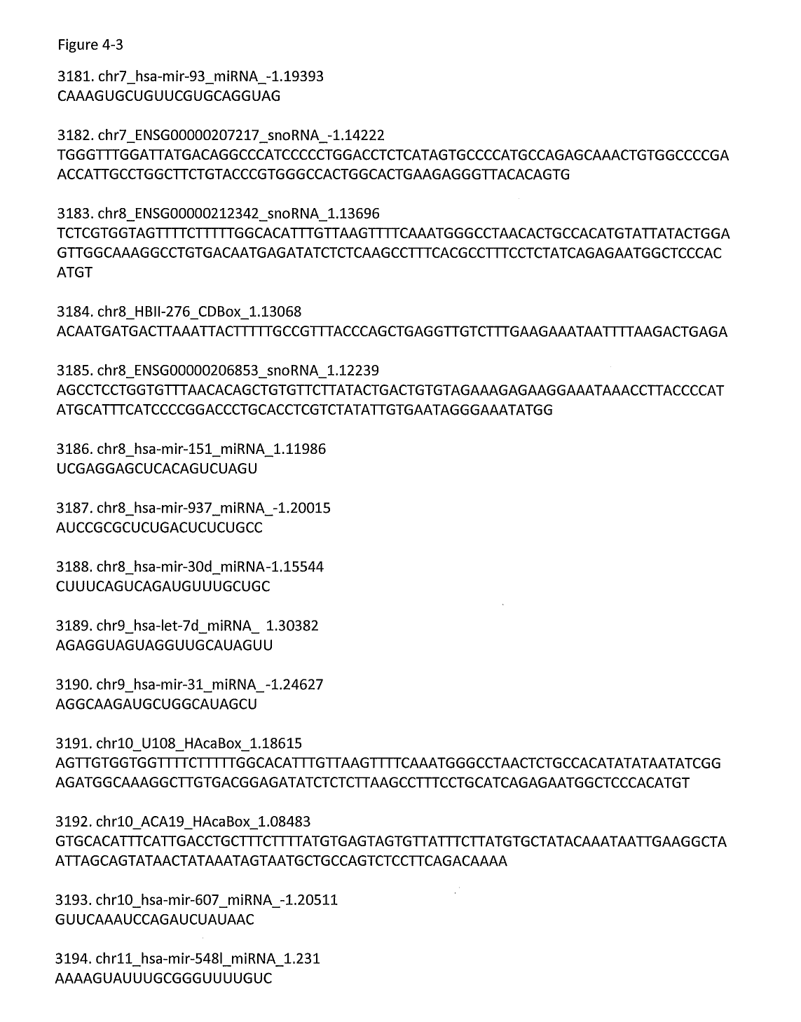

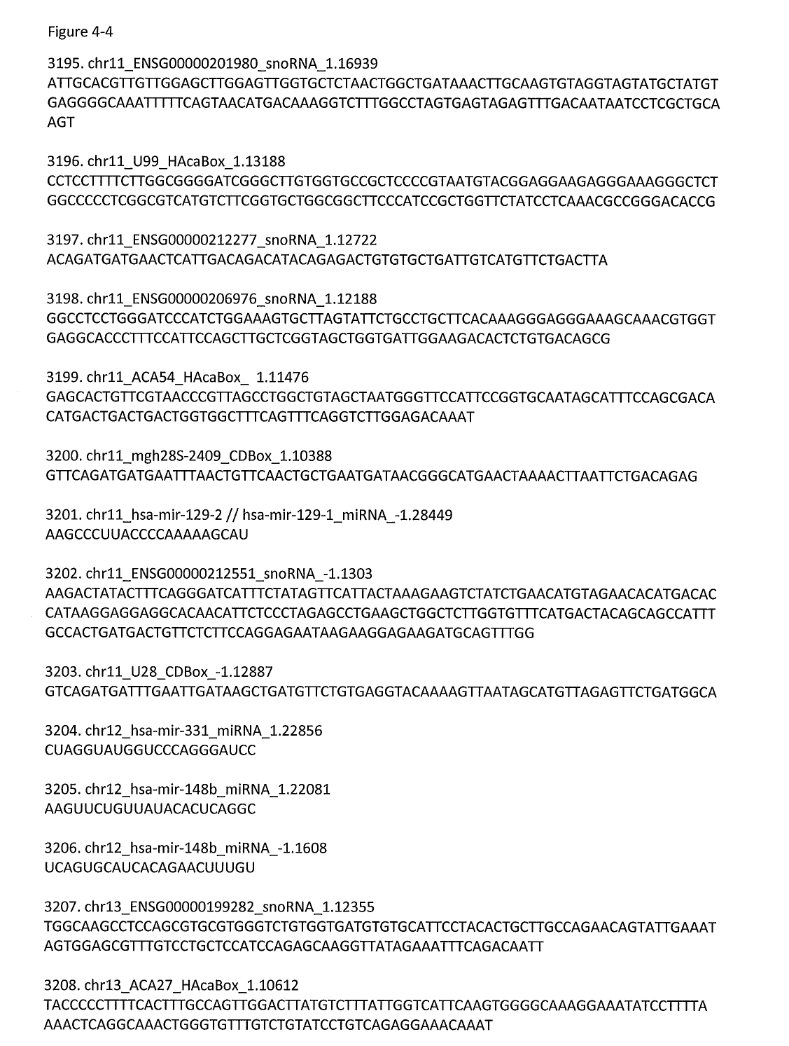

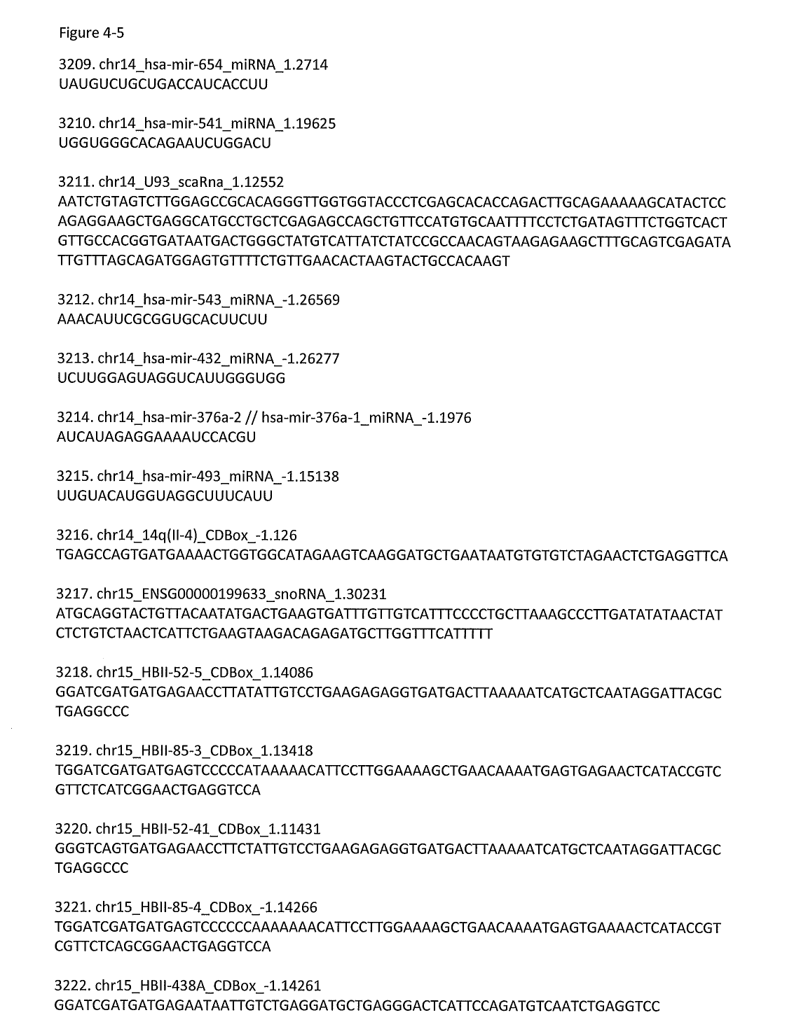

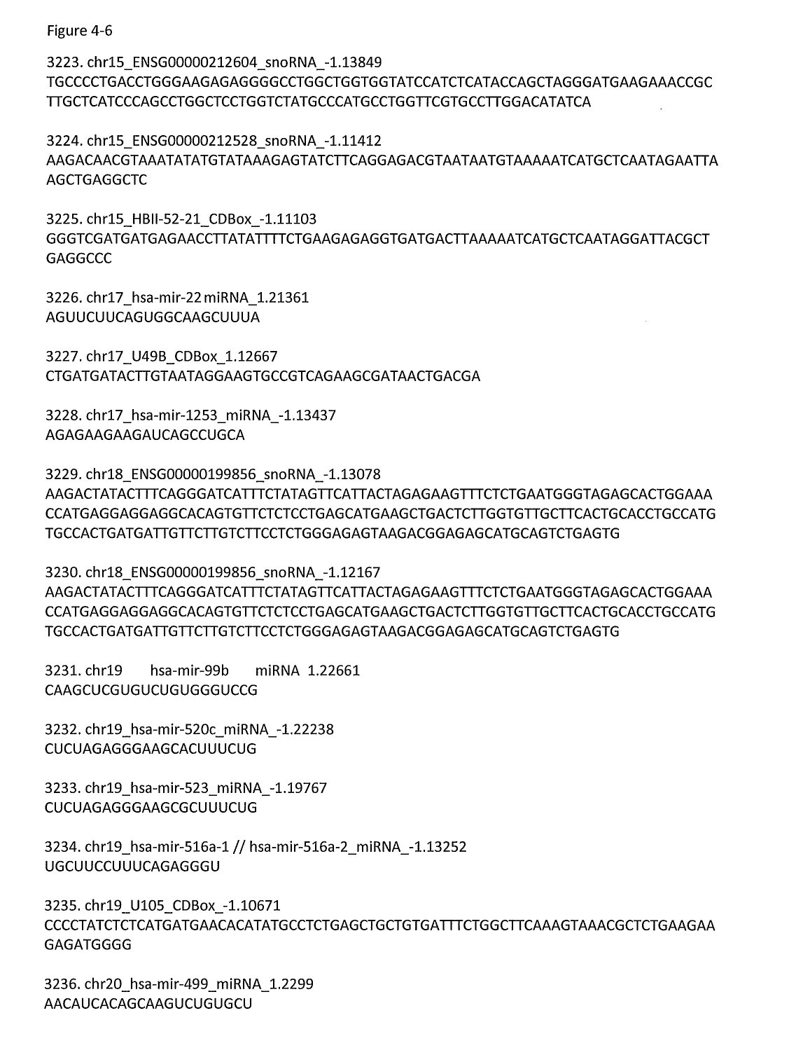

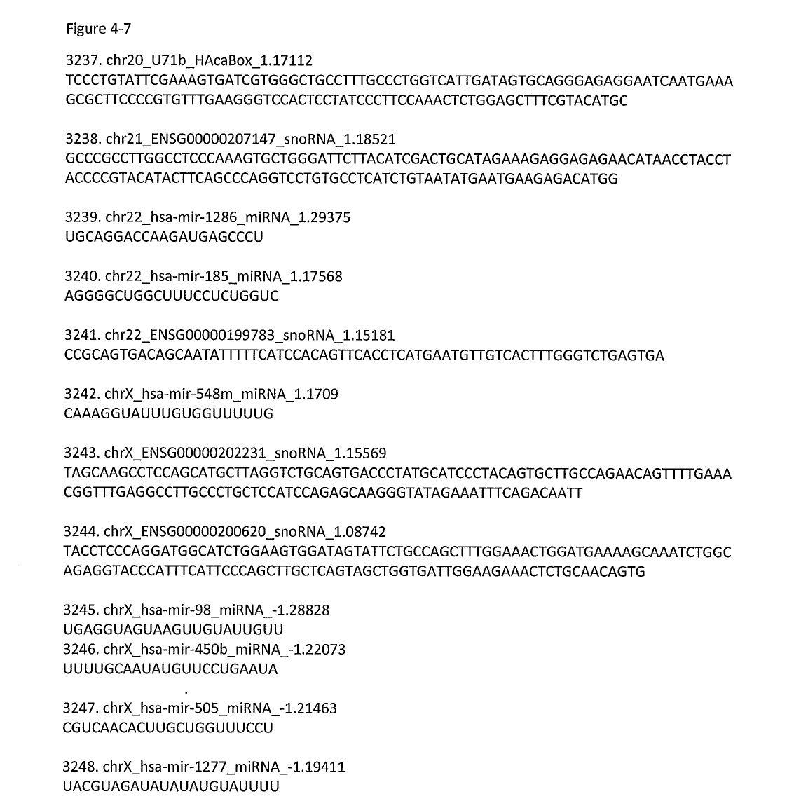

The panel of T21 biomarkers includes a subset encoded by chromosome 21 (SEQ ID NOs:3,028-3,065 and 3,238, see FIG. 2A and 2B). That subset includes polynucleotides found to be up-regulated in a pregnant mother carrying a fetus that is T21 when compared to a pregnant mother carrying a normal fetus. That subset also includes polynucleotides found to be down-regulated in a pregnant mother carrying a fetus that is T21 when compared to a pregnant mother carrying a normal fetus. The panel of biomarkers includes a subset encoded by chromosomes other than chromosome 21, e.g., chromosomes 1-20, 22, and/or x (SEQ ID NOs:8-3,027, 3,066-3,227 and 3,239-3,248, see FIG. 2A and 2B). That subset includes polynucleotides found to be up-regulated in a pregnant mother carrying a fetus that is T21 when compared to a pregnant mother carrying a normal fetus. That subset also includes polynucleotides found to be down-regulated in a pregnant mother carrying a fetus that is T21 when compared to a pregnant mother carrying a normal fetus. The panel of T21 biomarkers includes a subset that are mRNAs (SEQ ID NO:8-3,250) and a subset that are small non-coding RNAs (SEQ ID NO:3,251-3,248). An expression level of a T21 biomarker may include polynucleotide expression level information for one polynucleotide chosen from SEQ ID NO:8-3,248, obtained from a biological sample from a subject. A sample expression profile may include polynucleotide expression level information for two or more polynucleotides chosen from SEQ ID NO:8-3,248, obtained from a biological sample from a subject, for instance, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30. A sample expression profile may include polynucleotide expression level information for no greater than 30 polynucleotides chosen from SEQ ID NO:8-3,248, obtained from a biological sample from a subject, for instance, no greater than 30, no greater than 29, no greater than 28, no greater than 27, no greater than 26, no greater than 25, no greater than 24, no greater than 23, no greater than 22, no greater than 21, no greater than 20, no greater than 19, no greater than 18, no greater than 17, no greater than 16, no greater than 15, no greater than 14, no greater than 13, no greater than 12, no greater than 11, no greater than 10, no greater than 9, no greater than 8, no greater than 7, no greater than 6, or no greater than 5.

The skilled person will recognize that detecting a T21 biomarker present in a subject may not require use of an entire nucleotide sequence disclosed at any of SEQ ID NO:8-3,248. A nucleotide sequence used in a method provided herein is of a length that is at least substantially unique for a T21 biomarker to specifically hybridize with a RNA, such as a CFP RNA, present in a biological sample. A nucleotide sequence used in a method provided herein may be RNA, DNA, or RNA/DNA hybrid.

In one embodiment, a T21 biomarker present in a biological sample may be a polynucleotide that contains or consists of the sequence which defines the T21 biomarker target or complement thereof. The T21 biomarker may be identical to one of SEQ ID NOs:8-3,248, or can be a complement thereof, sense or antisense, as well as a sequence that hybridizes therewith under suitable conditions. In one embodiment, a T21 biomarker used to detect a RNA present in a biological sample, such as a CFP RNA, may be at least 15, at least 20, at least 25, at least 30, at least 35, or at least 40 nucleotides in length, and so on, of a sequence selected from SEQ ID NO:8-3,248, or the complement thereof. In one embodiment, a T21 biomarker may include a sequence selected from SEQ ID NO:8-3,248, or the complement thereof, that is from 15 nucleotides to the full sequence, from 16 nucleotides to 100 nucleotides, from 17 nucleotides to 50 nucleotides, from 18 nucleotides to 30 nucleotides, from 19 nucleotides to 25 nucleotides, or from 20 to 22 nucleotides. A T21 biomarker selected from SEQ ID NO:8-3,248 may have perfect identity, at least 95% identity, at least 90% identity, at least 85% identity, or at least 80% identity with a sequence disclosed herein. A T21 biomarker selected from SEQ ID NO:8-3,248 may have perfect complementarity or at least 95% complementarity, at least 90% complementarity, at least 85% complementarity, or at least 80% complementarity with a sequence disclosed herein. A T21 biomarker may be continuous or it can have one or more bulges or mismatches upon hybridization. A T21 biomarker used to detect a RNA in a biological sample may also include one or more chemical modifications, such as a 2′ carbon modification. A T21 biomarker may or may not form an overhang upon hybridization when detecting a RNA present in a biological sample.

“Hybridization” includes any process by which a strand of a nucleic acid sequence joins with a second nucleic acid sequence strand through base-pairing. Hybridization of polynucleotides is affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringency conditions depend on the length and base composition of the nucleic acid, which can be determined by techniques well known in the art. Generally, stringency can be altered or controlled by, for example, manipulating temperature and salt concentration during hybridization and washing. For example, a combination of high temperature and low salt concentration increases stringency. The degree of stringency may be based, for example, on the calculated (estimated) melting temperature (Tₘ) of the polynucleotide. Calculation of Tₘ is well known in the art. For example, “maximum stringency” typically occurs at around Tₘ-5° C. (5° below the Tₘ of the probe); “high stringency” at around 5-10° below the Tₘ; “intermediate stringency” at around 10-20° below the Tₘ of the probe; and “low stringency” at around 20-25° below the Tₘ. Maximum stringency conditions may be used to identify a polynucleotide present in a biological sample having strict identity or near-strict identity with a T21 biomarker selected from SEQ ID NO:8-3,248; while high stringency conditions are used to identify a polynucleotide present in a biological sample having about 80% or more sequence identity with a T21 biomarker. Such conditions are known to those skilled in the art and can be found in, for example, Strauss, W. M. “Hybridization With Radioactive Probes,” in Current Protocols in Molecular Biology 6.3.1-6.3.6, (John Wiley & Sons, N.Y. 2000). Both aqueous and nonaqueous conditions as described in the art can be used.

Expression levels of any one or more of the T21 biomarkers described herein may be used to determine the presence, absence, or type of T21 in a subject. In one embodiment, expression levels of one or more T21 biomarkers encoded by chromosome 21 may be used to determine the presence, absence, or type of T21 in a subject. In one embodiment, expression levels of one or more T21 biomarkers encoded by the remaining 21 autosomes (chromosomes 1-22 exclusive of chromosome 21) and X may be used to determine the presence, absence, or type of T21 in a subject. In one embodiment, expression levels of one or more T21 biomarkers encoded by any combination of chromosomes 1-22 and X may be used to determine the presence, absence, or type of T21 in a subject. In one embodiment, expression levels of one or more T21 biomarkers encoded by one chromosome selected from 1-22 and X may be used to determine the presence, absence, or type of T21 in a subject. In one embodiment, expression levels of one or more T21 biomarkers that are mRNAs (SEQ ID NO:8-3,250) may be used to determine the presence, absence, or type of T21 in a subject. In one embodiment, expression levels of one or more T21 biomarkers that are small non-coding RNAs (SEQ ID NO:3,251-3,248) may be used to determine the presence, absence, or type of T21 in a subject. In one embodiment, the T21 biomarkers used may be those that are up-regulated in a pregnant mother carrying a fetus that is T21 when compared to a pregnant mother carrying a normal fetus. In one embodiment, the T21 biomarkers used may be those that are down-regulated in a in a pregnant mother carrying a fetus that is T21 when compared to a pregnant mother carrying a normal fetus. In one embodiment, the T21 biomarkers used may be a combination of those that are up-regulated and those that are down-regulated in a pregnant mother carrying a fetus that is T21 when compared to a pregnant mother carrying a normal fetus.

The number of T21 biomarkers used in an assay to determine the presence, absence, or type or T21 in a subject may vary. The skilled person will appreciate that, generally, the more biomarkers examined, the more accurate the determination of the presence, absence, or type of T21 in a subject; however, the skilled person will also appreciate that there is a minimum number of biomarkers useful for an accurate diagnosis of T21. In one embodiment, the number of T21 biomarkers evaluated in practicing a method provided herein may be at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30. In one embodiment, the number of T21 biomarkers evaluated in practicing a method provided herein may be no greater than 30, no greater than 29, no greater than 28, no greater than 27, no greater than 26, no greater than 25, no greater than 24, no greater than 23, no greater than 22, no greater than 21, no greater than 20, no greater than 19, no greater than 18, no greater than 17, no greater than 16, no greater than 15, no greater than 14, no greater than 13, no greater than 12, no greater than 11, no greater than 10, no greater than 9, no greater than 8, no greater than 7, no greater than 6, or no greater than 5. In one embodiment, the number of CFP RNAs detected varies depending upon whether the fetus or subject is normal or abnormal.

All the T21 biomarkers measured in a subject having T21 may not show altered expression levels when compared to a healthy subject. A subject may be considered to have T21 when at least 60%, at least 65%, at least 70%, at least 75%, at least 80%, at least 85%, at least 90%, at least 95%, or 100% of the T21 biomarkers in a sample expression profile from the subject's biological sample show altered expression when compared to those T21 biomarkers in a control expression profile from a healthy subject. For instance, in an embodiment where 10 biomarkers are measured in a biological sample from a subject, such as a pregnant mother carrying a fetus with T21, the subject may be considered to have T21 when at least 6 of the biomarkers in a sample expression profile show altered expression when compared to those T21 biomarkers in a control expression profile from a healthy subject. Some of the T21 biomarkers in a subject not having T21 may show altered expression levels when compared to another healthy subject. A subject may be considered not to have T21 when no greater than 40%, no greater than 35%, no greater than 30%, no greater than 25%, no greater than 20%, no greater than 15%, no greater than 10%, no greater than 5%, or none of the T21 biomarkers in a sample expression profile from the subject's biological sample show altered expression when compared to those T21 biomarkers in a control expression profile from another healthy subject. For instance, in an embodiment where 10 biomarkers are measured in a biological sample from a subject, such as a pregnant mother carrying a normal fetus, the subject may be considered to have a normal fetus when no more than 4 of the biomarkers in a sample expression profile show altered expression when compared to the normal range for the population of healthy fetuses.

Whether the expression level or quantity of a biomarker in a subject having T21 is greater than or less than the expression level or quantity of the biomarker in a healthy subject is determined using routine statistical methods by applying accepted confidence levels. The expression level or quantity of a T21 biomarker in a biological sample is considered to be altered if the difference in amount of the biomarker in a test sample is increased or decreased to a statistically significant degree compared to the amount of the biomarker in a control sample. The term “statistically significant” refers to a result, namely a difference in numbers of positive results between a test and a control that is not likely due to chance. The minimum chance level for statistical significance herein is 95% probability that the result is not due to chance, i.e., random variations in the data. A 95% confidence interval means that if the procedure for computing a 95% confidence interval is used over and over, 95% of the time the interval will contain the true parameter value. In one embodiment, the minimum chance level for statistical significance is 97% probability, 99% probability, or 99.9% probability. Various methods, as is known, can be used to calculate statistical significance. Examples include, but are not limited to, binomial probabilities, the Poisson distribution, chi-square, and t-test. The skilled person will recognize that one may use sufficient numbers of results to obtain a confidence interval of at least 95%, or higher, in order to determine statistical significance of a difference in expression level or quantity of a biomarker in a subject having T21 and the expression level or quantity of the biomarker in a healthy subject.

In one embodiment, a subject is considered to have T21 when comparison of expression of at least one T21 biomarker, or a plurality of T21 biomarkers, with the expression level of the at least one T21 biomarker, or a plurality of T21 biomarkers, in a biological sample from a subject not having T21 shows a difference, and that difference is indicative of the presence of T21 in the subject. In one embodiment, a subject is considered to have T21 when expression of at least one T21 biomarker, or a plurality of T21 biomarkers, is altered to a statistically significant degree in a biological sample from the subject compared to a biological sample from a subject not having trisomy 21. In one embodiment, a subject is considered to have T21 when comparison of expression of at least one T21 biomarker with the expression level of the at least one T21 biomarker in a biological sample from a subject not having T21 shows that the expression level or quantity of a biomarker in the subject is outside the 95% confidence interval for the biomarker. In one embodiment, a subject is considered to have T21 when comparison of expression of a plurality of T21 biomarkers with the expression level of the plurality of T21 biomarkers in a biological sample from a subject not having T21 shows that the expression level or quantity of the plurality of biomarker in the subject is outside the 95% confidence interval for the plurality of the biomarkers.

Accordingly, in one embodiment, a method provided herein includes measuring a plurality of T21 biomarkers in a biological sample obtained from a subject, such as a pregnant female. The plurality of T21 biomarkers measured may be selected from any combination of SEQ ID NO:8-3,248, or a complement thereof, or a portion thereof. The plurality of T21 biomarkers measured may be polynucleotides that hybridize to a sequence selected from any one of SEQ ID NO:8-3,248 under suitable conditions. In one embodiment, a method provided herein includes detecting T21 biomarkers in a biological sample to yield an expression level of each detected T21 biomarker. The T21 biomarkers may be selected from any combination of SEQ ID NO:8-3,248, or a complement thereof, or a portion thereof. The T21 biomarkers detected may be polynucleotides that hybridize to a sequence selected from any one of SEQ ID NO:8-3,248 under suitable conditions. The biological sample may include plasma from a pregnant female. In one embodiment, a method disclosed herein includes detecting T21 biomarkers in a biological sample to yield a sample expression profile. The T21 biomarkers may be selected from any combination of SEQ ID NO:8-3,248, or a complement thereof, or a portion thereof. The T21 biomarkers detected may be selected from SEQ ID NO:8-3,248, or a complement thereof, or a portion thereof. The T21 biomarkers detected may be polynucleotides that hybridize to a sequence selected from any one of SEQ ID NO:8-3,248 under suitable conditions. The biological sample may include plasma from a pregnant female.

In one embodiment, such as one where the subject is a pregnant female, a method disclosed herein may include identifying the fetus as i) having trisomy 21 if expression of the plurality of biomarkers is altered to a statistically significant degree in the biological sample compared to a biological sample from a second pregnant female carrying a fetus not having trisomy 21, or ii) not having trisomy 21 if expression of the plurality of biomarkers is not altered to a statistically significant degree in the biological sample compared to a biological sample from a second pregnant female carrying a fetus not having trisomy 21. In one embodiment, such as one where the subject is a pregnant female, the method may further include comparing the expression level of a detected T21 biomarker to the expression level of the T21 biomarker in pregnant females carrying a fetus without T21, wherein an expression level of a detected T21 biomarker that is outside the 95% confidence interval for that T21 biomarker indicates the expression level of the T21 biomarker is altered. In one embodiment, such as one where the subject is a pregnant female, the method may further include comparing the sample expression profile with a reference expression profile; wherein a difference between the sample expression profile and the reference expression profile is indicative of the presence or absence of trisomy 21 in the fetus.

In one embodiment, such as one where the fetus is diagnosed as having T21, a method may further include recommending to the pregnant female a genetic test chosen from amniocentesis, cordocentesis, and chorionic villus sampling, ultrasound, or a combination thereof.

Amounts of T21 biomarkers in a biological sample may be determined in absolute or relative terms. If expressed in relative terms, amounts can be expressed as normalized amounts with reference to one or more normalization sequences present in a biological sample.

It is expected that this method will have a sensitivity (percent of fetuses or subjects having T21 correctly identified, also referred to as detection rate) of at least 98%, at least 99%, or 100% when enough T21 biomarkers present in a biological sample are detected. It is also expected that this method will have a specificity (percent of fetuses or subjects not having T21 correctly identified) of at least 98%, at least 99%, or 100% when enough T21 biomarkers present in a biological sample are detected.

Measuring the expression level or quantity of any single T21 biomarker or a plurality of T21 biomarkers may be accomplished by use of techniques that are known in the art and routine. In one embodiment, the expression level or quantity of a T21 biomarker or a plurality of T21 biomarkers may be monitored directly by detecting RNA present in a biological sample. RNA may be obtained from a biological sample using routine techniques known in the art. In one embodiment, the RNA is cell-free RNA obtained from biological tissue and/or fluid. In one embodiment, the RNA is cell-free plasma RNA obtained from whole blood, blood plasma, or serum. In one embodiment, the RNA is isolated. As used herein, the term “isolated” refers to a polynucleotide that has been removed from its natural environment.

Detecting one or more T21 biomarkers that are present as a RNA polynucleotide may be accomplished by a variety of methods. Some methods are quantitative and allow estimation of the original levels of RNA between the levels present in a test sample and a control, such as a control expression level for a T21 biomarker and/or a control expression profile, whereas other methods are merely qualitative. In one embodiment, a method for detecting one or more T21 biomarkers may include the use of polynucleotides that are in solution, and may be in any format, including, but not limited to, the use of individual tubes or a high throughput device, such as a PCR-card.

Quantitative real-time PCR (QRT-PCR) may be used to measure the differential expression of any T21 biomarker in a test sample and a control. In QRT-PCR, the RNA template is generally reverse transcribed into cDNA, which is then amplified via a PCR reaction. The primers used for amplification may be selected by determining which T21 biomarker(s) described at SEQ ID NO:8-3,248 is to be amplified, and then designing primers using routine methods known in the art. The PCR amplification process is catalyzed by a thermostable DNA polymerase. Non-limiting examples of suitable thermostable DNA polymerases include Taq DNA polymerase, Pfu DNA polymerase, Tli (also known as Vent) DNA polymerase, Tfl DNA polymerase, and Tth DNA polymerase. The PCR process may include three steps (i.e., denaturation, annealing, and extension) or two steps (i.e., denaturation and annealing/extension). The temperature of the annealing or annealing/extension step may vary, depending upon the amplification primers and other parameters such as concentration. The temperature of the annealing or annealing/extending step may range from about 50° C. to about 75° C. The amount of PCR product is followed cycle-by-cycle in real time, which allows for determination of the initial concentrations of mRNA. The reaction may be performed in the presence of a dye that binds to double-stranded DNA, such as SYBR Green. The reaction may also be performed with fluorescent reporter probes, such as TAQMAN probes (Applied Biosystems, Foster City, Calif.) that fluoresce when the quencher is removed during the PCR extension cycle. Fluorescence values are recorded during each cycle and represent the amount of product amplified to that point in the amplification reaction. The cycle when the fluorescent signal is first recorded as statistically significant is the threshold cycle (Ct). To minimize errors and reduce any sample-to-sample variation, QRT-PCR is typically performed using one or more normalization sequences.

Reverse-transcriptase PCR (RT-PCR) may also be used to measure the expression of a T21 biomarker. As described above, the RNA template is reverse transcribed into cDNA, which is then amplified via a typical PCR reaction. After a set number of cycles the amplified DNA products are typically separated by gel electrophoresis. Comparison of the relative amount of PCR product amplified in different samples will reveal whether the expression of a T21 biomarker is altered in a test sample.

Expression of a T21 biomarker may also be measured using a nucleic acid microarray (also referred to in the art as a DNA chip or biochip). In this method, single-stranded polynucleotides selected from at least a portion of SEQ ID NO:8-3,248, or a complement thereof, are plated, or arrayed, on a solid support. The solid support may be a material such as, for instance, glass, silica-based, silicon-based, a synthetic polymer, a biological polymer, a copolymer, a metal, or a membrane. The form or shape of the solid support may vary, depending on the application. Suitable examples include, but are not limited to, slides, strips, plates, wells, microparticles, fibers (such as optical fibers), gels, and combinations thereof. The arrayed immobilized sequences are generally hybridized with specific DNA probes obtained from the test sample. As described above, RNA present in a sample, including T21 biomarkers, is generally reverse transcribed into cDNA. Fluorescently labeled cDNA probes may be generated through incorporation of fluorescently labeled deoxynucleotides during the reverse transcription step. The cDNA probes are hybridized to the immobilized nucleic acids on the solid support under highly stringent conditions. After stringent washing to remove non-specifically bound probes, the solid support is scanned using routine methods, for instance, by confocal laser microscopy or by another detection method, such as a CCD camera. Quantitation of hybridization of each arrayed element allows for assessment of corresponding RNA abundance. With multiple color fluorescence, separately labeled cDNA probes may be hybridized pairwise to the array. The relative abundance of the transcripts from the two sources corresponding to each specified T21 biomarker may then be determined simultaneously. Microarray analysis may be performed by commercially available equipment, following manufacturer's protocols, such as by using the Affymetrix GenChip technology, or Incyte's microarray technology.

Differential expression of a T21 biomarker may also be measured using Northern blotting. For this, RNA samples are first separated by size via electrophoresis in an agarose gel under denaturing conditions. The RNA is then transferred to a membrane, crosslinked, and hybridized, under highly stringent conditions, to a labeled DNA probe. After washing to remove the non-specifically bound probe, the hybridized labeled species are detected using routine techniques known in the art. The probe may be labeled with, for instance, a radioactive element, a chemical that fluoresces when exposed to ultraviolet light, a tag that is detected with an antibody, or an enzyme that catalyses the formation of a colored or a fluorescent product. A comparison of the relative amounts of RNA detected in a control sample and a test sample will reveal whether the expression of one or more T21 biomarkers or changed in the test sample.

Nuclease protection assays may also be used to monitor the altered expression of a T21 biomarker in a test sample and a control. In nuclease protection assays, an antisense probe hybridizes in solution to a RNA sample. The antisense probe may be labeled with an isotope, a fluorophore, an enzyme, or another tag. Following hybridization, nucleases are added to degrade the single-stranded, unhybridized probe and RNA. An acrylamide gel is used to separate the remaining protected double-stranded fragments, which are then detected using techniques well known in the art. Again, qualitative differences in expression may be detected.

In one embodiment, expression of a T21 biomarker may be examined in vivo in a subject. One or more RNA polynucleotides may be labeled with fluorescent dye, a bioluminescent marker, a fluorescent semiconductor nanocrystal, or a short-lived radioisotope, and then the subject may be imaged or scanned using a variety of techniques, depending upon the type of label.

In one embodiment, the detection of a RNA, such as a CFP RNA, uses the nucleotides of a specific exon as described in SEQ ID NO:8-3,248. Thus, if QRT-PCR is used to detect a specific CFP RNA, the primers used to amplify the CFP RNA will amplify all or a portion of an exon described in SEQ ID NO:8-3,248. If a microarray is used to detect a specific CFP RNA, the arrayed immobilized sequence used to detect the CFP RNA will be based on all or a portion of an exon described in SEQ ID NO:8-3,248, or a complement thereof. A person skilled in the art will know which parameters may be manipulated to optimize detection of a RNA of interest using one or more of the polynucleotides listed at SEQ ID NO:8-3,248.

When determining whether the expression of a T21 biomarker or a plurality of T21 biomarkers are altered in a test sample compared to a control expression level or a control expression profile, it can be helpful to use a normalization sequence. A normalization sequence is a polynucleotide that can be used to normalize the relative amounts of polynucleotides, and/or data obtained from the polynucleotides, from one sample to the next. A normalization sequence can be RNA that has an expression level or quantity that is generally stable under the conditions studied. That is, the normalization sequence can have an expression level or quantity that is substantially unaffected by physiological circumstances present in a subject, and thus the normalization sequence can be used to normalize the amount of polynucleotides in separate samples for comparison. The separate samples can be from different subjects or the same subject at different time points, such as different time points in pregnancy. For example, the normalization sequence can be used to normalize the amount of RNA in QRT-PCR studies, such as by normalizing the amount of a RNA sequence of interest. The normalization sequences described herein can be used alone or in combination, and may be used to normalize samples to be assayed for T21 biomarkers. Thus, the normalization sequences provided herein can be for quantification of cell-free RNA, including CFP RNA, present in a biological sample.

It has been determined that previously reported normalization sequences utilized in other tissues for quantification of isolated RNA (e.g., mRNA: 18s RNA, RPLP0, and GAPDH; miRNA: miR-103, miR-146a, and miR-197) were either expressed inconsistently in control plasma samples or were altered by either pregnancy, gestational age or disease (see Dong and Weiner, WO 12/075150). The normalization sequences described can include cell-free plasma RNA sequences (including coding sequences, e.g., mRNA, and/or non-coding sequences, e.g., miRNA) that are substantially unchanged by a condition. In one embodiment, the normalization sequences are substantially unchanged during the course of pregnancy.

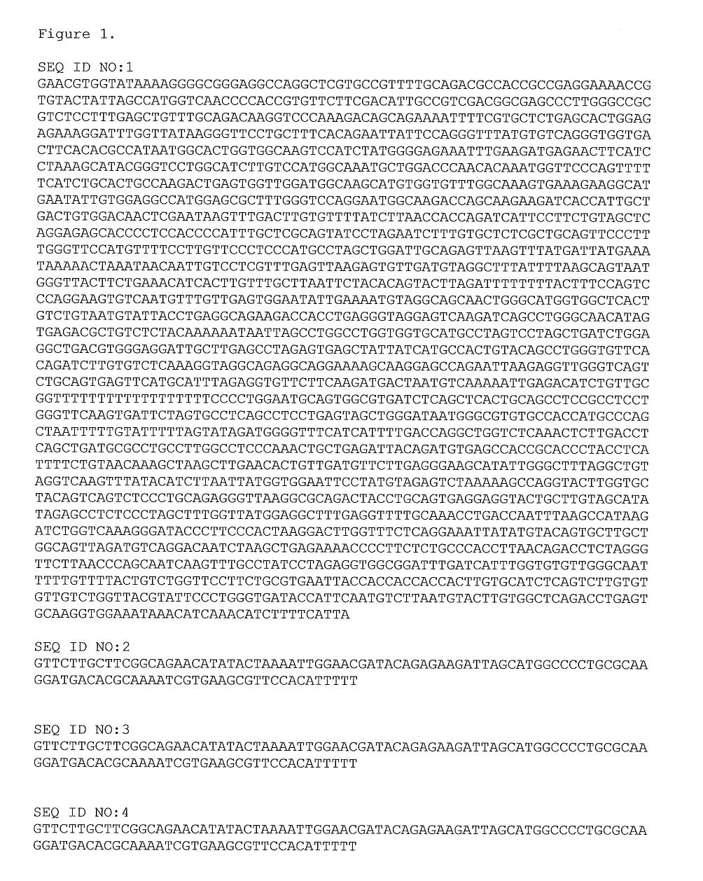

Normalization sequences appropriate for use in the methods provided herein may be identified as described in Dong and Weiner (WO 12/075150). In one embodiment, the normalization sequence includes a circulating RNA. Such a normalization sequence can be described as human (i.e., Homo sapiens) peptidylprolyl isomerase A (i.e., cyclophilin A, rotmase A), which is encoded by a PPIA coding region. The normalization sequence can be an mRNA for peptidylprolyl isomerase. An example of a peptidylprolyl isomerase normalization sequence can be found at accession number: NM_{—}021130 and/or NM_{—}001008741. An example of a peptidylprolyl isomerase normalization sequence that may be useful for normalization of mRNA is depicted at SEQ ID NO: 1 (see FIG. 1). In one embodiment, the normalization sequence may include miRNA. Such a normalization sequence may be a Drosophila melanogaster small nuclear RNA, such as snRNA:U6. The snRNA:U6 normalization sequence can be snRNA:U6 at 96Aa, 96:Ab, and/or 96Ac. Examples of these normalization sequences include snRNA:U6:96Aa (SEQ ID NO: 2 for miRNA), snRNA:U6:96Ab (SEQ ID NO: 3 for miRNA), and/or snRNA:U6:96Ac (SEQ ID NO: 4 for miRNA) (see FIG. 1), and can be found at the following accession numbers, respectively: NR_{—}002081 (snRNA:U6:96Aa); NR_{—}002082 (snRNA:U6:96Ab); and NR_{—}002083 (snRNA:U6:96Ac). Accordingly, SEQ ID NO:1 may be used for normalization of mRNA, and SEQ ID NOs: 2-4 may be used for normalization of miRNA. More than one normalization sequence may be used.

Primers and probes for these sequences can be readily obtained by one of ordinary skill in the art. For example, sequences for the forward primer, reverse primer, and probe for

SEQ ID NO:1 (e.g., for an mRNA normalization sequence) may be: Forward primer: GCTTTGGGTCCAGGAATGG (SEQ ID NO:5); Reverse primer: GTTGTCCACAGTCAGCAATGGT (SEQ ID NO:6); and Probe: AGACCAGCAAGAAGAT (SEQ ID NO:7). These polynucleotides may also be used as normalization sequences in the methods provided herein.

In one embodiment, a normalization sequence may be a polynucleotide that contains or consists of the sequence. The normalization sequence can be identical to one of SEQ ID NO:1-7, or can be a complement thereof, sense or antisense, as well as a sequence that hybridizes therewith under suitable conditions. In one embodiment, a normalization sequence may include a sequence selected from SEQ ID NO:1-7, or the complement thereof, that is at least 15 nucleotides, at least 20 nucleotides, at least 25 nucleotides, at least 30 nucleotides, at least 35 nucleotides, at least 40 nucleotides, at least 45 nucleotides, at least 50 nucleotides, or at least 55 nucleotides, to the full sequence. In one embodiment, the normalization sequence can include a sequence of SEQ ID NO:1, 2, 3, 4, 5, 6, or 7. A normalization sequence may have perfect identity, at least 95% identity, at least 90% identity, at least 85% identity, or at least 80% identity with a sequence selected from SEQ ID NO:1-7. A normalization sequence may have perfect complementarity or at least 95% complementarity, at least 90% complementarity, at least 85% complementarity, or at least 80% complementarity with a sequence selected from SEQ ID NO:1-7. A normalization sequence may be continuous or it can have one or more bulges or mismatches upon hybridization. A normalization sequence may also include one or more chemical modifications, such as a 2′ carbon modification. A normalization sequence may or may not form an overhang upon hybridization when detecting a RNA present in a biological sample.

Provided herein is an article that includes a substrate and a plurality of individual polynucleotides. The individual polynucleotides may be selected from SEQ ID NO:8-3,248, or a complement thereof, or a portion thereof. The polynucleotides are immobilized onto a surface of the substrate. In one embodiment, the polynucleotides are immobilized on the substrate surface to form a microarray.

Provided herein are kits. A kit may include one or more polynucleotides for measuring the expression of at least one T21 biomarker, wherein alteration in the expression of the one or more T21 biomarkers in a subject relative to a control is indicative of the presence, absence, or type of T21. A kit may include one or more polynucleotides that are specific to a selected T21 biomarker

A polynucleotide present in a kit may have a sequence that is identical to a polynucleotide listed at SEQ ID NO:8-3,248, or the complement thereof In one embodiment, polynucleotide present in a kit may have a portion of a sequence that is identical to a polynucleotide listed at SEQ ID NO:8-3,248, or the complement thereof The polynucleotides to be used in the measurement of the expression of one or more T21 biomarkers can, depending upon the type of technique to be used. For example, the kit may include polynucleotides useful as primers for QRT-PCR. Polynucleotides useful as probes may be included in a kit and are optionally provided together with a solid substrate, such as but not limited to a bead, a chip, a plate, and a microarray. Polynucleotides may be immobilized on the surface of such a substrate. A kit may also further include a reverse transcriptase, a thermostable DNA polymerase , appropriate buffers and salts, or the combination thereof

Additional reagents useful in the methods described herein, for example determining the presence, absence, or type of T21 in a subject, may be provided in a kit. Depending on the technique or procedure, the kit may further include one or more additional reagents such as, but not limited to, buffers such as amplification buffers, hybridization buffers, labeling buffers, or any equivalent reagent. Reagents may be supplied in solid (e.g., lyophilized) or liquid form, and these may optionally be provided in individual packages using containers such as vials, packets, bottles and the like, for each individual reagent. Each component can for example be provided in an amount appropriate for direct use or may be provided in a reduced or concentrated form that can be reconstituted.

A kit may further include materials and tools useful for carrying out methods described herein. A kit can be used for example in diagnostic laboratories, clinical settings, or research settings. The kit may further include instructions for use, including for example any procedural protocols and instructions for using the various reagents in the kit for performing different steps of the process. Instructions for using the kit according to one or more methods of the invention may include instructions for processing a biological sample obtained from a subject and/or for performing the test, and instructions for analyzing or interpreting the results. Instructions may be provided in printed form or stored on any computer readable medium including but not limited to DVDs, CDs, hard disk drives, magnetic tape and servers capable of communicating over computer networks. A kit may further include one or more normalization sequences.

It will be understood that generally, components of a kit are conveniently packaged or bound together for ease of handling in commercial distribution and sale.

The present invention is illustrated by the following examples. It is to be understood that the particular examples, materials, amounts, and procedures are to be interpreted broadly in accordance with the scope and spirit of the invention as set forth herein.

### EXAMPLE 1

### T21 Cell Free Plasma Biomarker Confirmation Protocol

Presented here is an example of an assay that was used to confirm the microarray biomarker identification for those biomarkers identified as altered in pregnant mothers carrying a fetus that has trisomy 21. More than 1 million exon clusters and 1769 non-coding small RNAs were screened by Affymetrix GeneChip Human Exon ST and Affymetrix

GeneChip miRNA Arrays, respectively. A total of 3143 mRNA biomarkers (38 encoded by chromosome 21, 3105 encoded by a chromosome other than 21) and 98 non-coding small RNAs (miRNA and snoRNA) were identified as informative. Fifteen of these markers (10 from a gene on chromosome 21 and 5 from one of the other autosomes) were selected for confirmation based on their predictive profiles. These data confirm that the microarray analysis functioned as designed and identified biomarkers that were informative of the trisomy 21 status of a fetus.

Blood Plasma Cell Free RNA isolation. A blood sample was taken from pregnant mothers at approximately 12-16 weeks of pregnancy. The blood sample was spun 200×g for 5 minutes, at 4° C., to separate the cells from plasma. Plasma was transferred to eight tubes (250 microliters (ul) per tube). TRIzol LS (Invitrogen, Grand Island, N.Y., Cat. no 10296-010) (750 ul) was added to each tube, shaken vigorously, and incubated for 5 minutes at room temperature. Two hundred microliters of chloroform were added to each tube, shaken vigorously, and incubated for 10 minutes at room temperature. The tubes were centrifuged at 10,000×g for 15 minutes at 4° C. The resulting upper phase contained RNA, and the lower phase contained DNA and protein.

The upper aqueous phase (300 ul/tube) was transferred to a new tube. Thirty microliters of 3M Sodium acetate (Ambion, Grand Island, N.Y., cat. No. AM9740), p11:5.5, and 3 volumes of 100% ice cold EtOH (Sigma, St. Louis, Mo., cat. no. E702) (900 ul) were added and the tubes incubated at −20° C., overnight. After centrifugation at 12,000×g for 75 minutes (4° C.), all liquid was carefully removed. The pellet was washed with 50 ul per tube with ice cold 80% EtOH, and all the tubes from one patient were combined into one tube. This tube was centrifuged for 60 minutes at 12,000×g at 4° C. All liquid was removed, and the tube was incubated at 37° C. for 40 minutes.

The pellet was suspended in 20 to 40 ul of Diethylpyrocarbonate-(Sigma, cat. no. D5758) (DEPC) treated water, incubated at 56° C. for 10 minutes to dissolve the RNA, then transferred to ice for 30 minutes.

RNA concentration. RNA concentration was measured by using a Qubit® 2.0 Fluorometer (Life Technologies, Grand Island, N.Y.) as recommended by the manufacturer. Briefly, calibration of the Qubit® 2.0 Fluorometer was done using Standard #1 and #2. Working solution was prepared by diluting the Qubit™ RNA reagent at 1:200 in Qubit™ RNA buffer. Working solution (190 ul) and 10 ul of standard or RNA sample were mixed, then incubate at room temperature for 2 minutes. The RNA concentration was determined.

Reverse Transcription. Reverse transcription (RT) of the RNA was prepared using a SuperScript® VILO cDNA synthesis kit (Life Technologies, cat. no. 11755050) and ERCC RNA Spike-in control mixes (Life Technologies, cat. no. 11755050) diluted 1:100. Each RT reaction contained SuperScript® VILO™ MasterMix (4 ul), ERCC RNA Spike-in control (2 ul), 1,000 nanograms (ng) RNA, and H₂O to a final volume of 20 ul. The RT reaction was run using the following conditions: step 1 was 25° C., 10 minutes; step 2 was 42° C., 60 minutes; and step 3 was 85° C., 5 minutes. After the cDNA was produced the tubes were held at 4 until transfer to −20° C.

Preamplification. Preamplification of cDNAs to expand the signal were prepared by mixing a preamplification reaction in a 0.2 ml PCR tube as follows: cDNA, 100 ng; PreAmp Mix (ABI, cat. no. 4391128), 15 ul; probes, final concentration of 100 nM; H₂O to a final volume of 30 ul. The preamplification reaction was run using the following conditions: the reaction was held at 95° C. for 10 minutes, and then cycled through 14 cycles of 95° C. for 15 seconds followed by 60° C. for 15 seconds. The reaction was then held at 99.9° C. for 10 minutes, transferred to 4° C., and then stored at −20 ° C.

Real-time PCR: PCR reactions were prepared by mixing 10 ul of a preamplified reaction, 45 ul of H₂O, and 55 ul of TaqMan Universal PCR Master Mix (ABI, cat. no. 4440040). The thermal cycling reaction was run using the following conditions: the reaction was held at 50° C. for 2 minutes, held at 95° C. for 10 minutes, and then cycled through 40 cycles of 95° C. for 15 seconds followed by 60° C. for 1 minute.

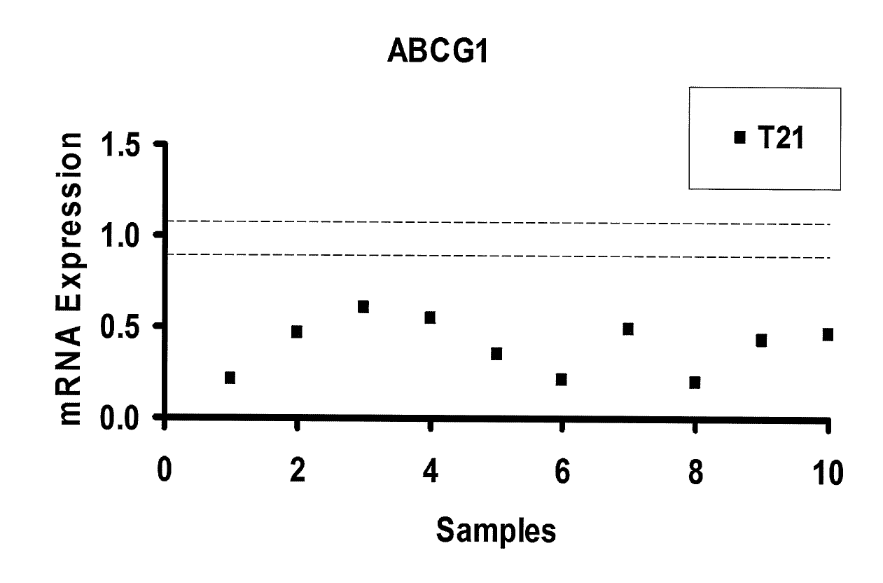

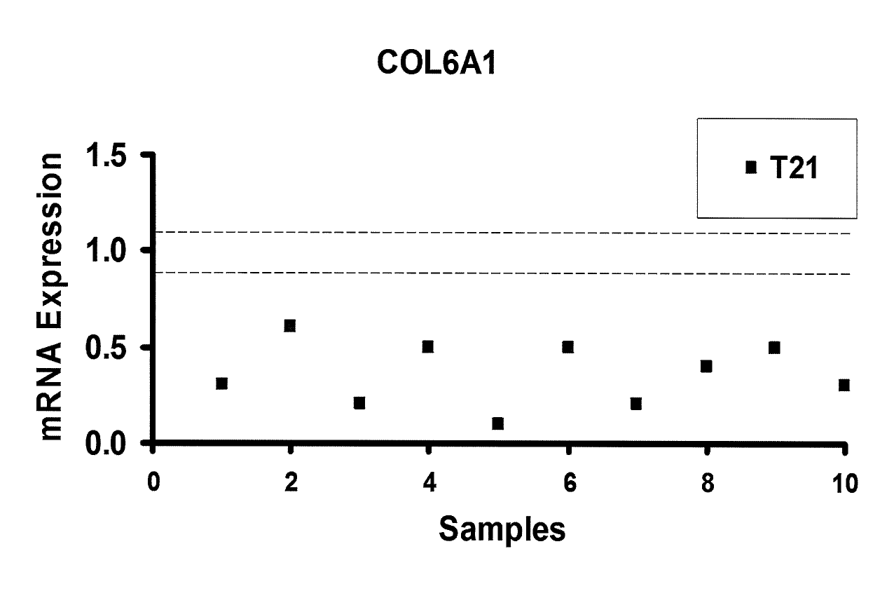

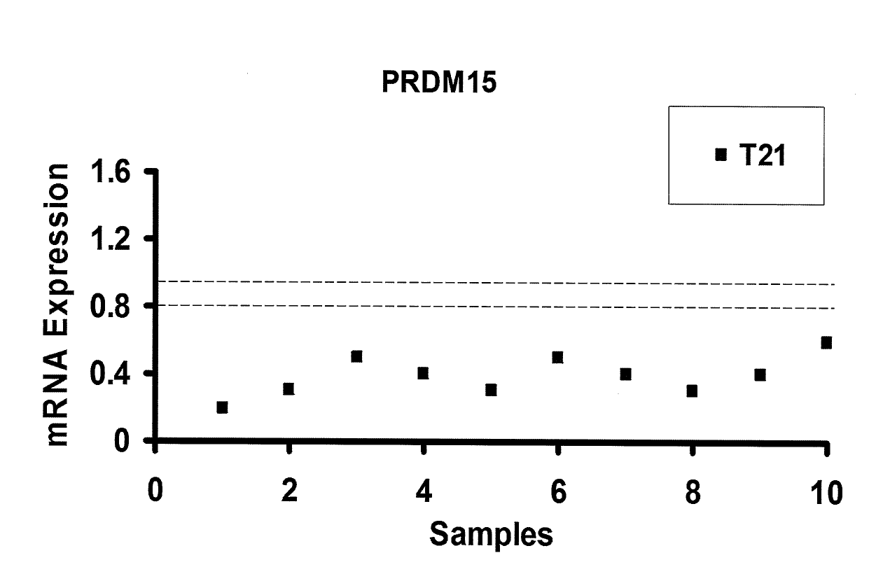

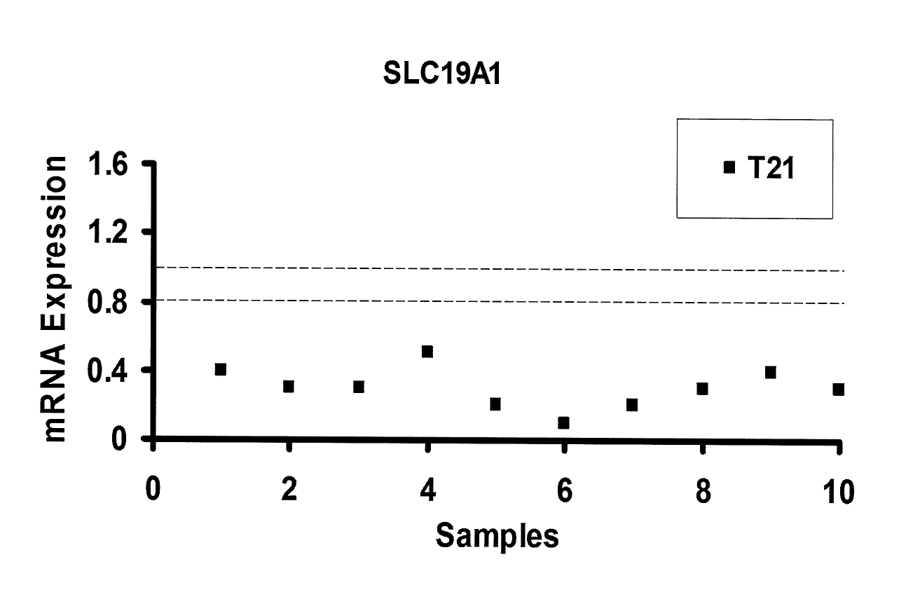

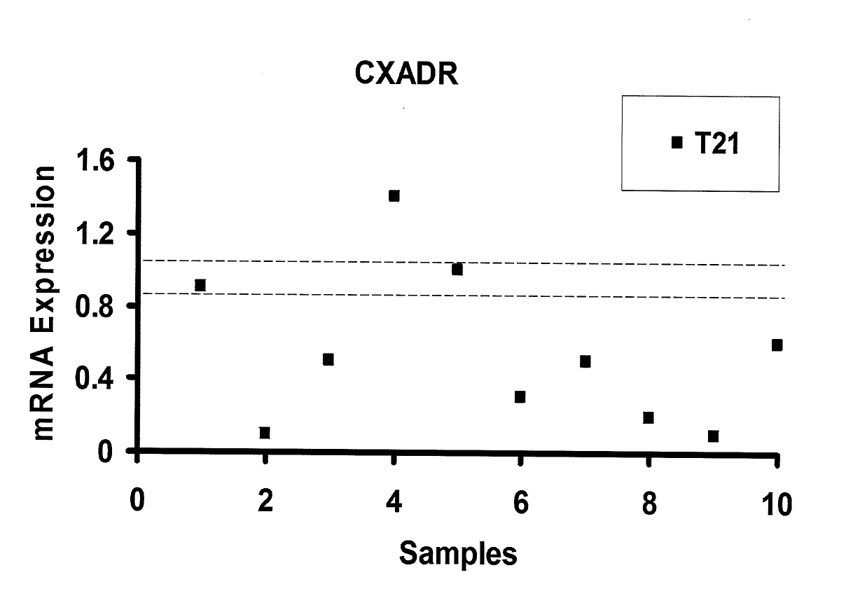

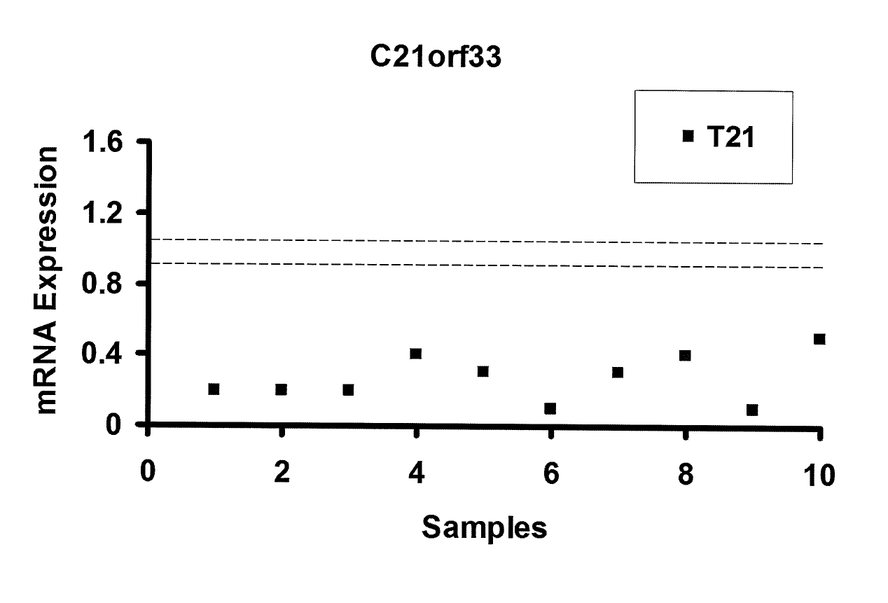

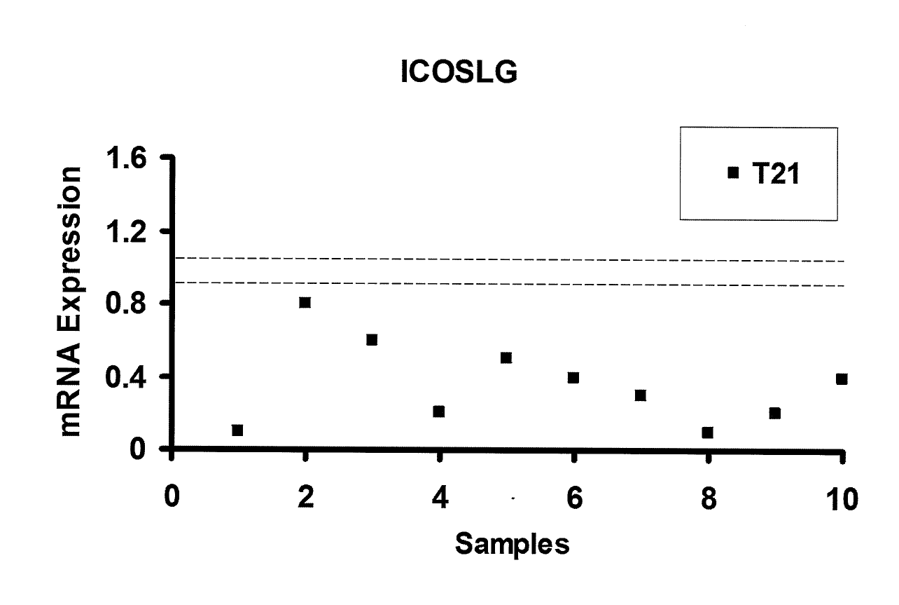

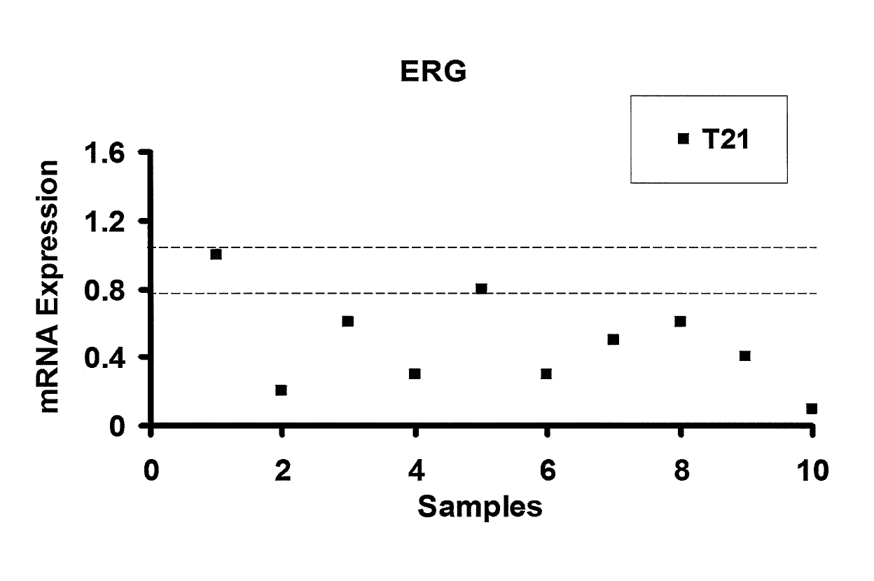

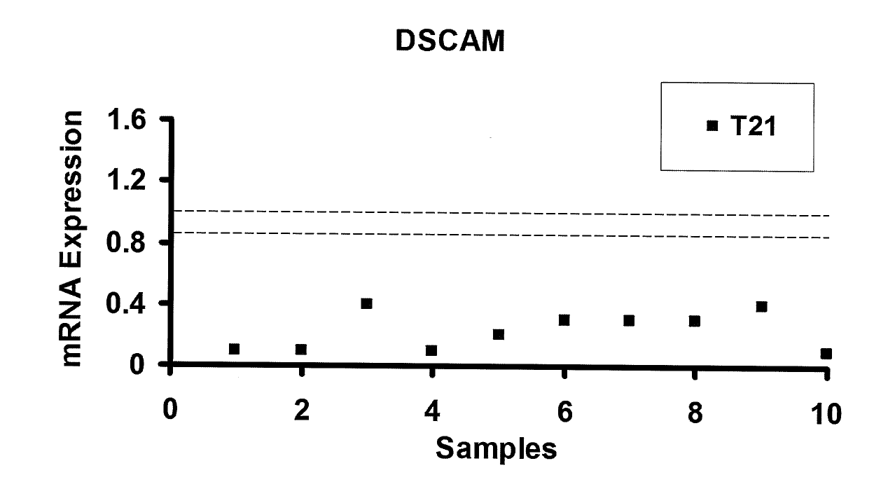

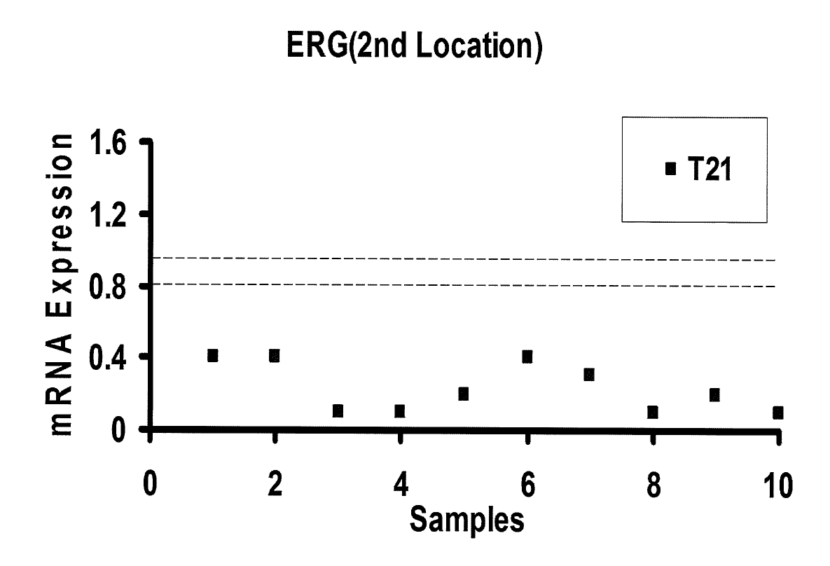

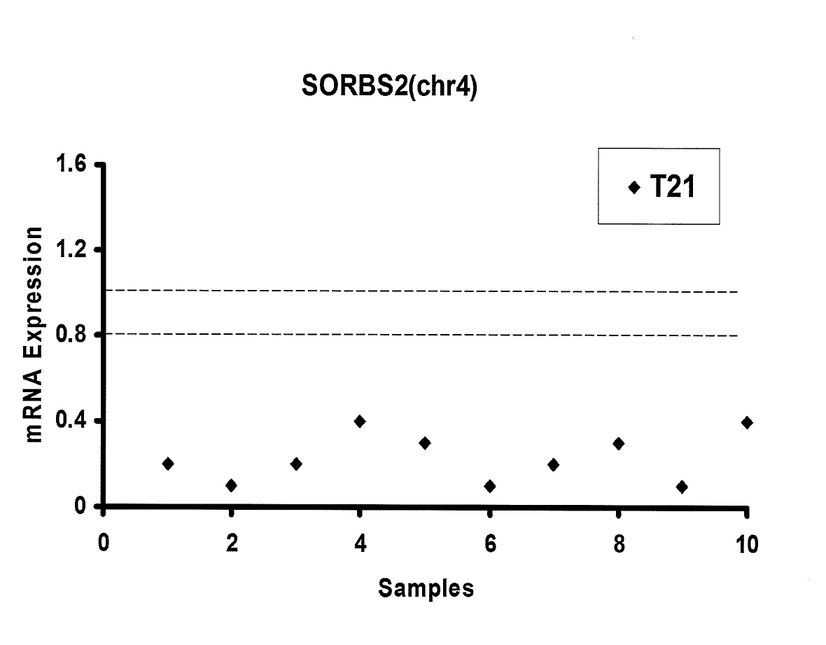

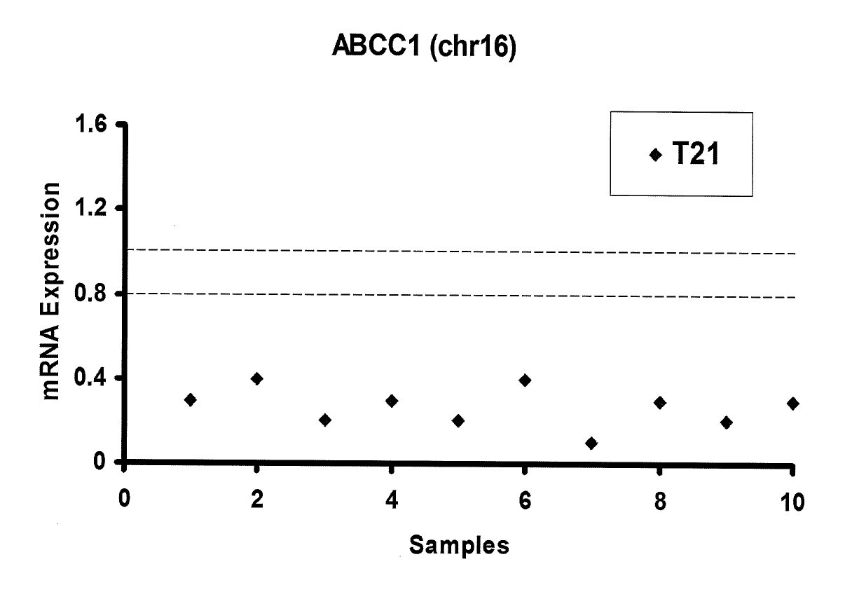

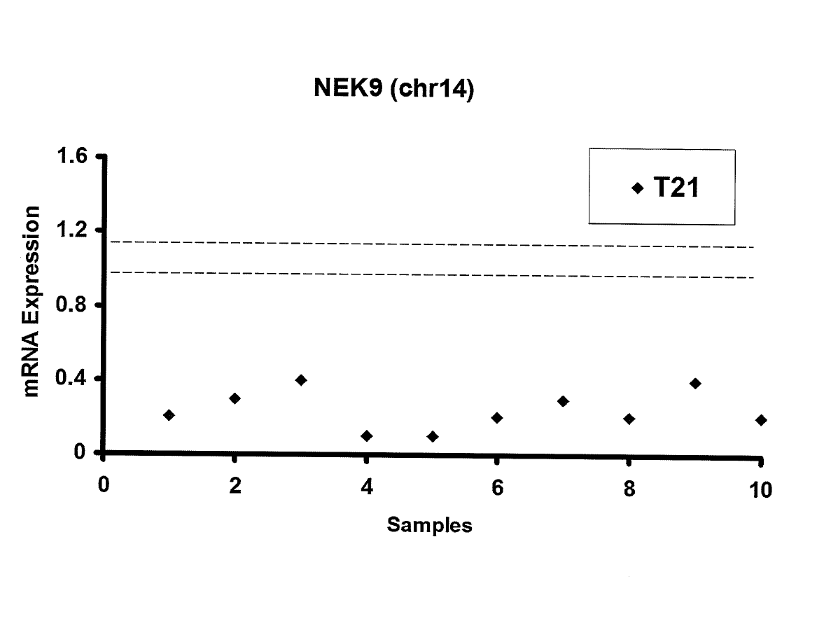

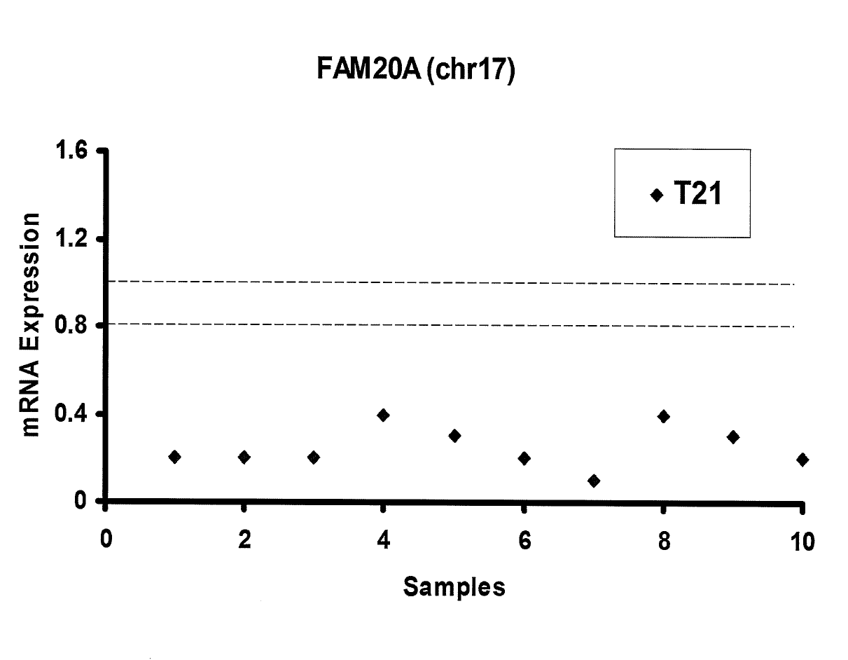

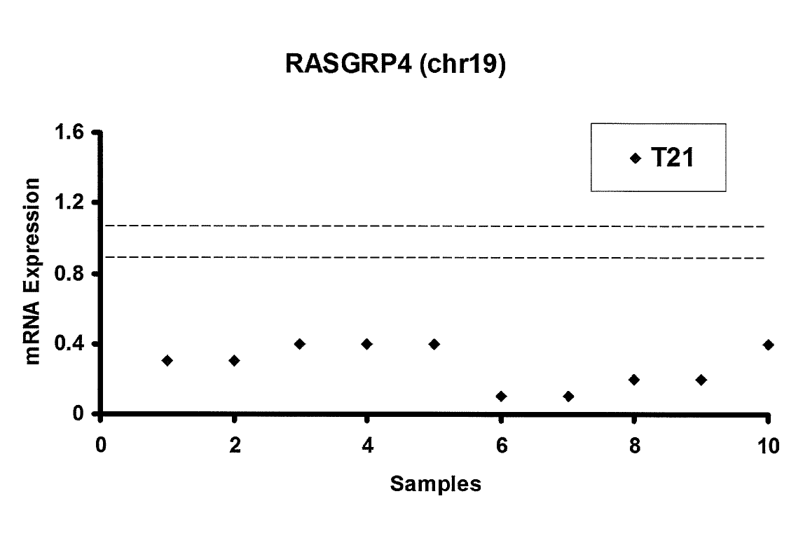

Data analysis. For the initial analysis, the 95% confidence interval for expression (normalized to the described normalization sequences) of each of the fifteen (15) selected T21 markers at 12 weeks gestation was calculated (FIG. 5, area between the dotted lines of each graph). Expression levels of the fifteen T21 biomarkers were then measured and plotted (squares) against the normal range for 10 affected pregnancies at 12 weeks gestation.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for screening a fetus for trisomy 21 comprising:
measuring a plurality of T21 biomarkers in a biological sample obtained from a first pregnant female, wherein the plurality of T21 biomarkers comprise polynucleotides chosen from SEQ ID NO:8-3,248, or a complement thereof; and
identifying the fetus as i) having trisomy 21 if expression of the plurality of biomarkers is altered to a statistically significant degree in the biological sample compared to a biological sample from a second pregnant female carrying a fetus not having trisomy 21, or ii) not having trisomy 21 if expression of the plurality of biomarkers is not altered to a statistically significant degree in the biological sample compared to a biological sample from a second pregnant female carrying a fetus not having trisomy 21.

2. The method of claim 1 wherein the plurality of T21 biomarkers comprises at least 10 polynucleotides chosen from SEQ ID NO:8-3,248, or a complement thereof, and wherein the first pregnant mother having at least 6 biomarkers altered to a statistically significant degree is identified as carrying a fetus having trisomy 21.

3. The method of claim 1 wherein the plurality of T21 biomarkers comprises at least 10 polynucleotides chosen from SEQ ID NO:8-3,248, or a complement thereof, and wherein the first pregnant mother having no greater than 4 biomarkers altered to a statistically significant degree is identified as carrying a fetus not having trisomy 21.

4. The method of claim 1 wherein the T21 biomarkers are selected from polynucleotides encoded by chromosome 21.

5. The method of claim 1 wherein the T21 biomarkers are selected from polynucleotides encoded by any of chromosomes 1-20, 22 or X.

6. The method of claim 1 wherein the T21 biomarkers are selected from polynucleotides encoded by any of chromosomes 1-20, 22 or X.

7. The method of claim 1 wherein the T21 biomarkers are selected from polynucleotides that are up-regulated in the first pregnant female carrying a fetus with trisomy 21 compared to the second pregnant female carrying a fetus not having trisomy 21.

8. The method of claim 1 wherein the T21 biomarkers are selected from polynucleotides that are down-regulated in the first pregnant female carrying a fetus with trisomy 21 compared to the second pregnant female carrying a fetus not having trisomy 21.

9. The method of claim 1 wherein expression of a T21 biomarker is altered to a statistically significant degree if it is outside the 95% confidence interval for that T21 biomarker.

10. The method of claim 1 wherein the method further comprises obtaining the biological sample from the first pregnant female.

11. The method of claim 1 wherein the obtaining comprises obtaining a blood sample.

12. The method of claim 11 wherein the blood sample is processed to remove cells from the blood sample.

13. The method of claim 11 wherein the blood sample is processed to isolate cell free plasma RNA.

14. The method of claim 1 wherein the method further comprises converting RNA polynucleotides present in the biological sample into cDNA molecules.

15. The method of claim 14 wherein the measuring comprises hybridization between a cDNA molecule and a complementary T21 biomarker.

16. The method of claim 15 wherein the complementary T21 biomarker is in solution during the hybridization.

17. The method of claim 15 wherein the complementary T21 biomarker is immobilized on a solid support.

18. The method of claim 1 wherein the fetus is identified as having trisomy 21, the method further comprising recommending a genetic test chosen from amniocentesis, cordocentesis, and chorionic villus sampling, ultrasound, or a combination thereof.

19. The method of claim 1 wherein the fetus of the first pregnant female is between 8 weeks and 16 weeks post-implantation.

20. A method for detecting trisomy 21 in a fetus comprising:
detecting T21 biomarkers in a biological sample to yield an expression level of each detected T21 biomarker, wherein the biological sample comprises plasma from a pregnant female, wherein the T21 biomarkers are selected from SEQ ID NO:8-3,248, or a complement thereof; and
comparing the expression level of a detected T21 biomarker to the expression level of the T21 biomarker in pregnant females carrying a fetus without T21, wherein an expression level of a detected T21 biomarker that is outside the 95% confidence interval for that T21 biomarker indicates the expression level of the T21 biomarker is altered.

21. The method of claim 20 wherein at least 10 T21 biomarkers are detected.

22. The method of claim 21 wherein a fetus carried by the pregnant female is identified as carrying a fetus having T21 when at least 6 biomarkers are outside the 95% confidence interval.

23. The method of claim 20 wherein the T21 biomarkers are selected from polynucleotides encoded by chromosome 21.

24. The method of claim 20 wherein the T21 biomarkers are selected from polynucleotides encoded by any of chromosomes 1-20, 22, or X.

25. The method of claim 20 wherein the T21 biomarkers are selected from polynucleotides encoded by any of chromosomes 1-20, 22, or X.

26. The method of claim 20 wherein the T21 biomarkers are selected from polynucleotides that are up-regulated in the pregnant female carrying a fetus with trisomy 21 compared to the pregnant females carrying a fetus not having trisomy 21.

27. The method of claim 20 wherein the T21 biomarkers are selected from polynucleotides that are down-regulated in the first pregnant female carrying a fetus with trisomy 21 compared to the pregnant females carrying a fetus not having trisomy 21.

28. The method of claim 20 wherein expression of a T21 biomarker is altered to a statistically significant degree if it is outside the 95% confidence interval for that T21 biomarker.

29. The method of claim 20 wherein the method further comprises obtaining the biological sample from the pregnant female.

30. The method of claim 29 wherein the obtaining comprises obtaining a blood sample.

31. The method of claim 30 wherein the blood sample is processed to remove cells from the blood sample.

32. The method of claim 30 wherein the blood sample is processed to isolate cell free plasma RNA.

33. The method of claim 20 wherein the method further comprises converting RNA polynucleotides present in the biological sample into cDNA molecules.

34. The method of claim 33 wherein the detecting comprises hybridization between a cDNA molecule and a complementary T21 biomarker.

35. The method of claim 34 wherein the complementary T21 biomarker is in solution during the hybridization.

36. The method of claim 34 wherein the complementary T21 biomarker is immobilized on a solid support.

37. The method of claim 20 further comprising recommending a genetic test chosen from amniocentesis, cordocentesis, and chorionic villus sampling, ultrasound, or a combination thereof.

38. The method of claim 20 wherein the fetus of the pregnant female is between 8 weeks and 16 weeks post-implantation.

39. A method for detecting trisomy 21 in a fetus comprising:
detecting T21 biomarkers in a biological sample from a pregnant female to yield a sample expression profile, wherein the T21 biomarkers are selected from SEQ ID NO:8-3,248, or a complement thereof; and
comparing the sample expression profile with a reference expression profile; wherein a difference between the sample expression profile and the reference expression profile is indicative of the presence or absence of T21 in the fetus.

40. The method of claim 39 herein the reference expression profile is from at least one second pregnant female carrying a fetus without T21, and wherein a difference between the sample expression profile and the reference expression profile is indicative of the presence of T21.

41. The method of claim 39 wherein the reference expression profile is from at least one second pregnant female carrying a fetus with T21, and wherein a difference between the sample expression profile and the reference expression profile is indicative of the absence of T21.

42. The method of claim 39 wherein the sample expression profile comprises at least 10 polynucleotides chosen from SEQ ID NO:8-3,248, or a complement thereof, wherein the difference between the sample expression profile and the reference expression profile is statistically significant

43. The method of claim 39 wherein the T21 biomarkers are selected from polynucleotides encoded by chromosome 21.

44. The method of claim 39 wherein the T21 biomarkers are selected from polynucleotides encoded by any of chromosomes 1-20, 22, or X.

45. The method of claim 39 wherein the T21 biomarkers are selected from polynucleotides encoded by any of chromosomes 1-20, 22, or X.

46. The method of claim 39 wherein the T21 biomarkers are selected from polynucleotides that are up-regulated in the first pregnant female carrying a fetus with T21 compared to the second pregnant female carrying a fetus not having T21.

47. The method of claim 39 wherein the T21 biomarkers are selected from polynucleotides that are down-regulated in the first pregnant female carrying a fetus with T21 compared to the second pregnant female carrying a fetus not having T21.

48. The method of claim 39 wherein the difference between the sample expression profile and the reference expression profile is statistically significant

49. The method of claim 39 wherein the method further comprises obtaining the biological sample from the first pregnant female.

50. The method of claim 49 wherein the obtaining comprises obtaining a blood sample.

51. The method of claim 50 wherein the blood sample is processed to remove cells from the blood sample.

52. The method of claim 50 wherein the blood sample is processed to isolate cell free plasma RNA.

53. The method of claim 39 wherein the method further comprises converting RNA polynucleotides present in the biological sample into cDNA molecules.

54. The method of claim 53 wherein the detecting comprises hybridization between a cDNA molecule and a complementary T21 biomarker.

55. The method of claim 54 wherein the complementary T21 biomarker is in solution during the hybridization.

56. The method of claim 54 wherein the complementary T21 biomarker is immobilized on a solid support.

57. The method of claim 39 wherein the fetus is identified as having trisomy 21, the method further comprising recommending a genetic test chosen from amniocentesis, cordocentesis, and chorionic villus sampling, ultrasound, or a combination thereof.

58. The method of claim 39 wherein the fetus of the first pregnant female is between 8 weeks and 16 weeks post-implantation.

59. An article comprising:
a substrate; and
a plurality of different polynucleotides selected from SEQ ID NO:8-3,248, or a complement thereof, wherein the polynucleotides are immobilized onto a surface of the substrate.

60. The article of claim 59 wherein the polynucleotides are immobilized on the substrate surface to form a microarray.

61. The article of claim 59 wherein at least 10 polynucleotides are immobilized on the substrate surface

62. A kit for diagnosis trisomy 21 in a subject comprising:
an article comprising a substrate and a plurality of polynucleotides selected from SEQ ID NO:8-3,248, or a complement thereof, wherein the polynucleotides are immobilized onto a surface of the substrate; and
packaging materials and instructions for use.

63. The kit of claim 62 wherein the polynucleotides are immobilized on the substrate surface to form a microarray.
